(19) United States
(12) Patent Application Publication
(10) Pub. No.: US 2023/0257477 A1
(43) Pub. Date: Aug. 17, 2023

(54) DUAL CHIMERIC ANTIGEN RECEPTOR T CELLS TARGETING CCD99- AND CLEC12A-EXPRESSING CANCERS

(71) Applicant: H. Lee Moffitt Cancer Center and Research Institute Inc., Tampa, FL (US)

(72) Inventor: Marco L. Davila, Tampa, FL (US)

(21) Appl. No.: 18/003,922

(22) PCT Filed: Jun. 2, 2021
(86) PCT No.: PCT/US2021/035375

§ 371 Date: Dec. 30, 2022

Related U.S. Application Data
(60) Provisional application No. 63/047,573, filed on Jul. 2, 2020.

(51) Int. Cl. C07K 16/28; C12N 5/0783; C07K 14/725; A61P 35/02

(52) U.S. Cl. CPC C07K 16/2896; C12N 5/0636; C07K 16/2851; C07K 14/7051; C07K 16/2827; A61P 35/02; C07K 2317/565; C07K 2317/622

(57) ABSTRACT

Disclosed herein are compositions and methods for targeted treatment of cancers co-expressing CD99 and CLEC12A. In particular, disclosed herein are immune effector cells genetically modified to express at least two chimeric antigen receptor (CAR) polypeptides that can be used with adoptive cell transfer to target cancers co-expressing CD99 and CLEC12A.

### CROSS-REFERENCE TO RELATED APPLICATIONS

This application claims benefit of U.S. Provisional Application No. 63/047,573, filed Jul. 2, 2020, which is hereby incorporated herein by reference in its entirety.

### SEQUENCE LISTING

This application contains a sequence listing filed in electronic form as an ASCII.txt file entitled “320103 2100_Sequence_Listing_ST25” created on May 11, 2021. The content of the sequence listing is incorporated herein in its entirety.

### BACKGROUND

Surgery, radiation therapy, and chemotherapy have been the standard accepted approaches for treatment of cancers including leukemia, solid tumors, and metastases. Immunotherapy (sometimes called biological therapy, biotherapy, or biological response modifier therapy), which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed.

### SUMMARY

Compositions and methods for targeted treatment of cancers co-expressing CD99 and/or CLEC12A. In particular, disclosed herein are immune effector cells genetically modified to express at least two chimeric antigen receptor (CAR) polypeptides that can be used with adoptive cell transfer to target cancers co-expressing CD99 and/or CLEC12A.

The first CAR polypeptide can contain in an ectodomain an antigen binding domain that can bind CD99 on cells (anti-CD99 agent). The second CAR polypeptide can contain in an ectodomain an antigen binding domain that can bind CLEC12A on cells (anti-CLEC12A CD99 agent). In some embodiments, the immune effector cell can be selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, and a regulatory T cell (Treg). In some embodiments, the cell expressing the herein described CAR polypeptides may be a cell that has the ability to differentiate into a cytotoxic T cell such as a pluripotent stem cell and including an induced pluripotent stem cell (iPSC).

The first CAR polypeptide can contain in an ectodomain an anti-CD99 binding agent that can bind CD99-expressing cancer cells. The second CAR polypeptide can contain in an ectodomain an antigen binding domain that can bind CLEC12A-expressing cancer cells.

The anti-CD99 agent or anti-CLEC12A agent is in some embodiments an antibody fragment that specifically binds CD99 or CLEC12A. For example, the antigen binding domain can be a Fab or a single-chain variable fragment (scFv) of an antibody that specifically binds CD99 or CLEC12A. The anti-CD99 agent or anti-CLEC12A agent is in some embodiments an aptamer that specifically binds CD99 or CLEC12A. For example, the anti-CD99 agent or anti-CLEC12A agent can be a peptide aptamer selected from a random sequence pool based on its ability to bind CD99 or CLEC12A. The anti-CD99 agent or anti-CLEC12A agent can also be a natural ligand of CD99 or CLEC12A, or a variant and/or fragment thereof capable of binding CD99 or CLEC12A.

As with other CARs, the disclosed polypeptides can also contain a transmembrane domain and an endodomain capable of activating an immune effector cell. For example, the endodomain can contain a signaling domain and one or more co-stimulatory signaling regions.

In some embodiments, the intracellular signaling domain is a CD3zeta (CD3ζ) signaling domain, or a mutant or variant thereof. In some embodiments, the costimulatory signaling region comprises the cytoplasmic domain of CD28, 4-1 BB, or a combination thereof. In some cases, the costimulatory signaling region contains 1, 2, 3, or 4 cytoplasmic domains of one or more intracellular signaling and/or costimulatory molecules. In some embodiments, the co-stimulatory signaling region contains one or more mutations in the cytoplasmic domains of CD28 and/or 4-1 BB that enhance signaling.

In some embodiments, the CAR polypeptides contain an incomplete endodomain. For example, the CAR polypeptide can contain only an intracellular signaling domain or a co-stimulatory domain, but not both. In these embodiments, the immune effector cell is not activated unless it and a second CAR polypeptide (or endogenous T-cell receptor) that contains the missing domain both bind their respective antigens. Therefore, in some embodiments, the CAR polypeptide contains a CD3 zeta (CD3) signaling domain but does not contain a costimulatory signaling region (CSR). In other embodiments, the CAR polypeptide contains the cytoplasmic domain of CD28, 4-1BB, or a combination thereof, but does not contain a CD3 zeta (CD3ζ) signaling domain (SD).

Also disclosed are isolated nucleic acid sequences encoding the disclosed CAR polypeptides, vectors comprising these isolated nucleic acids, and cells containing these vectors. For example, the cell can be an immune effector cell selected from the group consisting of an alpha-beta T cells, a gamma-delta T cell, a Natural Killer (NK) cells, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, and a pluripotent stem cell capable of differentiating into a cytotoxic T cell.

In some embodiments, the cell exhibits an anti-tumor immunity when the antigen binding domain of the CARs bind to both CD99 and CLEC12A. In some embodiments, the CARs are designed to work only when both CAR that binds their antigen. For example, in these embodiments, the endodomain of the anti-CD99 CAR can contain only an signaling domain (SD) or a co-stimulatory signaling region (CSR), but not both. The anti-CLEC12A CAR provides the missing signal if it is activated. For example, if the anti-CD99 CAR contains an SD but not a CSR, then the immune effector cell containing this CAR is only activated if the anti-CLEC12A CAR containing a CSR binds its respective antigen. Likewise, if the anti-CD99 CAR contains a CSR but not a SD, then the immune effector cell containing this CAR is only activated if the anti-CLEC12A CAR containing an SD binds its respective antigen.

Also disclosed is a method of providing an anti-tumor immunity in a subject with a CD99- and/or CLEC12A-expressing cancer that involves administering to the subject an effective amount of an immune effector cell genetically modified with disclosed CD99-specific and CLEC12A-specific CARs.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 contains a flow cytometry plot showing gate used for live cells in CD99-PE analysis.

FIG. 2 contains flow cytometry plots showing positive (right) and negative (left) controls used for CD99-PE analysis

FIG. 3 contains flow cytometry plots showing hybridomas positive for CD99.

FIG. 4 contains a plot depicting CD99 binding by ELISA absorption for each hybridoma.

FIG. 5 contains flow cytometry plots showing secondary screening of 1H3H7, IH3E9, 4C5E2, 4C5H10, 9G12C9, and 9G12G6.

FIGS. 6A to 6D show cytotoxic activities of anti-CD99 CARs. CHO cells overexpressing CD99 (CHO-CD99) were used as target cells. Gammaretroviruses expressing anti-CD99 CARs were transduced into primary T cells isolated from healthy PBMCs. Transduction efficiency of each CAR was determined by flow cytometric analysis of mCherry expression (FIGS. 6A and 6B). CAR positive cells were added to target cells at effector to target ratios of either 1:1 (FIG. 6C) or 1:5 (FIG. 6D). UT=Untransduced, MFI=median fluorescent intensity.

FIGS. 7A to 7C show cytokine secretion by anti-CD99 CARs. CAR positive cells were co-incubated with CHO-CD99 target cells at an effector to target ratios of 1:1 overnight. Following co-incubation, supernatants were collected and production of the cytokines IFNγ (FIG. 7A), IL-2 (FIG. 7B), and IL-6 (FIG. 7C) was analyzed. UT=Untransduced.

FIGS. 8A to 8I show immunephenotype of anti-CD99 CARs. Healthy T cells isolated from PBMCs were transduced with anti-CD99 CARs. Following 1-week of culturing without antigen stimulation, cells were stained for CD3, CD4, CD8, PD1, CCR7, and CD45RA, and data were collected on a flow cytometer. Transduction efficiency was determined based on mCherry expression (FIGS. 8A and 8B). Live, CAR positive T cells were analyzed for CD4, CD8, and PD1 expression (FIGS. 8C-8H). T cells subsets were also analyzed based on CCR7 and CD45RA expression (FIG. 8I). EFF=effector, EM=effector memory, CM=central memory, N=Naïve.

FIGS. 9A to 9F show CD4 and CD8 immunephenotype of anti-CD99 CARs. CD4 and CD8 T cells were analyzed for expression of PD1 (FIGS. 9A & 9B, 9D & 9E) and for T cells subsets (FIGS. 9C & 9F). EFF=effector, EM=effector memory, CM=central memory, N=Naïve.

FIGS. 10A to 10D shows CHO cells overexpressing CLEC12A (CHO-CLEC12A) were used as target cells. Gammaretroviruses expressing anti-CLEC12A CARs were transduced into primary T cells isolated from healthy PBMCs. Transduction efficiency of each CAR was determined by flow cytometric analysis of mCherry expression (FIGS. 10A and 10B). CAR positive cells were added to target cells at effector to target ratios of either 1:1 (FIG. 10C) or 1:5 (FIG. 10D). UT=Untransduced, MFI=median fluorescent intensity.

FIGS. 11A to 11I show immunephenotype of anti-CLEC12A CARs. Healthy T cells isolated from PBMCs were transduced with anti-CLEC12A CARs. Following 1-week of culturing without antigen stimulation, cells were stained for CD3, CD4, CD8, PD1, CCR7, and CD45RA, and data were collected on a flow cytometer. Transduction efficiency was determined based on mCherry expression (FIGS. 11A and 2B). Live, CAR positive T cells were analyzed for CD4, CD8, and PD1 expression (FIGS. 11C-11H). T cells subsets were also analyzed based on CCR7 and CD45RA expression (FIG. 111). EFF=effector, EM=effector memory, CM=central memory, N=Naïve.

FIGS. 12A to 12F show CD4 and CD8 immunephenotype of anti-CLEC12A CARs. CD4 and CD8 T cells were analyzed for expression of PD1 (FIGS. 12A & 12B, 12D & 12E) and for T cells subsets (FIGS. 12C & 12F). EFF=effector, EM=effector memory, CM=central memory, N=Naïve.

FIGS. 13A and 13B show hematopoietic stem cell compartment safety assay results for AML CAR-T IND candidates.

FIG. 14 illustrate dual-targeted AML CAR-T constructs using proprietary scFvs and multiple costimulatory domains nominated for in vivo experiments.

FIGS. 15A and 15B show dual-targeted AML CAR-T IND candidates show good transduction efficiency and surface expression.

FIGS. 16A to 16C show dual-targeted AML CAR-T IND candidates demonstrate differential retention of central memory phenotype.

FIG. 17 shows three IND candidates for dual-targeted AML CAR demonstrate tumor stasis in AML model.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of chemistry, biology, and the like, which are within the skill of the art.

The following examples are put forth so as to provide those of ordinary skill in the art with a complete disclosure and description of how to perform the methods and use the probes disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is at or near atmospheric. Standard temperature and pressure are defined as 20° C. and 1 atmosphere.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only, and is not intended to be limiting. It is also possible in the present disclosure that steps can be executed in different sequence where this is logically possible.

It must be noted that, as used in the specification and the appended claims, the singular forms “a,” “an,” and “the” include plural referents unless the context clearly dictates otherwise.

Compositions and methods for targeted treatment of cancers co-expressing CD99 and CLEC12A are disclosed herein. In particular, disclosed herein are immune effector cells genetically modified to express at least two chimeric antigen receptor (CAR) polypeptides that can be used with adoptive cell transfer to target cancers co-expressing CD99 and CLEC12A, or expressing either CD99 or CLEC12A.

CARs generally incorporate an antigen recognition domain from the single-chain variable fragments (scFv) of a monoclonal antibody (mAb) with transmembrane signaling motifs involved in lymphocyte activation (Sadelain M, et al. Nat Rev Cancer 2003 3:35-45).

The disclosed CARs are generally made up of three domains: an ectodomain, a transmembrane domain, and an endodomain. The ectodomain comprises the CD99-binding region or the CLEC12A-binding region and is responsible for antigen recognition. It also optionally contains a signal peptide (SP) so that the CAR can be glycosylated and anchored in the cell membrane of the immune effector cell. The transmembrane domain (TD), is as its name suggests, connects the ectodomain to the endodomain and resides within the cell membrane when expressed by a cell. The endodomain is the business end of the CAR that transmits an activation signal to the immune effector cell after antigen recognition. For example, the endodomain can contain a signaling domain (ISD) and a co-stimulatory signaling region (CSR).

A “signaling domain (SD)” generally contains immunoreceptor tyrosine-based activation motifs (ITAMs) that activate a signaling cascade when the ITAM is phosphorylated. The term “co-stimulatory signaling region (CSR)” refers to intracellular signaling domains from costimulatory protein receptors, such as CD28, 41 BB, and ICOS, that are able to enhance T-cell activation by T-cell receptors.

In some embodiments, the endodomain contains an SD or a CSR, but not both. In these embodiments, an immune effector cell containing the disclosed CAR is only activated if another CAR (or a T-cell receptor) containing the missing domain also binds its respective antigen.

In some embodiments, the disclosed CAR is defined by the formula:

SP-CD99-HG-TM-CSR-SD; or

SP-CD99-HG-TM-SD-CSR;

SP-CLEC12A-HG-TM-CSR-SD; or

SP-CLEC12A-HG-TM-SD-CSR;

wherein “SP” represents an optional signal peptide,

wherein “CD99” represents a CD99-binding region,

wherein “CLEC12A” represents a CLEC12A-binding region,

wherein “HG” represents an optional hinge domain,

wherein “TM” represents a transmembrane domain,

wherein “CSR” represents one or more co-stimulatory signaling regions,

wherein “SD” represents a signaling domain, and

wherein “-” represents a peptide bond or linker.

Additional CAR constructs are described, for example, in Fesnak A D, et al. Engineered T cells: the promise and challenges of cancer immunotherapy. Nat Rev Cancer. 2016 Aug. 23; 16(9):566-81, which is incorporated by reference in its entirety for the teaching of these CAR models.

For example, the CAR can be a TRUCK, Universal CAR, Self-driving CAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR T cell is by default unresponsive, or switched ‘off’, until the addition of a small molecule to complete the circuit, enabling full transduction of both signal 1 and signal 2, thereby activating the CAR T cell. Alternatively, T cells may be engineered to express an adaptor-specific receptor with affinity for subsequently administered secondary antibodies directed at target antigen.

Marked CAR T cells express a CAR plus a tumor epitope to which an existing monoclonal antibody agent binds. In the setting of intolerable adverse effects, administration of the monoclonal antibody clears the CAR T cells and alleviates symptoms with no additional off-tumor effects.

A tandem CAR (TanCAR) T cell expresses a single CAR consisting of two linked single-chain variable fragments (scFvs) that have different affinities fused to intracellular co-stimulatory domain(s) and a CD3ζ domain. TanCAR T cell activation is achieved only when target cells co-express both targets.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor.

A safety CAR (sCAR) consists of an extracellular scFv fused to an intracellular inhibitory domain. sCAR T cells co-expressing a standard CAR become activated only when encountering target cells that possess the standard CAR target but lack the sCAR target.

The antigen recognition domain of the disclosed CAR is usually an scFv. There are however many alternatives. An antigen recognition domain from native T-cell receptor (TCR) alpha and beta single chains have been described, as have simple ectodomains (e.g. CD4 ectodomain to recognize HIV infected cells) and more exotic recognition components such as a linked cytokine (which leads to recognition of cells bearing the cytokine receptor). In fact, almost anything that binds a given target with high affinity can be used as an antigen recognition region.

The endodomain is the business end of the CAR that after antigen recognition transmits a signal to the immune effector cell, activating at least one of the normal effector functions of the immune effector cell. Effector function of a T cell, for example, may be cytolytic activity or helper activity including the secretion of cytokines. Therefore, the endodomain may comprise the “intracellular signaling domain” of a T cell receptor (TCR) and optional co-receptors. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal.

Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (Fc gamma RIIa), DAP10, DAP12, CD79a, CD79b, FcγRIγ, FcγRIIIγ, FcεRIβ (FCERIB), and FcεRlγ (FCERIG).

In particular embodiments, the intracellular signaling domain is derived from CD3 zeta (CD3ζ) (TCR zeta, GenBank accno. BAG36664.1). T-cell surface glycoprotein CD3 zeta (CD3ζ) chain, also known as T-cell receptor T3 zeta chain or CD247 (Cluster of Differentiation 247), is a protein that in humans is encoded by the CD247 gene. The intracellular tails of the CD3 molecules contain a single ITAM, which is essential for the signaling capacity of the TCR. The intracellular tail of the ζ chain (CD3ζ) contains 3 ITAMs. In some embodiments, the CD3ζ chain employed in the presently described CARs is a mutant CD3ζ chain. For example, the mutant CD3ζ chain comprises a mutation, such as a point mutation, in at least one ITAM so as to render said ITAM non-functional. In some such embodiments, either the membrane-proximal ITAM (ITAM1), the membrane-distal ITAM (C-terminal third ITAM, ITAM3), or both are non-functional. In further embodiments, either two membrane-proximal ITAMs (ITAM1 and ITAM2) or two membrane-distal ITAMs (ITAM2 and ITAM3) are non-functional. In yet further embodiments, only ITAM2 is non-functional. In some embodiments, the mutant CD3ζ chain comprises a deletion (e.g., truncation) mutation such that at least one ITAM is missing. In some such embodiments, the CD3ζ chain is missing the membrane-proximal ITAM (ITAM1), the membrane-distal ITAM (ITAM3), or both. In other embodiments, the CD3ζ chain is missing either two membrane-proximal ITAMs (ITAM1 and ITAM2) or two membrane-distal ITAMs (ITAM2 and ITAM3). In further embodiments, the CD3ζ chain is missing ITAM2. Such mutant CD3ζ domains and methods to produce them are known to those skilled in the art (see, e.g., WO 2019/133969, herein incorporated by reference). Removing at least one ITAM from the introduced CAR may reduce CD3-mediated apoptosis. Alternatively, removing at least one ITAM from the introduced CAR can reduce its size without loss of function. CARs comprising such altered CD3ζ domains are contemplated by the present invention. Therefore, cytoplasmic signaling domains that also find use in the CARs of the present invention include mutants and variants of CD3ζ, including those specifically described in WO 2019/133969 (incorporated herein by reference) and preferably the CD3ζ variant described therein as “1XX”.

Also contemplated are CARs comprising an altered CD28 domain that imparts unique functional properties to the CAR. In this regard, the native CD28 domain comprises three intracellular subdomains consisting of the amino acid sequences YMNM, PRRP, and PYAP that regulate signaling pathways post stimulation (see, e.g., WO 2019/010383 and WO 2018/140725 incorporated herein by reference for this teaching). The CAR constructs described herein may comprise a modified CD28 domain wherein one or more of the YMNM, PRRP, and/or PYAP subdomains are mutated or deleted, so as to amplify, attenuate, or inactivate said subdomain(s), thereby modulating CAR-T function. In a preferred embodiment, the altered CD28 domain employed is Mut06 as described in WO 2019/010383. Another preferred embodiment comprises a “YNFM” mutant CD28 subdomain as taught by WO 2018/140725.

First-generation CARs typically had the intracellular domain from the CD3ζ chain, which is the primary transmitter of signals from endogenous TCRs. Second-generation CARs add intracellular signaling domains from various costimulatory protein receptors (e.g., CD28, 41BB, ICOS) to the endodomain of the CAR to provide additional signals to the T cell. Preclinical studies have indicated that the second generation of CAR designs improves the antitumor activity of T cells. More recent, third-generation CARs combine multiple signaling domains to further augment potency. T cells grafted with these CARs have demonstrated improved expansion, activation, persistence, and tumor-eradicating efficiency independent of costimulatory receptor/ligand interaction (Imai C, et al. Leukemia 2004 18:676-84; Maher J, et al. Nat Biotechnol 2002 20:70-5).

For example, the endodomain of the CAR can be designed to comprise the CD3ζ signaling domain by itself or combined with any other desired cytoplasmic domain(s) useful in the context of the CAR of the invention. For example, the cytoplasmic domain of the CAR can comprise a CD3ζ chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1 BB (CD137; see U.S. Pat. No. 8,399,645), OX40, CD30, CD40, ICOS, lymphocyte function-associated antigen-1 (LFA-1), CD2, CD7, LIGHT, NKG2C, B7-H3, and a ligand that specifically binds with CD83, CD8, CD4, b2c, CD80, CD86, DAP10, DAP12, MyD88, BTNL3, and NKG2D. Thus, while the CAR is exemplified primarily with CD28 as the co-stimulatory signaling element, other costimulatory elements can be used alone or in combination with other co-stimulatory signaling elements.

In some embodiments, the CAR comprises a hinge sequence. A hinge sequence is a short sequence of amino acids that facilitates antibody flexibility (see, e.g., Woof et al., Nat. Rev. Immunol., 4(2): 89-99 (2004)). The hinge sequence may be positioned between the antigen recognition moiety (e.g., anti-CD99 scFv or anti-CLEC12A scFv) and the transmembrane domain. The hinge sequence can be any suitable sequence derived or obtained from any suitable molecule. In some embodiments, for example, the hinge sequence is derived from a CD8a molecule or a CD28 molecule.

The transmembrane domain may be derived either from a natural or from a synthetic source. Where the source is natural, the domain may be derived from any membrane-bound or transmembrane protein. For example, the transmembrane region may be derived from (i.e. comprise at least the transmembrane region(s) of) the alpha, beta or zeta chain of the T-cell receptor, CD28, CD3 epsilon, CD45, CD4, CD5, CD8 (e.g., CD8 alpha, CD8 beta), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11 b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp. Alternatively, the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In some embodiments, the CAR has more than one transmembrane domain, which can be a repeat of the same transmembrane domain or can be different transmembrane domains.

In some embodiments, the CAR is a multi-chain CAR, as described in WO2014/039523, which is incorporated by reference for this teaching. A multi-chain CAR can comprise separate extracellular ligand binding and signaling domains in different transmembrane polypeptides. The signaling domains can be designed to assemble in juxtamembrane position, which forms flexible architecture closer to natural receptors, that confers optimal signal transduction. For example, the multi-chain CAR can comprise a part of an FCERI alpha chain and a part of an FCERI beta chain such that the FCERI chains spontaneously dimerize together to form a CAR.

CD99 CARs

The anti-CD99 binding agent is in some embodiments an antibody fragment that specifically binds CD99. For example, the antigen binding domain can be a Fab or a single-chain variable fragment (scFv) of an antibody that specifically binds CD99. The anti-CD99 binding agent is in some embodiments an aptamer that specifically binds CD99. For example, the anti-CD99 binding agent can be a peptide aptamer selected from a random sequence pool based on its ability to bind CD99. The anti-CD99 binding agent can also be a natural ligand of CD99, or a variant and/or fragment thereof capable of binding CD99.

In some embodiments, the anti-CD99 region of the disclosed antibody or CAR is derived from hybridoma 1H3, 4C5, 9G12, 3C7, 2F11, 4D5, 4F4, 6A10, or combinations thereof. In some embodiments, the anti-CD99 region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFDIKDTY (SEQ ID NO:1), TYAMY (SEQ ID NO:2), TFWM (SEQ ID NO:3), or TFWMQ (SEQ ID NO:4); the CDR2 sequence of the V_{H} domain comprises the amino acid sequence IDPANGDT (SEQ ID NO:5), RIRSKVNNYATYYADSVKDRFT (SEQ ID NO:6), or TIYPGDDDTRYTQKFKGRAT (SEQ ID NO:7); the CDR3 sequence of the V_{H} domain comprises the amino acid sequence ARRGGLS (SEQ ID NO:8), DPMDY (SEQ ID NO:9), or SGYERGPYYFDS (SEQ ID NO:10), or SGYERGPYYF (SEQ ID NO:11); the CDR1 sequence of the V_{L} comprises the amino acid sequence GNIHNY (SEQ ID NO:12), GSSKSLLHSNGNTYLY (SEQ ID NO:13), KSSQSLLCRSNQKNYLA (SEQ ID NO:14), or KSSQSLLYRSNQKNYLA (SEQ ID NO:15); the CDR2 sequence of the V_{L} domain comprises the amino acid sequence NAKX (SEQ ID NO:16), RVSNLAS (SEQ ID NO:17), or WASTRES (SEQ ID NO:18); and the CDR3 sequence of the V_{L} domain comprises the amino acid sequence QHFWSTPVVT (SEQ ID NO:19), MQHLEYPYT (SEQ ID NO:20), or QQYYSYPLT (SEQ ID NO:21).

Therefore, in some embodiments, the anti-CD99
V_{H} domain comprises the amino acid sequence
(SEQ ID NO: 22, 1H3H7, 1H3H9)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSS.
Therefore, in some embodiments, the anti-CD99
heavy chain is encoded by the nucleic acid
sequence:
(SEQ ID NO: 23, 1H3H7, 1H3H9)
GAGGTTCAACTGCAACAGTCTGGGGCAGAGCTTGTGAAGCCAGGGGCCTC
AGTCAAGTTGTCCTGCACAGCTTCTGGCTTCGACATTAAAGACACCTATA
TCCACTGGGTGAAACAGAGGCCTGAACAGGGCCTGGAGTGGATTGGAAGG
ATTGATCCTGCGAATGGTGATACTAGATATGACCCGGAATTCCAGGGCAA
GGCCTCTCTAACAGCTGACACATCCTCCAATACAGCCTACCTACAATTCA
GCAACCTGACATCTGAAGACACTGCCGTCTATTACTGTGCTAGAAGAGGC
GGCCTCTCCTGGGGCCAAGGCACCACTCTCACAGTCTCCTCA.
Therefore, in some embodiments, the anti-CD99
V_{H} domain comprises the amino acid sequence:
(SEQ ID NO: 24, 4C5E2)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSS.
Therefore, in some embodiments, the anti-CD99
heavy chain is encoded by the nucleic acid
sequence:
(SEQ ID NO: 25, 4C5E2)
GAGGTGCAGCTGGAGGAGTCTGGTGGAGGATTGGTGCAGCCTAAAGGATC
ATTGAAACTCTCATGTGCCGCCTCCGGTTTCACCTTCAATACCTATGCCA
TGTACTGGGTCTGCCAGGCTCCAGGAAAGGGTTTGAAATGGGTTGCTCGC
ATAAGAAGTAAAGTTAATAATTATGCAACATATTATGCCGATTCAGTGAA
AGACAGATTCACCATCTCCAGAGATGATTCACAAAACATGCTCTTTCTGC
ACATGAACAACCTGAAAACTGAGGACACTGCCATATATTTCTGTGTGAGA
GATCCTATGGACTACTGGGGTCAAGGAATCTCAGTCACCGTCTCCTCA.
Therefore, in some embodiments, the anti-CD99
V_{H} domain comprises the amino acid sequence:
(SEQ ID NO: 26, 4C5H10)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSS.
Therefore, in some embodiments, the anti-CD99
heavy chain is encoded by the nucleic acid
sequence:
(SEQ ID NO: 27, 4C5H10)
GAGGTGCAGCTTGTTGAGTCTGGTGGAGGATTGGTGCAGCCTAAAGGATC
ATTGAAACTCTCATGTGCCGCCTCCGGTTTCACCTTCAATACCTATGCCA
TGTACTGGGTCTGCCAGGCTCCAGGAAAGGGTTTGAAATGGGTTGCTCGC
ATAAGAAGTAAAGTTAATAATTATGCAACATATTATGCCGATTCAGTGAA
AGACAGATTCACCATCTCCAGAGATGATTCACAAAACATGCTCTTTCTGC
ACATGAACAACCTGAAAACTGAGGACACTGCCATATATTTCTGTGTGAGA
GATCCTATGGACTACTGGGGTCAAGGAATCTCAGTCACCGTCTCCTCA.
Therefore, in some embodiments, the anti-CD99
V_{H} domain comprises the amino acid sequence:
(SEQ ID NO: 28, 9G12C9)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSS.
Therefore, in some embodiments, the anti-CD99
heavy chain is encoded by the nucleic acid
sequence:
(SEQ ID NO: 29, 9G12C9)
CAGGTTCAGCTCCAGCAGTCTGGGGCTGAGCTGGCAAGACCTGGGGCTTC
AGTGAAGTTGTCCTGCAAGGCTTCTGGCTACACGTTTACTACTTTCTGGA
TGCAGTGGGTAAAACAGAGGCCTGGACAGGGTCTGGAATGGATTGGGACT
ATTTATCCTGGAGATGATGATACTAGGTACACTCAGAAATTCAAGGGCAG
GGCCACATTGACTGCAGATAAATCGTCCACCACAGCCTACATGCAACTCA
GCAACTTGTCATCTGAGGACTCTGCGGTCTATTACTGTGCAAGATCGGGG
TATGAGAGGGGCCCATACTACTTTGACTCCTGGGGCCAAGGCACCACTCT
CACAGTCTCCTCA.
Therefore, in some embodiments, the anti-CD99
V_{H} domain comprises the amino acid
sequence:
(SEQ ID NO: 30, 9G12G6 HB1)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSS.
Therefore, in some embodiments, the anti-CD99
heavy chain is encoded by the nucleic acid
sequence:
(SEQ ID NO: 31, 9G12G6 HB1)
GATGTGAAGCTTCAGGAGTCTGGGGCTGAGCTGGCAAGACCTGGGGCTTC
AGTGAAGTTGTCCTGCAAGGCTTCTGGCTACACGTTTACTACTTTCTGGA
TGCAGCGGGTAAAACAGAGGCCTGGACAGGGTCTGGAATGGATTGGGACT
ATTTATCCTGGAGATGATGATACTAGGTACACTCAGAAATTCAAGGGCAG
GGCCACATTGACTGCAGATAAATCGTCCACCACAGCCTACATGCAACTCA
GCAACTTGTCATCTGAGGACTCTGCGGTCTATTACTGTGCAAGATCGGGG
TATGAGAGGGGCCCATACTACTTTGACTCCTGGGGCCAAGGCACCACTCT
CACAGTCTCCTCA.
In some embodiments, the anti-CD99 V_{H} domain
comprises the amino acid sequence:
(SEQ ID NO: 32, 9G12G6 HB3)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSS.
Therefore, in some embodiments, the anti-CD99
heavy chain is encoded by the nucleic acid
sequence:
(SEQ ID NO: 33, 9G12G6 HB3)
CAGGTGCAGCTGAAGGAGTCTGGGGCTGAGCTGGCAAGACCTGGGGCTTC
AGTGAAGTTGTCCTGCAAGGCTTCTGGCTACACGTTTACTACTTTCTGGA
TGCAGTGGGCAAAACAGAGGCCTGGACAGGGTCTGGAATGGATTGGGACT
ATTTATCCTGGAGATGATGATACTAGGTACACTCAGAAATTCAAGGGCAG
GGCCACATTGACTGCAGATAAATCGTCCACCACAGCCTACATGCAACTCA
GCAACTTGTCATCTGAGGACTCTGCGGTCTATTACTGTGCAAGATCGGGG
TATGAGAGGGGCCCATACTACTTTGACTCCTGGGGCCAAGGCACCACTCT
CACAGTCTCCTCA.
In some embodiments, the anti-CD99 V_{L} domain
comprises the amino acid sequence:
(SEQ ID NO: 34, 1H3H9)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIK.
Therefore, in some embodiments, the anti-CD99
light chain is encoded by the nucleic acid
sequence:
(SEQ ID NO: 35, 1H3H9)
GACATCCAGATGACTCAGTCTCCAGCCTCCCTATCTGCATCTGTGGGAGA
AACTGTCACCATCACATGTCGAGCAAGTGGGAATATTCACAATTATTTAG
CATGGTATCAGCAGAAACAGGGAAAATCTCCTCAGCTCCTGGTCTATAAT
GCAAAAACCTTAGCAGATGGTGTGCCATCAAGGTTCAGTGGCAGTGGATC
AGGAACACAATATTCTCTCAAGATCAACAGCCTGCAGCCTGAAGATTTTG
GGAGTTATTACTGTCAACATTTTTGGAGTACTCCGTGGACGTTCGGTGGA
GGCACCAAGCTGGAAATCAAA.
In some embodiments, the anti-CD99 V_{L} domain
comprises the amino acid sequence:
(SEQ ID NO: 36, 1H3H7 LC1)
GNSWSHSLRSLSVTIGQPASISCKSSQSLLDGNGKTYLNWLLQRPGQSPK
RLLYLVSKLDSGVPDRFTGSGSGTDFTLKISRVEAEDLGIYYCWQGTHFP
RTFGGGTKLEIK.
In some embodiments, the anti-CD99 VL domain
comprises the amino acid sequence:
(SEQ ID NO: 37, 1H3H7 LC2)
GNSWRHSPRSLSVTIGQPASISCKSSQSLLDGNGKTYLNWLLQRPGQSPK
RLLYLVSKLDSGVPDRFTGSGSGTDFTLKISRVEAEDLGIYYCWQGTHFP
RTFGGGTKLEIK.
In some embodiments, the anti-CD99 VL domain
comprises the amino acid sequence:
(SEQ ID NO: 38, 4C5E2)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIK.
Therefore, in some embodiments, the anti-CD99
light chain is encoded by the nucleic acid
sequence:
(SEQ ID NO: 39, 4C5E2)
GATATTGTGATGACTCAGGCTGCACCCTCTGTACCTGTCACTCCTGGGGA
GTCAGTATCCATCTCCTGCGGGTCTAGTAAGAGTCTCCTGCATAGTAATG
GCAACACTTACTTGTATTGGTTCCTGCAGAGGCCAGGCCAGTCTCCTCAG
CTCCTGATATATCGGGTGTCCAACCTTGCCTCAGGAGTCCCAGACAGGTT
CAGTGGCAGTGGGTCAGGAACTGCTTTCACACTGAGAATCAGTAGAGTGG
AGGCTGAGGATGTGGGTGTTTATTACTGTATGCAACATCTAGAATATCCG
TACACGTTCGGAGGGGGGACCAGGCTGGAAATAAAA.
In some embodiments, the anti-CD99 V_{L} domain
comprises the amino acid sequence:
(SEQ ID NO: 40, 4C5H10)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIK.
Therefore, in some embodiments, the anti-CD99
light chain is encoded by the nucleic acid
sequence:
(SEQ ID NO: 41, 4C5H10)
GATATTGTGATGACTCAGGCTGCACCCTCTGTACCTGTCACTCCTGGGGA
GTCAGTATCCATCTCCTGCGGGTCTAGTAAGAGTCTCCTGCATAGTAATG
GCAACACTTACTTGTATTGGTTCCTGCAGAGGCCAGGCCAGTCTCCTCAG
CTCCTGATATATCGGGTGTCCAACCTTGCCTCAGGAGTCCCAGACAGGTT
CAGTGGCAGTGGGTCAGGAACTGCTTTCACACTGAGAATCAGTAGAGTGG
AGGCTGAGGATGTGGGTGTTTATTACTGTATGCAACATCTAGAATATCCG
TACACGTTCGGAGGGGGGACCAGGCTGGAAATAAAA.
In some embodiments, the anti-CD99 V_{L} domain
comprises the amino acid sequence:
(SEQ ID NO: 42, 9G12C9)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELK.
Therefore, in some embodiments, the anti-CD99
light chain is encoded by the nucleic acid
sequence:
(SEQ ID NO: 43, 9G12C9)
GACACTGTGATGTCACAGTCCCCATCCTCCCTAGCTGTTTCAGTTGGAGA
GAAGATAACTATGAGCTGCAAGTCCAGTCAGAGTCTTTTATGTCGTAGCA
ATCAAAAGAACTACTTGGCCTGGTACCAGCAGAAACCAGGGCAGTCTCCT
AAACAGCTGATTTACTGGGCATCTACTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAGTTAT
CCGCTCACGTTCGGTGCTGGCACCAAGCTGGAGCTGAAA.
In some embodiments, the anti-CD99 V_{L} domain
comprises the amino acid sequence:
(SEQ ID NO: 44, 9G12G6)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELK.
Therefore, in some embodiments, the anti-CD99
light chain is encoded by the nucleic acid
sequence:
(SEQ ID NO: 45, 9G12G6)
GACACTGTGATGTCACAGTCCCCATCCTCCCTAGCTGTTTCAGTTGGAGA
GAAGATAACTATGAGCTGCAAGTCCAGTCAGAGTCTTTTATATCGTAGCA
ATCAAAAGAACTACTTGGCCTGGTACCAGCAGAAACCAGGGCAGTCTCCT
AAACAGCTGATTTACTGGGCATCTACTAGGGAATCTGGGGTCCCTGATCG
CTTCACAGGCAGTGGATCTGGGACAGATTTCACTCTCACCATCAGCAGTG
TGAAGGCTGAAGACCTGGCAGTTTATTACTGTCAGCAATATTATAGTTAT
CCGCTCACGTTCGGTGCTGGCACCAAGCTGGAGCTGAAA.
The heavy and light chains are preferably
separated by a linker. Suitable linkers for
scFv antibodies are known in the art. In some
embodiments, the linker comprises the amino
acid sequence
(SEQ ID NO: 46)
GGGGSGGGGSGGGGS.
The scFv can have the formula NH₃-V_{H}-linker-
V_{L}-COOH or NH₃-V_{L}-linker-V_{H}-COOH.
In some embodiments, the anti-CD99 scFv
comprises an amino acid sequence:
(SEQ ID NO: 47, 1H3H9 v1)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSDIQMTQSPASLSASVGETVTI
TCRASGNIHNYLAWYQQKQGKSPQLLVYNAKTLADGVPSRFSGSGSGTQY
SLKINSLQPEDFGSYYCQHFWSTPWTFGGGTKLEIK.
In some embodiments, the anti-CD99 scFv
comprises an amino acid sequence:
(SEQ ID NO: 48)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSGNSWSHSLRSLSVTIGQPASI
SCKSSQSLLDGNGKTYLNWLLQRPGQSPKRLLYLVSKLDSGVPDRFTGSG
SGTDFTLKISRVEAEDLGIYYCWQGTHFPRTFGGGTKLEIK.
In some embodiments, the anti-CD99 scFv
comprises an amino acid sequence:
(SEQ ID NO: 49)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSGNSWRHSPRSLSVTIGQPASI
SCKSSQSLLDGNGKTYLNWLLQRPGQSPKRLLYLVSKLDSGVPDRFTGSG
SGTDFTLKISRVEAEDLGIYYCWQGTHFPRTFGGGTKLEIK.
In some embodiments, the anti-CD99 scFv
comprises an amino acid sequence:
(SEQ ID NO: 50)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSDIVMTQAAPSVPVTPGESVSI
SCGSSKSLLHSNGNTYLYWFLQRPGQSPQLLIYRVSNLASGVPDRFSGSG
SGTAFTLRISRVEAEDVGVYYCMQHLEYPYTFGGGTRLEIK.
In some embodiments, the anti-CD99 scFv
comprises an amino acid sequence:
(SEQ ID NO: 51)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSDTVMSQSPSSLAVSVGEKITM
SCKSSQSLLCRSNQKNYLAWYQQKPGQSPKQLIYWASTRESGVPDRFTGS
GSGTDFTLTISSVKAEDLAVYYCQQYYSYPLTFGAGTKLELK.
In some embodiments, the anti-CD99 scFv
comprises an amino acid sequence:
(SEQ ID NO: 52)
EVQLQQSGAELVKPGASVKLSCTASGFDIKDTYIHWVKQRPEQGLEWIGR
IDPANGDTRYDPEFQGKASLTADTSSNTAYLQFSNLTSEDTAVYYCARRG
GLSWGQGTTLTVSSGGGGSGGGGSGGGGSDTVMSQSPSSLAVSVGEKITM
SCKSSQSLLYRSNQKNYLAWYQQKPGQSPKQLIYWASTRESGVPDRFTGS
GSGTDFTLTISSVKAEDLAVYYCQQYYSYPLTFGAGTKLELK.
In some embodiments, the anti-CD99 scFv
comprises an amino acid sequence:
(SEQ ID NO: 53)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDIQMTQSPASLSASVGETV
TITCRASGNIHNYLAWYQQKQGKSPQLLVYNAKTLADGVPSRFSGSGSGT
QYSLKINSLQPEDFGSYYCQHFWSTPWTFGGGTKLEIK.
In some embodiments, the anti-CD99 scFv
comprises an amino acid sequence:
(SEQ ID NO: 54)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSGNSWSHSLRSLSVTIGQPA
SISCKSSQSLLDGNGKTYLNWLLQRPGQSPKRLLYLVSKLDSGVPDRFTG
SGSGTDFTLKISRVEAEDLGIYYCWQGTHFPRTFGGGTKLEIK.
In some embodiments, the anti-CD99 scFv
comprises an amino acid sequence:
(SEQ ID NO: 55)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSGNSWRHSPRSLSVTIGQPA
SISCKSSQSLLDGNGKTYLNWLLQRPGQSPKRLLYLVSKLDSGVPDRFTG
SGSGTDFTLKISRVEAEDLGIYYCWQGTHFPRTFGGGTKLEIK.
In some embodiments, the anti-CD99 scFv
comprises an amino acid sequence:
(SEQ ID NO: 56, 4C5E2 v1)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDIVMTQAAPSVPVTPGESV
SISCGSSKSLLHSNGNTYLYWFLQRPGQSPQLLIYRVSNLASGVPDRFSG
SGSGTAFTLRISRVEAEDVGVYYCMQHLEYPYTFGGGTRLEIK.
In some embodiments, the anti-CD99 scFv
comprises an amino acid sequence:
(SEQ ID NO: 57)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDTVMSQSPSSLAVSVGEKI
TMSCKSSQSLLCRSNQKNYLAWYQQKPGQSPKQLIYWASTRESGVPDRFT
GSGSGTDFTLTISSVKAEDLAVYYCQQYYSYPLTFGAGTKLELK.
In some embodiments, the anti-CD99 scFv
comprises an amino acid sequence:
(SEQ ID NO: 58)
EVQLEESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDTVMSQSPSSLAVSVGEKI
TMSCKSSQSLLYRSNQKNYLAWYQQKPGQSPKQLIYWASTRESGVPDRFT
GSGSGTDFTLTISSVKAEDLAVYYCQQYYSYPLTFGAGTKLELK.
In some embodiments, the anti-CD99 scFv
comprises an amino acid sequence:
(SEQ ID NO: 59)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDIQMTQSPASLSASVGETV
TITCRASGNIHNYLAWYQQKQGKSPQLLVYNAKTLADGVPSRFSGSGSGT
QYSLKINSLQPEDFGSYYCQHFWSTPWTFGGGTKLEIK.
In some embodiments, the anti-CD99 scFv
comprises an amino acid sequence:
(SEQ ID NO: 60)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSGNSWSHSLRSLSVTIGQPA
SISCKSSQSLLDGNGKTYLNWLLQRPGQSPKRLLYLVSKLDSGVPDRFTG
SGSGTDFTLKISRVEAEDLGIYYCWQGTHFPRTFGGGTKLEIK.
In some embodiments, the anti-CD99 scFv
comprises an amino acid sequence:
(SEQ ID NO: 61)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSGNSWRHSPRSLSVTIGQPA
SISCKSSQSLLDGNGKTYLNWLLQRPGQSPKRLLYLVSKLDSGVPDRFTG
SGSGTDFTLKISRVEAEDLGIYYCWQGTHFPRTFGGGTKLEIK.
In some embodiments, the anti-CD99 scFv
comprises an amino acid sequence:
(SEQ ID NO: 62, 4C5H10 v1)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDIVMTQAAPSVPVTPGESV
SISCGSSKSLLHSNGNTYLYWFLQRPGQSPQLLIYRVSNLASGVPDRFSG
SGSGTAFTLRISRVEAEDVGVYYCMQHLEYPYTFGGGTRLEIK.
In some embodiments, the anti-CD99 scFv
comprises an amino acid sequence:
(SEQ ID NO: 63)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDTVMSQSPSSLAVSVGEKI
TMSCKSSQSLLCRSNQKNYLAWYQQKPGQSPKQLIYWASTRESGVPDRFT
GSGSGTDFTLTISSVKAEDLAVYYCQQYYSYPLTFGAGTKLELK.
In some embodiments, the anti-CD99 scFv
comprises an amino acid sequence:
(SEQ ID NO: 64)
EVQLVESGGGLVQPKGSLKLSCAASGFTFNTYAMYWVCQAPGKGLKWVAR
IRSKVNNYATYYADSVKDRFTISRDDSQNMLFLHMNNLKTEDTAIYFCVR
DPMDYWGQGISVTVSSGGGGSGGGGSGGGGSDTVMSQSPSSLAVSVGEKI
TMSCKSSQSLLYRSNQKNYLAWYQQKPGQSPKQLIYWASTRESGVPDRFT
GSGSGTDFTLTISSVKAEDLAVYYCQQYYSYPLTFGAGTKLELK.
In some embodiments, the anti-CD99 scFv
comprises an amino acid sequence:
(SEQ ID NO: 65)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIQMTQSPASLSAS
VGETVTITCRASGNIHNYLAWYQQKQGKSPQLLVYNAKTLADGVPSRFSG
SGSGTQYSLKINSLQPEDFGSYYCQHFWSTPWTFGGGTKLEIK.
In some embodiments, the anti-CD99 scFv
comprises an amino acid sequence:
(SEQ ID NO: 66)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWSHSLRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 67)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWRHSPRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 68)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.
In some embodiments, the anti-CD99 scFv
comprises an amino acid sequence:
(SEQ ID NO: 69, 9G12C9 v1)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLCRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 70)
QVQLQQSGAELARPGASVKLSCKASGYTFTTFWMQWVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLYRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 71)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIQMTQSPASLSAS
VGETVTITCRASGNIHNYLAWYQQKQGKSPQLLVYNAKTLADGVPSRFSG
SGSGTQYSLKINSLQPEDFGSYYCQHFWSTPWTFGGGTKLEIK.
In some embodiments, the anti-CD99 scFv
comprises an amino acid sequence:
(SEQ ID NO: 72)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWSHSLRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 73)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWRHSPRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 74)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.
In some embodiments, the anti-CD99 scFv
comprises an amino acid sequence:
(SEQ ID NO: 75)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.
In some embodiments, the anti-CD99 scFv
comprises an amino acid sequence:
(SEQ ID NO: 76)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLCRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 77)
DVKLQESGAELARPGASVKLSCKASGYTFTTFWMQRVKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLYRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 78)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIQMTQSPASLSAS
VGETVTITCRASGNIHNYLAWYQQKQGKSPQLLVYNAKTLADGVPSRFSG
SGSGTQYSLKINSLQPEDFGSYYCQHFWSTPWTFGGGTKLEIK.
In some embodiments, the anti-CD99 scFv
comprises an amino acid sequence:
(SEQ ID NO: 79)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWSHSLRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 80)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSGNSWRHSPRSLSVT
IGQPASISCKSSQSLLDGNGKTYLNWLLQRPGQSPKRLLYLVSKLDSGVP
DRFTGSGSGTDFTLKISRVEAEDLGIYYCWQGTHFPRTFGGGTKLEIK.
In some embodiments, the anti-CD99 scFv
comprises an amino acid sequence:
(SEQ ID NO: 81)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.
In some embodiments, the anti-CD99 scFv
comprises an amino acid sequence:
(SEQ ID NO: 82)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDIVMTQAAPSVPVT
PGESVSISCGSSKSLLHSNGNTYLYWFLQRPGQSPQLLIYRVSNLASGVP
DRFSGSGSGTAFTLRISRVEAEDVGVYYCMQHLEYPYTFGGGTRLEIK.
In some embodiments, the anti-CD99 scFv
comprises an amino acid sequence:
(SEQ ID NO: 83)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLCRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 84)
QVQLKESGAELARPGASVKLSCKASGYTFTTFWMQWAKQRPGQGLEWIGT
IYPGDDDTRYTQKFKGRATLTADKSSTTAYMQLSNLSSEDSAVYYCARSG
YERGPYYFDSWGQGTTLTVSSGGGGSGGGGSGGGGSDTVMSQSPSSLAVS
VGEKITMSCKSSQSLLYRSNQKNYLAWYQQKPGQSPKQLIYWASTRESGV
PDRFTGSGSGTDFTLTISSVKAEDLAVYYCQQYYSYPLTFGAGTKLELK.
In some embodiments, the anti-CD99 scFv
comprises an amino acid sequence:
(SEQ ID NO: 85, 1H3H9 v2)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLQQSGAELVKPGASVKLSCTASGFD
IKDTYIHWVKQRPEQGLEWIGRIDPANGDTRYDPEFQGKASLTADTSSNT
AYLQFSNLTSEDTAVYYCARRGGLSWGQGTTLTVSS.
In some embodiments, the anti-CD99 scFv
comprises an amino acid sequence:
(SEQ ID NO: 86)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLEESGGGLVQPKGSLKLSCAASGFT
FNTYAMYWVCQAPGKGLKWVARIRSKVNNYATYYADSVKDRFTISRDDSQ
NMLFLHMNNLKTEDTAIYFCVRDPMDYWGQGISVTVSS.
In some embodiments, the anti-CD99 scFv
comprises an amino acid sequence:
(SEQ ID NO: 87)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSEVQLVESGGGLVQPKGSLKLSCAASGFT
FNTYAMYWVCQAPGKGLKWVARIRSKVNNYATYYADSVKDRFTISRDDSQ
NMLFLHMNNLKTEDTAIYFCVRDPMDYWGQGISVTVSS.
In some embodiments, the anti-CD99 scFv
comprises an amino acid sequence:
(SEQ ID NO: 88)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSQVQLQQSGAELARPGASVKLSCKASGYT
FTTFWMQWVKQRPGQGLEWIGTIYPGDDDTRYTQKFKGRATLTADKSSTT
AYMQLSNLSSEDSAVYYCARSGYERGPYYFDSWGQGTTLTVSS.
In some embodiments, the anti-CD99 scFv
comprises an amino acid sequence:
(SEQ ID NO: 89)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSDVKLQESGAELARPGASVKLSCKASGYT
FTTFWMQRVKQRPGQGLEWIGTIYPGDDDTRYTQKFKGRATLTADKSSTT
AYMQLSNLSSEDSAVYYCARSGYERGPYYFDSWGQGTTLTVSS.
In some embodiments, the anti-CD99 scFv
comprises an amino acid sequence:
(SEQ ID NO: 90)
DIQMTQSPASLSASVGETVTITCRASGNIHNYLAWYQQKQGKSPQLLVYN
AKTLADGVPSRFSGSGSGTQYSLKINSLQPEDFGSYYCQHFWSTPWTFGG
GTKLEIKGGGGSGGGGSGGGGSQVQLKESGAELARPGASVKLSCKASGYT
FTTFWMQWAKQRPGQGLEWIGTIYPGDDDTRYTQKFKGRATLTADKSSTT
AYMQLSNLSSEDSAVYYCARSGYERGPYYFDSWGQGTTLTVSS.
In some embodiments, the anti-CD99 scFv
comprises an amino acid sequence:
(SEQ ID NO: 91)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSEVQLQQSGAELVKPGASVKLSCT
ASGFDIKDTYIHWVKQRPEQGLEWIGRIDPANGDTRYDPEFQGKASLTAD
TSSNTAYLQFSNLTSEDTAVYYCARRGGLSWGQGTTLTVSS.
In some embodiments, the anti-CD99 scFv
comprises an amino acid sequence:
(SEQ ID NO: 92, 4C5E2 v2)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSEVQLEESGGGLVQPKGSLKLSCA
ASGFTFNTYAMYWVCQAPGKGLKWVARIRSKVNNYATYYADSVKDRFTIS
RDDSQNMLFLHMNNLKTEDTAIYFCVRDPMDYWGQGISVTVSS.
In some embodiments, the anti-CD99 scFv
comprises an amino acid sequence:
(SEQ ID NO: 93, 4C5H10 v2)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSEVQLVESGGGLVQPKGSLKLSCA
ASGFTFNTYAMYWVCQAPGKGLKWVARIRSKVNNYATYYADSVKDRFTIS
RDDSQNMLFLHMNNLKTEDTAIYFCVRDPMDYWGQGISVTVSS.
In some embodiments, the anti-CD99 scFv
comprises an amino acid sequence:
(SEQ ID NO: 94)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSQVQLQQSGAELARPGASVKLSCK
ASGYTFTTFWMQWVKQRPGQGLEWIGTIYPGDDDTRYTQKFKGRATLTAD
KSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 95)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSDVKLQESGAELARPGASVKLSCK
ASGYTFTTFWMQRVKQRPGQGLEWIGTIYPGDDDTRYTQKFKGRATLTAD
KSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 96)
DIVMTQAAPSVPVTPGESVSISCGSSKSLLHSNGNTYLYWFLQRPGQSPQ
LLIYRVSNLASGVPDRFSGSGSGTAFTLRISRVEAEDVGVYYCMQHLEYP
YTFGGGTRLEIKGGGGSGGGGSGGGGSQVQLKESGAELARPGASVKLSCK
ASGYTFTTFWMQWAKQRPGQGLEWIGTIYPGDDDTRYTQKFKGRATLTAD
KSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 97)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLQQSGAELVKPGASVKLSC
TASGFDIKDTYIHWVKQRPEQGLEWIGRIDPANGDTRYDPEFQGKASLTA
DTSSNTAYLQFSNLTSEDTAVYYCARRGGLSWGQGTTLTVSS.
In some embodiments, the anti-CD99 scFv
comprises an amino acid sequence:
(SEQ ID NO: 98)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLEESGGGLVQPKGSLKLSC
AASGFTFNTYAMYWVCQAPGKGLKWVARIRSKVNNYATYYADSVKDRFTI
SRDDSQNMLFLHMNNLKTEDTAIYFCVRDPMDYWGQGISVTVSS.
In some embodiments, the anti-CD99 scFv
comprises an amino acid sequence:
(SEQ ID NO: 99)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLVESGGGLVQPKGSLKLSC
AASGFTFNTYAMYWVCQAPGKGLKWVARIRSKVNNYATYYADSVKDRFTI
SRDDSQNMLFLHMNNLKTEDTAIYFCVRDPMDYWGQGISVTVSS.
In some embodiments, the anti-CD99 scFv
comprises an amino acid sequence:
(SEQ ID NO: 100, 9G12C9 v2)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSQVQLQQSGAELARPGASVKLSC
KASGYTFTTFWMQWVKQRPGQGLEWIGTIYPGDDDTRYTQKFKGRATLTA
DKSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 101)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSDVKLQESGAELARPGASVKLSC
KASGYTFTTFWMQRVKQRPGQGLEWIGTIYPGDDDTRYTQKFKGRATLTA
DKSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 102)
DTVMSQSPSSLAVSVGEKITMSCKSSQSLLCRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSQVQLKESGAELARPGASVKLSC
KASGYTFTTFWMQWAKQRPGQGLEWIGTIYPGDDDTRYTQKFKGRATLTA
DKSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 103)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLQQSGAELVKPGASVKLSC
TASGFDIKDTYIHWVKQRPEQGLEWIGRIDPANGDTRYDPEFQGKASLTA
DTSSNTAYLQFSNLTSEDTAVYYCARRGGLSWGQGTTLTVSS.
In some embodiments, the anti-CD99 scFv
comprises an amino acid sequence:
(SEQ ID NO: 104)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLEESGGGLVQPKGSLKLSC
AASGFTFNTYAMYWVCQAPGKGLKWVARIRSKVNNYATYYADSVKDRFTI
SRDDSQNMLFLHMNNLKTEDTAIYFCVRDPMDYWGQGISVTVSS.
In some embodiments, the anti-CD99 scFv
comprises an amino acid sequence:
(SEQ ID NO: 105)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSEVQLVESGGGLVQPKGSLKLSC
AASGFTFNTYAMYWVCQAPGKGLKWVARIRSKVNNYATYYADSVKDRFTI
SRDDSQNMLFLHMNNLKTEDTAIYFCVRDPMDYWGQGISVTVSS.
In some embodiments, the anti-CD99 scFv
comprises an amino acid sequence:
(SEQ ID NO: 106)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSQVQLQQSGAELARPGASVKLSC
KASGYTFTTFWMQWVKQRPGQGLEWIGTIYPGDDDTRYTQKFKGRATLTA
DKSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 107)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSDVKLQESGAELARPGASVKLSC
KASGYTFTTFWMQRVKQRPGQGLEWIGTIYPGDDDTRYTQKFKGRATLTA
DKSSTTAYMQLSNLSSEDSAVYYCARSGYERGPYYFDSWGQGTTLTVSS.
In some embodiments, the anti-CD99 scFv
comprises an amino acid sequence:
(SEQ ID NO: 108)
DTVMSQSPSSLAVSVGEKITMSCKSSQSLLYRSNQKNYLAWYQQKPGQSP
KQLIYWASTRESGVPDRFTGSGSGTDFTLTISSVKAEDLAVYYCQQYYSY
PLTFGAGTKLELKGGGGSGGGGSGGGGSQVQLKESGAELARPGASVKLSC
KASGYTFTTFWMQWAKQRPGQGLEWIGTIYPGDDDTRYTQKFKGRATLTA
DKSSTTAYMQLSNLSSEDSAVYYCARSGYERGPYYFDSWGQGTTLTVSS.

### CLEC12A CARs

In some embodiments, the anti-CLEC12A region of the disclosed antibody or CAR is derived from hybridoma 1F3, 1F8, 1G3, 2A10, 3F12, 4E3, 4E10, 5B2, 5F10, 6C7, 9A2, 11C7, 11H1, 12D6, or combinations thereof. In some embodiments, the anti-CLEC12A region (e.g. scFv) can comprise a variable heavy (V_{H}) domain having CDR1, CDR2 and CDR3 sequences and a variable light (V_{L}) domain having CDR1, CDR2 and CDR3 sequences.

In some embodiments, the CDR1 sequence of the V_{H} domain comprises the amino acid sequence GFTFSSFA (SEQ ID NO:109) SFAVS (SEQ ID NO:110), or SHDMS (SEQ ID NO:111); the CDR2 sequence of the V_{H} domain comprises the amino acid sequence ISSGGAYT (SEQ ID NO:112) or TISSGGAYTFYKDSVKGRFT (SEQ ID NO:113), or YISGGGTNIYYSDTVKGRFT (SEQ ID NO:114); the CDR3 sequence of the V_{H} domain comprises the amino acid sequence ARHSGYDGYYLYAMDY (SEQ ID NO:115), HSGYDGYYLYAMDY (SEQ ID NO:116), or PNYNYGGSWFAY (SEQ ID NO: 117); the CDR1 sequence of the V_{L} comprises the amino acid sequence SSVHY (SEQ ID NO:118), ASSSVHYMH (SEQ ID NO:119), or SASSSVHYMH (SEQ ID NO:120); the CDR2 sequence of the V_{L} domain comprises the amino acid sequence DTSX (SEQ ID NO:121) or DTSKLAS (SEQ ID NO:122); and the CDR3 sequence of the V_{L} domain comprises the amino acid sequence QQWTSNPPT (SEQ ID NO:123).

In some embodiments, the anti-CLEC12A V_{H} domain comprises the amino acid
sequence:
(SEQ ID NO: 124, 1F3H8)
ELILVESGGGLVKPGGSLKLSCAVSGFTFSSFAMSWRQTPEKRLEWATISSGGAYT
FYKDSVKGRFTISRDNAKNTLYLQMSSLRSEDSAMYYCARHSGYDGYYLYAMDYWGQ
GTSVTVSS.
Therefore, in some embodiments, the anti-CLEC12A V_{H} domain is encoded by
the nucleic acid sequence:
(SEQ ID NO: 125, 1F3H8)
GAACTAATACTGGTGGAGTCTGGGGGAGGCTTAGTGAAGCCTGGAGGGTCCCTGA
AACTCTCCTGTGCAGTCTCTGGATTCACTTTCAGTTCCTTTGCCATGTCTTGGGTTC
GCCAGACTCCGGAGAAGAGGCTGGAGTGGGTCGCAACCATTAGTAGTGGTGGAGC
TTACACCTTCTATAAAGACAGTGTGAAGGGGCGATTCACCATCTCCAGAGACAATG
CCAAGAATACCCTGTACCTGCAAATGAGCAGTCTGAGGTCTGAGGACTCGGCCATG
TATTACTGTGCAAGACATAGCGGCTATGATGGTTACTACCTCTATGCTATGGACTAC
TGGGGTCAAGGAACCTCAGTCACCGTCTCCTCA.
In some embodiments, the anti-CLEC12A V_{H} domain comprises the amino acid
sequence:
(SEQ ID NO: 126, 1F3A10)
GVQCELILVESGGGLVKPGGSLKLSCAVSGFTFSSFAVSWWRQTPEKRLEWATISSG
GAYTFYKDSVKGRFTISRDNAKNTLYLQMSSLRSEDSAMYYCARHSGYDGYYLYAMD
YWGQGTSVTVSS.
Therefore, in some embodiments, the anti-CLEC12A V_{H} domain is encoded by
the nucleic acid sequence:
(SEQ ID NO: 127, 1F3A10)
GGTGTCCAGTGTGAACTAATACTGGTGGAGTCTGGGGGAGGCTTAGTGAAGCCTG
GAGGGTCCCTGAAACTCTCCTGTGCAGTCTCTGGATTCACTTTCAGTTCCTTTGCC
GTGTCCTGGGTTCGCCAGACTCCGGAGAAGAGGCTGGAGTGGGTCGCAACCATTA
GTAGTGGTGGAGCTTACACCTTCTATAAAGACAGTGTGAAGGGGCGATTCACCATC
TCCAGAGACAATGCCAAGAATACCCTGTACCTGCAAATGAGCAGTCTGAGGTCTGA
GGACTCGGCCATGTATTACTGTGCAAGACATAGCGGCTATGATGGTTACTACCTCT
ATGCTATGGACTACTGGGGTCAAGGAACCTCAGTCACCGTCTCCTCA.
In some embodiments, the anti-CLEC12A V_{H} domain comprises the amino acid
sequence:
(SEQ ID NO: 128, 1F3F3)
EVQLEESGGGLVQPGGSLKVSCAVSGLAFSSHDMSWRQTPEKRLEWAYISGGGT
NIYYSDTVKGRFTISRDNAKNTLYLQMSSLKSEDTAIYYCARPNYNYGGSWFAYWGQG
TLVTVSA.
Therefore, in some embodiments, the anti-CLEC12A V_{H} domain is encoded by
the nucleic acid sequence:
(SEQ ID NO: 129, 1F3F3)
GAGGTGCAGCTGGAGGAGTCTGGGGGAGGCTTAGTGCAGCCGGGAGGGTCCCTG
AAAGTCTCCTGTGCAGTTTCCGGACTCGCTTTCAGCAGCCATGACATGTCTTGGGT
TCGCCAGACTCCGGAGAAGCGGCTGGAGTGGGTCGCATACATTAGTGGAGGTGGT
ACTAATATCTATTATTCAGACACTGTGAAGGGCCGATTCACCATCTCCAGAGACAAT
GCCAAGAACACCCTGTACCTGCAAATGAGCAGTCTGAAGTCTGAAGACACAGCCAT
TTATTACTGTGCAAGACCCAATTATAATTACGGCGGTTCCTGGTTTGCTTACTGGGG
CCAAGGGACTCTGGTCACTGTCTCTGCA.
In some embodiments, the anti-CLEC12A V_{L} domain comprises the amino acid
sequence:
(SEQ ID NO: 130, 1F3H8, 1F3F3, 1F3A10)
QIVLTQSPEIMSASPGEKVTMTCSASSSVHYMHWYQQKSGTSPKRWIYDTSKLASGVP
GRFSGSGSGTSYSLTISSMESEDAATYYCQQWTSNPPTFGGGTKLEIK.
Therefore, in some embodiments, the anti-CLEC12A V_{L} domain is encoded by
the nucleic acid sequence:
(SEQ ID NO: 131, 1F3H8, 1F3F3, 1F3A10)
CAAATTGTTCTCACCCAGTCTCCAGAAATCATGTCTGCATCTCCAGGGGAGAAGGT
CACCATGACCTGCAGTGCCAGCTCAAGTGTACATTACATGCACTGGTACCAGCAGA
AGTCAGGCACCTCCCCCAAAAGATGGATTTATGACACATCCAAACTGGCTTCTGGA
GTCCCTGGTCGCTTCAGTGGCAGTGGGTCTGGGACCTCTTACTCTCTCACAATCAG
CAGCATGGAGTCTGAAGATGCTGCCACTTATTACTGCCAGCAGTGGACTAGTAACC
CACCCACGTTCGGAGGGGGGACCAAGCTGGAAATTAAACG.

The heavy and light chains are preferably separated by a linker. Suitable linkers for scFv antibodies are known in the art. In some embodiments, the linker comprises the amino acid sequence GGGGSGGGGSGGGGS (SEQ ID NO:46). The scFv can have the formula NH₃—V_{H}-linker-V_{L}—COOH or NH₃—V_{L}-linker-V_{H}—COOH.

In some embodiments, the anti-CLEC12A scFv comprises an amino acid
sequence:
(SEQ ID NO: 132, 1F3H8 v1)
ELILVESGGGLVKPGGSLKLSCAVSGFTFSSFAMSWRQTPEKRLEWATISSGGAYT
FYKDSVKGRFTISRDNAKNTLYLQMSSLRSEDSAMYYCARHSGYDGYYLYAMDYWGQ
GTSVTVSSGGGGSGGGGSGGGGSQIVLTQSPEIMSASPGEKVTMTCSASSSVHYMH
WYQQKSGTSPKRWIYDTSKLASGVPGRFSGSGSGTSYSLTISSMESEDAATYYCQQW
TSNPPTFGGGTKLEIK.
In some embodiments, the anti-CLEC12A scFv comprises an amino acid
sequence:
(SEQ ID NO: 133, 1F3A10 v1)
GVQCELILVESGGGLVKPGGSLKLSCAVSGFTFSSFAVSWRQTPEKRLEWATISSG
GAYTFYKDSVKGRFTISRDNAKNTLYLQMSSLRSEDSAMYYCARHSGYDGYYLYAMD
YWGQGTSVTVSSGGGGSGGGGSGGGGSQIVLTQSPEIMSASPGEKVTMTCSASSSV
HYMHWYQQKSGTSPKRWIYDTSKLASGVPGRFSGSGSGTSYSLTISSMESEDAATYY
CQQWTSNPPTFGGGTKLEIK.
In some embodiments, the anti-CLEC12A scFv comprises an amino acid
sequence:
(SEQ ID NO: 134, 1F3F3 v1)
EVQLEESGGGLVQPGGSLKVSCAVSGLAFSSHDMSWVRQTPEKRLEWAYISGGGT
NIYYSDTVKGRFTISRDNAKNTLYLQMSSLKSEDTAIYYCARPNYNYGGSWFAYWGQG
TLVTVSAGGGGSGGGGSGGGGSQIVLTQSPEIMSASPGEKVTMTCSASSSVHYMHW
YQQKSGTSPKRWIYDTSKLASGVPGRFSGSGSGTSYSLTISSMESEDAATYYCQQWT
SNPPTFGGGTKLEIK.
In some embodiments, the anti-CLEC12A scFv comprises an amino acid
sequence:
(SEQ ID NO: 135, 1F3H8 v2)
QIVLTQSPEIMSASPGEKVTMTCSASSSVHYMHWYQQKSGTSPKRWIYDTSKLASGVP
GRFSGSGSGTSYSLTISSMESEDAATYYCQQWTSNPPTFGGGTKLEIKGGGGSGGGG
SGGGGSELILVESGGGLVKPGGSLKLSCAVSGFTFSSFAMSWWRQTPEKRLEWVATIS
SGGAYTFYKDSVKGRFTISRDNAKNTLYLQMSSLRSEDSAMYYCARHSGYDGYYLYA
MDYWGQGTSVTVSS.
In some embodiments, the anti-CLEC12A scFv comprises an amino acid
sequence:
(SEQ ID NO: 136, 1F3A10 v2)
QIVLTQSPEIMSASPGEKVTMTCSASSSVHYMHWYQQKSGTSPKRWIYDTSKLASGVP
GRFSGSGSGTSYSLTISSMESEDAATYYCQQWTSNPPTFGGGTKLEIKGGGGSGGGG
SGGGGSGVQCELILVESGGGLVKPGGSLKLSCAVSGFTFSSFAVSWRQTPEKRLEW
VATISSGGAYTFYKDSVKGRFTISRDNAKNTLYLQMSSLRSEDSAMYYCARHSGYDGY
YLYAMDYWGQGTSVTVSS.
In some embodiments, the anti-CLEC12A scFv comprises an amino acid
sequence:
(SEQ ID NO: 137, 1F3F3 v2)
QIVLTQSPEIMSASPGEKVTMTCSASSSVHYMHWYQQKSGTSPKRWIYDTSKLASGVP
GRFSGSGSGTSYSLTISSMESEDAATYYCQQWTSNPPTFGGGTKLEIKGGGGSGGGG
SGGGGSEVQLEESGGGLVQPGGSLKVSCAVSGLAFSSHDMSWVRQTPEKRLEWVAY
ISGGGTNIYYSDTVKGRFTISRDNAKNTLYLQMSSLKSEDTAIYYCARPNYNYGGSWFA
YWGQGTLVTVSA.

A dual CAR T cell expresses two separate CARs with different ligand binding targets; one CAR includes only the CD3ζ domain and the other CAR includes only the co-stimulatory domain(s). Dual CAR T cell activation requires co-expression of both targets on the tumor. In some embodiments, the two CARs are expressed separately. In some embodiments, the two CARs are co-expressed by a single expression construct. In some embodiments, the two CARs are co-expressed in a single fusion protein separated by a self-cleavable peptide.

Therefore, in some embodiments, the disclosed a dual CAR fusion protein is defined by the formula:

SP-CD99V_{L}-CLV_{H}-HG-TM-CSD-scp-SP-CD99V_{H}-CLV_{L}-HG-TM-CD3ζ-SD;

SP-CD99V_{L}-CLV_{H}-HG-TM-CD3ζ-scp-SP-CD99V_{H}-CLV_{L}-HG-TM-CSD-SD;

SP-CD99V_{H}-CLV_{L}-HG-TM-CSD-scp-SP-CD99V_{L}-CLV_{H}-HG-TM-CD3ζ-SD;

SP-CD99V_{H}-CLV_{L}-HG-TM-CD3ζ-scp-SP-CD99V_{L}-CLV_{H}-HG-TM-CSD-SD;

SP-CD99V_{H}-CLV_{H}-HG-TM-CSD-scp-SP-CD99V_{L}-CLV_{L}-HG-TM-CD3ζ-SD;

SP-CD99V_{L}-CLV_{L}-HG-TM-CD3ζ-scp-SP-CD99V_{H}-CLV_{H}-HG-TM-CSD-SD;

SP-CD99V_{L}-CLV_{L}-HG-TM-CSD-scp-SP-CD99V_{H}-CLV_{H}-HG-TM-CD3ζ-SD;

SP-CD99V_{H}-CLV_{H}-HG-TM-CD3ζ-scp-SP-CD99V_{L}-CLV_{L}-HG-TM-CSD-SD;

SP-CLV_{H}-CD99V_{L}-HG-TM-CSD-scp-SP-CLV_{L}-CD99V_{H}-HG-TM-CD3ζ-SD;

SP-CLV_{H}-CD99V_{L}-HG-TM-CD3ζ-scp-SP-CLV_{L}-CD99V_{H}-HG-TM-CSD-SD;

SP-CLV_{L}-CD99V_{H}-HG-TM-CSD-scp-SP-CLV_{H}-CD99V_{L}-HG-TM-CD3ζ-SD;

SP-CLV_{L}-CD99V_{H}-HG-TM-CD3ζ-scp-SP-CLV_{H}-CD99V_{L}-HG-TM-CSD-SD;

SP-CLV_{H}-CD99V_{H}-HG-TM-CSD-scp-SP-CLV_{L}-CD99V_{L}-HG-TM-CD3ζ-SD;

SP-CLV_{L}-CD99V_{L}-HG-TM-CD3ζ-scp-SP-CLV_{H}-CD99V_{H}-HG-TM-CSD-SD;

SP-CLV_{L}-CD99V_{L}-HG-TM-CSD-scp-SP-CLV_{H}-CD99V_{H}-HG-TM-CD3ζ-SD;

or

SP-CLV_{H}-CD99V_{H}-HG-TM-CD3ζ-scp-SP-CLV_{L}-CD99V_{L}-HG-TM-CSD-SD;

wherein “SP” represents an optional signal peptide,

wherein “CD99V_{H}” represents a CD99 variable heavy domain,

wherein “CD99V_{L}” represents a CD99 variable light domain,

wherein “CLV_{H}” represents a CLEC12A variable heavy domain,

wherein “CLV_{L}” represents a CLEC12A variable light domain,

wherein “HG” represents an optional hinge domain,

wherein “TM” represents a transmembrane domain,

wherein “scp” represents a self-cleaving peptide domain,

wherein “CD3ζ” represents a CD3 domain,

wherein “CSD” represents a costimulatory-domain, and

wherein “-” represents a peptide bond or linker.

As an example, in some embodiments, the dual CAR fusion protein has the
amino acid sequence:
(SEQ ID NO: 138)
MALPVTALLLPLALLLHAARPDIVMTQAAPSVPVTPGESVSISCGSSKSLLHSNGNTYLY
WFLQRPGQSPQLLIYRVSNLASGVPDRFSGSGSGTAFTLRISRVEAEDVGVYYCMQHL
EYPYTFGGGTRLEIKGGGGSGGGGSGGGGSEVQLEESGGGLVQPKGSLKLSCAASG
FTFNTYAMYWVCQAPGKGLKWVARIRSKVNNYATYYADSVKDRFTISRDDSQNMLFLH
MNNLKTEDTAIYFCVRDPMDYWGQGISVTVSSAAATTTPAPRPPTPAPTIASQPLSLRP
EACRPAAGGAVHTRGLDFACDIYIWAPLAGTCGVLLLSLVITLYCKRGRKKLLYIFKQPF
MRPVQTTQEEDGCSCRFPEEEEGGCELRSKRSRLLHSDFMNMTARRAGPTRKHYQP
YAPPRDFAAYRSGSGATNFSLLKQAGDVEENPGPMALPVTALLLPLALLLHAARPELILV
ESGGGLVKPGGSLKLSCAVSGFTFSSFAMSWVRQTPEKRLEWVATISSGGAYTFYKD
SVKGRFTISRDNAKNTLYLQMSSLRSEDSAMYYCARHSGYDGYYLYAMDYWGQGTSV
TVSSGGGGSGGGGSGGGGSQIVLTQSPEIMSASPGEKVTMTCSASSSVHYMHWYQQ
KSGTSPKRWIYDTSKLASGVPGRFSGSGSGTSYSLTISSMESEDAATYYCQQWTSNPP
TFGGGTKLEIKAAATTTPAPRPPTPAPTIASQPLSLRPEACRPAAGGAVHTRGLDFACDI
YIWAPLAGTCGVLLLSLVITLYCRVKFSRSADAPAYKQGQNQLYNELNLGRREEYDVLD
KRRGRDPEMGGKPRRKNPQEGLYNELQKDKMAEAYSEIGMKGERRRGKGHDGLYQ
GLSTATKDTYDALHMQALPPR.
Therefore, in some embodiments, the dual CAR fusion protein is encoded by the
nucleic acid sequence:
(SEQ ID NO: 139)
CCATGGCTTTACCCGTTACCGCTCTGCTGCTGCCTCTGGCTCTGCTGCTCCACGCT
GCTAGGCCCGACATCGTGATGACACAAGCTGCCCCCTCCGTGCCCGTTACACCCG
GTGAGAGCGTCAGCATCTCTTGTGGCTCCAGCAAGTCTTTACTGCACAGCAACGGC
AACACCTATTTATACTGGTTTCTGCAGAGGCCCGGCCAGAGCCCTCAGCTGCTGAT
TTACAGAGTCAGCAATTTAGCCAGCGGCGTGCCCGATAGATTCTCCGGCAGCGGC
AGCGGCACAGCCTTCACTTTAAGGATCTCTCGTGTCGAGGCCGAAGACGTGGGAG
TGTACTATTGCATGCAGCATTTAGAGTACCCTTACACCTTCGGCGGAGGCACCAGA
CTGGAGATCAAGGGCGGCGGCGGCAGCGGCGGTGGCGGCAGCGGAGGAGGCGG
TAGCGAGGTGCAGCTGGAAGAGTCCGGCGGAGGTTTAGTGCAGCCCAAAGGATCT
TTAAAGCTGAGCTGCGCCGCCAGCGGCTTCACCTTCAACACCTACGCCATGTACTG
GGTGTGCCAAGCTCCCGGTAAGGGTTTAAAGTGGGTGGCTCGTATTCGTTCCAAG
GTGAACAACTACGCCACCTACTACGCCGATTCCGTCAAGGATCGTTTCACCATCTC
TCGTGACGACAGCCAGAACATGCTGTTTTTACACATGAACAATTTAAAGACCGAGG
ACACCGCCATCTATTTCTGCGTGAGGGACCCTATGGATTACTGGGGACAAGGTATC
AGCGTGACAGTGTCCTCCGCGGCCGCCACAACAACCCCCGCTCCTAGACCTCCTA
CCCCCGCCCCCACCATCGCCAGCCAACCTCTGTCTCTGAGACCCGAAGCTTGTAG
ACCCGCCGCTGGCGGAGCCGTGCATACAAGAGGACTGGACTTTGCTTGCGACATC
TACATTTGGGCTCCCCTCGCCGGAACATGTGGCGTGCTGCTGCTGTCTCTGGTGAT
CACACTCTATTGCAAGAGGGGAAGAAAAAAGCTGCTGTACATCTTTAAGCAGCCCT
TCATGAGGCCCGTCCAGACCACACAAGAAGAGGATGGCTGCAGCTGCAGATTCCC
CGAGGAAGAAGAGGGCGGATGTGAGCTGAGGTCCAAAAGATCTAGACTGCTGCAC
AGCGACTTCATGAACATGACCGCTAGAAGGGCCGGACCTACAAGGAAACATTACCA
GCCTTACGCCCCCCCTAGAGACTTTGCCGCCTACAGATCCGGATCCGGAGCCACC
AACTTTTCTCTGCTGAAGCAAGCCGGAGATGTGGAAGAGAATCCCGGCCCTATGGC
CCTCCCCGTGACAGCTCTGCTGCTGCCTCTGGCTCTGCTGCTCCATGCCGCTAGG
CCCGAGCTGATTCTGGTCGAAAGCGGCGGAGGACTGGTCAAGCCCGGAGGATCTC
TGAAACTGAGCTGCGCCGTGAGCGGCTTCACCTTCTCCAGCTTTGCTATGAGCTGG
GTGAGACAGACACCCGAAAAGAGGCTGGAATGGGTGGCCACAATTTCCAGCGGCG
GAGCCTACACCTTTTACAAGGATTCCGTGAAGGGAAGATTCACCATCTCTAGAGAC
AATGCTAAAAACACACTGTATCTGCAGATGTCCTCTCTGAGGTCCGAAGACTCCGC
CATGTATTACTGCGCCAGACACAGCGGCTACGACGGCTACTATCTGTACGCTATGG
ACTACTGGGGCCAAGGCACAAGCGTCACAGTCAGCTCCGGAGGCGGAGGCAGCG
GAGGAGGAGGAAGCGGAGGCGGAGGCAGCCAGATTGTGCTGACCCAGAGCCCCG
AAATCATGTCCGCTTCCCCCGGCGAGAAAGTCACAATGACATGTTCCGCCTCCAGC
AGCGTCCACTACATGCACTGGTACCAACAGAAGAGCGGCACCAGCCCCAAGAGGT
GGATTTACGACACCTCCAAACTCGCCTCCGGCGTGCCCGGAAGATTCAGCGGAAG
CGGCTCCGGCACCAGCTACTCCCTCACAATCTCCAGCATGGAGAGCGAAGATGCC
GCTACCTACTACTGCCAGCAATGGACCTCCAACCCCCCTACCTTCGGAGGCGGCA
CCAAACTGGAGATTAAAGCCGCCGCTACCACCACCCCCGCCCCTAGGCCCCCTAC
ACCCGCCCCCACAATCGCTTCCCAACCTCTGTCTCTGAGACCCGAGGCTTGTAGAC
CCGCCGCTGGAGGAGCCGTCCATACAAGAGGCCTCGACTTTGCTTGCGACATTTA
CATCTGGGCCCCTCTGGCTGGCACATGCGGAGTGCTGCTGCTGTCTCTGGTCATC
ACACTGTACTGCAGAGTGAAGTTCAGCAGATCCGCCGATGCCCCCGCTTATAAGCA
AGGCCAGAACCAGCTGTACAACGAGCTGAATCTGGGAAGGAGGGAGGAGTATGAC
GTGCTGGATAAAAGGAGAGGAAGAGATCCCGAGATGGGCGGCAAGCCTAGAAGAA
AAAACCCCCAAGAGGGCCTCTACAACGAACTGCAGAAGGACAAGATGGCCGAGGC
CTACTCCGAGATCGGCATGAAGGGCGAAAGGAGAAGAGGCAAGGGACACGATGG
CCTCTACCAAGGCCTCTCCACCGCCACAAAGGACACATACGACGCCCTCCATATGC
AAGCTCTGCCCCCCAGATAACTCGAGATCCGGATTAGTCCAATTTGTTAAAGACAG
GATATCAGTGGTCCAGGCTCTAGTTTTGACTCAACAATATCACCAGCTGAAGCCTAT
AGAGTACGAGCCATAGATAAAATAAAAGATTTTATTTAGTCTCCAGAAAAAGGGGGG
AATGAAAGACCCCACCTGTAGGTTTGGCAAGCTAGCTTAAGTAACGCCATTTTGCA
AGGCATGGAAAAATACATAACTGAGAATAGAGAAGTTCAGATCAAGGTCAGGAACA
GATGGAACAGCTGAATATGGGCCAAACAGGATATCTGTGGTAAGCAGTTCCTGCCC
CGGCTCAGGGCCAAGAACAGATGGAACAGCTGAATATGGGCCAAACAGGATATCT
GTGGTAAGCAGTTCCTGCCCCGGCTCAGGGCCAAGAACAGATGGTCCCCAGATGC
GGTCCAGCCCTCAGCAGTTTCTAGAGAACCATCAGATGTTTCCAGGGTGCCCCAAG
GACCTGAAATGACCCTGTGCCTTATTTGAACTAACCAATCAGTTCGCTTCTCGCTTC
TGTTCGCGCGCTTCTGCTCCCCGAGCTCAATAAAAGAGCCCACAACCCCTCACTCG
GGGCGCCAGTCCTCCGATTGACTGAGTCGCCCGGGTACCCGTGTATCCAATAAAC
CCTCTTGCAGTTGCATCCGACTTGTGGTCTCGCTGTTCCTTGGGAGGGTCTCCTCT
GAGTGATTGACTACCCGTCAGCGGGGGTCTTTCACACATGCAGCATGTATCAAAAT
TAATTTGGTTTTTTTTCTTAAGTATTTACATTAAATGGCCATAGTACTTAAAGTTACAT
TGGCTTCCTTGAAATAAACATGGAGTATTCAGAATGTGTCATAAATATTTCTAATTTT
AAGATAGTATCTCCATTGGCTTTCTACTTTTTCTTTTATTTTTTTTTGTCCTCTGTCTT
CCATTTGTTGTTGTTGTTGTTTGTTTGTTTGTTTGTTGGTTGGTTGGTTAATTTTTTTT
TAAAGATCCTACACTATAGTTCAAGCTAGACTATTAGCTACTCTGTAACCCAGGGTG
ACCTTGAAGTCATGGGTAGCCTGCTGTTTTAGCCTTCCCACATCTAAGATTACAGGT
ATGAGCTATCATTTTTGGTATATTGATTGATTGATTGATTGATGTGTGTGTGTGTGAT
TGTGTTTGTGTGTGTGACTGTGAAAATGTGTGTATGGGTGTGTGTGAATGTGTGTAT
GTATGTGTGTGTGTGAGTGTGTGTGTGTGTGTGTGCATGTGTGTGTGTGTGACTGT
GTCTATGTGTATGACTGTGTGTGTGTGTGTGTGTGTGTGTGTGTGTGTGTGTGTGT
GTGTGTTGTGAAAAAATATTCTATGGTAGTGAGAGCCAACGCTCCGGCTCAGGTGT
CAGGTTGGTTTTTGAGACAGAGTCTTTCACTTAGCTTGGAATTCACTGGCCGTCGTT
TTACAACGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATCGCCCT
TCCCAACAGTTGCGCAGCCTGAATGGCGAATGGCGCCTGATGCGGTATTTTCTCCT
TACGCATCTGTGCGGTATTTCACACCGCATATGGTGCACTCTCAGTACAATCTGCTC
TGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGATGACGAA
AGGGCCTCGTGATACGCCTATTTTTATAGGTTAATGTCATGATAATAATGGTTTCTTA
GACGTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGA
GCAACTCGGTCGCCGCATACACTATTCTCAGAATGACTTGGTTGAGTACTCACCAG
TCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACC
GAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCA
CTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCC
CGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTTCC
ACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGC
GCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCATTGAGAAAGCGC
CACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAACCGCC
TCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCA
CCCCAGGCTTTACACTTTATGCTTCCGGCTCGTATGTTGTGTGGAATTGTGAGCGG
ATAACAATTTCACACAGGAAACAGCTATGACCATGATTACGCCAAGCTTTGCTCTTA
GGAGTTTCCTAATACATCCCAAACTCAAATATATAAAGCATTTGACTTGTTCTATGCC
CTAGGGGGCGGGGGGAAGCTAAGCCAGCTTTTTTTAACATTTAAAATGTTAATTCCA
TTTTAAATGCACAGATGTTTTTATTTCATAAGGGTTTCAATGTGCATGAATGCTGCAA
TATTCCTGTTACCAAAGCTAGTATAAATAAAAATAGATAAACGTGGAAATTACTTAGA
GTTTCTGTCATTAACGTTTCCTTCCTCAGTTGACAACATAAATGCGCTGCTGAGCAA
GCCAGTTTGCATCTGTCAGGATCAATTTCCCATTATGCCAGTCATATTAATTACTAGT
CAATTAGTTGATTTTTATTTTTGACATATACATGTGAATGAAAGACCCCACCTGTAGG
TTTGGCAAGCTAGCTTAAGTAACGCCATTTTGCAAGGCATGGAAAAATACATAACTG
AGAATAGAAAAGTTCAGATCAAGGTCAGGAACAGATGGAACAGCTGAATATGGGCC
AAACAGGATATCTGTGGTAAGCAGTTCCTGCCCCGGCTCAGGGCCAAGAACAGAT
GGAACAGCTGAATATGGGCCAAACAGGATATCTGTGGTAAGCAGTTCCTGCCCCG
GCTCAGGGCCAAGAACAGATGGTCCCCAGATGCGGTCCAGCCCTCAGCAGTTTCT
AGAGAACCATCAGATGTTTCCAGGGTGCCCCAAGGACCTGAAATGACCCTGTGCCT
TATTTGAACTAACCAATCAGTTCGCTTCTCGCTTCTGTTCGCGCGCTTATGCTCCCC
GAGCTCAATAAAAGAGCCCACAACCCCTCACTCGGGGCGCCAGTCCTCCGATTGA
CTGAGTCGCCCGGGTACCCGTGTATCCAATAAACCCTCTTGCAGTTGCATCCGACT
TGTGGTCTCGCTGTTCCTTGGGAGGGTCTCCTCTGAGTGATTGACTACCCGTCAGC
GGGGGTCTTTCATTTGGGGGCTCGTCCGGGATCGGGAGACCCCTGCCCAGGGAC
CACCGACCCACCACCGGGAGGTAAGCTGGCCAGCAACTTATCTGTGTCTGTCCGA
TTGTCTAGTGTCTATGACTGATTTTATGCGCCTGCGTCGGTACTAGTTAGCTAACTA
GCTCTGTATCTGGCGGACCCGTGGTGGAACTGACGAGTTCGGAACACCCGGCCGC
AACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATA
TGTGGTTCTGGTAGGAGACGAGAACCTAAAACAGTTCCCGCCTCCGTCTGAATTTT
TGCTTTCGGTTTGGGACCGAAGCCGCGCCGCGCGTCTTGTCTGCTGCAGCATCGT
TCTGTGTTGTCTCTGTCTGACTGTGTTTCTGTATTTGTCTGAAAATATGGGCCCGGG
CTAGACTGTTACCACTCCCTTAAGTTTGACCTTAGGTCACTGGAAAGATGTCGAGC
GGATCGCTCACAACCAGTCGGTAGATGTCAAGAAGAGACGTTGGGTTACCTTCTGC
TCTGCAGAATGGCCAACCTTTAACGTCGGATGGCCGCGAGACGGCACCTTTAACC
GAGACCTCATCACCCAGGTTAAGATCAAGGTCTTTTCACCTGGCCCGCATGGACAC
CCAGACCAGGTCCCCTACATCGTGACCTGGGAAGCCTTGGCTTTTGACCCCCCTC
CCTGGGTCAAGCCCTTTGTACACCCTAAGCCTCCGCCTCCTCTTCCTCCATCCGCC
CCGTCTCTCCCCCTTGAACCTCCTCGTTCGACCCCGCCTCGATCCTCCCTTTATCC
AGCCCTCACTCCTTCTCTAGGCGCCCCCATATGGCCATATGAGATCTTATATGGGG
CACCCCCGCCCCTTGTAAACTTCCCTGACCCTGACATGACAAGAGTTACTAACAGC
CCCTCTCTCCAAGCTCACTTACAGGCTCTCTACTTAGTCCAGCACGAAGTCTGGAG
ACCTCTGGCGGCAGCCTACCAAGAACAACTGGACCGACCGGTGGTACCTCACCCT
TACCGAGTCGGCGACACAGTGTGGGTCCGCCGACACCAGACTAAGAACCTAGAAC
CTCGCTGGAAAGGACCTTACACAGTCCTGCTGACCACCCCCACCGCCCTCAAAGTA
GACGGCATCGCAGCTTGGATACACGCCGCCCA.
As an example, in some embodiments, the dual CAR fusion protein has the
amino acid sequence:
(SEQ ID NO: 140)
MALPVTALLLPLALLLHAARPDIVMTQAAPSVPVTPGESVSISCGSSKSLLHSNGNTYLY
WFLQRPGQSPQLLIYRVSNLASGVPDRFSGSGSGTAFTLRISRVEAEDVGVYYCMQHL
EYPYTFGGGTRLEIKGGGGSGGGGSGGGGSEVQLVESGGGLVQPKGSLKLSCAASG
FTFNTYAMYWVCQAPGKGLKWVARIRSKVNNYATYYADSVKDRFTISRDDSQNMLFLH
MNNLKTEDTAIYFCVRDPMDYWGQGISVTVSSAAATTTPAPRPPTPAPTIASQPLSLRP
EACRPAAGGAVHTRGLDFACDIYIWAPLAGTCGVLLLSLVITLYCKRGRKKLLYIFKQPF
MRPVQTTQEEDGCSCRFPEEEEGGCELRSKRSRLLHSDFMNMTARRAGPTRKHYQP
YAPPRDFAAYRSGSGATNFSLLKQAGDVEENPGPMALPVTALLLPLALLLHAARPELILV
ESGGGLVKPGGSLKLSCAVSGFTFSSFAMSWWRQTPEKRLEWVATISSGGAYTFYKD
SVKGRFTISRDNAKNTLYLQMSSLRSEDSAMYYCARHSGYDGYYLYAMDYWGQGTSV
TVSSGGGGSGGGGSGGGGSQIVLTQSPEIMSASPGEKVTMTCSASSSVHYMHWYQQ
KSGTSPKRWIYDTSKLASGVPGRFSGSGSGTSYSLTISSMESEDAATYYCQQWTSNPP
TFGGGTKLEIKAAATTTPAPRPPTPAPTIASQPLSLRPEACRPAAGGAVHTRGLDFACDI
YIWAPLAGTCGVLLLSLVITLYCRVKFSRSADAPAYKQGQNQLYNELNLGRREEYDVLD
KRRGRDPEMGGKPRRKNPQEGLYNELQKDKMAEAYSEIGMKGERRRGKGHDGLYQ
GLSTATKDTYDALHMQALPPR.
Therefore, in some embodiments, the dual CAR fusion protein is encoded by the
nucleic acid sequence:
(SEQ ID NO: 141)
CCATGGCTCTGCCCGTTACCGCTTTACTGCTGCCTCTGGCTCTGCTGCTGCATGCC
GCTAGGCCCGACATCGTGATGACCCAAGCTGCTCCTTCCGTGCCCGTGACACCCG
GTGAGAGCGTCTCCATCTCTTGTGGCAGCAGCAAATCTTTACTGCACTCCAACGGC
AACACCTATCTGTATTGGTTTTTACAGAGGCCCGGTCAGTCCCCCCAGCTGCTGAT
CTATCGTGTGAGCAATCTCGCTAGCGGCGTGCCCGACAGATTTTCCGGCAGCGGC
AGCGGAACAGCCTTCACTTTAAGGATCTCCAGAGTGGAGGCCGAGGACGTGGGCG
TGTACTACTGCATGCAGCATTTAGAGTACCCCTACACCTTTGGAGGCGGCACTCGT
CTGGAAATTAAGGGCGGCGGCGGCTCCGGCGGAGGCGGCTCCGGAGGCGGAGG
AAGCGAAGTGCAGCTGGTGGAAAGCGGAGGCGGTTTAGTGCAGCCCAAAGGATCT
TTAAAACTGTCTTGTGCCGCCAGCGGCTTCACCTTCAACACCTACGCTATGTACTG
GGTGTGCCAAGCTCCCGGTAAGGGTTTAAAATGGGTGGCTCGTATTCGTAGCAAAG
TGAACAACTACGCCACCTACTACGCCGACAGCGTGAAAGATAGGTTCACCATCTCT
CGTGACGACTCCCAGAACATGCTGTTTTTACACATGAACAATCTCAAAACAGAGGA
CACCGCCATCTACTTCTGCGTGAGGGACCCTATGGATTACTGGGGCCAAGGTATTA
GCGTGACCGTGTCCAGCGCGGCCGCCACAACAACCCCCGCTCCTAGACCTCCTAC
CCCCGCCCCCACCATCGCCAGCCAACCTCTGTCTCTGAGACCCGAAGCTTGTAGA
CCCGCCGCTGGCGGAGCCGTGCATACAAGAGGACTGGACTTTGCTTGCGACATCT
ACATTTGGGCTCCCCTCGCCGGAACATGTGGCGTGCTGCTGCTGTCTCTGGTGAT
CACACTCTATTGCAAGAGGGGAAGAAAAAAGCTGCTGTACATCTTTAAGCAGCCCT
TCATGAGGCCCGTCCAGACCACACAAGAAGAGGATGGCTGCAGCTGCAGATTCCC
CGAGGAAGAAGAGGGCGGATGTGAGCTGAGGTCCAAAAGATCTAGACTGCTGCAC
AGCGACTTCATGAACATGACCGCTAGAAGGGCCGGACCTACAAGGAAACATTACCA
GCCTTACGCCCCCCCTAGAGACTTTGCCGCCTACAGATCCGGATCCGGAGCCACC
AACTTTTCTCTGCTGAAGCAAGCCGGAGATGTGGAAGAGAATCCCGGCCCTATGGC
CCTCCCCGTGACAGCTCTGCTGCTGCCTCTGGCTCTGCTGCTCCATGCCGCTAGG
CCCGAGCTGATTCTGGTCGAAAGCGGCGGAGGACTGGTCAAGCCCGGAGGATCTC
TGAAACTGAGCTGCGCCGTGAGCGGCTTCACCTTCTCCAGCTTTGCTATGAGCTGG
GTGAGACAGACACCCGAAAAGAGGCTGGAATGGGTGGCCACAATTTCCAGCGGCG
GAGCCTACACCTTTTACAAGGATTCCGTGAAGGGAAGATTCACCATCTCTAGAGAC
AATGCTAAAAACACACTGTATCTGCAGATGTCCTCTCTGAGGTCCGAAGACTCCGC
CATGTATTACTGCGCCAGACACAGCGGCTACGACGGCTACTATCTGTACGCTATGG
ACTACTGGGGCCAAGGCACAAGCGTCACAGTCAGCTCCGGAGGCGGAGGCAGCG
GAGGAGGAGGAAGCGGAGGCGGAGGCAGCCAGATTGTGCTGACCCAGAGCCCCG
AAATCATGTCCGCTTCCCCCGGCGAGAAAGTCACAATGACATGTTCCGCCTCCAGC
AGCGTCCACTACATGCACTGGTACCAACAGAAGAGCGGCACCAGCCCCAAGAGGT
GGATTTACGACACCTCCAAACTCGCCTCCGGCGTGCCCGGAAGATTCAGCGGAAG
CGGCTCCGGCACCAGCTACTCCCTCACAATCTCCAGCATGGAGAGCGAAGATGCC
GCTACCTACTACTGCCAGCAATGGACCTCCAACCCCCCTACCTTCGGAGGCGGCA
CCAAACTGGAGATTAAAGCCGCCGCTACCACCACCCCCGCCCCTAGGCCCCCTAC
ACCCGCCCCCACAATCGCTTCCCAACCTCTGTCTCTGAGACCCGAGGCTTGTAGAC
CCGCCGCTGGAGGAGCCGTCCATACAAGAGGCCTCGACTTTGCTTGCGACATTTA
CATCTGGGCCCCTCTGGCTGGCACATGCGGAGTGCTGCTGCTGTCTCTGGTCATC
ACACTGTACTGCAGAGTGAAGTTCAGCAGATCCGCCGATGCCCCCGCTTATAAGCA
AGGCCAGAACCAGCTGTACAACGAGCTGAATCTGGGAAGGAGGGAGGAGTATGAC
GTGCTGGATAAAAGGAGAGGAAGAGATCCCGAGATGGGCGGCAAGCCTAGAAGAA
AAAACCCCCAAGAGGGCCTCTACAACGAACTGCAGAAGGACAAGATGGCCGAGGC
CTACTCCGAGATCGGCATGAAGGGCGAAAGGAGAAGAGGCAAGGGACACGATGG
CCTCTACCAAGGCCTCTCCACCGCCACAAAGGACACATACGACGCCCTCCATATGC
AAGCTCTGCCCCCCAGATAACTCGAGATCCGGATTAGTCCAATTTGTTAAAGACAG
GATATCAGTGGTCCAGGCTCTAGTTTTGACTCAACAATATCACCAGCTGAAGCCTAT
AGAGTACGAGCCATAGATAAAATAAAAGATTTTATTTAGTCTCCAGAAAAAGGGGGG
AATGAAAGACCCCACCTGTAGGTTTGGCAAGCTAGCTTAAGTAACGCCATTTTGCA
AGGCATGGAAAAATACATAACTGAGAATAGAGAAGTTCAGATCAAGGTCAGGAACA
GATGGAACAGCTGAATATGGGCCAAACAGGATATCTGTGGTAAGCAGTTCCTGCCC
CGGCTCAGGGCCAAGAACAGATGGAACAGCTGAATATGGGCCAAACAGGATATCT
GTGGTAAGCAGTTCCTGCCCCGGCTCAGGGCCAAGAACAGATGGTCCCCAGATGC
GGTCCAGCCCTCAGCAGTTTCTAGAGAACCATCAGATGTTTCCAGGGTGCCCCAAG
GACCTGAAATGACCCTGTGCCTTATTTGAACTAACCAATCAGTTCGCTTCTCGCTTC
TGTTCGCGCGCTTCTGCTCCCCGAGCTCAATAAAAGAGCCCACAACCCCTCACTCG
GGGCGCCAGTCCTCCGATTGACTGAGTCGCCCGGGTACCCGTGTATCCAATAAAC
CCTCTTGCAGTTGCATCCGACTTGTGGTCTCGCTGTTCCTTGGGAGGGTCTCCTCT
GAGTGATTGACTACCCGTCAGCGGGGGTCTTTCACACATGCAGCATGTATCAAAAT
TAATTTGGTTTTTTTTCTTAAGTATTTACATTAAATGGCCATAGTACTTAAAGTTACAT
TGGCTTCCTTGAAATAAACATGGAGTATTCAGAATGTGTCATAAATATTTCTAATTTT
AAGATAGTATCTCCATTGGCTTTCTACTTTTTCTTTTATTTTTTTTTGTCCTCTGTCTT
CCATTTGTTGTTGTTGTTGTTTGTTTGTTTGTTTGTTGGTTGGTTGGTTAATTTTTTTT
TAAAGATCCTACACTATAGTTCAAGCTAGACTATTAGCTACTCTGTAACCCAGGGTG
ACCTTGAAGTCATGGGTAGCCTGCTGTTTTAGCCTTCCCACATCTAAGATTACAGGT
ATGAGCTATCATTTTTGGTATATTGATTGATTGATTGATTGATGTGTGTGTGTGTGAT
TGTGTTTGTGTGTGTGACTGTGAAAATGTGTGTATGGGTGTGTGTGAATGTGTGTAT
GTATGTGTGTGTGTGAGTGTGTGTGTGTGTGTGTGCATGTGTGTGTGTGTGACTGT
GTCTATGTGTATGACTGTGTGTGTGTGTGTGTGTGTGTGTGTGTGTGTGTGTGTGT
GTGTGTTGTGAAAAAATATTCTATGGTAGTGAGAGCCAACGCTCCGGCTCAGGTGT
CAGGTTGGTTTTTGAGACAGAGTCTTTCACTTAGCTTGGAATTCACTGGCCGTCGTT
TTACAACGTCGTGACTGGGAAAACCCTGGCGTTACCCAACTTAATCGCCTTGCAGC
ACATCCCCCTTTCGCCAGCTGGCGTAATAGCGAAGAGGCCCGCACCGATCGCCCT
TCCCAACAGTTGCGCAGCCTGAATGGCGAATGGCGCCTGATGCGGTATTTTCTCCT
TACGCATCTGTGCGGTATTTCACACCGCATATGGTGCACTCTCAGTACAATCTGCTC
TGATGCCGCATAGTTAAGCCAGCCCCGACACCCGCCAACACCCGCTGACGCGCCC
TGACGGGCTTGTCTGCTCCCGGCATCCGCTTACAGACAAGCTGTGACCGTCTCCG
GGAGCTGCATGTGTCAGAGGTTTTCACCGTCATCACCGAAACGCGCGATGACGAA
AGGGCCTCGTGATACGCCTATTTTTATAGGTTAATGTCATGATAATAATGGTTTCTTA
GACGTCAGGTGGCACTTTTCGGGGAAATGTGCGCGGAACCCCTATTTGTTTATTTT
TCTAAATACATTCAAATATGTATCCGCTCATGAGACAATAACCCTGATAAATGCTTCA
ATAATATTGAAAAAGGAAGAGTATGAGTATTCAACATTTCCGTGTCGCCCTTATTCC
CTTTTTTGCGGCATTTTGCCTTCCTGTTTTTGCTCACCCAGAAACGCTGGTGAAAGT
AAAAGATGCTGAAGATCAGTTGGGTGCACGAGTGGGTTACATCGAACTGGATCTCA
ACAGCGGTAAGATCCTTGAGAGTTTTCGCCCCGAAGAACGTTTTCCAATGATGAGC
ACTTTTAAAGTTCTGCTATGTGGCGCGGTATTATCCCGTATTGACGCCGGGCAAGA
GCAACTCGGTCGCCGCATACACTATTCTCAGAATGACTTGGTTGAGTACTCACCAG
TCACAGAAAAGCATCTTACGGATGGCATGACAGTAAGAGAATTATGCAGTGCTGCC
ATAACCATGAGTGATAACACTGCGGCCAACTTACTTCTGACAACGATCGGAGGACC
GAAGGAGCTAACCGCTTTTTTGCACAACATGGGGGATCATGTAACTCGCCTTGATC
GTTGGGAACCGGAGCTGAATGAAGCCATACCAAACGACGAGCGTGACACCACGAT
GCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCA
CTTCTGCGCTCGGCCCTTCCGGCTGGCTGGTTTATTGCTGATAAATCTGGAGCCGG
TGAGCGTGGGTCTCGCGGTATCATTGCAGCACTGGGGCCAGATGGTAAGCCCTCC
CGTATCGTAGTTATCTACACGACGGGGAGTCAGGCAACTATGGATGAACGAAATAG
ACAGATCGCTGAGATAGGTGCCTCACTGATTAAGCATTGGTAACTGTCAGACCAAG
TTTACTCATATATACTTTAGATTGATTTAAAACTTCATTTTTAATTTAAAAGGATCTAG
GTGAAGATCCTTTTTGATAATCTCATGACCAAAATCCCTTAACGTGAGTTTTCGTTCC
ACTGAGCGTCAGACCCCGTAGAAAAGATCAAAGGATCTTCTTGAGATCCTTTTTTTC
TGCGCGTAATCTGCTGCTTGCAAACAAAAAAACCACCGCTACCAGCGGTGGTTTGT
TTGCCGGATCAAGAGCTACCAACTCTTTTTCCGAAGGTAACTGGCTTCAGCAGAGC
GCAGATACCAAATACTGTCCTTCTAGTGTAGCCGTAGTTAGGCCACCACTTCAAGA
ACTCTGTAGCACCGCCTACATACCTCGCTCTGCTAATCCTGTTACCAGTGGCTGCT
GCCAGTGGCGATAAGTCGTGTCTTACCGGGTTGGACTCAAGACGATAGTTACCGG
ATAAGGCGCAGCGGTCGGGCTGAACGGGGGGTTCGTGCACACAGCCCAGCTTGG
AGCGAACGACCTACACCGAACTGAGATACCTACAGCGTGAGCATTGAGAAAGCGC
CACGCTTCCCGAAGGGAGAAAGGCGGACAGGTATCCGGTAAGCGGCAGGGTCGG
AACAGGAGAGCGCACGAGGGAGCTTCCAGGGGGAAACGCCTGGTATCTTTATAGT
CCTGTCGGGTTTCGCCACCTCTGACTTGAGCGTCGATTTTTGTGATGCTCGTCAGG
GGGGCGGAGCCTATGGAAAAACGCCAGCAACGCGGCCTTTTTACGGTTCCTGGCC
TTTTGCTGGCCTTTTGCTCACATGTTCTTTCCTGCGTTATCCCCTGATTCTGTGGATA
ACCGTATTACCGCCTTTGAGTGAGCTGATACCGCTCGCCGCAGCCGAACGACCGA
GCGCAGCGAGTCAGTGAGCGAGGAAGCGGAAGAGCGCCCAATACGCAAACCGCC
TCTCCCCGCGCGTTGGCCGATTCATTAATGCAGCTGGCACGACAGGTTTCCCGACT
GGAAAGCGGGCAGTGAGCGCAACGCAATTAATGTGAGTTAGCTCACTCATTAGGCA
CCCCAGGCTTTACACTTTATGCTTCCGGCTCGTATGTTGTGTGGAATTGTGAGCGG
ATAACAATTTCACACAGGAAACAGCTATGACCATGATTACGCCAAGCTTTGCTCTTA
GGAGTTTCCTAATACATCCCAAACTCAAATATATAAAGCATTTGACTTGTTCTATGCC
CTAGGGGGCGGGGGGAAGCTAAGCCAGCTTTTTTTAACATTTAAAATGTTAATTCCA
TTTTAAATGCACAGATGTTTTTATTTCATAAGGGTTTCAATGTGCATGAATGCTGCAA
TATTCCTGTTACCAAAGCTAGTATAAATAAAAATAGATAAACGTGGAAATTACTTAGA
GTTTCTGTCATTAACGTTTCCTTCCTCAGTTGACAACATAAATGCGCTGCTGAGCAA
GCCAGTTTGCATCTGTCAGGATCAATTTCCCATTATGCCAGTCATATTAATTACTAGT
CAATTAGTTGATTTTTATTTTTGACATATACATGTGAATGAAAGACCCCACCTGTAGG
TTTGGCAAGCTAGCTTAAGTAACGCCATTTTGCAAGGCATGGAAAAATACATAACTG
AGAATAGAAAAGTTCAGATCAAGGTCAGGAACAGATGGAACAGCTGAATATGGGCC
AAACAGGATATCTGTGGTAAGCAGTTCCTGCCCCGGCTCAGGGCCAAGAACAGAT
GGAACAGCTGAATATGGGCCAAACAGGATATCTGTGGTAAGCAGTTCCTGCCCCG
GCTCAGGGCCAAGAACAGATGGTCCCCAGATGCGGTCCAGCCCTCAGCAGTTTCT
AGAGAACCATCAGATGTTTCCAGGGTGCCCCAAGGACCTGAAATGACCCTGTGCCT
TATTTGAACTAACCAATCAGTTCGCTTCTCGCTTCTGTTCGCGCGCTTATGCTCCCC
GAGCTCAATAAAAGAGCCCACAACCCCTCACTCGGGGCGCCAGTCCTCCGATTGA
CTGAGTCGCCCGGGTACCCGTGTATCCAATAAACCCTCTTGCAGTTGCATCCGACT
TGTGGTCTCGCTGTTCCTTGGGAGGGTCTCCTCTGAGTGATTGACTACCCGTCAGC
GGGGGTCTTTCATTTGGGGGCTCGTCCGGGATCGGGAGACCCCTGCCCAGGGAC
CACCGACCCACCACCGGGAGGTAAGCTGGCCAGCAACTTATCTGTGTCTGTCCGA
TTGTCTAGTGTCTATGACTGATTTTATGCGCCTGCGTCGGTACTAGTTAGCTAACTA
GCTCTGTATCTGGCGGACCCGTGGTGGAACTGACGAGTTCGGAACACCCGGCCGC
AACCCTGGGAGACGTCCCAGGGACTTCGGGGGCCGTTTTTGTGGCCCGACCTGAG
TCCTAAAATCCCGATCGTTTAGGACTCTTTGGTGCACCCCCCTTAGAGGAGGGATA
TGTGGTTCTGGTAGGAGACGAGAACCTAAAACAGTTCCCGCCTCCGTCTGAATTTT
TGCTTTCGGTTTGGGACCGAAGCCGCGCCGCGCGTCTTGTCTGCTGCAGCATCGT
TCTGTGTTGTCTCTGTCTGACTGTGTTTCTGTATTTGTCTGAAAATATGGGCCCGGG
CTAGACTGTTACCACTCCCTTAAGTTTGACCTTAGGTCACTGGAAAGATGTCGAGC
GGATCGCTCACAACCAGTCGGTAGATGTCAAGAAGAGACGTTGGGTTACCTTCTGC
TCTGCAGAATGGCCAACCTTTAACGTCGGATGGCCGCGAGACGGCACCTTTAACC
GAGACCTCATCACCCAGGTTAAGATCAAGGTCTTTTCACCTGGCCCGCATGGACAC
CCAGACCAGGTCCCCTACATCGTGACCTGGGAAGCCTTGGCTTTTGACCCCCCTC
CCTGGGTCAAGCCCTTTGTACACCCTAAGCCTCCGCCTCCTCTTCCTCCATCCGCC
CCGTCTCTCCCCCTTGAACCTCCTCGTTCGACCCCGCCTCGATCCTCCCTTTATCC
AGCCCTCACTCCTTCTCTAGGCGCCCCCATATGGCCATATGAGATCTTATATGGGG
CACCCCCGCCCCTTGTAAACTTCCCTGACCCTGACATGACAAGAGTTACTAACAGO
CCCTCTCTCCAAGCTCACTTACAGGCTCTCTACTTAGTCCAGCACGAAGTCTGGAG
ACCTCTGGCGGCAGCCTACCAAGAACAACTGGACCGACCGGTGGTACCTCACCCT
TACCGAGTCGGCGACACAGTGTGGGTCCGCCGACACCAGACTAAGAACCTAGAAC
CTCGCTGGAAAGGACCTTACACAGTCCTGCTGACCACCCCCACCGCCCTCAAAGTA
GACGGCATCGCAGCTTGGATACACGCCGCCCA.

Tables 1, 2, and 3 below provide some example combinations of CD99-binding region or CLEC12A-binding region (scFv), co-stimulatory signaling regions, and intracellular signaling domain that can occur in the disclosed CARs.

TABLE 1
First Generation CARs

| ScFv | Signal Domain |
|---|---|
| ScFv | CD8 |
| ScFv | CD3ζ |
| ScFv | CD3δ |
| ScFv | CD3γ |
| ScFv | CD3ε |
| ScFv | FcγRI-γ |
| ScFv | FcγRIII-γ |
| ScFv | FcεRIβ |
| ScFv | FcεRIγ |
| ScFv | DAP10 |
| ScFv | DAP12 |
| ScFv | CD32 |
| ScFv | CD79a |

TABLE 2
Second Generation CARs

| | Co-stimulatory | Signal |
|---|---|---|
| ScFv | Signal | Domain |
| ScFv | CD28 | CD8 |
| ScFv | CD28 | CD3ζ |
| ScFv | CD28 | CD3δ |
| ScFv | CD28 | CD3γ |
| ScFv | CD28 | CD3ε |
| ScFv | CD28 | FcγRI-γ |
| ScFv | CD28 | FcγRIII-γ |
| ScFv | CD28 | FcεRIβ |
| ScFv | CD28 | FcεRIγ |
| ScFv | CD28 | DAP10 |
| ScFv | CD28 | DAP12 |
| ScFv | CD28 | CD32 |
| ScFv | CD28 | CD79a |
| ScFv | CD28 | CD79b |
| ScFv | CD8 | CD8 |
| ScFv | CD8 | CD3ζ |
| ScFv | CD8 | CD3δ |
| ScFv | CD8 | CD3γ |
| ScFv | CD8 | CD3ε |
| ScFv | CD8 | FcγRI-γ |
| ScFv | CD8 | FcγRIII-γ |
| ScFv | CD8 | FcεRIβ |
| ScFv | CD8 | FcεRIγ |
| ScFv | CD8 | DAP10 |
| ScFv | CD8 | DAP12 |
| ScFv | CD8 | CD32 |
| ScFv | CD8 | CD79a |
| ScFv | CD8 | CD79b |
| ScFv | CD4 | CD8 |
| ScFv | CD4 | CD3ζ |
| ScFv | CD4 | CD3δ |
| ScFv | CD4 | CD3γ |
| ScFv | CD4 | CD3ε |
| ScFv | CD4 | FcγRI-γ |
| ScFv | CD4 | FcγRIII-γ |
| ScFv | CD4 | FcεRIβ |
| ScFv | CD4 | FcεRIγ |
| ScFv | CD4 | DAP10 |
| ScFv | CD4 | DAP12 |
| ScFv | CD4 | CD32 |
| ScFv | CD4 | CD79a |
| ScFv | CD4 | CD79b |
| ScFv | b2c | CD8 |
| ScFv | b2c | CD3ζ |
| ScFv | b2c | CD3δ |
| ScFv | b2c | CD3γ |
| ScFv | b2c | CD3ε |
| ScFv | b2c | FcγRI-γ |
| ScFv | b2c | FcγRIII-γ |
| ScFv | b2c | FcεRIβ |
| ScFv | b2c | FcεRIγ |
| ScFv | b2c | DAP10 |
| ScFv | b2c | DAP12 |
| ScFv | b2c | CD32 |
| ScFv | b2c | CD79a |
| ScFv | b2c | CD79b |
| ScFv | CD137/41BB | CD8 |
| ScFv | CD137/41BB | CD3ζ |
| ScFv | CD137/41BB | CD3δ |
| ScFv | CD137/41BB | CD3γ |
| ScFv | CD137/41BB | CD3ε |
| ScFv | CD137/41BB | FcγRI-γ |
| ScFv | CD137/41BB | FcγRIII-γ |
| ScFv | CD137/41BB | FcεRIβ |
| ScFv | CD137/41BB | FcεRIγ |
| ScFv | CD137/41BB | DAP10 |
| ScFv | CD137/41BB | DAP12 |
| ScFv | CD137/41BB | CD32 |
| ScFv | CD137/41BB | CD79a |
| ScFv | CD137/41BB | CD79b |
| ScFv | ICOS | CD8 |
| ScFv | ICOS | CD3ζ |
| ScFv | ICOS | CD3δ |
| ScFv | ICOS | CD3γ |
| ScFv | ICOS | CD3ε |
| ScFv | ICOS | FcγRI-γ |
| ScFv | ICOS | FcγRIII-γ |
| ScFv | ICOS | FcεRIβ |
| ScFv | ICOS | FcεRIγ |
| ScFv | ICOS | DAP10 |
| ScFv | ICOS | DAP12 |
| ScFv | ICOS | CD32 |
| ScFv | ICOS | CD79a |
| ScFv | ICOS | CD79b |
| ScFv | CD27 | CD8 |
| ScFv | CD27 | CD3ζ |
| ScFv | CD27 | CD3δ |
| ScFv | CD27 | CD3γ |
| ScFv | CD27 | CD3ε |
| ScFv | CD27 | FcγRI-γ |
| ScFv | CD27 | FcγRIII-γ |
| ScFv | CD27 | FcεRIβ |
| ScFv | CD27 | FcεRIγ |
| ScFv | CD27 | DAP10 |
| ScFv | CD27 | DAP12 |
| ScFv | CD27 | CD32 |
| ScFv | CD27 | CD79a |
| ScFv | CD27 | CD79b |
| ScFv | CD28δ | CD8 |
| ScFv | CD28δ | CD3ζ |
| ScFv | CD28δ | CD3δ |
| ScFv | CD28δ | CD3γ |
| ScFv | CD28δ | CD3ε |
| ScFv | CD28δ | FcγRI-γ |
| ScFv | CD28δ | FcγRIII-γ |
| ScFv | CD28δ | FcεRIβ |
| ScFv | CD28δ | FcεRIγ |
| ScFv | CD28δ | DAP10 |
| ScFv | CD28δ | DAP12 |
| ScFv | CD28δ | CD32 |
| ScFv | CD28δ | CD79a |
| ScFv | CD28δ | CD79b |
| ScFv | CD80 | CD8 |
| ScFv | CD80 | CD3ζ |
| ScFv | CD80 | CD3δ |
| ScFv | CD80 | CD3γ |
| ScFv | CD80 | CD3ε |
| ScFv | CD80 | FcγRI-γ |
| ScFv | CD80 | FcγRIII-γ |
| ScFv | CD80 | FcεRIβ |
| ScFv | CD80 | FcεRIγ |
| ScFv | CD80 | DAP10 |
| ScFv | CD80 | DAP12 |
| ScFv | CD80 | CD32 |
| ScFv | CD80 | CD79a |
| ScFv | CD80 | CD79b |
| ScFv | CD86 | CD8 |
| ScFv | CD86 | CD3ζ |
| ScFv | CD86 | CD3δ |
| ScFv | CD86 | CD3γ |
| ScFv | CD86 | CD3ε |
| ScFv | CD86 | FcγRI-γ |
| ScFv | CD86 | FcγRIII-γ |
| ScFv | CD86 | FcεRIβ |
| ScFv | CD86 | FcεRIγ |
| ScFv | CD86 | DAP10 |
| ScFv | CD86 | DAP12 |
| ScFv | CD86 | CD32 |
| ScFv | CD86 | CD79a |
| ScFv | CD86 | CD79b |
| ScFv | OX40 | CD8 |
| ScFv | OX40 | CD3ζ |
| ScFv | OX40 | CD3δ |
| ScFv | OX40 | CD3γ |
| ScFv | OX40 | CD3ε |
| ScFv | OX40 | FcγRI-γ |
| ScFv | OX40 | FcγRIII-γ |
| ScFv | OX40 | FcεRIβ |
| ScFv | OX40 | FcεRIγ |
| ScFv | OX40 | DAP10 |
| ScFv | OX40 | DAP12 |
| ScFv | OX40 | CD32 |
| ScFv | OX40 | CD79a |
| ScFv | OX40 | CD79b |
| ScFv | DAP10 | CD8 |
| ScFv | DAP10 | CD3ζ |
| ScFv | DAP10 | CD3δ |
| ScFv | DAP10 | CD3γ |
| ScFv | DAP10 | CD3ε |
| ScFv | DAP10 | FcγRI-γ |
| ScFv | DAP10 | FcγRIII-γ |
| ScFv | DAP10 | FcεRIβ |
| ScFv | DAP10 | FcεRIγ |
| ScFv | DAP10 | DAP10 |
| ScFv | DAP10 | DAP12 |
| ScFv | DAP10 | CD32 |
| ScFv | DAP10 | CD79a |
| ScFv | DAP10 | CD79b |
| ScFv | DAP12 | CD8 |
| ScFv | DAP12 | CD3ζ |
| ScFv | DAP12 | CD3δ |
| ScFv | DAP12 | CD3γ |
| ScFv | DAP12 | CD3ε |
| ScFv | DAP12 | FcγRI-γ |
| ScFv | DAP12 | FcγRIII-γ |
| ScFv | DAP12 | FcεRIβ |
| ScFv | DAP12 | FcεRIγ |
| ScFv | DAP12 | DAP10 |
| ScFv | DAP12 | DAP12 |
| ScFv | DAP12 | CD32 |
| ScFv | DAP12 | CD79a |
| ScFv | DAP12 | CD79b |
| ScFv | MyD88 | CD8 |
| ScFv | MyD88 | CD3ζ |
| ScFv | MyD88 | CD3δ |
| ScFv | MyD88 | CD3γ |
| ScFv | MyD88 | CD3ε |
| ScFv | MyD88 | FcγRI-γ |
| ScFv | MyD88 | FcγRIII-γ |
| ScFv | MyD88 | FcεRIβ |
| ScFv | MyD88 | FcεRIγ |
| ScFv | MyD88 | DAP10 |
| ScFv | MyD88 | DAP12 |
| ScFv | MyD88 | CD32 |
| ScFv | MyD88 | CD79a |
| ScFv | MyD88 | CD79b |
| ScFv | CD7 | CD8 |
| ScFv | CD7 | CD3ζ |
| ScFv | CD7 | CD3δ |
| ScFv | CD7 | CD3γ |
| ScFv | CD7 | CD3ε |
| ScFv | CD7 | FcγRI-γ |
| ScFv | CD7 | FcγRIII-γ |
| ScFv | CD7 | FcεRIβ |
| ScFv | CD7 | FcεRIγ |
| ScFv | CD7 | DAP10 |
| ScFv | CD7 | DAP12 |
| ScFv | CD7 | CD32 |
| ScFv | CD7 | CD79a |
| ScFv | CD7 | CD79b |
| ScFv | BTNL3 | CD8 |
| ScFv | BTNL3 | CD3ζ |
| ScFv | BTNL3 | CD3δ |
| ScFv | BTNL3 | CD3γ |
| ScFv | BTNL3 | CD3ε |
| ScFv | BTNL3 | FcγRI-γ |
| ScFv | BTNL3 | FcγRIII-γ |
| ScFv | BTNL3 | FcεRIβ |
| ScFv | BTNL3 | FcεRIγ |
| ScFv | BTNL3 | DAP10 |
| ScFv | BTNL3 | DAP12 |
| ScFv | BTNL3 | CD32 |
| ScFv | BTNL3 | CD79a |
| ScFv | BTNL3 | CD79b |
| ScFv | NKG2D | CD8 |
| ScFv | NKG2D | CD3ζ |
| ScFv | NKG2D | CD3δ |
| ScFv | NKG2D | CD3γ |
| ScFv | NKG2D | CD3ε |
| ScFv | NKG2D | FcγRI-γ |
| ScFv | NKG2D | FcγRIII-γ |
| ScFv | NKG2D | FcεRIβ |
| ScFv | NKG2D | FcεRIγ |
| ScFv | NKG2D | DAP10 |
| ScFv | NKG2D | DAP12 |
| ScFv | NKG2D | CD32 |
| ScFv | NKG2D | CD79a |
| ScFv | NKG2D | CD79b |

TABLE 3
Third Generation CARs

| | Co-stimulatory | Co-stimulatory | Signal |
|---|---|---|---|
| ScFv | Signal | Signal | Domain |
| ScFv | CD28 | CD28 | CD8 |
| ScFv | CD28 | CD28 | CD3ζ |
| ScFv | CD28 | CD28 | CD3δ |
| ScFv | CD28 | CD28 | CD3γ |
| ScFv | CD28 | CD28 | CD3ε |
| ScFv | CD28 | CD28 | FcγRI-γ |
| ScFv | CD28 | CD28 | FcγRIII-γ |
| ScFv | CD28 | CD28 | FcεRIβ |
| ScFv | CD28 | CD28 | FcεRIγ |
| ScFv | CD28 | CD28 | DAP10 |
| ScFv | CD28 | CD28 | DAP12 |
| ScFv | CD28 | CD28 | CD32 |
| ScFv | CD28 | CD28 | CD79a |
| ScFv | CD28 | CD28 | CD79b |
| ScFv | CD28 | CD8 | CD8 |
| ScFv | CD28 | CD8 | CD3ζ |
| ScFv | CD28 | CD8 | CD3δ |
| ScFv | CD28 | CD8 | CD3γ |
| ScFv | CD28 | CD8 | CD3ε |
| ScFv | CD28 | CD8 | FcγRI-γ |
| ScFv | CD28 | CD8 | FcγRIII-γ |
| ScFv | CD28 | CD8 | FcεRIβ |
| ScFv | CD28 | CD8 | FcεRIγ |
| ScFv | CD28 | CD8 | DAP10 |
| ScFv | CD28 | CD8 | DAP12 |
| ScFv | CD28 | CD8 | CD32 |
| ScFv | CD28 | CD8 | CD79a |
| ScFv | CD28 | CD8 | CD79b |
| ScFv | CD28 | CD4 | CD8 |
| ScFv | CD28 | CD4 | CD3ζ |
| ScFv | CD28 | CD4 | CD3δ |
| ScFv | CD28 | CD4 | CD3γ |
| ScFv | CD28 | CD4 | CD3ε |
| ScFv | CD28 | CD4 | FcγRI-γ |
| ScFv | CD28 | CD4 | FcγRIII-γ |
| ScFv | CD28 | CD4 | FcεRIβ |
| ScFv | CD28 | CD4 | FcεRIγ |
| ScFv | CD28 | CD4 | DAP10 |
| ScFv | CD28 | CD4 | DAP12 |
| ScFv | CD28 | CD4 | CD32 |
| ScFv | CD28 | CD4 | CD79a |
| ScFv | CD28 | CD4 | CD79b |
| ScFv | CD28 | b2c | CD8 |
| ScFv | CD28 | b2c | CD3ζ |
| ScFv | CD28 | b2c | CD3δ |
| ScFv | CD28 | b2c | CD3γ |
| ScFv | CD28 | b2c | CD3ε |
| ScFv | CD28 | b2c | FcγRI-γ |
| ScFv | CD28 | b2c | FcγRIII-γ |
| ScFv | CD28 | b2c | FcεRIβ |
| ScFv | CD28 | b2c | FcεRIγ |
| ScFv | CD28 | b2c | DAP10 |
| ScFv | CD28 | b2c | DAP12 |
| ScFv | CD28 | b2c | CD32 |
| ScFv | CD28 | b2c | CD79a |
| ScFv | CD28 | b2c | CD79b |
| ScFv | CD28 | CD137/41BB | CD8 |
| ScFv | CD28 | CD137/41BB | CD3ζ |
| ScFv | CD28 | CD137/41BB | CD3δ |
| ScFv | CD28 | CD137/41BB | CD3γ |
| ScFv | CD28 | CD137/41BB | CD3ε |
| ScFv | CD28 | CD137/41BB | FcγRI-γ |
| ScFv | CD28 | CD137/41BB | FcγRIII-γ |
| ScFv | CD28 | CD137/41BB | FcεRIβ |
| ScFv | CD28 | CD137/41BB | FcεRIγ |
| ScFv | CD28 | CD137/41BB | DAP10 |
| ScFv | CD28 | CD137/41BB | DAP12 |
| ScFv | CD28 | CD137/41BB | CD32 |
| ScFv | CD28 | CD137/41BB | CD79a |
| ScFv | CD28 | CD137/41BB | CD79b |
| ScFv | CD28 | ICOS | CD8 |
| ScFv | CD28 | ICOS | CD3ζ |
| ScFv | CD28 | ICOS | CD3δ |
| ScFv | CD28 | ICOS | CD3γ |
| ScFv | CD28 | ICOS | CD3ε |
| ScFv | CD28 | ICOS | FcγRI-γ |
| ScFv | CD28 | ICOS | FcγRIII-γ |
| ScFv | CD28 | ICOS | FcεRIβ |
| ScFv | CD28 | ICOS | FcεRIγ |
| ScFv | CD28 | ICOS | DAP10 |
| ScFv | CD28 | ICOS | DAP12 |
| ScFv | CD28 | ICOS | CD32 |
| ScFv | CD28 | ICOS | CD79a |
| ScFv | CD28 | ICOS | CD79b |
| ScFv | CD28 | CD27 | CD8 |
| ScFv | CD28 | CD27 | CD3ζ |
| ScFv | CD28 | CD27 | CD3δ |
| ScFv | CD28 | CD27 | CD3γ |
| ScFv | CD28 | CD27 | CD3ε |
| ScFv | CD28 | CD27 | FcγRI-γ |
| ScFv | CD28 | CD27 | FcγRIII-γ |
| ScFv | CD28 | CD27 | FcεRIβ |
| ScFv | CD28 | CD27 | FcεRIγ |
| ScFv | CD28 | CD27 | DAP10 |
| ScFv | CD28 | CD27 | DAP12 |
| ScFv | CD28 | CD27 | CD32 |
| ScFv | CD28 | CD27 | CD79a |
| ScFv | CD28 | CD27 | CD79b |
| ScFv | CD28 | CD28δ | CD8 |
| ScFv | CD28 | CD28δ | CD3ζ |
| ScFv | CD28 | CD28δ | CD3δ |
| ScFv | CD28 | CD28δ | CD3γ |
| ScFv | CD28 | CD28δ | CD3ε |
| ScFv | CD28 | CD28δ | FcγRI-γ |
| ScFv | CD28 | CD28δ | FcγRIII-γ |
| ScFv | CD28 | CD28δ | FcεRIβ |
| ScFv | CD28 | CD28δ | FcεRIγ |
| ScFv | CD28 | CD28δ | DAP10 |
| ScFv | CD28 | CD28δ | DAP12 |
| ScFv | CD28 | CD28δ | CD32 |
| ScFv | CD28 | CD28δ | CD79a |
| ScFv | CD28 | CD28δ | CD79b |
| ScFv | CD28 | CD80 | CD8 |
| ScFv | CD28 | CD80 | CD3ζ |
| ScFv | CD28 | CD80 | CD3δ |
| ScFv | CD28 | CD80 | CD3γ |
| ScFv | CD28 | CD80 | CD3ε |
| ScFv | CD28 | CD80 | FcγRI-γ |
| ScFv | CD28 | CD80 | FcγRIII-γ |
| ScFv | CD28 | CD80 | FcεRIβ |
| ScFv | CD28 | CD80 | FcεRIγ |
| ScFv | CD28 | CD80 | DAP10 |
| ScFv | CD28 | CD80 | DAP12 |
| ScFv | CD28 | CD80 | CD32 |
| ScFv | CD28 | CD80 | CD79a |
| ScFv | CD28 | CD80 | CD79b |
| ScFv | CD28 | CD86 | CD8 |
| ScFv | CD28 | CD86 | CD3ζ |
| ScFv | CD28 | CD86 | CD3δ |
| ScFv | CD28 | CD86 | CD3γ |
| ScFv | CD28 | CD86 | CD3ε |
| ScFv | CD28 | CD86 | FcγRI-γ |
| ScFv | CD28 | CD86 | FcγRIII-γ |
| ScFv | CD28 | CD86 | FcεRIβ |
| ScFv | CD28 | CD86 | FcεRIγ |
| ScFv | CD28 | CD86 | DAP10 |
| ScFv | CD28 | CD86 | DAP12 |
| ScFv | CD28 | CD86 | CD32 |
| ScFv | CD28 | CD86 | CD79a |
| ScFv | CD28 | CD86 | CD79b |
| ScFv | CD28 | OX40 | CD8 |
| ScFv | CD28 | OX40 | CD3ζ |
| ScFv | CD28 | OX40 | CD3δ |
| ScFv | CD28 | OX40 | CD3γ |
| ScFv | CD28 | OX40 | CD3ε |
| ScFv | CD28 | OX40 | FcγRI-γ |
| ScFv | CD28 | OX40 | FcγRIII-γ |
| ScFv | CD28 | OX40 | FcεRIβ |
| ScFv | CD28 | OX40 | FcεRIγ |
| ScFv | CD28 | OX40 | DAP10 |
| ScFv | CD28 | OX40 | DAP12 |
| ScFv | CD28 | OX40 | CD32 |
| ScFv | CD28 | OX40 | CD79a |
| ScFv | CD28 | OX40 | CD79b |
| ScFv | CD28 | DAP10 | CD8 |
| ScFv | CD28 | DAP10 | CD3ζ |
| ScFv | CD28 | DAP10 | CD3δ |
| ScFv | CD28 | DAP10 | CD3γ |
| ScFv | CD28 | DAP10 | CD3ε |
| ScFv | CD28 | DAP10 | FcγRI-γ |
| ScFv | CD28 | DAP10 | FcγRIII-γ |
| ScFv | CD28 | DAP10 | FcεRIβ |
| ScFv | CD28 | DAP10 | FcεRIγ |
| ScFv | CD28 | DAP10 | DAP10 |
| ScFv | CD28 | DAP10 | DAP12 |
| ScFv | CD28 | DAP10 | CD32 |
| ScFv | CD28 | DAP10 | CD79a |
| ScFv | CD28 | DAP10 | CD79b |
| ScFv | CD28 | DAP12 | CD8 |
| ScFv | CD28 | DAP12 | CD3ζ |
| ScFv | CD28 | DAP12 | CD3δ |
| ScFv | CD28 | DAP12 | CD3γ |
| ScFv | CD28 | DAP12 | CD3ε |
| ScFv | CD28 | DAP12 | FcγRI-γ |
| ScFv | CD28 | DAP12 | FcγRIII-γ |
| ScFv | CD28 | DAP12 | FcεRIβ |
| ScFv | CD28 | DAP12 | FcεRIγ |
| ScFv | CD28 | DAP12 | DAP10 |
| ScFv | CD28 | DAP12 | DAP12 |
| ScFv | CD28 | DAP12 | CD32 |
| ScFv | CD28 | DAP12 | CD79a |
| ScFv | CD28 | DAP12 | CD79b |
| ScFv | CD28 | MyD88 | CD8 |
| ScFv | CD28 | MyD88 | CD3ζ |
| ScFv | CD28 | MyD88 | CD3δ |
| ScFv | CD28 | MyD88 | CD3γ |
| ScFv | CD28 | MyD88 | CD3ε |
| ScFv | CD28 | MyD88 | FcγRI-γ |
| ScFv | CD28 | MyD88 | FcγRIII-γ |
| ScFv | CD28 | MyD88 | FcεRIβ |
| ScFv | CD28 | MyD88 | FcεRIγ |
| ScFv | CD28 | MyD88 | DAP10 |
| ScFv | CD28 | MyD88 | DAP12 |
| ScFv | CD28 | MyD88 | CD32 |
| ScFv | CD28 | MyD88 | CD79a |
| ScFv | CD28 | MyD88 | CD79b |
| ScFv | CD28 | CD7 | CD8 |
| ScFv | CD28 | CD7 | CD3ζ |
| ScFv | CD28 | CD7 | CD3δ |
| ScFv | CD28 | CD7 | CD3γ |
| ScFv | CD28 | CD7 | CD3ε |
| ScFv | CD28 | CD7 | FcγRI-γ |
| ScFv | CD28 | CD7 | FcγRIII-γ |
| ScFv | CD28 | CD7 | FcεRIβ |
| ScFv | CD28 | CD7 | FcεRIγ |
| ScFv | CD28 | CD7 | DAP10 |
| ScFv | CD28 | CD7 | DAP12 |
| ScFv | CD28 | CD7 | CD32 |
| ScFv | CD28 | CD7 | CD79a |
| ScFv | CD28 | CD7 | CD79b |
| ScFv | CD28 | BTNL3 | CD8 |
| ScFv | CD28 | BTNL3 | CD3ζ |
| ScFv | CD28 | BTNL3 | CD3δ |
| ScFv | CD28 | BTNL3 | CD3γ |
| ScFv | CD28 | BTNL3 | CD3ε |
| ScFv | CD28 | BTNL3 | FcγRI-γ |
| ScFv | CD28 | BTNL3 | FcγRIII-γ |
| ScFv | CD28 | BTNL3 | FcεRIβ |
| ScFv | CD28 | BTNL3 | FcεRIγ |
| ScFv | CD28 | BTNL3 | DAP10 |
| ScFv | CD28 | BTNL3 | DAP12 |
| ScFv | CD28 | BTNL3 | CD32 |
| ScFv | CD28 | BTNL3 | CD79a |
| ScFv | CD28 | BTNL3 | CD79b |
| ScFv | CD28 | NKG2D | CD8 |
| ScFv | CD28 | NKG2D | CD3ζ |
| ScFv | CD28 | NKG2D | CD3δ |
| ScFv | CD28 | NKG2D | CD3γ |
| ScFv | CD28 | NKG2D | CD3ε |
| ScFv | CD28 | NKG2D | FcγRI-γ |
| ScFv | CD28 | NKG2D | FcγRIII-γ |
| ScFv | CD28 | NKG2D | FcεRIβ |
| ScFv | CD28 | NKG2D | FcεRIγ |
| ScFv | CD28 | NKG2D | DAP10 |
| ScFv | CD28 | NKG2D | DAP12 |
| ScFv | CD28 | NKG2D | CD32 |
| ScFv | CD28 | NKG2D | CD79a |
| ScFv | CD28 | NKG2D | CD79b |
| ScFv | CD8 | CD28 | CD8 |
| ScFv | CD8 | CD28 | CD3ζ |
| ScFv | CD8 | CD28 | CD3δ |
| ScFv | CD8 | CD28 | CD3γ |
| ScFv | CD8 | CD28 | CD3ε |
| ScFv | CD8 | CD28 | FcγRI-γ |
| ScFv | CD8 | CD28 | FcγRIII-γ |
| ScFv | CD8 | CD28 | FcεRIβ |
| ScFv | CD8 | CD28 | FcεRIγ |
| ScFv | CD8 | CD28 | DAP10 |
| ScFv | CD8 | CD28 | DAP12 |
| ScFv | CD8 | CD28 | CD32 |
| ScFv | CD8 | CD28 | CD79a |
| ScFv | CD8 | CD28 | CD79b |
| ScFv | CD8 | CD8 | CD8 |
| ScFv | CD8 | CD8 | CD3ζ |
| ScFv | CD8 | CD8 | CD3δ |
| ScFv | CD8 | CD8 | CD3γ |
| ScFv | CD8 | CD8 | CD3ε |
| ScFv | CD8 | CD8 | FcγRI-γ |
| ScFv | CD8 | CD8 | FcγRIII-γ |
| ScFv | CD8 | CD8 | FcεRIβ |
| ScFv | CD8 | CD8 | FcεRIγ |
| ScFv | CD8 | CD8 | DAP10 |
| ScFv | CD8 | CD8 | DAP12 |
| ScFv | CD8 | CD8 | CD32 |
| ScFv | CD8 | CD8 | CD79a |
| ScFv | CD8 | CD8 | CD79b |
| ScFv | CD8 | CD4 | CD8 |
| ScFv | CD8 | CD4 | CD3ζ |
| ScFv | CD8 | CD4 | CD3δ |
| ScFv | CD8 | CD4 | CD3γ |
| ScFv | CD8 | CD4 | CD3ε |
| ScFv | CD8 | CD4 | FcγRI-γ |
| ScFv | CD8 | CD4 | FcγRIII-γ |
| ScFv | CD8 | CD4 | FcεRIβ |
| ScFv | CD8 | CD4 | FcεRIγ |
| ScFv | CD8 | CD4 | DAP10 |
| ScFv | CD8 | CD4 | DAP12 |
| ScFv | CD8 | CD4 | CD32 |
| ScFv | CD8 | CD4 | CD79a |
| ScFv | CD8 | CD4 | CD79b |
| ScFv | CD8 | b2c | CD8 |
| ScFv | CD8 | b2c | CD3ζ |
| ScFv | CD8 | b2c | CD3δ |
| ScFv | CD8 | b2c | CD3γ |
| ScFv | CD8 | b2c | CD3ε |
| ScFv | CD8 | b2c | FcγRI-γ |
| ScFv | CD8 | b2c | FcγRIII-γ |
| ScFv | CD8 | b2c | FcεRIβ |
| ScFv | CD8 | b2c | FcεRIγ |
| ScFv | CD8 | b2c | DAP10 |
| ScFv | CD8 | b2c | DAP12 |
| ScFv | CD8 | b2c | CD32 |
| ScFv | CD8 | b2c | CD79a |
| ScFv | CD8 | b2c | CD79b |
| ScFv | CD8 | CD137/41BB | CD8 |
| ScFv | CD8 | CD137/41BB | CD3ζ |
| ScFv | CD8 | CD137/41BB | CD3δ |
| ScFv | CD8 | CD137/41BB | CD3γ |
| ScFv | CD8 | CD137/41BB | CD3ε |
| ScFv | CD8 | CD137/41BB | FcγRI-γ |
| ScFv | CD8 | CD137/41BB | FcγRIII-γ |
| ScFv | CD8 | CD137/41BB | FcεRIβ |
| ScFv | CD8 | CD137/41BB | FcεRIγ |
| ScFv | CD8 | CD137/41BB | DAP10 |
| ScFv | CD8 | CD137/41BB | DAP12 |
| ScFv | CD8 | CD137/41BB | CD32 |
| ScFv | CD8 | CD137/41BB | CD79a |
| ScFv | CD8 | CD137/41BB | CD79b |
| ScFv | CD8 | ICOS | CD8 |
| ScFv | CD8 | ICOS | CD3ζ |
| ScFv | CD8 | ICOS | CD3δ |
| ScFv | CD8 | ICOS | CD3γ |
| ScFv | CD8 | ICOS | CD3ε |
| ScFv | CD8 | ICOS | FcγRI-γ |
| ScFv | CD8 | ICOS | FcγRIII-γ |
| ScFv | CD8 | ICOS | FcεRIβ |
| ScFv | CD8 | ICOS | FcεRIγ |
| ScFv | CD8 | ICOS | DAP10 |
| ScFv | CD8 | ICOS | DAP12 |
| ScFv | CD8 | ICOS | CD32 |
| ScFv | CD8 | ICOS | CD79a |
| ScFv | CD8 | ICOS | CD79b |
| ScFv | CD8 | CD27 | CD8 |
| ScFv | CD8 | CD27 | CD3ζ |
| ScFv | CD8 | CD27 | CD3δ |
| ScFv | CD8 | CD27 | CD3γ |
| ScFv | CD8 | CD27 | CD3ε |
| ScFv | CD8 | CD27 | FcγRI-γ |
| ScFv | CD8 | CD27 | FcγRIII-γ |
| ScFv | CD8 | CD27 | FcεRIβ |
| ScFv | CD8 | CD27 | FcεRIγ |
| ScFv | CD8 | CD27 | DAP10 |
| ScFv | CD8 | CD27 | DAP12 |
| ScFv | CD8 | CD27 | CD32 |
| ScFv | CD8 | CD27 | CD79a |
| ScFv | CD8 | CD27 | CD79b |
| ScFv | CD8 | CD28δ | CD8 |
| ScFv | CD8 | CD28δ | CD3ζ |
| ScFv | CD8 | CD28δ | CD3δ |
| ScFv | CD8 | CD28δ | CD3γ |
| ScFv | CD8 | CD28δ | CD3ε |
| ScFv | CD8 | CD28δ | FcγRI-γ |
| ScFv | CD8 | CD28δ | FcγRIII-γ |
| ScFv | CD8 | CD28δ | FcεRIβ |
| ScFv | CD8 | CD28δ | FcεRIγ |
| ScFv | CD8 | CD28δ | DAP10 |
| ScFv | CD8 | CD28δ | DAP12 |
| ScFv | CD8 | CD28δ | CD32 |
| ScFv | CD8 | CD28δ | CD79a |
| ScFv | CD8 | CD28δ | CD79b |
| ScFv | CD8 | CD80 | CD8 |
| ScFv | CD8 | CD80 | CD3ζ |
| ScFv | CD8 | CD80 | CD3δ |
| ScFv | CD8 | CD80 | CD3γ |
| ScFv | CD8 | CD80 | CD3ε |
| ScFv | CD8 | CD80 | FcγRI-γ |
| ScFv | CD8 | CD80 | FcγRIII-γ |
| ScFv | CD8 | CD80 | FcεRIβ |
| ScFv | CD8 | CD80 | FcεRIγ |
| ScFv | CD8 | CD80 | DAP10 |
| ScFv | CD8 | CD80 | DAP12 |
| ScFv | CD8 | CD80 | CD32 |
| ScFv | CD8 | CD80 | CD79a |
| ScFv | CD8 | CD80 | CD79b |
| ScFv | CD8 | CD86 | CD8 |
| ScFv | CD8 | CD86 | CD3ζ |
| ScFv | CD8 | CD86 | CD3δ |
| ScFv | CD8 | CD86 | CD3γ |
| ScFv | CD8 | CD86 | CD3ε |
| ScFv | CD8 | CD86 | FcγRI-γ |
| ScFv | CD8 | CD86 | FcγRIII-γ |
| ScFv | CD8 | CD86 | FcεRIβ |
| ScFv | CD8 | CD86 | FcεRIγ |
| ScFv | CD8 | CD86 | DAP10 |
| ScFv | CD8 | CD86 | DAP12 |
| ScFv | CD8 | CD86 | CD32 |
| ScFv | CD8 | CD86 | CD79a |
| ScFv | CD8 | CD86 | CD79b |
| ScFv | CD8 | OX40 | CD8 |
| ScFv | CD8 | OX40 | CD3ζ |
| ScFv | CD8 | OX40 | CD3δ |
| ScFv | CD8 | OX40 | CD3γ |
| ScFv | CD8 | OX40 | CD3ε |
| ScFv | CD8 | OX40 | FcγRI-γ |
| ScFv | CD8 | OX40 | FcγRIII-γ |
| ScFv | CD8 | OX40 | FcεRIβ |
| ScFv | CD8 | OX40 | FcεRIγ |
| ScFv | CD8 | OX40 | DAP10 |
| ScFv | CD8 | OX40 | DAP12 |
| ScFv | CD8 | OX40 | CD32 |
| ScFv | CD8 | OX40 | CD79a |
| ScFv | CD8 | OX40 | CD79b |
| ScFv | CD8 | DAP10 | CD8 |
| ScFv | CD8 | DAP10 | CD3ζ |
| ScFv | CD8 | DAP10 | CD3δ |
| ScFv | CD8 | DAP10 | CD3γ |
| ScFv | CD8 | DAP10 | CD3ε |
| ScFv | CD8 | DAP10 | FcγRI-γ |
| ScFv | CD8 | DAP10 | FcγRIII-γ |
| ScFv | CD8 | DAP10 | FcεRIβ |
| ScFv | CD8 | DAP10 | FcεRIγ |
| ScFv | CD8 | DAP10 | DAP10 |
| ScFv | CD8 | DAP10 | DAP12 |
| ScFv | CD8 | DAP10 | CD32 |
| ScFv | CD8 | DAP10 | CD79a |
| ScFv | CD8 | DAP10 | CD79b |
| ScFv | CD8 | DAP12 | CD8 |
| ScFv | CD8 | DAP12 | CD3ζ |
| ScFv | CD8 | DAP12 | CD3δ |
| ScFv | CD8 | DAP12 | CD3γ |
| ScFv | CD8 | DAP12 | CD3ε |
| ScFv | CD8 | DAP12 | FcγRI-γ |
| ScFv | CD8 | DAP12 | FcγRIII-γ |
| ScFv | CD8 | DAP12 | FcεRIβ |
| ScFv | CD8 | DAP12 | FcεRIγ |
| ScFv | CD8 | DAP12 | DAP10 |
| ScFv | CD8 | DAP12 | DAP12 |
| ScFv | CD8 | DAP12 | CD32 |
| ScFv | CD8 | DAP12 | CD79a |
| ScFv | CD8 | DAP12 | CD79b |
| ScFv | CD8 | MyD88 | CD8 |
| ScFv | CD8 | MyD88 | CD3ζ |
| ScFv | CD8 | MyD88 | CD3δ |
| ScFv | CD8 | MyD88 | CD3γ |
| ScFv | CD8 | MyD88 | CD3ε |
| ScFv | CD8 | MyD88 | FcγRI-γ |
| ScFv | CD8 | MyD88 | FcγRIII-γ |
| ScFv | CD8 | MyD88 | FcεRIβ |
| ScFv | CD8 | MyD88 | FcεRIγ |
| ScFv | CD8 | MyD88 | DAP10 |
| ScFv | CD8 | MyD88 | DAP12 |
| ScFv | CD8 | MyD88 | CD32 |
| ScFv | CD8 | MyD88 | CD79a |
| ScFv | CD8 | MyD88 | CD79b |
| ScFv | CD8 | CD7 | CD8 |
| ScFv | CD8 | CD7 | CD3ζ |
| ScFv | CD8 | CD7 | CD3δ |
| ScFv | CD8 | CD7 | CD3γ |
| ScFv | CD8 | CD7 | CD3ε |
| ScFv | CD8 | CD7 | FcγRI-γ |
| ScFv | CD8 | CD7 | FcγRIII-γ |
| ScFv | CD8 | CD7 | FcεRIβ |
| ScFv | CD8 | CD7 | FcεRIγ |
| ScFv | CD8 | CD7 | DAP10 |
| ScFv | CD8 | CD7 | DAP12 |
| ScFv | CD8 | CD7 | CD32 |
| ScFv | CD8 | CD7 | CD79a |
| ScFv | CD8 | CD7 | CD79b |
| ScFv | CD8 | BTNL3 | CD8 |
| ScFv | CD8 | BTNL3 | CD3ζ |
| ScFv | CD8 | BTNL3 | CD3δ |
| ScFv | CD8 | BTNL3 | CD3γ |
| ScFv | CD8 | BTNL3 | CD3ε |
| ScFv | CD8 | BTNL3 | FcγRI-γ |
| ScFv | CD8 | BTNL3 | FcγRIII-γ |
| ScFv | CD8 | BTNL3 | FcεRIβ |
| ScFv | CD8 | BTNL3 | FcεRIγ |
| ScFv | CD8 | BTNL3 | DAP10 |
| ScFv | CD8 | BTNL3 | DAP12 |
| ScFv | CD8 | BTNL3 | CD32 |
| ScFv | CD8 | BTNL3 | CD79a |
| ScFv | CD8 | BTNL3 | CD79b |
| ScFv | CD8 | NKG2D | CD8 |
| ScFv | CD8 | NKG2D | CD3ζ |
| ScFv | CD8 | NKG2D | CD3δ |
| ScFv | CD8 | NKG2D | CD3γ |
| ScFv | CD8 | NKG2D | CD3ε |
| ScFv | CD8 | NKG2D | FcγRI-γ |
| ScFv | CD8 | NKG2D | FcγRIII-γ |
| ScFv | CD8 | NKG2D | FcεRIβ |
| ScFv | CD8 | NKG2D | FcεRIγ |
| ScFv | CD8 | NKG2D | DAP10 |
| ScFv | CD8 | NKG2D | DAP12 |
| ScFv | CD8 | NKG2D | CD32 |
| ScFv | CD8 | NKG2D | CD79a |
| ScFv | CD8 | NKG2D | CD79b |
| ScFv | CD4 | CD28 | CD8 |
| ScFv | CD4 | CD28 | CD3ζ |
| ScFv | CD4 | CD28 | CD3δ |
| ScFv | CD4 | CD28 | CD3γ |
| ScFv | CD4 | CD28 | CD3ε |
| ScFv | CD4 | CD28 | FcγRI-γ |
| ScFv | CD4 | CD28 | FcγRIII-γ |
| ScFv | CD4 | CD28 | FcεRIβ |
| ScFv | CD4 | CD28 | FcεRIγ |
| ScFv | CD4 | CD28 | DAP10 |
| ScFv | CD4 | CD28 | DAP12 |
| ScFv | CD4 | CD28 | CD32 |
| ScFv | CD4 | CD28 | CD79a |
| ScFv | CD4 | CD28 | CD79b |
| ScFv | CD4 | CD8 | CD8 |
| ScFv | CD4 | CD8 | CD3ζ |
| ScFv | CD4 | CD8 | CD3δ |
| ScFv | CD4 | CD8 | CD3γ |
| ScFv | CD4 | CD8 | CD3ε |
| ScFv | CD4 | CD8 | FcγRI-γ |
| ScFv | CD4 | CD8 | FcγRIII-γ |
| ScFv | CD4 | CD8 | FcεRIβ |
| ScFv | CD4 | CD8 | FcεRIγ |
| ScFv | CD4 | CD8 | DAP10 |
| ScFv | CD4 | CD8 | DAP12 |
| ScFv | CD4 | CD8 | CD32 |
| ScFv | CD4 | CD8 | CD79a |
| ScFv | CD4 | CD8 | CD79b |
| ScFv | CD4 | CD4 | CD8 |
| ScFv | CD4 | CD4 | CD3ζ |
| ScFv | CD4 | CD4 | CD3δ |
| ScFv | CD4 | CD4 | CD3γ |
| ScFv | CD4 | CD4 | CD3ε |
| ScFv | CD4 | CD4 | FcγRI-γ |
| ScFv | CD4 | CD4 | FcγRIII-γ |
| ScFv | CD4 | CD4 | FcεRIβ |
| ScFv | CD4 | CD4 | FcεRIγ |
| ScFv | CD4 | CD4 | DAP10 |
| ScFv | CD4 | CD4 | DAP12 |
| ScFv | CD4 | CD4 | CD32 |
| ScFv | CD4 | CD4 | CD79a |
| ScFv | CD4 | CD4 | CD79b |
| ScFv | CD4 | b2c | CD8 |
| ScFv | CD4 | b2c | CD3ζ |
| ScFv | CD4 | b2c | CD3δ |
| ScFv | CD4 | b2c | CD3γ |
| ScFv | CD4 | b2c | CD3ε |
| ScFv | CD4 | b2c | FcγRI-γ |
| ScFv | CD4 | b2c | FcγRIII-γ |
| ScFv | CD4 | b2c | FcεRIβ |
| ScFv | CD4 | b2c | FcεRIγ |
| ScFv | CD4 | b2c | DAP10 |
| ScFv | CD4 | b2c | DAP12 |
| ScFv | CD4 | b2c | CD32 |
| ScFv | CD4 | b2c | CD79a |
| ScFv | CD4 | b2c | CD79b |
| ScFv | CD4 | CD137/41BB | CD8 |
| ScFv | CD4 | CD137/41BB | CD3ζ |
| ScFv | CD4 | CD137/41BB | CD3δ |
| ScFv | CD4 | CD137/41BB | CD3γ |
| ScFv | CD4 | CD137/41BB | CD3ε |
| ScFv | CD4 | CD137/41BB | FcγRI-γ |
| ScFv | CD4 | CD137/41BB | FcγRIII-γ |
| ScFv | CD4 | CD137/41BB | FcεRIβ |
| ScFv | CD4 | CD137/41BB | FcεRIγ |
| ScFv | CD4 | CD137/41BB | DAP10 |
| ScFv | CD4 | CD137/41BB | DAP12 |
| ScFv | CD4 | CD137/41BB | CD32 |
| ScFv | CD4 | CD137/41BB | CD79a |
| ScFv | CD4 | CD137/41BB | CD79b |
| ScFv | CD4 | ICOS | CD8 |
| ScFv | CD4 | ICOS | CD3ζ |
| ScFv | CD4 | ICOS | CD3δ |
| ScFv | CD4 | ICOS | CD3γ |
| ScFv | CD4 | ICOS | CD3ε |
| ScFv | CD4 | ICOS | FcγRI-γ |
| ScFv | CD4 | ICOS | FcγRIII-γ |
| ScFv | CD4 | ICOS | FcεRIβ |
| ScFv | CD4 | ICOS | FcεRIγ |
| ScFv | CD4 | ICOS | DAP10 |
| ScFv | CD4 | ICOS | DAP12 |
| ScFv | CD4 | ICOS | CD32 |
| ScFv | CD4 | ICOS | CD79a |
| ScFv | CD4 | ICOS | CD79b |
| ScFv | CD4 | CD27 | CD8 |
| ScFv | CD4 | CD27 | CD3ζ |
| ScFv | CD4 | CD27 | CD3δ |
| ScFv | CD4 | CD27 | CD3γ |
| ScFv | CD4 | CD27 | CD3ε |
| ScFv | CD4 | CD27 | FcγRI-γ |
| ScFv | CD4 | CD27 | FcγRIII-γ |
| ScFv | CD4 | CD27 | FcεRIβ |
| ScFv | CD4 | CD27 | FcεRIγ |
| ScFv | CD4 | CD27 | DAP10 |
| ScFv | CD4 | CD27 | DAP12 |
| ScFv | CD4 | CD27 | CD32 |
| ScFv | CD4 | CD27 | CD79a |
| ScFv | CD4 | CD27 | CD79b |
| ScFv | CD4 | CD28δ | CD8 |
| ScFv | CD4 | CD28δ | CD3ζ |
| ScFv | CD4 | CD28δ | CD3δ |
| ScFv | CD4 | CD28δ | CD3γ |
| ScFv | CD4 | CD28δ | CD3ε |
| ScFv | CD4 | CD28δ | FcγRI-γ |
| ScFv | CD4 | CD28δ | FcγRIII-γ |
| ScFv | CD4 | CD28δ | FcεRIβ |
| ScFv | CD4 | CD28δ | FcεRIγ |
| ScFv | CD4 | CD28δ | DAP10 |
| ScFv | CD4 | CD28δ | DAP12 |
| ScFv | CD4 | CD28δ | CD32 |
| ScFv | CD4 | CD28δ | CD79a |
| ScFv | CD4 | CD28δ | CD79b |
| ScFv | CD4 | CD80 | CD8 |
| ScFv | CD4 | CD80 | CD3ζ |
| ScFv | CD4 | CD80 | CD3δ |
| ScFv | CD4 | CD80 | CD3γ |
| ScFv | CD4 | CD80 | CD3ε |
| ScFv | CD4 | CD80 | FcγRI-γ |
| ScFv | CD4 | CD80 | FcγRIII-γ |
| ScFv | CD4 | CD80 | FcεRIβ |
| ScFv | CD4 | CD80 | FcεRIγ |
| ScFv | CD4 | CD80 | DAP10 |
| ScFv | CD4 | CD80 | DAP12 |
| ScFv | CD4 | CD80 | CD32 |
| ScFv | CD4 | CD80 | CD79a |
| ScFv | CD4 | CD80 | CD79b |
| ScFv | CD4 | CD86 | CD8 |
| ScFv | CD4 | CD86 | CD3ζ |
| ScFv | CD4 | CD86 | CD3δ |
| ScFv | CD4 | CD86 | CD3γ |
| ScFv | CD4 | CD86 | CD3ε |
| ScFv | CD4 | CD86 | FcγRI-γ |
| ScFv | CD4 | CD86 | FcγRIII-γ |
| ScFv | CD4 | CD86 | FcεRIβ |
| ScFv | CD4 | CD86 | FcεRIγ |
| ScFv | CD4 | CD86 | DAP10 |
| ScFv | CD4 | CD86 | DAP12 |
| ScFv | CD4 | CD86 | CD32 |
| ScFv | CD4 | CD86 | CD79a |
| ScFv | CD4 | CD86 | CD79b |
| ScFv | CD4 | OX40 | CD8 |
| ScFv | CD4 | OX40 | CD3ζ |
| ScFv | CD4 | OX40 | CD3δ |
| ScFv | CD4 | OX40 | CD3γ |
| ScFv | CD4 | OX40 | CD3ε |
| ScFv | CD4 | OX40 | FcγRI-γ |
| ScFv | CD4 | OX40 | FcγRIII-γ |
| ScFv | CD4 | OX40 | FcεRIβ |
| ScFv | CD4 | OX40 | FcεRIγ |
| ScFv | CD4 | OX40 | DAP10 |
| ScFv | CD4 | OX40 | DAP12 |
| ScFv | CD4 | OX40 | CD32 |
| ScFv | CD4 | OX40 | CD79a |
| ScFv | CD4 | OX40 | CD79b |
| ScFv | CD4 | DAP10 | CD8 |
| ScFv | CD4 | DAP10 | CD3ζ |
| ScFv | CD4 | DAP10 | CD3δ |
| ScFv | CD4 | DAP10 | CD3γ |
| ScFv | CD4 | DAP10 | CD3ε |
| ScFv | CD4 | DAP10 | FcγRI-γ |
| ScFv | CD4 | DAP10 | FcγRIII-γ |
| ScFv | CD4 | DAP10 | FcεRIβ |
| ScFv | CD4 | DAP10 | FcεRIγ |
| ScFv | CD4 | DAP10 | DAP10 |
| ScFv | CD4 | DAP10 | DAP12 |
| ScFv | CD4 | DAP10 | CD32 |
| ScFv | CD4 | DAP10 | CD79a |
| ScFv | CD4 | DAP10 | CD79b |
| ScFv | CD4 | DAP12 | CD8 |
| ScFv | CD4 | DAP12 | CD3ζ |
| ScFv | CD4 | DAP12 | CD3δ |
| ScFv | CD4 | DAP12 | CD3γ |
| ScFv | CD4 | DAP12 | CD3ε |
| ScFv | CD4 | DAP12 | FcγRI-γ |
| ScFv | CD4 | DAP12 | FcγRIII-γ |
| ScFv | CD4 | DAP12 | FcεRIβ |
| ScFv | CD4 | DAP12 | FcεRIγ |
| ScFv | CD4 | DAP12 | DAP10 |
| ScFv | CD4 | DAP12 | DAP12 |
| ScFv | CD4 | DAP12 | CD32 |
| ScFv | CD4 | DAP12 | CD79a |
| ScFv | CD4 | DAP12 | CD79b |
| ScFv | CD4 | MyD88 | CD8 |
| ScFv | CD4 | MyD88 | CD3ζ |
| ScFv | CD4 | MyD88 | CD3δ |
| ScFv | CD4 | MyD88 | CD3γ |
| ScFv | CD4 | MyD88 | CD3ε |
| ScFv | CD4 | MyD88 | FcγRI-γ |
| ScFv | CD4 | MyD88 | FcγRIII-γ |
| ScFv | CD4 | MyD88 | FcεRIβ |
| ScFv | CD4 | MyD88 | FcεRIγ |
| ScFv | CD4 | MyD88 | DAP10 |
| ScFv | CD4 | MyD88 | DAP12 |
| ScFv | CD4 | MyD88 | CD32 |
| ScFv | CD4 | MyD88 | CD79a |
| ScFv | CD4 | MyD88 | CD79b |
| ScFv | CD4 | CD7 | CD8 |
| ScFv | CD4 | CD7 | CD3ζ |
| ScFv | CD4 | CD7 | CD3δ |
| ScFv | CD4 | CD7 | CD3γ |
| ScFv | CD4 | CD7 | CD3ε |
| ScFv | CD4 | CD7 | FcγRI-γ |
| ScFv | CD4 | CD7 | FcγRIII-γ |
| ScFv | CD4 | CD7 | FcεRIβ |
| ScFv | CD4 | CD7 | FcεRIγ |
| ScFv | CD4 | CD7 | DAP10 |
| ScFv | CD4 | CD7 | DAP12 |
| ScFv | CD4 | CD7 | CD32 |
| ScFv | CD4 | CD7 | CD79a |
| ScFv | CD4 | CD7 | CD79b |
| ScFv | CD4 | BTNL3 | CD8 |
| ScFv | CD4 | BTNL3 | CD3ζ |
| ScFv | CD4 | BTNL3 | CD3δ |
| ScFv | CD4 | BTNL3 | CD3γ |
| ScFv | CD4 | BTNL3 | CD3ε |
| ScFv | CD4 | BTNL3 | FcγRI-γ |
| ScFv | CD4 | BTNL3 | FcγRIII-γ |
| ScFv | CD4 | BTNL3 | FcεRIβ |
| ScFv | CD4 | BTNL3 | FcεRIγ |
| ScFv | CD4 | BTNL3 | DAP10 |
| ScFv | CD4 | BTNL3 | DAP12 |
| ScFv | CD4 | BTNL3 | CD32 |
| ScFv | CD4 | BTNL3 | CD79a |
| ScFv | CD4 | BTNL3 | CD79b |
| ScFv | CD4 | NKG2D | CD8 |
| ScFv | CD4 | NKG2D | CD3ζ |
| ScFv | CD4 | NKG2D | CD3δ |
| ScFv | CD4 | NKG2D | CD3γ |
| ScFv | CD4 | NKG2D | CD3ε |
| ScFv | CD4 | NKG2D | FcγRI-γ |
| ScFv | CD4 | NKG2D | FcγRIII-γ |
| ScFv | CD4 | NKG2D | FcεRIβ |
| ScFv | CD4 | NKG2D | FcεRIγ |
| ScFv | CD4 | NKG2D | DAP10 |
| ScFv | CD4 | NKG2D | DAP12 |
| ScFv | CD4 | NKG2D | CD32 |
| ScFv | CD4 | NKG2D | CD79a |
| ScFv | CD4 | NKG2D | CD79b |
| ScFv | b2c | CD28 | CD8 |
| ScFv | b2c | CD28 | CD3ζ |
| ScFv | b2c | CD28 | CD3δ |
| ScFv | b2c | CD28 | CD3γ |
| ScFv | b2c | CD28 | CD3ε |
| ScFv | b2c | CD28 | FcγRI-γ |
| ScFv | b2c | CD28 | FcγRIII-γ |
| ScFv | b2c | CD28 | FcεRIβ |
| ScFv | b2c | CD28 | FcεRIγ |
| ScFv | b2c | CD28 | DAP10 |
| ScFv | b2c | CD28 | DAP12 |
| ScFv | b2c | CD28 | CD32 |
| ScFv | b2c | CD28 | CD79a |
| ScFv | b2c | CD28 | CD79b |
| ScFv | b2c | CD8 | CD8 |
| ScFv | b2c | CD8 | CD3ζ |
| ScFv | b2c | CD8 | CD3δ |
| ScFv | b2c | CD8 | CD3γ |
| ScFv | b2c | CD8 | CD3ε |
| ScFv | b2c | CD8 | FcγRI-γ |
| ScFv | b2c | CD8 | FcγRIII-γ |
| ScFv | b2c | CD8 | FcεRIβ |
| ScFv | b2c | CD8 | FcεRIγ |
| ScFv | b2c | CD8 | DAP10 |
| ScFv | b2c | CD8 | DAP12 |
| ScFv | b2c | CD8 | CD32 |
| ScFv | b2c | CD8 | CD79a |
| ScFv | b2c | CD8 | CD79b |
| ScFv | b2c | CD4 | CD8 |
| ScFv | b2c | CD4 | CD3ζ |
| ScFv | b2c | CD4 | CD3δ |
| ScFv | b2c | CD4 | CD3γ |
| ScFv | b2c | CD4 | CD3ε |
| ScFv | b2c | CD4 | FcγRI-γ |
| ScFv | b2c | CD4 | FcγRIII-γ |
| ScFv | b2c | CD4 | FcεRIβ |
| ScFv | b2c | CD4 | FcεRIγ |
| ScFv | b2c | CD4 | DAP10 |
| ScFv | b2c | CD4 | DAP12 |
| ScFv | b2c | CD4 | CD32 |
| ScFv | b2c | CD4 | CD79a |
| ScFv | b2c | CD4 | CD79b |
| ScFv | b2c | b2c | CD8 |
| ScFv | b2c | b2c | CD3ζ |
| ScFv | b2c | b2c | CD3δ |
| ScFv | b2c | b2c | CD3γ |
| ScFv | b2c | b2c | CD3ε |
| ScFv | b2c | b2c | FcγRI-γ |
| ScFv | b2c | b2c | FcγRIII-γ |
| ScFv | b2c | b2c | FcεRIβ |
| ScFv | b2c | b2c | FcεRIγ |
| ScFv | b2c | b2c | DAP10 |
| ScFv | b2c | b2c | DAP12 |
| ScFv | b2c | b2c | CD32 |
| ScFv | b2c | b2c | CD79a |
| ScFv | b2c | b2c | CD79b |
| ScFv | b2c | CD137/41BB | CD8 |
| ScFv | b2c | CD137/41BB | CD3ζ |
| ScFv | b2c | CD137/41BB | CD3δ |
| ScFv | b2c | CD137/41BB | CD3γ |
| ScFv | b2c | CD137/41BB | CD3ε |
| ScFv | b2c | CD137/41BB | FcγRI-γ |
| ScFv | b2c | CD137/41BB | FcγRIII-γ |
| ScFv | b2c | CD137/41BB | FcεRIβ |
| ScFv | b2c | CD137/41BB | FcεRIγ |
| ScFv | b2c | CD137/41BB | DAP10 |
| ScFv | b2c | CD137/41BB | DAP12 |
| ScFv | b2c | CD137/41BB | CD32 |
| ScFv | b2c | CD137/41BB | CD79a |
| ScFv | b2c | CD137/41BB | CD79b |
| ScFv | b2c | ICOS | CD8 |
| ScFv | b2c | ICOS | CD3ζ |
| ScFv | b2c | ICOS | CD3δ |
| ScFv | b2c | ICOS | CD3γ |
| ScFv | b2c | ICOS | CD3ε |
| ScFv | b2c | ICOS | FcγRI-γ |
| ScFv | b2c | ICOS | FcγRIII-γ |
| ScFv | b2c | ICOS | FcεRIβ |
| ScFv | b2c | ICOS | FcεRIγ |
| ScFv | b2c | ICOS | DAP10 |
| ScFv | b2c | ICOS | DAP12 |
| ScFv | b2c | ICOS | CD32 |
| ScFv | b2c | ICOS | CD79a |
| ScFv | b2c | ICOS | CD79b |
| ScFv | b2c | CD27 | CD8 |
| ScFv | b2c | CD27 | CD3ζ |
| ScFv | b2c | CD27 | CD3δ |
| ScFv | b2c | CD27 | CD3γ |
| ScFv | b2c | CD27 | CD3ε |
| ScFv | b2c | CD27 | FcγRI-γ |
| ScFv | b2c | CD27 | FcγRIII-γ |
| ScFv | b2c | CD27 | FcεRIβ |
| ScFv | b2c | CD27 | FcεRIγ |
| ScFv | b2c | CD27 | DAP10 |
| ScFv | b2c | CD27 | DAP12 |
| ScFv | b2c | CD27 | CD32 |
| ScFv | b2c | CD27 | CD79a |
| ScFv | b2c | CD27 | CD79b |
| ScFv | b2c | CD28δ | CD8 |
| ScFv | b2c | CD28δ | CD3ζ |
| ScFv | b2c | CD28δ | CD3δ |
| ScFv | b2c | CD28δ | CD3γ |
| ScFv | b2c | CD28δ | CD3ε |
| ScFv | b2c | CD28δ | FcγRI-γ |
| ScFv | b2c | CD28δ | FcγRIII-γ |
| ScFv | b2c | CD28δ | FcεRIβ |
| ScFv | b2c | CD28δ | FcεRIγ |
| ScFv | b2c | CD28δ | DAP10 |
| ScFv | b2c | CD28δ | DAP12 |
| ScFv | b2c | CD28δ | CD32 |
| ScFv | b2c | CD28δ | CD79a |
| ScFv | b2c | CD28δ | CD79b |
| ScFv | b2c | CD80 | CD8 |
| ScFv | b2c | CD80 | CD3ζ |
| ScFv | b2c | CD80 | CD3δ |
| ScFv | b2c | CD80 | CD3γ |
| ScFv | b2c | CD80 | CD3ε |
| ScFv | b2c | CD80 | FcγRI-γ |
| ScFv | b2c | CD80 | FcγRIII-γ |
| ScFv | b2c | CD80 | FcεRIβ |
| ScFv | b2c | CD80 | FcεRIγ |
| ScFv | b2c | CD80 | DAP10 |
| ScFv | b2c | CD80 | DAP12 |
| ScFv | b2c | CD80 | CD32 |
| ScFv | b2c | CD80 | CD79a |
| ScFv | b2c | CD80 | CD79b |
| ScFv | b2c | CD86 | CD8 |
| ScFv | b2c | CD86 | CD3ζ |
| ScFv | b2c | CD86 | CD3δ |
| ScFv | b2c | CD86 | CD3γ |
| ScFv | b2c | CD86 | CD3ε |
| ScFv | b2c | CD86 | FcγRI-γ |
| ScFv | b2c | CD86 | FcγRIII-γ |
| ScFv | b2c | CD86 | FcεRIβ |
| ScFv | b2c | CD86 | FcεRIγ |
| ScFv | b2c | CD86 | DAP10 |
| ScFv | b2c | CD86 | DAP12 |
| ScFv | b2c | CD86 | CD32 |
| ScFv | b2c | CD86 | CD79a |
| ScFv | b2c | CD86 | CD79b |
| ScFv | b2c | OX40 | CD8 |
| ScFv | b2c | OX40 | CD3ζ |
| ScFv | b2c | OX40 | CD3δ |
| ScFv | b2c | OX40 | CD3γ |
| ScFv | b2c | OX40 | CD3ε |
| ScFv | b2c | OX40 | FcγRI-γ |
| ScFv | b2c | OX40 | FcγRIII-γ |
| ScFv | b2c | OX40 | FcεRIβ |
| ScFv | b2c | OX40 | FcεRIγ |
| ScFv | b2c | OX40 | DAP10 |
| ScFv | b2c | OX40 | DAP12 |
| ScFv | b2c | OX40 | CD32 |
| ScFv | b2c | OX40 | CD79a |
| ScFv | b2c | OX40 | CD79b |
| ScFv | b2c | DAP10 | CD8 |
| ScFv | b2c | DAP10 | CD3ζ |
| ScFv | b2c | DAP10 | CD3δ |
| ScFv | b2c | DAP10 | CD3γ |
| ScFv | b2c | DAP10 | CD3ε |
| ScFv | b2c | DAP10 | FcγRI-γ |
| ScFv | b2c | DAP10 | FcγRIII-γ |
| ScFv | b2c | DAP10 | FcεRIβ |
| ScFv | b2c | DAP10 | FcεRIγ |
| ScFv | b2c | DAP10 | DAP10 |
| ScFv | b2c | DAP10 | DAP12 |
| ScFv | b2c | DAP10 | CD32 |
| ScFv | b2c | DAP10 | CD79a |
| ScFv | b2c | DAP10 | CD79b |
| ScFv | b2c | DAP12 | CD8 |
| ScFv | b2c | DAP12 | CD3ζ |
| ScFv | b2c | DAP12 | CD3δ |
| ScFv | b2c | DAP12 | CD3γ |
| ScFv | b2c | DAP12 | CD3ε |
| ScFv | b2c | DAP12 | FcγRI-γ |
| ScFv | b2c | DAP12 | FcγRIII-γ |
| ScFv | b2c | DAP12 | FcεRIβ |
| ScFv | b2c | DAP12 | FcεRIγ |
| ScFv | b2c | DAP12 | DAP10 |
| ScFv | b2c | DAP12 | DAP12 |
| ScFv | b2c | DAP12 | CD32 |
| ScFv | b2c | DAP12 | CD79a |
| ScFv | b2c | DAP12 | CD79b |
| ScFv | b2c | MyD88 | CD8 |
| ScFv | b2c | MyD88 | CD3ζ |
| ScFv | b2c | MyD88 | CD3δ |
| ScFv | b2c | MyD88 | CD3γ |
| ScFv | b2c | MyD88 | CD3ε |
| ScFv | b2c | MyD88 | FcγRI-γ |
| ScFv | b2c | MyD88 | FcγRIII-γ |
| ScFv | b2c | MyD88 | FcεRIβ |
| ScFv | b2c | MyD88 | FcεRIγ |
| ScFv | b2c | MyD88 | DAP10 |
| ScFv | b2c | MyD88 | DAP12 |
| ScFv | b2c | MyD88 | CD32 |
| ScFv | b2c | MyD88 | CD79a |
| ScFv | b2c | MyD88 | CD79b |
| ScFv | b2c | CD7 | CD8 |
| ScFv | b2c | CD7 | CD3ζ |
| ScFv | b2c | CD7 | CD3δ |
| ScFv | b2c | CD7 | CD3γ |
| ScFv | b2c | CD7 | CD3ε |
| ScFv | b2c | CD7 | FcγRI-γ |
| ScFv | b2c | CD7 | FcγRIII-γ |
| ScFv | b2c | CD7 | FcεRIβ |
| ScFv | b2c | CD7 | FcεRIγ |
| ScFv | b2c | CD7 | DAP10 |
| ScFv | b2c | CD7 | DAP12 |
| ScFv | b2c | CD7 | CD32 |
| ScFv | b2c | CD7 | CD79a |
| ScFv | b2c | CD7 | CD79b |
| ScFv | b2c | BTNL3 | CD8 |
| ScFv | b2c | BTNL3 | CD3ζ |
| ScFv | b2c | BTNL3 | CD3δ |
| ScFv | b2c | BTNL3 | CD3γ |
| ScFv | b2c | BTNL3 | CD3ε |
| ScFv | b2c | BTNL3 | FcγRI-γ |
| ScFv | b2c | BTNL3 | FcγRIII-γ |
| ScFv | b2c | BTNL3 | FcεRIβ |
| ScFv | b2c | BTNL3 | FcεRIγ |
| ScFv | b2c | BTNL3 | DAP10 |
| ScFv | b2c | BTNL3 | DAP12 |
| ScFv | b2c | BTNL3 | CD32 |
| ScFv | b2c | BTNL3 | CD79a |
| ScFv | b2c | BTNL3 | CD79b |
| ScFv | b2c | NKG2D | CD8 |
| ScFv | b2c | NKG2D | CD3ζ |
| ScFv | b2c | NKG2D | CD3δ |
| ScFv | b2c | NKG2D | CD3γ |
| ScFv | b2c | NKG2D | CD3ε |
| ScFv | b2c | NKG2D | FcγRI-γ |
| ScFv | b2c | NKG2D | FcγRIII-γ |
| ScFv | b2c | NKG2D | FcεRIβ |
| ScFv | b2c | NKG2D | FcεRIγ |
| ScFv | b2c | NKG2D | DAP10 |
| ScFv | b2c | NKG2D | DAP12 |
| ScFv | b2c | NKG2D | CD32 |
| ScFv | b2c | NKG2D | CD79a |
| ScFv | b2c | NKG2D | CD79b |
| ScFv | CD137/41BB | CD28 | CD8 |
| ScFv | CD137/41BB | CD28 | CD3ζ |
| ScFv | CD137/41BB | CD28 | CD3δ |
| ScFv | CD137/41BB | CD28 | CD3γ |
| ScFv | CD137/41BB | CD28 | CD3ε |
| ScFv | CD137/41BB | CD28 | FcγRI-γ |
| ScFv | CD137/41BB | CD28 | FcγRIII-γ |
| ScFv | CD137/41BB | CD28 | FcεRIβ |
| ScFv | CD137/41BB | CD28 | FcεRIγ |
| ScFv | CD137/41BB | CD28 | DAP10 |
| ScFv | CD137/41BB | CD28 | DAP12 |
| ScFv | CD137/41BB | CD28 | CD32 |
| ScFv | CD137/41BB | CD28 | CD79a |
| ScFv | CD137/41BB | CD28 | CD79b |
| ScFv | CD137/41BB | CD8 | CD8 |
| ScFv | CD137/41BB | CD8 | CD3ζ |
| ScFv | CD137/41BB | CD8 | CD3δ |
| ScFv | CD137/41BB | CD8 | CD3γ |
| ScFv | CD137/41BB | CD8 | CD3ε |
| ScFv | CD137/41BB | CD8 | FcγRI-γ |
| ScFv | CD137/41BB | CD8 | FcγRIII-γ |
| ScFv | CD137/41BB | CD8 | FcεRIβ |
| ScFv | CD137/41BB | CD8 | FcεRIγ |
| ScFv | CD137/41BB | CD8 | DAP10 |
| ScFv | CD137/41BB | CD8 | DAP12 |
| ScFv | CD137/41BB | CD8 | CD32 |
| ScFv | CD137/41BB | CD8 | CD79a |
| ScFv | CD137/41BB | CD8 | CD79b |
| ScFv | CD137/41BB | CD4 | CD8 |
| ScFv | CD137/41BB | CD4 | CD3ζ |
| ScFv | CD137/41BB | CD4 | CD3δ |
| ScFv | CD137/41BB | CD4 | CD3γ |
| ScFv | CD137/41BB | CD4 | CD3ε |
| ScFv | CD137/41BB | CD4 | FcγRI-γ |
| ScFv | CD137/41BB | CD4 | FcγRIII-γ |
| ScFv | CD137/41BB | CD4 | FcεRIβ |
| ScFv | CD137/41BB | CD4 | FcεRIγ |
| ScFv | CD137/41BB | CD4 | DAP10 |
| ScFv | CD137/41BB | CD4 | DAP12 |
| ScFv | CD137/41BB | CD4 | CD32 |
| ScFv | CD137/41BB | CD4 | CD79a |
| ScFv | CD137/41BB | CD4 | CD79b |
| ScFv | CD137/41BB | b2c | CD8 |
| ScFv | CD137/41BB | b2c | CD3ζ |
| ScFv | CD137/41BB | b2c | CD3δ |
| ScFv | CD137/41BB | b2c | CD3γ |
| ScFv | CD137/41BB | b2c | CD3ε |
| ScFv | CD137/41BB | b2c | FcγRI-γ |
| ScFv | CD137/41BB | b2c | FcγRIII-γ |
| ScFv | CD137/41BB | b2c | FcεRIβ |
| ScFv | CD137/41BB | b2c | FcεRIγ |
| ScFv | CD137/41BB | b2c | DAP10 |
| ScFv | CD137/41BB | b2c | DAP12 |
| ScFv | CD137/41BB | b2c | CD32 |
| ScFv | CD137/41BB | b2c | CD79a |
| ScFv | CD137/41BB | b2c | CD79b |
| ScFv | CD137/41BB | CD137/41BB | CD8 |
| ScFv | CD137/41BB | CD137/41BB | CD3ζ |
| ScFv | CD137/41BB | CD137/41BB | CD3δ |
| ScFv | CD137/41BB | CD137/41BB | CD3γ |
| ScFv | CD137/41BB | CD137/41BB | CD3ε |
| ScFv | CD137/41BB | CD137/41BB | FcγRI-γ |
| ScFv | CD137/41BB | CD137/41BB | FcγRIII-γ |
| ScFv | CD137/41BB | CD137/41BB | FcεRIβ |
| ScFv | CD137/41BB | CD137/41BB | FcεRIγ |
| ScFv | CD137/41BB | CD137/41BB | DAP10 |
| ScFv | CD137/41BB | CD137/41BB | DAP12 |
| ScFv | CD137/41BB | CD137/41BB | CD32 |
| ScFv | CD137/41BB | CD137/41BB | CD79a |
| ScFv | CD137/41BB | CD137/41BB | CD79b |
| ScFv | CD137/41BB | ICOS | CD8 |
| ScFv | CD137/41BB | ICOS | CD3ζ |
| ScFv | CD137/41BB | ICOS | CD3δ |
| ScFv | CD137/41BB | ICOS | CD3γ |
| ScFv | CD137/41BB | ICOS | CD3ε |
| ScFv | CD137/41BB | ICOS | FcγRI-γ |
| ScFv | CD137/41BB | ICOS | FcγRIII-γ |
| ScFv | CD137/41BB | ICOS | FcεRIβ |
| ScFv | CD137/41BB | ICOS | FcεRIγ |
| ScFv | CD137/41BB | ICOS | DAP10 |
| ScFv | CD137/41BB | ICOS | DAP12 |
| ScFv | CD137/41BB | ICOS | CD32 |
| ScFv | CD137/41BB | ICOS | CD79a |
| ScFv | CD137/41BB | ICOS | CD79b |
| ScFv | CD137/41BB | CD27 | CD8 |
| ScFv | CD137/41BB | CD27 | CD3ζ |
| ScFv | CD137/41BB | CD27 | CD3δ |
| ScFv | CD137/41BB | CD27 | CD3γ |
| ScFv | CD137/41BB | CD27 | CD3ε |
| ScFv | CD137/41BB | CD27 | FcγRI-γ |
| ScFv | CD137/41BB | CD27 | FcγRIII-γ |
| ScFv | CD137/41BB | CD27 | FcεRIβ |
| ScFv | CD137/41BB | CD27 | FcεRIγ |
| ScFv | CD137/41BB | CD27 | DAP10 |
| ScFv | CD137/41BB | CD27 | DAP12 |
| ScFv | CD137/41BB | CD27 | CD32 |
| ScFv | CD137/41BB | CD27 | CD79a |
| ScFv | CD137/41BB | CD27 | CD79b |
| ScFv | CD137/41BB | CD28δ | CD8 |
| ScFv | CD137/41BB | CD28δ | CD3ζ |
| ScFv | CD137/41BB | CD28δ | CD3δ |
| ScFv | CD137/41BB | CD28δ | CD3γ |
| ScFv | CD137/41BB | CD28δ | CD3ε |
| ScFv | CD137/41BB | CD28δ | FcγRI-γ |
| ScFv | CD137/41BB | CD28δ | FcγRIII-γ |
| ScFv | CD137/41BB | CD28δ | FcεRIβ |
| ScFv | CD137/41BB | CD28δ | FcεRIγ |
| ScFv | CD137/41BB | CD28δ | DAP10 |
| ScFv | CD137/41BB | CD28δ | DAP12 |
| ScFv | CD137/41BB | CD28δ | CD32 |
| ScFv | CD137/41BB | CD28δ | CD79a |
| ScFv | CD137/41BB | CD28δ | CD79b |
| ScFv | CD137/41BB | CD80 | CD8 |
| ScFv | CD137/41BB | CD80 | CD3ζ |
| ScFv | CD137/41BB | CD80 | CD3δ |
| ScFv | CD137/41BB | CD80 | CD3γ |
| ScFv | CD137/41BB | CD80 | CD3ε |
| ScFv | CD137/41BB | CD80 | FcγRI-γ |
| ScFv | CD137/41BB | CD80 | FcγRIII-γ |
| ScFv | CD137/41BB | CD80 | FcεRIβ |
| ScFv | CD137/41BB | CD80 | FcεRIγ |
| ScFv | CD137/41BB | CD80 | DAP10 |
| ScFv | CD137/41BB | CD80 | DAP12 |
| ScFv | CD137/41BB | CD80 | CD32 |
| ScFv | CD137/41BB | CD80 | CD79a |
| ScFv | CD137/41BB | CD80 | CD79b |
| ScFv | CD137/41BB | CD86 | CD8 |
| ScFv | CD137/41BB | CD86 | CD3ζ |
| ScFv | CD137/41BB | CD86 | CD3δ |
| ScFv | CD137/41BB | CD86 | CD3γ |
| ScFv | CD137/41BB | CD86 | CD3ε |
| ScFv | CD137/41BB | CD86 | FcγRI-γ |
| ScFv | CD137/41BB | CD86 | FcγRIII-γ |
| ScFv | CD137/41BB | CD86 | FcεRIβ |
| ScFv | CD137/41BB | CD86 | FcεRIγ |
| ScFv | CD137/41BB | CD86 | DAP10 |
| ScFv | CD137/41BB | CD86 | DAP12 |
| ScFv | CD137/41BB | CD86 | CD32 |
| ScFv | CD137/41BB | CD86 | CD79a |
| ScFv | CD137/41BB | CD86 | CD79b |
| ScFv | CD137/41BB | OX40 | CD8 |
| ScFv | CD137/41BB | OX40 | CD3ζ |
| ScFv | CD137/41BB | OX40 | CD3δ |
| ScFv | CD137/41BB | OX40 | CD3γ |
| ScFv | CD137/41BB | OX40 | CD3ε |
| ScFv | CD137/41BB | OX40 | FcγRI-γ |
| ScFv | CD137/41BB | OX40 | FcγRIII-γ |
| ScFv | CD137/41BB | OX40 | FcεRIβ |
| ScFv | CD137/41BB | OX40 | FcεRIγ |
| ScFv | CD137/41BB | OX40 | DAP10 |
| ScFv | CD137/41BB | OX40 | DAP12 |
| ScFv | CD137/41BB | OX40 | CD32 |
| ScFv | CD137/41BB | OX40 | CD79a |
| ScFv | CD137/41BB | OX40 | CD79b |
| ScFv | CD137/41BB | DAP10 | CD8 |
| ScFv | CD137/41BB | DAP10 | CD3ζ |
| ScFv | CD137/41BB | DAP10 | CD3δ |
| ScFv | CD137/41BB | DAP10 | CD3γ |
| ScFv | CD137/41BB | DAP10 | CD3ε |
| ScFv | CD137/41BB | DAP10 | FcγRI-γ |
| ScFv | CD137/41BB | DAP10 | FcγRIII-γ |
| ScFv | CD137/41BB | DAP10 | FcεRIβ |
| ScFv | CD137/41BB | DAP10 | FcεRIγ |
| ScFv | CD137/41BB | DAP10 | DAP10 |
| ScFv | CD137/41BB | DAP10 | DAP12 |
| ScFv | CD137/41BB | DAP10 | CD32 |
| ScFv | CD137/41BB | DAP10 | CD79a |
| ScFv | CD137/41BB | DAP10 | CD79b |
| ScFv | CD137/41BB | DAP12 | CD8 |
| ScFv | CD137/41BB | DAP12 | CD3ζ |
| ScFv | CD137/41BB | DAP12 | CD3δ |
| ScFv | CD137/41BB | DAP12 | CD3γ |
| ScFv | CD137/41BB | DAP12 | CD3ε |
| ScFv | CD137/41BB | DAP12 | FcγRI-γ |
| ScFv | CD137/41BB | DAP12 | FcγRIII-γ |
| ScFv | CD137/41BB | DAP12 | FcεRIβ |
| ScFv | CD137/41BB | DAP12 | FcεRIγ |
| ScFv | CD137/41BB | DAP12 | DAP10 |
| ScFv | CD137/41BB | DAP12 | DAP12 |
| ScFv | CD137/41BB | DAP12 | CD32 |
| ScFv | CD137/41BB | DAP12 | CD79a |
| ScFv | CD137/41BB | DAP12 | CD79b |
| ScFv | CD137/41BB | MyD88 | CD8 |
| ScFv | CD137/41BB | MyD88 | CD3ζ |
| ScFv | CD137/41BB | MyD88 | CD3δ |
| ScFv | CD137/41BB | MyD88 | CD3γ |
| ScFv | CD137/41BB | MyD88 | CD3ε |
| ScFv | CD137/41BB | MyD88 | FcγRI-γ |
| ScFv | CD137/41BB | MyD88 | FcγRIII-γ |
| ScFv | CD137/41BB | MyD88 | FcεRIβ |
| ScFv | CD137/41BB | MyD88 | FcεRIγ |
| ScFv | CD137/41BB | MyD88 | DAP10 |
| ScFv | CD137/41BB | MyD88 | DAP12 |
| ScFv | CD137/41BB | MyD88 | CD32 |
| ScFv | CD137/41BB | MyD88 | CD79a |
| ScFv | CD137/41BB | MyD88 | CD79b |
| ScFv | CD137/41BB | CD7 | CD8 |
| ScFv | CD137/41BB | CD7 | CD3ζ |
| ScFv | CD137/41BB | CD7 | CD3δ |
| ScFv | CD137/41BB | CD7 | CD3γ |
| ScFv | CD137/41BB | CD7 | CD3ε |
| ScFv | CD137/41BB | CD7 | FcγRI-γ |
| ScFv | CD137/41BB | CD7 | FcγRIII-γ |
| ScFv | CD137/41BB | CD7 | FcεRIβ |
| ScFv | CD137/41BB | CD7 | FcεRIγ |
| ScFv | CD137/41BB | CD7 | DAP10 |
| ScFv | CD137/41BB | CD7 | DAP12 |
| ScFv | CD137/41BB | CD7 | CD32 |
| ScFv | CD137/41BB | CD7 | CD79a |
| ScFv | CD137/41BB | CD7 | CD79b |
| ScFv | CD137/41BB | BTNL3 | CD8 |
| ScFv | CD137/41BB | BTNL3 | CD3ζ |
| ScFv | CD137/41BB | BTNL3 | CD3δ |
| ScFv | CD137/41BB | BTNL3 | CD3γ |
| ScFv | CD137/41BB | BTNL3 | CD3ε |
| ScFv | CD137/41BB | BTNL3 | FcγRI-γ |
| ScFv | CD137/41BB | BTNL3 | FcγRIII-γ |
| ScFv | CD137/41BB | BTNL3 | FcεRIβ |
| ScFv | CD137/41BB | BTNL3 | FcεRIγ |
| ScFv | CD137/41BB | BTNL3 | DAP10 |
| ScFv | CD137/41BB | BTNL3 | DAP12 |
| ScFv | CD137/41BB | BTNL3 | CD32 |
| ScFv | CD137/41BB | BTNL3 | CD79a |
| ScFv | CD137/41BB | BTNL3 | CD79b |
| ScFv | CD137/41BB | NKG2D | CD8 |
| ScFv | CD137/41BB | NKG2D | CD3ζ |
| ScFv | CD137/41BB | NKG2D | CD3δ |
| ScFv | CD137/41BB | NKG2D | CD3γ |
| ScFv | CD137/41BB | NKG2D | CD3ε |
| ScFv | CD137/41BB | NKG2D | FcγRI-γ |
| ScFv | CD137/41BB | NKG2D | FcγRIII-γ |
| ScFv | CD137/41BB | NKG2D | FcεRIβ |
| ScFv | CD137/41BB | NKG2D | FcεRIγ |
| ScFv | CD137/41BB | NKG2D | DAP10 |
| ScFv | CD137/41BB | NKG2D | DAP12 |
| ScFv | CD137/41BB | NKG2D | CD32 |
| ScFv | CD137/41BB | NKG2D | CD79a |
| ScFv | CD137/41BB | NKG2D | CD79b |
| ScFv | ICOS | CD28 | CD8 |
| ScFv | ICOS | CD28 | CD3ζ |
| ScFv | ICOS | CD28 | CD3δ |
| ScFv | ICOS | CD28 | CD3γ |
| ScFv | ICOS | CD28 | CD3ε |
| ScFv | ICOS | CD28 | FcγRI-γ |
| ScFv | ICOS | CD28 | FcγRIII-γ |
| ScFv | ICOS | CD28 | FcεRIβ |
| ScFv | ICOS | CD28 | FcεRIγ |
| ScFv | ICOS | CD28 | DAP10 |
| ScFv | ICOS | CD28 | DAP12 |
| ScFv | ICOS | CD28 | CD32 |
| ScFv | ICOS | CD28 | CD79a |
| ScFv | ICOS | CD28 | CD79b |
| ScFv | ICOS | CD8 | CD8 |
| ScFv | ICOS | CD8 | CD3ζ |
| ScFv | ICOS | CD8 | CD3δ |
| ScFv | ICOS | CD8 | CD3γ |
| ScFv | ICOS | CD8 | CD3ε |
| ScFv | ICOS | CD8 | FcγRI-γ |
| ScFv | ICOS | CD8 | FcγRIII-γ |
| ScFv | ICOS | CD8 | FcεRIβ |
| ScFv | ICOS | CD8 | FcεRIγ |
| ScFv | ICOS | CD8 | DAP10 |
| ScFv | ICOS | CD8 | DAP12 |
| ScFv | ICOS | CD8 | CD32 |
| ScFv | ICOS | CD8 | CD79a |
| ScFv | ICOS | CD8 | CD79b |
| ScFv | ICOS | CD4 | CD8 |
| ScFv | ICOS | CD4 | CD3ζ |
| ScFv | ICOS | CD4 | CD3δ |
| ScFv | ICOS | CD4 | CD3γ |
| ScFv | ICOS | CD4 | CD3ε |
| ScFv | ICOS | CD4 | FcγRI-γ |
| ScFv | ICOS | CD4 | FcγRIII-γ |
| ScFv | ICOS | CD4 | FcεRIβ |
| ScFv | ICOS | CD4 | FcεRIγ |
| ScFv | ICOS | CD4 | DAP10 |
| ScFv | ICOS | CD4 | DAP12 |
| ScFv | ICOS | CD4 | CD32 |
| ScFv | ICOS | CD4 | CD79a |
| ScFv | ICOS | CD4 | CD79b |
| ScFv | ICOS | b2c | CD8 |
| ScFv | ICOS | b2c | CD3ζ |
| ScFv | ICOS | b2c | CD3δ |
| ScFv | ICOS | b2c | CD3γ |
| ScFv | ICOS | b2c | CD3ε |
| ScFv | ICOS | b2c | FcγRI-γ |
| ScFv | ICOS | b2c | FcγRIII-γ |
| ScFv | ICOS | b2c | FcεRIβ |
| ScFv | ICOS | b2c | FcεRIγ |
| ScFv | ICOS | b2c | DAP10 |
| ScFv | ICOS | b2c | DAP12 |
| ScFv | ICOS | b2c | CD32 |
| ScFv | ICOS | b2c | CD79a |
| ScFv | ICOS | b2c | CD79b |
| ScFv | ICOS | CD137/41BB | CD8 |
| ScFv | ICOS | CD137/41BB | CD3ζ |
| ScFv | ICOS | CD137/41BB | CD3δ |
| ScFv | ICOS | CD137/41BB | CD3γ |
| ScFv | ICOS | CD137/41BB | CD3ε |
| ScFv | ICOS | CD137/41BB | FcγRI-γ |
| ScFv | ICOS | CD137/41BB | FcγRIII-γ |
| ScFv | ICOS | CD137/41BB | FcεRIβ |
| ScFv | ICOS | CD137/41BB | FcεRIγ |
| ScFv | ICOS | CD137/41BB | DAP10 |
| ScFv | ICOS | CD137/41BB | DAP12 |
| ScFv | ICOS | CD137/41BB | CD32 |
| ScFv | ICOS | CD137/41BB | CD79a |
| ScFv | ICOS | CD137/41BB | CD79b |
| ScFv | ICOS | ICOS | CD8 |
| ScFv | ICOS | ICOS | CD3ζ |
| ScFv | ICOS | ICOS | CD3δ |
| ScFv | ICOS | ICOS | CD3γ |
| ScFv | ICOS | ICOS | CD3ε |
| ScFv | ICOS | ICOS | FcγRI-γ |
| ScFv | ICOS | ICOS | FcγRIII-γ |
| ScFv | ICOS | ICOS | FcεRIβ |
| ScFv | ICOS | ICOS | FcεRIγ |
| ScFv | ICOS | ICOS | DAP10 |
| ScFv | ICOS | ICOS | DAP12 |
| ScFv | ICOS | ICOS | CD32 |
| ScFv | ICOS | ICOS | CD79a |
| ScFv | ICOS | ICOS | CD79b |
| ScFv | ICOS | CD27 | CD8 |
| ScFv | ICOS | CD27 | CD3ζ |
| ScFv | ICOS | CD27 | CD3δ |
| ScFv | ICOS | CD27 | CD3γ |
| ScFv | ICOS | CD27 | CD3ε |
| ScFv | ICOS | CD27 | FcγRI-γ |
| ScFv | ICOS | CD27 | FcγRIII-γ |
| ScFv | ICOS | CD27 | FcεRIβ |
| ScFv | ICOS | CD27 | FcεRIγ |
| ScFv | ICOS | CD27 | DAP10 |
| ScFv | ICOS | CD27 | DAP12 |
| ScFv | ICOS | CD27 | CD32 |
| ScFv | ICOS | CD27 | CD79a |
| ScFv | ICOS | CD27 | CD79b |
| ScFv | ICOS | CD28δ | CD8 |
| ScFv | ICOS | CD28δ | CD3ζ |
| ScFv | ICOS | CD28δ | CD3δ |
| ScFv | ICOS | CD28δ | CD3γ |
| ScFv | ICOS | CD28δ | CD3ε |
| ScFv | ICOS | CD28δ | FcγRI-γ |
| ScFv | ICOS | CD28δ | FcγRIII-γ |
| ScFv | ICOS | CD28δ | FcεRIβ |
| ScFv | ICOS | CD28δ | FcεRIγ |
| ScFv | ICOS | CD28δ | DAP10 |
| ScFv | ICOS | CD28δ | DAP12 |
| ScFv | ICOS | CD28δ | CD32 |
| ScFv | ICOS | CD28δ | CD79a |
| ScFv | ICOS | CD28δ | CD79b |
| ScFv | ICOS | CD80 | CD8 |
| ScFv | ICOS | CD80 | CD3ζ |
| ScFv | ICOS | CD80 | CD3δ |
| ScFv | ICOS | CD80 | CD3γ |
| ScFv | ICOS | CD80 | CD3ε |
| ScFv | ICOS | CD80 | FcγRI-γ |
| ScFv | ICOS | CD80 | FcγRIII-γ |
| ScFv | ICOS | CD80 | FcεRIβ |
| ScFv | ICOS | CD80 | FcεRIγ |
| ScFv | ICOS | CD80 | DAP10 |
| ScFv | ICOS | CD80 | DAP12 |
| ScFv | ICOS | CD80 | CD32 |
| ScFv | ICOS | CD80 | CD79a |
| ScFv | ICOS | CD80 | CD79b |
| ScFv | ICOS | CD86 | CD8 |
| ScFv | ICOS | CD86 | CD3ζ |
| ScFv | ICOS | CD86 | CD3δ |
| ScFv | ICOS | CD86 | CD3γ |
| ScFv | ICOS | CD86 | CD3ε |
| ScFv | ICOS | CD86 | FcγRI-γ |
| ScFv | ICOS | CD86 | FcγRIII-γ |
| ScFv | ICOS | CD86 | FcεRIβ |
| ScFv | ICOS | CD86 | FcεRIγ |
| ScFv | ICOS | CD86 | DAP10 |
| ScFv | ICOS | CD86 | DAP12 |
| ScFv | ICOS | CD86 | CD32 |
| ScFv | ICOS | CD86 | CD79a |
| ScFv | ICOS | CD86 | CD79b |
| ScFv | ICOS | OX40 | CD8 |
| ScFv | ICOS | OX40 | CD3ζ |
| ScFv | ICOS | OX40 | CD3δ |
| ScFv | ICOS | OX40 | CD3γ |
| ScFv | ICOS | OX40 | CD3ε |
| ScFv | ICOS | OX40 | FcγRI-γ |
| ScFv | ICOS | OX40 | FcγRIII-γ |
| ScFv | ICOS | OX40 | FcεRIβ |
| ScFv | ICOS | OX40 | FcεRIγ |
| ScFv | ICOS | OX40 | DAP10 |
| ScFv | ICOS | OX40 | DAP12 |
| ScFv | ICOS | OX40 | CD32 |
| ScFv | ICOS | OX40 | CD79a |
| ScFv | ICOS | OX40 | CD79b |
| ScFv | ICOS | DAP10 | CD8 |
| ScFv | ICOS | DAP10 | CD3ζ |
| ScFv | ICOS | DAP10 | CD3δ |
| ScFv | ICOS | DAP10 | CD3γ |
| ScFv | ICOS | DAP10 | CD3ε |
| ScFv | ICOS | DAP10 | FcγRI-γ |
| ScFv | ICOS | DAP10 | FcγRIII-γ |
| ScFv | ICOS | DAP10 | FcεRIβ |
| ScFv | ICOS | DAP10 | FcεRIγ |
| ScFv | ICOS | DAP10 | DAP10 |
| ScFv | ICOS | DAP10 | DAP12 |
| ScFv | ICOS | DAP10 | CD32 |
| ScFv | ICOS | DAP10 | CD79a |
| ScFv | ICOS | DAP10 | CD79b |
| ScFv | ICOS | DAP12 | CD8 |
| ScFv | ICOS | DAP12 | CD3ζ |
| ScFv | ICOS | DAP12 | CD3δ |
| ScFv | ICOS | DAP12 | CD3γ |
| ScFv | ICOS | DAP12 | CD3ε |
| ScFv | ICOS | DAP12 | FcγRI-γ |
| ScFv | ICOS | DAP12 | FcγRIII-γ |
| ScFv | ICOS | DAP12 | FcεRIβ |
| ScFv | ICOS | DAP12 | FcεRIγ |
| ScFv | ICOS | DAP12 | DAP10 |
| ScFv | ICOS | DAP12 | DAP12 |
| ScFv | ICOS | DAP12 | CD32 |
| ScFv | ICOS | DAP12 | CD79a |
| ScFv | ICOS | DAP12 | CD79b |
| ScFv | ICOS | MyD88 | CD8 |
| ScFv | ICOS | MyD88 | CD3ζ |
| ScFv | ICOS | MyD88 | CD3δ |
| ScFv | ICOS | MyD88 | CD3γ |
| ScFv | ICOS | MyD88 | CD3ε |
| ScFv | ICOS | MyD88 | FcγRI-γ |
| ScFv | ICOS | MyD88 | FcγRIII-γ |
| ScFv | ICOS | MyD88 | FcεRIβ |
| ScFv | ICOS | MyD88 | FcεRIγ |
| ScFv | ICOS | MyD88 | DAP10 |
| ScFv | ICOS | MyD88 | DAP12 |
| ScFv | ICOS | MyD88 | CD32 |
| ScFv | ICOS | MyD88 | CD79a |
| ScFv | ICOS | MyD88 | CD79b |
| ScFv | ICOS | CD7 | CD8 |
| ScFv | ICOS | CD7 | CD3ζ |
| ScFv | ICOS | CD7 | CD3δ |
| ScFv | ICOS | CD7 | CD3γ |
| ScFv | ICOS | CD7 | CD3ε |
| ScFv | ICOS | CD7 | FcγRI-γ |
| ScFv | ICOS | CD7 | FcγRIII-γ |
| ScFv | ICOS | CD7 | FcεRIβ |
| ScFv | ICOS | CD7 | FcεRIγ |
| ScFv | ICOS | CD7 | DAP10 |
| ScFv | ICOS | CD7 | DAP12 |
| ScFv | ICOS | CD7 | CD32 |
| ScFv | ICOS | CD7 | CD79a |
| ScFv | ICOS | CD7 | CD79b |
| ScFv | ICOS | BTNL3 | CD8 |
| ScFv | ICOS | BTNL3 | CD3ζ |
| ScFv | ICOS | BTNL3 | CD3δ |
| ScFv | ICOS | BTNL3 | CD3γ |
| ScFv | ICOS | BTNL3 | CD3ε |
| ScFv | ICOS | BTNL3 | FcγRI-γ |
| ScFv | ICOS | BTNL3 | FcγRIII-γ |
| ScFv | ICOS | BTNL3 | FcεRIβ |
| ScFv | ICOS | BTNL3 | FcεRIγ |
| ScFv | ICOS | BTNL3 | DAP10 |
| ScFv | ICOS | BTNL3 | DAP12 |
| ScFv | ICOS | BTNL3 | CD32 |
| ScFv | ICOS | BTNL3 | CD79a |
| ScFv | ICOS | BTNL3 | CD79b |
| ScFv | ICOS | NKG2D | CD8 |
| ScFv | ICOS | NKG2D | CD3ζ |
| ScFv | ICOS | NKG2D | CD3δ |
| ScFv | ICOS | NKG2D | CD3γ |
| ScFv | ICOS | NKG2D | CD3ε |
| ScFv | ICOS | NKG2D | FcγRI-γ |
| ScFv | ICOS | NKG2D | FcγRIII-γ |
| ScFv | ICOS | NKG2D | FcεRIβ |
| ScFv | ICOS | NKG2D | FcεRIγ |
| ScFv | ICOS | NKG2D | DAP10 |
| ScFv | ICOS | NKG2D | DAP12 |
| ScFv | ICOS | NKG2D | CD32 |
| ScFv | ICOS | NKG2D | CD79a |
| ScFv | ICOS | NKG2D | CD79b |
| ScFv | CD27 | CD28 | CD8 |
| ScFv | CD27 | CD28 | CD3ζ |
| ScFv | CD27 | CD28 | CD3δ |
| ScFv | CD27 | CD28 | CD3γ |
| ScFv | CD27 | CD28 | CD3ε |
| ScFv | CD27 | CD28 | FcγRI-γ |
| ScFv | CD27 | CD28 | FcγRIII-γ |
| ScFv | CD27 | CD28 | FcεRIβ |
| ScFv | CD27 | CD28 | FcεRIγ |
| ScFv | CD27 | CD28 | DAP10 |
| ScFv | CD27 | CD28 | DAP12 |
| ScFv | CD27 | CD28 | CD32 |
| ScFv | CD27 | CD28 | CD79a |
| ScFv | CD27 | CD28 | CD79b |
| ScFv | CD27 | CD8 | CD8 |
| ScFv | CD27 | CD8 | CD3ζ |
| ScFv | CD27 | CD8 | CD3δ |
| ScFv | CD27 | CD8 | CD3γ |
| ScFv | CD27 | CD8 | CD3ε |
| ScFv | CD27 | CD8 | FcγRI-γ |
| ScFv | CD27 | CD8 | FcγRIII-γ |
| ScFv | CD27 | CD8 | FcεRIβ |
| ScFv | CD27 | CD8 | FcεRIγ |
| ScFv | CD27 | CD8 | DAP10 |
| ScFv | CD27 | CD8 | DAP12 |
| ScFv | CD27 | CD8 | CD32 |
| ScFv | CD27 | CD8 | CD79a |
| ScFv | CD27 | CD8 | CD79b |
| ScFv | CD27 | CD4 | CD8 |
| ScFv | CD27 | CD4 | CD3ζ |
| ScFv | CD27 | CD4 | CD3δ |
| ScFv | CD27 | CD4 | CD3γ |
| ScFv | CD27 | CD4 | CD3ε |
| ScFv | CD27 | CD4 | FcγRI-γ |
| ScFv | CD27 | CD4 | FcγRIII-γ |
| ScFv | CD27 | CD4 | FcεRIβ |
| ScFv | CD27 | CD4 | FcεRIγ |
| ScFv | CD27 | CD4 | DAP10 |
| ScFv | CD27 | CD4 | DAP12 |
| ScFv | CD27 | CD4 | CD32 |
| ScFv | CD27 | CD4 | CD79a |
| ScFv | CD27 | CD4 | CD79b |
| ScFv | CD27 | b2c | CD8 |
| ScFv | CD27 | b2c | CD3ζ |
| ScFv | CD27 | b2c | CD3δ |
| ScFv | CD27 | b2c | CD3γ |
| ScFv | CD27 | b2c | CD3ε |
| ScFv | CD27 | b2c | FcγRI-γ |
| ScFv | CD27 | b2c | FcγRIII-γ |
| ScFv | CD27 | b2c | FcεRIβ |
| ScFv | CD27 | b2c | FcεRIγ |
| ScFv | CD27 | b2c | DAP10 |
| ScFv | CD27 | b2c | DAP12 |
| ScFv | CD27 | b2c | CD32 |
| ScFv | CD27 | b2c | CD79a |
| ScFv | CD27 | b2c | CD79b |
| ScFv | CD27 | CD137/41BB | CD8 |
| ScFv | CD27 | CD137/41BB | CD3ζ |
| ScFv | CD27 | CD137/41BB | CD3δ |
| ScFv | CD27 | CD137/41BB | CD3γ |
| ScFv | CD27 | CD137/41BB | CD3ε |
| ScFv | CD27 | CD137/41BB | FcγRI-γ |
| ScFv | CD27 | CD137/41BB | FcγRIII-γ |
| ScFv | CD27 | CD137/41BB | FcεRIβ |
| ScFv | CD27 | CD137/41BB | FcεRIγ |
| ScFv | CD27 | CD137/41BB | DAP10 |
| ScFv | CD27 | CD137/41BB | DAP12 |
| ScFv | CD27 | CD137/41BB | CD32 |
| ScFv | CD27 | CD137/41BB | CD79a |
| ScFv | CD27 | CD137/41BB | CD79b |
| ScFv | CD27 | ICOS | CD8 |
| ScFv | CD27 | ICOS | CD3ζ |
| ScFv | CD27 | ICOS | CD3δ |
| ScFv | CD27 | ICOS | CD3γ |
| ScFv | CD27 | ICOS | CD3ε |
| ScFv | CD27 | ICOS | FcγRI-γ |
| ScFv | CD27 | ICOS | FcγRIII-γ |
| ScFv | CD27 | ICOS | FcεRIβ |
| ScFv | CD27 | ICOS | FcεRIγ |
| ScFv | CD27 | ICOS | DAP10 |
| ScFv | CD27 | ICOS | DAP12 |
| ScFv | CD27 | ICOS | CD32 |
| ScFv | CD27 | ICOS | CD79a |
| ScFv | CD27 | ICOS | CD79b |
| ScFv | CD27 | CD27 | CD8 |
| ScFv | CD27 | CD27 | CD3ζ |
| ScFv | CD27 | CD27 | CD3δ |
| ScFv | CD27 | CD27 | CD3γ |
| ScFv | CD27 | CD27 | CD3ε |
| ScFv | CD27 | CD27 | FcγRI-γ |
| ScFv | CD27 | CD27 | FcγRIII-γ |
| ScFv | CD27 | CD27 | FcεRIβ |
| ScFv | CD27 | CD27 | FcεRIγ |
| ScFv | CD27 | CD27 | DAP10 |
| ScFv | CD27 | CD27 | DAP12 |
| ScFv | CD27 | CD27 | CD32 |
| ScFv | CD27 | CD27 | CD79a |
| ScFv | CD27 | CD27 | CD79b |
| ScFv | CD27 | CD28δ | CD8 |
| ScFv | CD27 | CD28δ | CD3ζ |
| ScFv | CD27 | CD28δ | CD3δ |
| ScFv | CD27 | CD28δ | CD3γ |
| ScFv | CD27 | CD28δ | CD3ε |
| ScFv | CD27 | CD28δ | FcγRI-γ |
| ScFv | CD27 | CD28δ | FcγRIII-γ |
| ScFv | CD27 | CD28δ | FcεRIβ |
| ScFv | CD27 | CD28δ | FcεRIγ |
| ScFv | CD27 | CD28δ | DAP10 |
| ScFv | CD27 | CD28δ | DAP12 |
| ScFv | CD27 | CD28δ | CD32 |
| ScFv | CD27 | CD28δ | CD79a |
| ScFv | CD27 | CD28δ | CD79b |
| ScFv | CD27 | CD80 | CD8 |
| ScFv | CD27 | CD80 | CD3ζ |
| ScFv | CD27 | CD80 | CD3δ |
| ScFv | CD27 | CD80 | CD3γ |
| ScFv | CD27 | CD80 | CD3ε |
| ScFv | CD27 | CD80 | FcγRI-γ |
| ScFv | CD27 | CD80 | FcγRIII-γ |
| ScFv | CD27 | CD80 | FcεRIβ |
| ScFv | CD27 | CD80 | FcεRIγ |
| ScFv | CD27 | CD80 | DAP10 |
| ScFv | CD27 | CD80 | DAP12 |
| ScFv | CD27 | CD80 | CD32 |
| ScFv | CD27 | CD80 | CD79a |
| ScFv | CD27 | CD80 | CD79b |
| ScFv | CD27 | CD86 | CD8 |
| ScFv | CD27 | CD86 | CD3ζ |
| ScFv | CD27 | CD86 | CD3δ |
| ScFv | CD27 | CD86 | CD3γ |
| ScFv | CD27 | CD86 | CD3ε |
| ScFv | CD27 | CD86 | FcγRI-γ |
| ScFv | CD27 | CD86 | FcγRIII-γ |
| ScFv | CD27 | CD86 | FcεRIβ |
| ScFv | CD27 | CD86 | FcεRIγ |
| ScFv | CD27 | CD86 | DAP10 |
| ScFv | CD27 | CD86 | DAP12 |
| ScFv | CD27 | CD86 | CD32 |
| ScFv | CD27 | CD86 | CD79a |
| ScFv | CD27 | CD86 | CD79b |
| ScFv | CD27 | OX40 | CD8 |
| ScFv | CD27 | OX40 | CD3ζ |
| ScFv | CD27 | OX40 | CD3δ |
| ScFv | CD27 | OX40 | CD3γ |
| ScFv | CD27 | OX40 | CD3ε |
| ScFv | CD27 | OX40 | FcγRI-γ |
| ScFv | CD27 | OX40 | FcγRIII-γ |
| ScFv | CD27 | OX40 | FcεRIβ |
| ScFv | CD27 | OX40 | FcεRIγ |
| ScFv | CD27 | OX40 | DAP10 |
| ScFv | CD27 | OX40 | DAP12 |
| ScFv | CD27 | OX40 | CD32 |
| ScFv | CD27 | OX40 | CD79a |
| ScFv | CD27 | OX40 | CD79b |
| ScFv | CD27 | DAP10 | CD8 |
| ScFv | CD27 | DAP10 | CD3ζ |
| ScFv | CD27 | DAP10 | CD3δ |
| ScFv | CD27 | DAP10 | CD3γ |
| ScFv | CD27 | DAP10 | CD3ε |
| ScFv | CD27 | DAP10 | FcγRI-γ |
| ScFv | CD27 | DAP10 | FcγRIII-γ |
| ScFv | CD27 | DAP10 | FcεRIβ |
| ScFv | CD27 | DAP10 | FcεRIγ |
| ScFv | CD27 | DAP10 | DAP10 |
| ScFv | CD27 | DAP10 | DAP12 |
| ScFv | CD27 | DAP10 | CD32 |
| ScFv | CD27 | DAP10 | CD79a |
| ScFv | CD27 | DAP10 | CD79b |
| ScFv | CD27 | DAP12 | CD8 |
| ScFv | CD27 | DAP12 | CD3ζ |
| ScFv | CD27 | DAP12 | CD3δ |
| ScFv | CD27 | DAP12 | CD3γ |
| ScFv | CD27 | DAP12 | CD3ε |
| ScFv | CD27 | DAP12 | FcγRI-γ |
| ScFv | CD27 | DAP12 | FcγRIII-γ |
| ScFv | CD27 | DAP12 | FcεRIβ |
| ScFv | CD27 | DAP12 | FcεRIγ |
| ScFv | CD27 | DAP12 | DAP10 |
| ScFv | CD27 | DAP12 | DAP12 |
| ScFv | CD27 | DAP12 | CD32 |
| ScFv | CD27 | DAP12 | CD79a |
| ScFv | CD27 | DAP12 | CD79b |
| ScFv | CD27 | MyD88 | CD8 |
| ScFv | CD27 | MyD88 | CD3ζ |
| ScFv | CD27 | MyD88 | CD3δ |
| ScFv | CD27 | MyD88 | CD3γ |
| ScFv | CD27 | MyD88 | CD3ε |
| ScFv | CD27 | MyD88 | FcγRI-γ |
| ScFv | CD27 | MyD88 | FcγRIII-γ |
| ScFv | CD27 | MyD88 | FcεRIβ |
| ScFv | CD27 | MyD88 | FcεRIγ |
| ScFv | CD27 | MyD88 | DAP10 |
| ScFv | CD27 | MyD88 | DAP12 |
| ScFv | CD27 | MyD88 | CD32 |
| ScFv | CD27 | MyD88 | CD79a |
| ScFv | CD27 | MyD88 | CD79b |
| ScFv | CD27 | CD7 | CD8 |
| ScFv | CD27 | CD7 | CD3ζ |
| ScFv | CD27 | CD7 | CD3δ |
| ScFv | CD27 | CD7 | CD3γ |
| ScFv | CD27 | CD7 | CD3ε |
| ScFv | CD27 | CD7 | FcγRI-γ |
| ScFv | CD27 | CD7 | FcγRIII-γ |
| ScFv | CD27 | CD7 | FcεRIβ |
| ScFv | CD27 | CD7 | FcεRIγ |
| ScFv | CD27 | CD7 | DAP10 |
| ScFv | CD27 | CD7 | DAP12 |
| ScFv | CD27 | CD7 | CD32 |
| ScFv | CD27 | CD7 | CD79a |
| ScFv | CD27 | CD7 | CD79b |
| ScFv | CD27 | BTNL3 | CD8 |
| ScFv | CD27 | BTNL3 | CD3ζ |
| ScFv | CD27 | BTNL3 | CD3δ |
| ScFv | CD27 | BTNL3 | CD3γ |
| ScFv | CD27 | BTNL3 | CD3ε |
| ScFv | CD27 | BTNL3 | FcγRI-γ |
| ScFv | CD27 | BTNL3 | FcγRIII-γ |
| ScFv | CD27 | BTNL3 | FcεRIβ |
| ScFv | CD27 | BTNL3 | FcεRIγ |
| ScFv | CD27 | BTNL3 | DAP10 |
| ScFv | CD27 | BTNL3 | DAP12 |
| ScFv | CD27 | BTNL3 | CD32 |
| ScFv | CD27 | BTNL3 | CD79a |
| ScFv | CD27 | BTNL3 | CD79b |
| ScFv | CD27 | NKG2D | CD8 |
| ScFv | CD27 | NKG2D | CD3ζ |
| ScFv | CD27 | NKG2D | CD3δ |
| ScFv | CD27 | NKG2D | CD3γ |
| ScFv | CD27 | NKG2D | CD3ε |
| ScFv | CD27 | NKG2D | FcγRI-γ |
| ScFv | CD27 | NKG2D | FcγRIII-γ |
| ScFv | CD27 | NKG2D | FcεRIβ |
| ScFv | CD27 | NKG2D | FcεRIγ |
| ScFv | CD27 | NKG2D | DAP10 |
| ScFv | CD27 | NKG2D | DAP12 |
| ScFv | CD27 | NKG2D | CD32 |
| ScFv | CD27 | NKG2D | CD79a |
| ScFv | CD27 | NKG2D | CD79b |
| ScFv | CD28δ | CD28 | CD8 |
| ScFv | CD28δ | CD28 | CD3ζ |
| ScFv | CD28δ | CD28 | CD3δ |
| ScFv | CD28δ | CD28 | CD3γ |
| ScFv | CD28δ | CD28 | CD3ε |
| ScFv | CD28δ | CD28 | FcγRI-γ |
| ScFv | CD28δ | CD28 | FcγRIII-γ |
| ScFv | CD28δ | CD28 | FcεRIβ |
| ScFv | CD28δ | CD28 | FcεRIγ |
| ScFv | CD28δ | CD28 | DAP10 |
| ScFv | CD28δ | CD28 | DAP12 |
| ScFv | CD28δ | CD28 | CD32 |
| ScFv | CD28δ | CD28 | CD79a |
| ScFv | CD28δ | CD28 | CD79b |
| ScFv | CD28δ | CD8 | CD8 |
| ScFv | CD28δ | CD8 | CD3ζ |
| ScFv | CD28δ | CD8 | CD3δ |
| ScFv | CD28δ | CD8 | CD3γ |
| ScFv | CD28δ | CD8 | CD3ε |
| ScFv | CD28δ | CD8 | FcγRI-γ |
| ScFv | CD28δ | CD8 | FcγRIII-γ |
| ScFv | CD28δ | CD8 | FcεRIβ |
| ScFv | CD28δ | CD8 | FcεRIγ |
| ScFv | CD28δ | CD8 | DAP10 |
| ScFv | CD28δ | CD8 | DAP12 |
| ScFv | CD28δ | CD8 | CD32 |
| ScFv | CD28δ | CD8 | CD79a |
| ScFv | CD28δ | CD8 | CD79b |
| ScFv | CD28δ | CD4 | CD8 |
| ScFv | CD28δ | CD4 | CD3ζ |
| ScFv | CD28δ | CD4 | CD3δ |
| ScFv | CD28δ | CD4 | CD3γ |
| ScFv | CD28δ | CD4 | CD3ε |
| ScFv | CD28δ | CD4 | FcγRI-γ |
| ScFv | CD28δ | CD4 | FcγRIII-γ |
| ScFv | CD28δ | CD4 | FcεRIβ |
| ScFv | CD28δ | CD4 | FcεRIγ |
| ScFv | CD28δ | CD4 | DAP10 |
| ScFv | CD28δ | CD4 | DAP12 |
| ScFv | CD28δ | CD4 | CD32 |
| ScFv | CD28δ | CD4 | CD79a |
| ScFv | CD28δ | CD4 | CD79b |
| ScFv | CD28δ | b2c | CD8 |
| ScFv | CD28δ | b2c | CD3ζ |
| ScFv | CD28δ | b2c | CD3δ |
| ScFv | CD28δ | b2c | CD3γ |
| ScFv | CD28δ | b2c | CD3ε |
| ScFv | CD28δ | b2c | FcγRI-γ |
| ScFv | CD28δ | b2c | FcγRIII-γ |
| ScFv | CD28δ | b2c | FcεRIβ |
| ScFv | CD28δ | b2c | FcεRIγ |
| ScFv | CD28δ | b2c | DAP10 |
| ScFv | CD28δ | b2c | DAP12 |
| ScFv | CD28δ | b2c | CD32 |
| ScFv | CD28δ | b2c | CD79a |
| ScFv | CD28δ | b2c | CD79b |
| ScFv | CD28δ | CD137/41BB | CD8 |
| ScFv | CD28δ | CD137/41BB | CD3ζ |
| ScFv | CD28δ | CD137/41BB | CD3δ |
| ScFv | CD28δ | CD137/41BB | CD3γ |
| ScFv | CD28δ | CD137/41BB | CD3ε |
| ScFv | CD28δ | CD137/41BB | FcγRI-γ |
| ScFv | CD28δ | CD137/41BB | FcγRIII-γ |
| ScFv | CD28δ | CD137/41BB | FcεRIβ |
| ScFv | CD28δ | CD137/41BB | FcεRIγ |
| ScFv | CD28δ | CD137/41BB | DAP10 |
| ScFv | CD28δ | CD137/41BB | DAP12 |
| ScFv | CD28δ | CD137/41BB | CD32 |
| ScFv | CD28δ | CD137/41BB | CD79a |
| ScFv | CD28δ | CD137/41BB | CD79b |
| ScFv | CD28δ | ICOS | CD8 |
| ScFv | CD28δ | ICOS | CD3ζ |
| ScFv | CD28δ | ICOS | CD3δ |
| ScFv | CD28δ | ICOS | CD3γ |
| ScFv | CD28δ | ICOS | CD3ε |
| ScFv | CD28δ | ICOS | FcγRI-γ |
| ScFv | CD28δ | ICOS | FcγRIII-γ |
| ScFv | CD28δ | ICOS | FcεRIβ |
| ScFv | CD28δ | ICOS | FcεRIγ |
| ScFv | CD28δ | ICOS | DAP10 |
| ScFv | CD28δ | ICOS | DAP12 |
| ScFv | CD28δ | ICOS | CD32 |
| ScFv | CD28δ | ICOS | CD79a |
| ScFv | CD28δ | ICOS | CD79b |
| ScFv | CD28δ | CD27 | CD8 |
| ScFv | CD28δ | CD27 | CD3ζ |
| ScFv | CD28δ | CD27 | CD3δ |
| ScFv | CD28δ | CD27 | CD3γ |
| ScFv | CD28δ | CD27 | CD3ε |
| ScFv | CD28δ | CD27 | FcγRI-γ |
| ScFv | CD28δ | CD27 | FcγRIII-γ |
| ScFv | CD28δ | CD27 | FcεRIβ |
| ScFv | CD28δ | CD27 | FcεRIγ |
| ScFv | CD28δ | CD27 | DAP10 |
| ScFv | CD28δ | CD27 | DAP12 |
| ScFv | CD28δ | CD27 | CD32 |
| ScFv | CD28δ | CD27 | CD79a |
| ScFv | CD28δ | CD27 | CD79b |
| ScFv | CD28δ | CD28δ | CD8 |
| ScFv | CD28δ | CD28δ | CD3ζ |
| ScFv | CD28δ | CD28δ | CD3δ |
| ScFv | CD28δ | CD28δ | CD3γ |
| ScFv | CD28δ | CD28δ | CD3ε |
| ScFv | CD28δ | CD28δ | FcγRI-γ |
| ScFv | CD28δ | CD28δ | FcγRIII-γ |
| ScFv | CD28δ | CD28δ | FcεRIβ |
| ScFv | CD28δ | CD28δ | FcεRIγ |
| ScFv | CD28δ | CD28δ | DAP10 |
| ScFv | CD28δ | CD28δ | DAP12 |
| ScFv | CD28δ | CD28δ | CD32 |
| ScFv | CD28δ | CD28δ | CD79a |
| ScFv | CD28δ | CD28δ | CD79b |
| ScFv | CD28δ | CD80 | CD8 |
| ScFv | CD28δ | CD80 | CD3ζ |
| ScFv | CD28δ | CD80 | CD3δ |
| ScFv | CD28δ | CD80 | CD3γ |
| ScFv | CD28δ | CD80 | CD3ε |
| ScFv | CD28δ | CD80 | FcγRI-γ |
| ScFv | CD28δ | CD80 | FcγRIII-γ |
| ScFv | CD28δ | CD80 | FcεRIβ |
| ScFv | CD28δ | CD80 | FcεRIγ |
| ScFv | CD28δ | CD80 | DAP10 |
| ScFv | CD28δ | CD80 | DAP12 |
| ScFv | CD28δ | CD80 | CD32 |
| ScFv | CD28δ | CD80 | CD79a |
| ScFv | CD28δ | CD80 | CD79b |
| ScFv | CD28δ | CD86 | CD8 |
| ScFv | CD28δ | CD86 | CD3ζ |
| ScFv | CD28δ | CD86 | CD3δ |
| ScFv | CD28δ | CD86 | CD3γ |
| ScFv | CD28δ | CD86 | CD3ε |
| ScFv | CD28δ | CD86 | FcγRI-γ |
| ScFv | CD28δ | CD86 | FcγRIII-γ |
| ScFv | CD28δ | CD86 | FcεRIβ |
| ScFv | CD28δ | CD86 | FcεRIγ |
| ScFv | CD28δ | CD86 | DAP10 |
| ScFv | CD28δ | CD86 | DAP12 |
| ScFv | CD28δ | CD86 | CD32 |
| ScFv | CD28δ | CD86 | CD79a |
| ScFv | CD28δ | CD86 | CD79b |
| ScFv | CD28δ | OX40 | CD8 |
| ScFv | CD28δ | OX40 | CD3ζ |
| ScFv | CD28δ | OX40 | CD3δ |
| ScFv | CD28δ | OX40 | CD3γ |
| ScFv | CD28δ | OX40 | CD3ε |
| ScFv | CD28δ | OX40 | FcγRI-γ |
| ScFv | CD28δ | OX40 | FcγRIII-γ |
| ScFv | CD28δ | OX40 | FcεRIβ |
| ScFv | CD28δ | OX40 | FcεRIγ |
| ScFv | CD28δ | OX40 | DAP10 |
| ScFv | CD28δ | OX40 | DAP12 |
| ScFv | CD28δ | OX40 | CD32 |
| ScFv | CD28δ | OX40 | CD79a |
| ScFv | CD28δ | OX40 | CD79b |
| ScFv | CD28δ | DAP10 | CD8 |
| ScFv | CD28δ | DAP10 | CD3ζ |
| ScFv | CD28δ | DAP10 | CD3δ |
| ScFv | CD28δ | DAP10 | CD3γ |
| ScFv | CD28δ | DAP10 | CD3ε |
| ScFv | CD28δ | DAP10 | FcγRI-γ |
| ScFv | CD28δ | DAP10 | FcγRIII-γ |
| ScFv | CD28δ | DAP10 | FcεRIβ |
| ScFv | CD28δ | DAP10 | FcεRIγ |
| ScFv | CD28δ | DAP10 | DAP10 |
| ScFv | CD28δ | DAP10 | DAP12 |
| ScFv | CD28δ | DAP10 | CD32 |
| ScFv | CD28δ | DAP10 | CD79a |
| ScFv | CD28δ | DAP10 | CD79b |
| ScFv | CD28δ | DAP12 | CD8 |
| ScFv | CD28δ | DAP12 | CD3ζ |
| ScFv | CD28δ | DAP12 | CD3δ |
| ScFv | CD28δ | DAP12 | CD3γ |
| ScFv | CD28δ | DAP12 | CD3ε |
| ScFv | CD28δ | DAP12 | FcγRI-γ |
| ScFv | CD28δ | DAP12 | FcγRIII-γ |
| ScFv | CD28δ | DAP12 | FcεRIβ |
| ScFv | CD28δ | DAP12 | FcεRIγ |
| ScFv | CD28δ | DAP12 | DAP10 |
| ScFv | CD28δ | DAP12 | DAP12 |
| ScFv | CD28δ | DAP12 | CD32 |
| ScFv | CD28δ | DAP12 | CD79a |
| ScFv | CD28δ | DAP12 | CD79b |
| ScFv | CD28δ | MyD88 | CD8 |
| ScFv | CD28δ | MyD88 | CD3ζ |
| ScFv | CD28δ | MyD88 | CD3δ |
| ScFv | CD28δ | MyD88 | CD3γ |
| ScFv | CD28δ | MyD88 | CD3ε |
| ScFv | CD28δ | MyD88 | FcγRI-γ |
| ScFv | CD28δ | MyD88 | FcγRIII-γ |
| ScFv | CD28δ | MyD88 | FcεRIβ |
| ScFv | CD28δ | MyD88 | FcεRIγ |
| ScFv | CD28δ | MyD88 | DAP10 |
| ScFv | CD28δ | MyD88 | DAP12 |
| ScFv | CD28δ | MyD88 | CD32 |
| ScFv | CD28δ | MyD88 | CD79a |
| ScFv | CD28δ | MyD88 | CD79b |
| ScFv | CD28δ | CD7 | CD8 |
| ScFv | CD28δ | CD7 | CD3ζ |
| ScFv | CD28δ | CD7 | CD3δ |
| ScFv | CD28δ | CD7 | CD3γ |
| ScFv | CD28δ | CD7 | CD3ε |
| ScFv | CD28δ | CD7 | FcγRI-γ |
| ScFv | CD28δ | CD7 | FcγRIII-γ |
| ScFv | CD28δ | CD7 | FcεRIβ |
| ScFv | CD28δ | CD7 | FcεRIγ |
| ScFv | CD28δ | CD7 | DAP10 |
| ScFv | CD28δ | CD7 | DAP12 |
| ScFv | CD28δ | CD7 | CD32 |
| ScFv | CD28δ | CD7 | CD79a |
| ScFv | CD28δ | CD7 | CD79b |
| ScFv | CD28δ | BTNL3 | CD8 |
| ScFv | CD28δ | BTNL3 | CD3ζ |
| ScFv | CD28δ | BTNL3 | CD3δ |
| ScFv | CD28δ | BTNL3 | CD3γ |
| ScFv | CD28δ | BTNL3 | CD3ε |
| ScFv | CD28δ | BTNL3 | FcγRI-γ |
| ScFv | CD28δ | BTNL3 | FcγRIII-γ |
| ScFv | CD28δ | BTNL3 | FcεRIβ |
| ScFv | CD28δ | BTNL3 | FcεRIγ |
| ScFv | CD28δ | BTNL3 | DAP10 |
| ScFv | CD28δ | BTNL3 | DAP12 |
| ScFv | CD28δ | BTNL3 | CD32 |
| ScFv | CD28δ | BTNL3 | CD79a |
| ScFv | CD28δ | BTNL3 | CD79b |
| ScFv | CD28δ | NKG2D | CD8 |
| ScFv | CD28δ | NKG2D | CD3ζ |
| ScFv | CD28δ | NKG2D | CD3δ |
| ScFv | CD28δ | NKG2D | CD3γ |
| ScFv | CD28δ | NKG2D | CD3ε |
| ScFv | CD28δ | NKG2D | FcγRI-γ |
| ScFv | CD28δ | NKG2D | FcγRIII-γ |
| ScFv | CD28δ | NKG2D | FcεRIβ |
| ScFv | CD28δ | NKG2D | FcεRIγ |
| ScFv | CD28δ | NKG2D | DAP10 |
| ScFv | CD28δ | NKG2D | DAP12 |
| ScFv | CD28δ | NKG2D | CD32 |
| ScFv | CD28δ | NKG2D | CD79a |
| ScFv | CD28δ | NKG2D | CD79b |
| ScFv | CD80 | CD28 | CD8 |
| ScFv | CD80 | CD28 | CD3ζ |
| ScFv | CD80 | CD28 | CD3δ |
| ScFv | CD80 | CD28 | CD3γ |
| ScFv | CD80 | CD28 | CD3ε |
| ScFv | CD80 | CD28 | FcγRI-γ |
| ScFv | CD80 | CD28 | FcγRIII-γ |
| ScFv | CD80 | CD28 | FcεRIβ |
| ScFv | CD80 | CD28 | FcεRIγ |
| ScFv | CD80 | CD28 | DAP10 |
| ScFv | CD80 | CD28 | DAP12 |
| ScFv | CD80 | CD28 | CD32 |
| ScFv | CD80 | CD28 | CD79a |
| ScFv | CD80 | CD28 | CD79b |
| ScFv | CD80 | CD8 | CD8 |
| ScFv | CD80 | CD8 | CD3ζ |
| ScFv | CD80 | CD8 | CD3δ |
| ScFv | CD80 | CD8 | CD3γ |
| ScFv | CD80 | CD8 | CD3ε |
| ScFv | CD80 | CD8 | FcγRI-γ |
| ScFv | CD80 | CD8 | FcγRIII-γ |
| ScFv | CD80 | CD8 | FcεRIβ |
| ScFv | CD80 | CD8 | FcεRIγ |
| ScFv | CD80 | CD8 | DAP10 |
| ScFv | CD80 | CD8 | DAP12 |
| ScFv | CD80 | CD8 | CD32 |
| ScFv | CD80 | CD8 | CD79a |
| ScFv | CD80 | CD8 | CD79b |
| ScFv | CD80 | CD4 | CD8 |
| ScFv | CD80 | CD4 | CD3ζ |
| ScFv | CD80 | CD4 | CD3δ |
| ScFv | CD80 | CD4 | CD3γ |
| ScFv | CD80 | CD4 | CD3ε |
| ScFv | CD80 | CD4 | FcγRI-γ |
| ScFv | CD80 | CD4 | FcγRIII-γ |
| ScFv | CD80 | CD4 | FcεRIβ |
| ScFv | CD80 | CD4 | FcεRIγ |
| ScFv | CD80 | CD4 | DAP10 |
| ScFv | CD80 | CD4 | DAP12 |
| ScFv | CD80 | CD4 | CD32 |
| ScFv | CD80 | CD4 | CD79a |
| ScFv | CD80 | CD4 | CD79b |
| ScFv | CD80 | b2c | CD8 |
| ScFv | CD80 | b2c | CD3ζ |
| ScFv | CD80 | b2c | CD3δ |
| ScFv | CD80 | b2c | CD3γ |
| ScFv | CD80 | b2c | CD3ε |
| ScFv | CD80 | b2c | FcγRI-γ |
| ScFv | CD80 | b2c | FcγRIII-γ |
| ScFv | CD80 | b2c | FcεRIβ |
| ScFv | CD80 | b2c | FcεRIγ |
| ScFv | CD80 | b2c | DAP10 |
| ScFv | CD80 | b2c | DAP12 |
| ScFv | CD80 | b2c | CD32 |
| ScFv | CD80 | b2c | CD79a |
| ScFv | CD80 | b2c | CD79b |
| ScFv | CD80 | CD137/41BB | CD8 |
| ScFv | CD80 | CD137/41BB | CD3ζ |
| ScFv | CD80 | CD137/41BB | CD3δ |
| ScFv | CD80 | CD137/41BB | CD3γ |
| ScFv | CD80 | CD137/41BB | CD3ε |
| ScFv | CD80 | CD137/41BB | FcγRI-γ |
| ScFv | CD80 | CD137/41BB | FcγRIII-γ |
| ScFv | CD80 | CD137/41BB | FcεRIβ |
| ScFv | CD80 | CD137/41BB | FcεRIγ |
| ScFv | CD80 | CD137/41BB | DAP10 |
| ScFv | CD80 | CD137/41BB | DAP12 |
| ScFv | CD80 | CD137/41BB | CD32 |
| ScFv | CD80 | CD137/41BB | CD79a |
| ScFv | CD80 | CD137/41BB | CD79b |
| ScFv | CD80 | ICOS | CD8 |
| ScFv | CD80 | ICOS | CD3ζ |
| ScFv | CD80 | ICOS | CD3δ |
| ScFv | CD80 | ICOS | CD3γ |
| ScFv | CD80 | ICOS | CD3ε |
| ScFv | CD80 | ICOS | FcγRI-γ |
| ScFv | CD80 | ICOS | FcγRIII-γ |
| ScFv | CD80 | ICOS | FcεRIβ |
| ScFv | CD80 | ICOS | FcεRIγ |
| ScFv | CD80 | ICOS | DAP10 |
| ScFv | CD80 | ICOS | DAP12 |
| ScFv | CD80 | ICOS | CD32 |
| ScFv | CD80 | ICOS | CD79a |
| ScFv | CD80 | ICOS | CD79b |
| ScFv | CD80 | CD27 | CD8 |
| ScFv | CD80 | CD27 | CD3ζ |
| ScFv | CD80 | CD27 | CD3δ |
| ScFv | CD80 | CD27 | CD3γ |
| ScFv | CD80 | CD27 | CD3ε |
| ScFv | CD80 | CD27 | FcγRI-γ |
| ScFv | CD80 | CD27 | FcγRIII-γ |
| ScFv | CD80 | CD27 | FcεRIβ |
| ScFv | CD80 | CD27 | FcεRIγ |
| ScFv | CD80 | CD27 | DAP10 |
| ScFv | CD80 | CD27 | DAP12 |
| ScFv | CD80 | CD27 | CD32 |
| ScFv | CD80 | CD27 | CD79a |
| ScFv | CD80 | CD27 | CD79b |
| ScFv | CD80 | CD28δ | CD8 |
| ScFv | CD80 | CD28δ | CD3ζ |
| ScFv | CD80 | CD28δ | CD3δ |
| ScFv | CD80 | CD28δ | CD3γ |
| ScFv | CD80 | CD28δ | CD3ε |
| ScFv | CD80 | CD28δ | FcγRI-γ |
| ScFv | CD80 | CD28δ | FcγRIII-γ |
| ScFv | CD80 | CD28δ | FcεRIβ |
| ScFv | CD80 | CD28δ | FcεRIγ |
| ScFv | CD80 | CD28δ | DAP10 |
| ScFv | CD80 | CD28δ | DAP12 |
| ScFv | CD80 | CD28δ | CD32 |
| ScFv | CD80 | CD28δ | CD79a |
| ScFv | CD80 | CD28δ | CD79b |
| ScFv | CD80 | CD80 | CD8 |
| ScFv | CD80 | CD80 | CD3ζ |
| ScFv | CD80 | CD80 | CD3δ |
| ScFv | CD80 | CD80 | CD3γ |
| ScFv | CD80 | CD80 | CD3ε |
| ScFv | CD80 | CD80 | FcγRI-γ |
| ScFv | CD80 | CD80 | FcγRIII-γ |
| ScFv | CD80 | CD80 | FcεRIβ |
| ScFv | CD80 | CD80 | FcεRIγ |
| ScFv | CD80 | CD80 | DAP10 |
| ScFv | CD80 | CD80 | DAP12 |
| ScFv | CD80 | CD80 | CD32 |
| ScFv | CD80 | CD80 | CD79a |
| ScFv | CD80 | CD80 | CD79b |
| ScFv | CD80 | CD86 | CD8 |
| ScFv | CD80 | CD86 | CD3ζ |
| ScFv | CD80 | CD86 | CD3δ |
| ScFv | CD80 | CD86 | CD3γ |
| ScFv | CD80 | CD86 | CD3ε |
| ScFv | CD80 | CD86 | FcγRI-γ |
| ScFv | CD80 | CD86 | FcγRIII-γ |
| ScFv | CD80 | CD86 | FcεRIβ |
| ScFv | CD80 | CD86 | FcεRIγ |
| ScFv | CD80 | CD86 | DAP10 |
| ScFv | CD80 | CD86 | DAP12 |
| ScFv | CD80 | CD86 | CD32 |
| ScFv | CD80 | CD86 | CD79a |
| ScFv | CD80 | CD86 | CD79b |
| ScFv | CD80 | OX40 | CD8 |
| ScFv | CD80 | OX40 | CD3ζ |
| ScFv | CD80 | OX40 | CD3δ |
| ScFv | CD80 | OX40 | CD3γ |
| ScFv | CD80 | OX40 | CD3ε |
| ScFv | CD80 | OX40 | FcγRI-γ |
| ScFv | CD80 | OX40 | FcγRIII-γ |
| ScFv | CD80 | OX40 | FcεRIβ |
| ScFv | CD80 | OX40 | FcεRIγ |
| ScFv | CD80 | OX40 | DAP10 |
| ScFv | CD80 | OX40 | DAP12 |
| ScFv | CD80 | OX40 | CD32 |
| ScFv | CD80 | OX40 | CD79a |
| ScFv | CD80 | OX40 | CD79b |
| ScFv | CD80 | DAP10 | CD8 |
| ScFv | CD80 | DAP10 | CD3ζ |
| ScFv | CD80 | DAP10 | CD3δ |
| ScFv | CD80 | DAP10 | CD3γ |
| ScFv | CD80 | DAP10 | CD3ε |
| ScFv | CD80 | DAP10 | FcγRI-γ |
| ScFv | CD80 | DAP10 | FcγRIII-γ |
| ScFv | CD80 | DAP10 | FcεRIβ |
| ScFv | CD80 | DAP10 | FcεRIγ |
| ScFv | CD80 | DAP10 | DAP10 |
| ScFv | CD80 | DAP10 | DAP12 |
| ScFv | CD80 | DAP10 | CD32 |
| ScFv | CD80 | DAP10 | CD79a |
| ScFv | CD80 | DAP10 | CD79b |
| ScFv | CD80 | DAP12 | CD8 |
| ScFv | CD80 | DAP12 | CD3ζ |
| ScFv | CD80 | DAP12 | CD3δ |
| ScFv | CD80 | DAP12 | CD3γ |
| ScFv | CD80 | DAP12 | CD3ε |
| ScFv | CD80 | DAP12 | FcγRI-γ |
| ScFv | CD80 | DAP12 | FcγRIII-γ |
| ScFv | CD80 | DAP12 | FcεRIβ |
| ScFv | CD80 | DAP12 | FcεRIγ |
| ScFv | CD80 | DAP12 | DAP10 |
| ScFv | CD80 | DAP12 | DAP12 |
| ScFv | CD80 | DAP12 | CD32 |
| ScFv | CD80 | DAP12 | CD79a |
| ScFv | CD80 | DAP12 | CD79b |
| ScFv | CD80 | MyD88 | CD8 |
| ScFv | CD80 | MyD88 | CD3ζ |
| ScFv | CD80 | MyD88 | CD3δ |
| ScFv | CD80 | MyD88 | CD3γ |
| ScFv | CD80 | MyD88 | CD3ε |
| ScFv | CD80 | MyD88 | FcγRI-γ |
| ScFv | CD80 | MyD88 | FcγRIII-γ |
| ScFv | CD80 | MyD88 | FcεRIβ |
| ScFv | CD80 | MyD88 | FcεRIγ |
| ScFv | CD80 | MyD88 | DAP10 |
| ScFv | CD80 | MyD88 | DAP12 |
| ScFv | CD80 | MyD88 | CD32 |
| ScFv | CD80 | MyD88 | CD79a |
| ScFv | CD80 | MyD88 | CD79b |
| ScFv | CD80 | CD7 | CD8 |
| ScFv | CD80 | CD7 | CD3ζ |
| ScFv | CD80 | CD7 | CD3δ |
| ScFv | CD80 | CD7 | CD3γ |
| ScFv | CD80 | CD7 | CD3ε |
| ScFv | CD80 | CD7 | FcγRI-γ |
| ScFv | CD80 | CD7 | FcγRIII-γ |
| ScFv | CD80 | CD7 | FcεRIβ |
| ScFv | CD80 | CD7 | FcεRIγ |
| ScFv | CD80 | CD7 | DAP10 |
| ScFv | CD80 | CD7 | DAP12 |
| ScFv | CD80 | CD7 | CD32 |
| ScFv | CD80 | CD7 | CD79a |
| ScFv | CD80 | CD7 | CD79b |
| ScFv | CD80 | BTNL3 | CD8 |
| ScFv | CD80 | BTNL3 | CD3ζ |
| ScFv | CD80 | BTNL3 | CD3δ |
| ScFv | CD80 | BTNL3 | CD3γ |
| ScFv | CD80 | BTNL3 | CD3ε |
| ScFv | CD80 | BTNL3 | FcγRI-γ |
| ScFv | CD80 | BTNL3 | FcγRIII-γ |
| ScFv | CD80 | BTNL3 | FcεRIβ |
| ScFv | CD80 | BTNL3 | FcεRIγ |
| ScFv | CD80 | BTNL3 | DAP10 |
| ScFv | CD80 | BTNL3 | DAP12 |
| ScFv | CD80 | BTNL3 | CD32 |
| ScFv | CD80 | BTNL3 | CD79a |
| ScFv | CD80 | BTNL3 | CD79b |
| ScFv | CD80 | NKG2D | CD8 |
| ScFv | CD80 | NKG2D | CD3ζ |
| ScFv | CD80 | NKG2D | CD3δ |
| ScFv | CD80 | NKG2D | CD3γ |
| ScFv | CD80 | NKG2D | CD3ε |
| ScFv | CD80 | NKG2D | FcγRI-γ |
| ScFv | CD80 | NKG2D | FcγRIII-γ |
| ScFv | CD80 | NKG2D | FcεRIβ |
| ScFv | CD80 | NKG2D | FcεRIγ |
| ScFv | CD80 | NKG2D | DAP10 |
| ScFv | CD80 | NKG2D | DAP12 |
| ScFv | CD80 | NKG2D | CD32 |
| ScFv | CD80 | NKG2D | CD79a |
| ScFv | CD80 | NKG2D | CD79b |
| ScFv | CD86 | CD28 | CD8 |
| ScFv | CD86 | CD28 | CD3ζ |
| ScFv | CD86 | CD28 | CD3δ |
| ScFv | CD86 | CD28 | CD3γ |
| ScFv | CD86 | CD28 | CD3ε |
| ScFv | CD86 | CD28 | FcγRI-γ |
| ScFv | CD86 | CD28 | FcγRIII-γ |
| ScFv | CD86 | CD28 | FcεRIβ |
| ScFv | CD86 | CD28 | FcεRIγ |
| ScFv | CD86 | CD28 | DAP10 |
| ScFv | CD86 | CD28 | DAP12 |
| ScFv | CD86 | CD28 | CD32 |
| ScFv | CD86 | CD28 | CD79a |
| ScFv | CD86 | CD28 | CD79b |
| ScFv | CD86 | CD8 | CD8 |
| ScFv | CD86 | CD8 | CD3ζ |
| ScFv | CD86 | CD8 | CD3δ |
| ScFv | CD86 | CD8 | CD3γ |
| ScFv | CD86 | CD8 | CD3ε |
| ScFv | CD86 | CD8 | FcγRI-γ |
| ScFv | CD86 | CD8 | FcγRIII-γ |
| ScFv | CD86 | CD8 | FcεRIβ |
| ScFv | CD86 | CD8 | FcεRIγ |
| ScFv | CD86 | CD8 | DAP10 |
| ScFv | CD86 | CD8 | DAP12 |
| ScFv | CD86 | CD8 | CD32 |
| ScFv | CD86 | CD8 | CD79a |
| ScFv | CD86 | CD8 | CD79b |
| ScFv | CD86 | CD4 | CD8 |
| ScFv | CD86 | CD4 | CD3ζ |
| ScFv | CD86 | CD4 | CD3δ |
| ScFv | CD86 | CD4 | CD3γ |
| ScFv | CD86 | CD4 | CD3ε |
| ScFv | CD86 | CD4 | FcγRI-γ |
| ScFv | CD86 | CD4 | FcγRIII-γ |
| ScFv | CD86 | CD4 | FcεRIβ |
| ScFv | CD86 | CD4 | FcεRIγ |
| ScFv | CD86 | CD4 | DAP10 |
| ScFv | CD86 | CD4 | DAP12 |
| ScFv | CD86 | CD4 | CD32 |
| ScFv | CD86 | CD4 | CD79a |
| ScFv | CD86 | CD4 | CD79b |
| ScFv | CD86 | b2c | CD8 |
| ScFv | CD86 | b2c | CD3ζ |
| ScFv | CD86 | b2c | CD3δ |
| ScFv | CD86 | b2c | CD3γ |
| ScFv | CD86 | b2c | CD3ε |
| ScFv | CD86 | b2c | FcγRI-γ |
| ScFv | CD86 | b2c | FcγRIII-γ |
| ScFv | CD86 | b2c | FcεRIβ |
| ScFv | CD86 | b2c | FcεRIγ |
| ScFv | CD86 | b2c | DAP10 |
| ScFv | CD86 | b2c | DAP12 |
| ScFv | CD86 | b2c | CD32 |
| ScFv | CD86 | b2c | CD79a |
| ScFv | CD86 | b2c | CD79b |
| ScFv | CD86 | CD137/41BB | CD8 |
| ScFv | CD86 | CD137/41BB | CD3ζ |
| ScFv | CD86 | CD137/41BB | CD3δ |
| ScFv | CD86 | CD137/41BB | CD3γ |
| ScFv | CD86 | CD137/41BB | CD3ε |
| ScFv | CD86 | CD137/41BB | FcγRI-γ |
| ScFv | CD86 | CD137/41BB | FcγRIII-γ |
| ScFv | CD86 | CD137/41BB | FcεRIβ |
| ScFv | CD86 | CD137/41BB | FcεRIγ |
| ScFv | CD86 | CD137/41BB | DAP10 |
| ScFv | CD86 | CD137/41BB | DAP12 |
| ScFv | CD86 | CD137/41BB | CD32 |
| ScFv | CD86 | CD137/41BB | CD79a |
| ScFv | CD86 | CD137/41BB | CD79b |
| ScFv | CD86 | ICOS | CD8 |
| ScFv | CD86 | ICOS | CD3ζ |
| ScFv | CD86 | ICOS | CD3δ |
| ScFv | CD86 | ICOS | CD3γ |
| ScFv | CD86 | ICOS | CD3ε |
| ScFv | CD86 | ICOS | FcγRI-γ |
| ScFv | CD86 | ICOS | FcγRIII-γ |
| ScFv | CD86 | ICOS | FcεRIβ |
| ScFv | CD86 | ICOS | FcεRIγ |
| ScFv | CD86 | ICOS | DAP10 |
| ScFv | CD86 | ICOS | DAP12 |
| ScFv | CD86 | ICOS | CD32 |
| ScFv | CD86 | ICOS | CD79a |
| ScFv | CD86 | ICOS | CD79b |
| ScFv | CD86 | CD27 | CD8 |
| ScFv | CD86 | CD27 | CD3ζ |
| ScFv | CD86 | CD27 | CD3δ |
| ScFv | CD86 | CD27 | CD3γ |
| ScFv | CD86 | CD27 | CD3ε |
| ScFv | CD86 | CD27 | FcγRI-γ |
| ScFv | CD86 | CD27 | FcγRIII-γ |
| ScFv | CD86 | CD27 | FcεRIβ |
| ScFv | CD86 | CD27 | FcεRIγ |
| ScFv | CD86 | CD27 | DAP10 |
| ScFv | CD86 | CD27 | DAP12 |
| ScFv | CD86 | CD27 | CD32 |
| ScFv | CD86 | CD27 | CD79a |
| ScFv | CD86 | CD27 | CD79b |
| ScFv | CD86 | CD28δ | CD8 |
| ScFv | CD86 | CD28δ | CD3ζ |
| ScFv | CD86 | CD28δ | CD3δ |
| ScFv | CD86 | CD28δ | CD3γ |
| ScFv | CD86 | CD28δ | CD3ε |
| ScFv | CD86 | CD28δ | FcγRI-γ |
| ScFv | CD86 | CD28δ | FcγRIII-γ |
| ScFv | CD86 | CD28δ | FcεRIβ |
| ScFv | CD86 | CD28δ | FcεRIγ |
| ScFv | CD86 | CD28δ | DAP10 |
| ScFv | CD86 | CD28δ | DAP12 |
| ScFv | CD86 | CD28δ | CD32 |
| ScFv | CD86 | CD28δ | CD79a |
| ScFv | CD86 | CD28δ | CD79b |
| ScFv | CD86 | CD80 | CD8 |
| ScFv | CD86 | CD80 | CD3ζ |
| ScFv | CD86 | CD80 | CD3δ |
| ScFv | CD86 | CD80 | CD3γ |
| ScFv | CD86 | CD80 | CD3ε |
| ScFv | CD86 | CD80 | FcγRI-γ |
| ScFv | CD86 | CD80 | FcγRIII-γ |
| ScFv | CD86 | CD80 | FcεRIβ |
| ScFv | CD86 | CD80 | FcεRIγ |
| ScFv | CD86 | CD80 | DAP10 |
| ScFv | CD86 | CD80 | DAP12 |
| ScFv | CD86 | CD80 | CD32 |
| ScFv | CD86 | CD80 | CD79a |
| ScFv | CD86 | CD80 | CD79b |
| ScFv | CD86 | CD86 | CD8 |
| ScFv | CD86 | CD86 | CD3ζ |
| ScFv | CD86 | CD86 | CD3δ |
| ScFv | CD86 | CD86 | CD3γ |
| ScFv | CD86 | CD86 | CD3ε |
| ScFv | CD86 | CD86 | FcγRI-γ |
| ScFv | CD86 | CD86 | FcγRIII-γ |
| ScFv | CD86 | CD86 | FcεRIβ |
| ScFv | CD86 | CD86 | FcεRIγ |
| ScFv | CD86 | CD86 | DAP10 |
| ScFv | CD86 | CD86 | DAP12 |
| ScFv | CD86 | CD86 | CD32 |
| ScFv | CD86 | CD86 | CD79a |
| ScFv | CD86 | CD86 | CD79b |
| ScFv | CD86 | OX40 | CD8 |
| ScFv | CD86 | OX40 | CD3ζ |
| ScFv | CD86 | OX40 | CD3δ |
| ScFv | CD86 | OX40 | CD3γ |
| ScFv | CD86 | OX40 | CD3ε |
| ScFv | CD86 | OX40 | FcγRI-γ |
| ScFv | CD86 | OX40 | FcγRIII-γ |
| ScFv | CD86 | OX40 | FcεRIβ |
| ScFv | CD86 | OX40 | FcεRIγ |
| ScFv | CD86 | OX40 | DAP10 |
| ScFv | CD86 | OX40 | DAP12 |
| ScFv | CD86 | OX40 | CD32 |
| ScFv | CD86 | OX40 | CD79a |
| ScFv | CD86 | OX40 | CD79b |
| ScFv | CD86 | DAP10 | CD8 |
| ScFv | CD86 | DAP10 | CD3ζ |
| ScFv | CD86 | DAP10 | CD3δ |
| ScFv | CD86 | DAP10 | CD3γ |
| ScFv | CD86 | DAP10 | CD3ε |
| ScFv | CD86 | DAP10 | FcγRI-γ |
| ScFv | CD86 | DAP10 | FcγRIII-γ |
| ScFv | CD86 | DAP10 | FcεRIβ |
| ScFv | CD86 | DAP10 | FcεRIγ |
| ScFv | CD86 | DAP10 | DAP10 |
| ScFv | CD86 | DAP10 | DAP12 |
| ScFv | CD86 | DAP10 | CD32 |
| ScFv | CD86 | DAP10 | CD79a |
| ScFv | CD86 | DAP10 | CD79b |
| ScFv | CD86 | DAP12 | CD8 |
| ScFv | CD86 | DAP12 | CD3ζ |
| ScFv | CD86 | DAP12 | CD3δ |
| ScFv | CD86 | DAP12 | CD3γ |
| ScFv | CD86 | DAP12 | CD3ε |
| ScFv | CD86 | DAP12 | FcγRI-γ |
| ScFv | CD86 | DAP12 | FcγRIII-γ |
| ScFv | CD86 | DAP12 | FcεRIβ |
| ScFv | CD86 | DAP12 | FcεRIγ |
| ScFv | CD86 | DAP12 | DAP10 |
| ScFv | CD86 | DAP12 | DAP12 |
| ScFv | CD86 | DAP12 | CD32 |
| ScFv | CD86 | DAP12 | CD79a |
| ScFv | CD86 | DAP12 | CD79b |
| ScFv | CD86 | MyD88 | CD8 |
| ScFv | CD86 | MyD88 | CD3ζ |
| ScFv | CD86 | MyD88 | CD3δ |
| ScFv | CD86 | MyD88 | CD3γ |
| ScFv | CD86 | MyD88 | CD3ε |
| ScFv | CD86 | MyD88 | FcγRI-γ |
| ScFv | CD86 | MyD88 | FcγRIII-γ |
| ScFv | CD86 | MyD88 | FcεRIβ |
| ScFv | CD86 | MyD88 | FcεRIγ |
| ScFv | CD86 | MyD88 | DAP10 |
| ScFv | CD86 | MyD88 | DAP12 |
| ScFv | CD86 | MyD88 | CD32 |
| ScFv | CD86 | MyD88 | CD79a |
| ScFv | CD86 | MyD88 | CD79b |
| ScFv | CD86 | CD7 | CD8 |
| ScFv | CD86 | CD7 | CD3ζ |
| ScFv | CD86 | CD7 | CD3δ |
| ScFv | CD86 | CD7 | CD3γ |
| ScFv | CD86 | CD7 | CD3ε |
| ScFv | CD86 | CD7 | FcγRI-γ |
| ScFv | CD86 | CD7 | FcγRIII-γ |
| ScFv | CD86 | CD7 | FcεRIβ |
| ScFv | CD86 | CD7 | FcεRIγ |
| ScFv | CD86 | CD7 | DAP10 |
| ScFv | CD86 | CD7 | DAP12 |
| ScFv | CD86 | CD7 | CD32 |
| ScFv | CD86 | CD7 | CD79a |
| ScFv | CD86 | CD7 | CD79b |
| ScFv | CD86 | BTNL3 | CD8 |
| ScFv | CD86 | BTNL3 | CD3ζ |
| ScFv | CD86 | BTNL3 | CD3δ |
| ScFv | CD86 | BTNL3 | CD3γ |
| ScFv | CD86 | BTNL3 | CD3ε |
| ScFv | CD86 | BTNL3 | FcγRI-γ |
| ScFv | CD86 | BTNL3 | FcγRIII-γ |
| ScFv | CD86 | BTNL3 | FcεRIβ |
| ScFv | CD86 | BTNL3 | FcεRIγ |
| ScFv | CD86 | BTNL3 | DAP10 |
| ScFv | CD86 | BTNL3 | DAP12 |
| ScFv | CD86 | BTNL3 | CD32 |
| ScFv | CD86 | BTNL3 | CD79a |
| ScFv | CD86 | BTNL3 | CD79b |
| ScFv | CD86 | NKG2D | CD8 |
| ScFv | CD86 | NKG2D | CD3ζ |
| ScFv | CD86 | NKG2D | CD3δ |
| ScFv | CD86 | NKG2D | CD3γ |
| ScFv | CD86 | NKG2D | CD3ε |
| ScFv | CD86 | NKG2D | FcγRI-γ |
| ScFv | CD86 | NKG2D | FcγRIII-γ |
| ScFv | CD86 | NKG2D | FcεRIβ |
| ScFv | CD86 | NKG2D | FcεRIγ |
| ScFv | CD86 | NKG2D | DAP10 |
| ScFv | CD86 | NKG2D | DAP12 |
| ScFv | CD86 | NKG2D | CD32 |
| ScFv | CD86 | NKG2D | CD79a |
| ScFv | CD86 | NKG2D | CD79b |
| ScFv | OX40 | CD28 | CD8 |
| ScFv | OX40 | CD28 | CD3ζ |
| ScFv | OX40 | CD28 | CD3δ |
| ScFv | OX40 | CD28 | CD3γ |
| ScFv | OX40 | CD28 | CD3ε |
| ScFv | OX40 | CD28 | FcγRI-γ |
| ScFv | OX40 | CD28 | FcγRIII-γ |
| ScFv | OX40 | CD28 | FcεRIβ |
| ScFv | OX40 | CD28 | FcεRIγ |
| ScFv | OX40 | CD28 | DAP10 |
| ScFv | OX40 | CD28 | DAP12 |
| ScFv | OX40 | CD28 | CD32 |
| ScFv | OX40 | CD28 | CD79a |
| ScFv | OX40 | CD28 | CD79b |
| ScFv | OX40 | CD8 | CD8 |
| ScFv | OX40 | CD8 | CD3ζ |
| ScFv | OX40 | CD8 | CD3δ |
| ScFv | OX40 | CD8 | CD3γ |
| ScFv | OX40 | CD8 | CD3ε |
| ScFv | OX40 | CD8 | FcγRI-γ |
| ScFv | OX40 | CD8 | FcγRIII-γ |
| ScFv | OX40 | CD8 | FcεRIβ |
| ScFv | OX40 | CD8 | FcεRIγ |
| ScFv | OX40 | CD8 | DAP10 |
| ScFv | OX40 | CD8 | DAP12 |
| ScFv | OX40 | CD8 | CD32 |
| ScFv | OX40 | CD8 | CD79a |
| ScFv | OX40 | CD8 | CD79b |
| ScFv | OX40 | CD4 | CD8 |
| ScFv | OX40 | CD4 | CD3ζ |
| ScFv | OX40 | CD4 | CD3δ |
| ScFv | OX40 | CD4 | CD3γ |
| ScFv | OX40 | CD4 | CD3ε |
| ScFv | OX40 | CD4 | FcγRI-γ |
| ScFv | OX40 | CD4 | FcγRIII-γ |
| ScFv | OX40 | CD4 | FcεRIβ |
| ScFv | OX40 | CD4 | FcεRIγ |
| ScFv | OX40 | CD4 | DAP10 |
| ScFv | OX40 | CD4 | DAP12 |
| ScFv | OX40 | CD4 | CD32 |
| ScFv | OX40 | CD4 | CD79a |
| ScFv | OX40 | CD4 | CD79b |
| ScFv | OX40 | b2c | CD8 |
| ScFv | OX40 | b2c | CD3ζ |
| ScFv | OX40 | b2c | CD3δ |
| ScFv | OX40 | b2c | CD3γ |
| ScFv | OX40 | b2c | CD3ε |
| ScFv | OX40 | b2c | FcγRI-γ |
| ScFv | OX40 | b2c | FcγRIII-γ |
| ScFv | OX40 | b2c | FcεRIβ |
| ScFv | OX40 | b2c | FcεRIγ |
| ScFv | OX40 | b2c | DAP10 |
| ScFv | OX40 | b2c | DAP12 |
| ScFv | OX40 | b2c | CD32 |
| ScFv | OX40 | b2c | CD79a |
| ScFv | OX40 | b2c | CD79b |
| ScFv | OX40 | CD137/41BB | CD8 |
| ScFv | OX40 | CD137/41BB | CD3ζ |
| ScFv | OX40 | CD137/41BB | CD3δ |
| ScFv | OX40 | CD137/41BB | CD3γ |
| ScFv | OX40 | CD137/41BB | CD3ε |
| ScFv | OX40 | CD137/41BB | FcγRI-γ |
| ScFv | OX40 | CD137/41BB | FcγRIII-γ |
| ScFv | OX40 | CD137/41BB | FcεRIβ |
| ScFv | OX40 | CD137/41BB | FcεRIγ |
| ScFv | OX40 | CD137/41BB | DAP10 |
| ScFv | OX40 | CD137/41BB | DAP12 |
| ScFv | OX40 | CD137/41BB | CD32 |
| ScFv | OX40 | CD137/41BB | CD79a |
| ScFv | OX40 | CD137/41BB | CD79b |
| ScFv | OX40 | ICOS | CD8 |
| ScFv | OX40 | ICOS | CD3ζ |
| ScFv | OX40 | ICOS | CD3δ |
| ScFv | OX40 | ICOS | CD3γ |
| ScFv | OX40 | ICOS | CD3ε |
| ScFv | OX40 | ICOS | FcγRI-γ |
| ScFv | OX40 | ICOS | FcγRIII-γ |
| ScFv | OX40 | ICOS | FcεRIβ |
| ScFv | OX40 | ICOS | FcεRIγ |
| ScFv | OX40 | ICOS | DAP10 |
| ScFv | OX40 | ICOS | DAP12 |
| ScFv | OX40 | ICOS | CD32 |
| ScFv | OX40 | ICOS | CD79a |
| ScFv | OX40 | ICOS | CD79b |
| ScFv | OX40 | CD27 | CD8 |
| ScFv | OX40 | CD27 | CD3ζ |
| ScFv | OX40 | CD27 | CD3δ |
| ScFv | OX40 | CD27 | CD3γ |
| ScFv | OX40 | CD27 | CD3ε |
| ScFv | OX40 | CD27 | FcγRI-γ |
| ScFv | OX40 | CD27 | FcγRIII-γ |
| ScFv | OX40 | CD27 | FcεRIβ |
| ScFv | OX40 | CD27 | FcεRIγ |
| ScFv | OX40 | CD27 | DAP10 |
| ScFv | OX40 | CD27 | DAP12 |
| ScFv | OX40 | CD27 | CD32 |
| ScFv | OX40 | CD27 | CD79a |
| ScFv | OX40 | CD27 | CD79b |
| ScFv | OX40 | CD28δ | CD8 |
| ScFv | OX40 | CD28δ | CD3ζ |
| ScFv | OX40 | CD28δ | CD3δ |
| ScFv | OX40 | CD28δ | CD3γ |
| ScFv | OX40 | CD28δ | CD3ε |
| ScFv | OX40 | CD28δ | FcγRI-γ |
| ScFv | OX40 | CD28δ | CD28δ |
| ScFv | OX40 | CD28δ | FcεRIβ |
| ScFv | OX40 | CD28δ | FcεRIγ |
| ScFv | OX40 | CD28δ | DAP10 |
| ScFv | OX40 | CD28δ | DAP12 |
| ScFv | OX40 | CD28δ | CD32 |
| ScFv | OX40 | CD28δ | CD79a |
| ScFv | OX40 | CD28δ | CD79b |
| ScFv | OX40 | CD80 | CD8 |
| ScFv | OX40 | CD80 | CD3ζ |
| ScFv | OX40 | CD80 | CD3δ |
| ScFv | OX40 | CD80 | CD3γ |
| ScFv | OX40 | CD80 | CD3ε |
| ScFv | OX40 | CD80 | FcγRI-γ |
| ScFv | OX40 | CD80 | FcγRIII-γ |
| ScFv | OX40 | CD80 | FcεRIβ |
| ScFv | OX40 | CD80 | FcεRIγ |
| ScFv | OX40 | CD80 | DAP10 |
| ScFv | OX40 | CD80 | DAP12 |
| ScFv | OX40 | CD80 | CD32 |
| ScFv | OX40 | CD80 | CD79a |
| ScFv | OX40 | CD80 | CD79b |
| ScFv | OX40 | CD86 | CD8 |
| ScFv | OX40 | CD86 | CD3ζ |
| ScFv | OX40 | CD86 | CD3δ |
| ScFv | OX40 | CD86 | CD3γ |
| ScFv | OX40 | CD86 | CD3ε |
| ScFv | OX40 | CD86 | FcγRI-γ |
| ScFv | OX40 | CD86 | FcγRIII-γ |
| ScFv | OX40 | CD86 | FcεRIβ |
| ScFv | OX40 | CD86 | FcεRIγ |
| ScFv | OX40 | CD86 | DAP10 |
| ScFv | OX40 | CD86 | DAP12 |
| ScFv | OX40 | CD86 | CD32 |
| ScFv | OX40 | CD86 | CD79a |
| ScFv | OX40 | CD86 | CD79b |
| ScFv | OX40 | OX40 | CD8 |
| ScFv | OX40 | OX40 | CD3ζ |
| ScFv | OX40 | OX40 | CD3δ |
| ScFv | OX40 | OX40 | CD3γ |
| ScFv | OX40 | OX40 | CD3ε |
| ScFv | OX40 | OX40 | FcγRI-γ |
| ScFv | OX40 | OX40 | FcγRIII-γ |
| ScFv | OX40 | OX40 | FcεRIβ |
| ScFv | OX40 | OX40 | FcεRIγ |
| ScFv | OX40 | OX40 | DAP10 |
| ScFv | OX40 | OX40 | DAP12 |
| ScFv | OX40 | OX40 | CD32 |
| ScFv | OX40 | OX40 | CD79a |
| ScFv | OX40 | OX40 | CD79b |
| ScFv | OX40 | DAP10 | CD8 |
| ScFv | OX40 | DAP10 | CD3ζ |
| ScFv | OX40 | DAP10 | CD3δ |
| ScFv | OX40 | DAP10 | CD3γ |
| ScFv | OX40 | DAP10 | CD3ε |
| ScFv | OX40 | DAP10 | FcγRI-γ |
| ScFv | OX40 | DAP10 | FcγRIII-γ |
| ScFv | OX40 | DAP10 | FcεRIβ |
| ScFv | OX40 | DAP10 | FcεRIγ |
| ScFv | OX40 | DAP10 | DAP10 |
| ScFv | OX40 | DAP10 | DAP12 |
| ScFv | OX40 | DAP10 | CD32 |
| ScFv | OX40 | DAP10 | CD79a |
| ScFv | OX40 | DAP10 | CD79b |
| ScFv | OX40 | DAP12 | CD8 |
| ScFv | OX40 | DAP12 | CD3ζ |
| ScFv | OX40 | DAP12 | CD3δ |
| ScFv | OX40 | DAP12 | CD3γ |
| ScFv | OX40 | DAP12 | CD3ε |
| ScFv | OX40 | DAP12 | FcγRI-γ |
| ScFv | OX40 | DAP12 | FcγRIII-γ |
| ScFv | OX40 | DAP12 | FcεRIβ |
| ScFv | OX40 | DAP12 | FcεRIγ |
| ScFv | OX40 | DAP12 | DAP10 |
| ScFv | OX40 | DAP12 | DAP12 |
| ScFv | OX40 | DAP12 | CD32 |
| ScFv | OX40 | DAP12 | CD79a |
| ScFv | OX40 | DAP12 | CD79b |
| ScFv | OX40 | MyD88 | CD8 |
| ScFv | OX40 | MyD88 | CD3ζ |
| ScFv | OX40 | MyD88 | CD3δ |
| ScFv | OX40 | MyD88 | CD3γ |
| ScFv | OX40 | MyD88 | CD3ε |
| ScFv | OX40 | MyD88 | FcγRI-γ |
| ScFv | OX40 | MyD88 | FcγRIII-γ |
| ScFv | OX40 | MyD88 | FcεRIβ |
| ScFv | OX40 | MyD88 | FcεRIγ |
| ScFv | OX40 | MyD88 | DAP10 |
| ScFv | OX40 | MyD88 | DAP12 |
| ScFv | OX40 | MyD88 | CD32 |
| ScFv | OX40 | MyD88 | CD79a |
| ScFv | OX40 | MyD88 | CD79b |
| ScFv | OX40 | CD7 | CD8 |
| ScFv | OX40 | CD7 | CD3ζ |
| ScFv | OX40 | CD7 | CD3δ |
| ScFv | OX40 | CD7 | CD3γ |
| ScFv | OX40 | CD7 | CD3ε |
| ScFv | OX40 | CD7 | FcγRI-γ |
| ScFv | OX40 | CD7 | FcγRIII-γ |
| ScFv | OX40 | CD7 | FcεRIβ |
| ScFv | OX40 | CD7 | FcεRIγ |
| ScFv | OX40 | CD7 | DAP10 |
| ScFv | OX40 | CD7 | DAP12 |
| ScFv | OX40 | CD7 | CD32 |
| ScFv | OX40 | CD7 | CD79a |
| ScFv | OX40 | CD7 | CD79b |
| ScFv | OX40 | BTNL3 | CD8 |
| ScFv | OX40 | BTNL3 | CD3ζ |
| ScFv | OX40 | BTNL3 | CD3δ |
| ScFv | OX40 | BTNL3 | CD3γ |
| ScFv | OX40 | BTNL3 | CD3ε |
| ScFv | OX40 | BTNL3 | FcγRI-γ |
| ScFv | OX40 | BTNL3 | FcγRIII-γ |
| ScFv | OX40 | BTNL3 | FcεRIβ |
| ScFv | OX40 | BTNL3 | FcεRIγ |
| ScFv | OX40 | BTNL3 | DAP10 |
| ScFv | OX40 | BTNL3 | DAP12 |
| ScFv | OX40 | BTNL3 | CD32 |
| ScFv | OX40 | BTNL3 | CD79a |
| ScFv | OX40 | BTNL3 | CD79b |
| ScFv | OX40 | NKG2D | CD8 |
| ScFv | OX40 | NKG2D | CD3ζ |
| ScFv | OX40 | NKG2D | CD3δ |
| ScFv | OX40 | NKG2D | CD3γ |
| ScFv | OX40 | NKG2D | CD3ε |
| ScFv | OX40 | NKG2D | FcγRI-γ |
| ScFv | OX40 | NKG2D | FcγRIII-γ |
| ScFv | OX40 | NKG2D | FcεRIβ |
| ScFv | OX40 | NKG2D | FcεRIγ |
| ScFv | OX40 | NKG2D | DAP10 |
| ScFv | OX40 | NKG2D | DAP12 |
| ScFv | OX40 | NKG2D | CD32 |
| ScFv | OX40 | NKG2D | CD79a |
| ScFv | OX40 | NKG2D | CD79b |
| ScFv | DAP10 | CD28 | CD8 |
| ScFv | DAP10 | CD28 | CD3ζ |
| ScFv | DAP10 | CD28 | CD3δ |
| ScFv | DAP10 | CD28 | CD3γ |
| ScFv | DAP10 | CD28 | CD3ε |
| ScFv | DAP10 | CD28 | FcγRI-γ |
| ScFv | DAP10 | CD28 | FcγRIII-γ |
| ScFv | DAP10 | CD28 | FcεRIβ |
| ScFv | DAP10 | CD28 | FcεRIγ |
| ScFv | DAP10 | CD28 | DAP10 |
| ScFv | DAP10 | CD28 | DAP12 |
| ScFv | DAP10 | CD28 | CD32 |
| ScFv | DAP10 | CD28 | CD79a |
| ScFv | DAP10 | CD28 | CD79b |
| ScFv | DAP10 | CD8 | CD8 |
| ScFv | DAP10 | CD8 | CD3ζ |
| ScFv | DAP10 | CD8 | CD3δ |
| ScFv | DAP10 | CD8 | CD3γ |
| ScFv | DAP10 | CD8 | CD3ε |
| ScFv | DAP10 | CD8 | FcγRI-γ |
| ScFv | DAP10 | CD8 | FcγRIII-γ |
| ScFv | DAP10 | CD8 | FcεRIβ |
| ScFv | DAP10 | CD8 | FcεRIγ |
| ScFv | DAP10 | CD8 | DAP10 |
| ScFv | DAP10 | CD8 | DAP12 |
| ScFv | DAP10 | CD8 | CD32 |
| ScFv | DAP10 | CD8 | CD79a |
| ScFv | DAP10 | CD8 | CD79b |
| ScFv | DAP10 | CD4 | CD8 |
| ScFv | DAP10 | CD4 | CD3ζ |
| ScFv | DAP10 | CD4 | CD3δ |
| ScFv | DAP10 | CD4 | CD3γ |
| ScFv | DAP10 | CD4 | CD3ε |
| ScFv | DAP10 | CD4 | FcγRI-γ |
| ScFv | DAP10 | CD4 | FcγRIII-γ |
| ScFv | DAP10 | CD4 | FcεRIβ |
| ScFv | DAP10 | CD4 | FcεRIγ |
| ScFv | DAP10 | CD4 | DAP10 |
| ScFv | DAP10 | CD4 | DAP12 |
| ScFv | DAP10 | CD4 | CD32 |
| ScFv | DAP10 | CD4 | CD79a |
| ScFv | DAP10 | CD4 | CD79b |
| ScFv | DAP10 | b2c | CD8 |
| ScFv | DAP10 | b2c | CD3ζ |
| ScFv | DAP10 | b2c | CD3δ |
| ScFv | DAP10 | b2c | CD3γ |
| ScFv | DAP10 | b2c | CD3ε |
| ScFv | DAP10 | b2c | FcγRI-γ |
| ScFv | DAP10 | b2c | FcγRIII-γ |
| ScFv | DAP10 | b2c | FcεRIβ |
| ScFv | DAP10 | b2c | FcεRIγ |
| ScFv | DAP10 | b2c | DAP10 |
| ScFv | DAP10 | b2c | DAP12 |
| ScFv | DAP10 | b2c | CD32 |
| ScFv | DAP10 | b2c | CD79a |
| ScFv | DAP10 | b2c | CD79b |
| ScFv | DAP10 | CD137/41BB | CD8 |
| ScFv | DAP10 | CD137/41BB | CD3ζ |
| ScFv | DAP10 | CD137/41BB | CD3δ |
| ScFv | DAP10 | CD137/41BB | CD3γ |
| ScFv | DAP10 | CD137/41BB | CD3ε |
| ScFv | DAP10 | CD137/41BB | FcγRI-γ |
| ScFv | DAP10 | CD137/41BB | FcγRIII-γ |
| ScFv | DAP10 | CD137/41BB | FcεRIβ |
| ScFv | DAP10 | CD137/41BB | FcεRIγ |
| ScFv | DAP10 | CD137/41BB | DAP10 |
| ScFv | DAP10 | CD137/41BB | DAP12 |
| ScFv | DAP10 | CD137/41BB | CD32 |
| ScFv | DAP10 | CD137/41BB | CD79a |
| ScFv | DAP10 | CD137/41BB | CD79b |
| ScFv | DAP10 | ICOS | CD8 |
| ScFv | DAP10 | ICOS | CD3ζ |
| ScFv | DAP10 | ICOS | CD3δ |
| ScFv | DAP10 | ICOS | CD3γ |
| ScFv | DAP10 | ICOS | CD3ε |
| ScFv | DAP10 | ICOS | FcγRI-γ |
| ScFv | DAP10 | ICOS | FcγRIII-γ |
| ScFv | DAP10 | ICOS | FcεRIβ |
| ScFv | DAP10 | ICOS | FcεRIγ |
| ScFv | DAP10 | ICOS | DAP10 |
| ScFv | DAP10 | ICOS | DAP12 |
| ScFv | DAP10 | ICOS | CD32 |
| ScFv | DAP10 | ICOS | CD79a |
| ScFv | DAP10 | ICOS | CD79b |
| ScFv | DAP10 | CD27 | CD8 |
| ScFv | DAP10 | CD27 | CD3ζ |
| ScFv | DAP10 | CD27 | CD3δ |
| ScFv | DAP10 | CD27 | CD3γ |
| ScFv | DAP10 | CD27 | CD3ε |
| ScFv | DAP10 | CD27 | FcγRI-γ |
| ScFv | DAP10 | CD27 | FcγRIII-γ |
| ScFv | DAP10 | CD27 | FcεRIβ |
| ScFv | DAP10 | CD27 | FcεRIγ |
| ScFv | DAP10 | CD27 | DAP10 |
| ScFv | DAP10 | CD27 | DAP12 |
| ScFv | DAP10 | CD27 | CD32 |
| ScFv | DAP10 | CD27 | CD79a |
| ScFv | DAP10 | CD27 | CD79b |
| ScFv | DAP10 | CD28δ | CD8 |
| ScFv | DAP10 | CD28δ | CD3ζ |
| ScFv | DAP10 | CD28δ | CD3δ |
| ScFv | DAP10 | CD28δ | CD3γ |
| ScFv | DAP10 | CD28δ | CD3ε |
| ScFv | DAP10 | CD28δ | FcγRI-γ |
| ScFv | DAP10 | CD28δ | FcγRIII-γ |
| ScFv | DAP10 | CD28δ | FcεRIβ |
| ScFv | DAP10 | CD28δ | FcεRIγ |
| ScFv | DAP10 | CD28δ | DAP10 |
| ScFv | DAP10 | CD28δ | DAP12 |
| ScFv | DAP10 | CD28δ | CD32 |
| ScFv | DAP10 | CD28δ | CD79a |
| ScFv | DAP10 | CD28δ | CD79b |
| ScFv | DAP10 | CD80 | CD8 |
| ScFv | DAP10 | CD80 | CD3ζ |
| ScFv | DAP10 | CD80 | CD3δ |
| ScFv | DAP10 | CD80 | CD3γ |
| ScFv | DAP10 | CD80 | CD3ε |
| ScFv | DAP10 | CD80 | FcγRI-γ |
| ScFv | DAP10 | CD80 | FcγRIII-γ |
| ScFv | DAP10 | CD80 | FcεRIβ |
| ScFv | DAP10 | CD80 | FcεRIγ |
| ScFv | DAP10 | CD80 | DAP10 |
| ScFv | DAP10 | CD80 | DAP12 |
| ScFv | DAP10 | CD80 | CD32 |
| ScFv | DAP10 | CD80 | CD79a |
| ScFv | DAP10 | CD80 | CD79b |
| ScFv | DAP10 | CD86 | CD8 |
| ScFv | DAP10 | CD86 | CD3ζ |
| ScFv | DAP10 | CD86 | CD3δ |
| ScFv | DAP10 | CD86 | CD3γ |
| ScFv | DAP10 | CD86 | CD3ε |
| ScFv | DAP10 | CD86 | FcγRI-γ |
| ScFv | DAP10 | CD86 | FcγRIII-γ |
| ScFv | DAP10 | CD86 | FcεRIβ |
| ScFv | DAP10 | CD86 | FcεRIγ |
| ScFv | DAP10 | CD86 | DAP10 |
| ScFv | DAP10 | CD86 | DAP12 |
| ScFv | DAP10 | CD86 | CD32 |
| ScFv | DAP10 | CD86 | CD79a |
| ScFv | DAP10 | CD86 | CD79b |
| ScFv | DAP10 | OX40 | CD8 |
| ScFv | DAP10 | OX40 | CD3ζ |
| ScFv | DAP10 | OX40 | CD3δ |
| ScFv | DAP10 | OX40 | CD3γ |
| ScFv | DAP10 | OX40 | CD3ε |
| ScFv | DAP10 | OX40 | FcγRI-γ |
| ScFv | DAP10 | OX40 | FcγRIII-γ |
| ScFv | DAP10 | OX40 | FcεRIβ |
| ScFv | DAP10 | OX40 | FcεRIγ |
| ScFv | DAP10 | OX40 | DAP10 |
| ScFv | DAP10 | OX40 | DAP12 |
| ScFv | DAP10 | OX40 | CD32 |
| ScFv | DAP10 | OX40 | CD79a |
| ScFv | DAP10 | OX40 | CD79b |
| ScFv | DAP10 | DAP10 | CD8 |
| ScFv | DAP10 | DAP10 | CD3ζ |
| ScFv | DAP10 | DAP10 | CD3δ |
| ScFv | DAP10 | DAP10 | CD3γ |
| ScFv | DAP10 | DAP10 | CD3ε |
| ScFv | DAP10 | DAP10 | FcγRI-γ |
| ScFv | DAP10 | DAP10 | FcγRIII-γ |
| ScFv | DAP10 | DAP10 | FcεRIβ |
| ScFv | DAP10 | DAP10 | FcεRIγ |
| ScFv | DAP10 | DAP10 | DAP10 |
| ScFv | DAP10 | DAP10 | DAP12 |
| ScFv | DAP10 | DAP10 | CD32 |
| ScFv | DAP10 | DAP10 | CD79a |
| ScFv | DAP10 | DAP10 | CD79b |
| ScFv | DAP10 | DAP12 | CD8 |
| ScFv | DAP10 | DAP12 | CD3ζ |
| ScFv | DAP10 | DAP12 | CD3δ |
| ScFv | DAP10 | DAP12 | CD3γ |
| ScFv | DAP10 | DAP12 | CD3ε |
| ScFv | DAP10 | DAP12 | FcγRI-γ |
| ScFv | DAP10 | DAP12 | FcγRIII-γ |
| ScFv | DAP10 | DAP12 | FcεRIβ |
| ScFv | DAP10 | DAP12 | FcεRIγ |
| ScFv | DAP10 | DAP12 | DAP10 |
| ScFv | DAP10 | DAP12 | DAP12 |
| ScFv | DAP10 | DAP12 | CD32 |
| ScFv | DAP10 | DAP12 | CD79a |
| ScFv | DAP10 | DAP12 | CD79b |
| ScFv | DAP10 | MyD88 | CD8 |
| ScFv | DAP10 | MyD88 | CD3ζ |
| ScFv | DAP10 | MyD88 | CD3δ |
| ScFv | DAP10 | MyD88 | CD3γ |
| ScFv | DAP10 | MyD88 | CD3ε |
| ScFv | DAP10 | MyD88 | FcγRI-γ |
| ScFv | DAP10 | MyD88 | FcγRIII-γ |
| ScFv | DAP10 | MyD88 | FcεRIβ |
| ScFv | DAP10 | MyD88 | FcεRIγ |
| ScFv | DAP10 | MyD88 | DAP10 |
| ScFv | DAP10 | MyD88 | DAP12 |
| ScFv | DAP10 | MyD88 | CD32 |
| ScFv | DAP10 | MyD88 | CD79a |
| ScFv | DAP10 | MyD88 | CD79b |
| ScFv | DAP10 | CD7 | CD8 |
| ScFv | DAP10 | CD7 | CD3ζ |
| ScFv | DAP10 | CD7 | CD3δ |
| ScFv | DAP10 | CD7 | CD3γ |
| ScFv | DAP10 | CD7 | CD3ε |
| ScFv | DAP10 | CD7 | FcγRI-γ |
| ScFv | DAP10 | CD7 | FcγRIII-γ |
| ScFv | DAP10 | CD7 | FcεRIβ |
| ScFv | DAP10 | CD7 | FcεRIγ |
| ScFv | DAP10 | CD7 | DAP10 |
| ScFv | DAP10 | CD7 | DAP12 |
| ScFv | DAP10 | CD7 | CD32 |
| ScFv | DAP10 | CD7 | CD79a |
| ScFv | DAP10 | CD7 | CD79b |
| ScFv | DAP10 | BTNL3 | CD8 |
| ScFv | DAP10 | BTNL3 | CD3ζ |
| ScFv | DAP10 | BTNL3 | CD3δ |
| ScFv | DAP10 | BTNL3 | CD3γ |
| ScFv | DAP10 | BTNL3 | CD3ε |
| ScFv | DAP10 | BTNL3 | FcγRI-γ |
| ScFv | DAP10 | BTNL3 | FcγRIII-γ |
| ScFv | DAP10 | BTNL3 | FcεRIβ |
| ScFv | DAP10 | BTNL3 | FcεRIγ |
| ScFv | DAP10 | BTNL3 | DAP10 |
| ScFv | DAP10 | BTNL3 | DAP12 |
| ScFv | DAP10 | BTNL3 | CD32 |
| ScFv | DAP10 | BTNL3 | CD79a |
| ScFv | DAP10 | BTNL3 | CD79b |
| ScFv | DAP10 | NKG2D | CD8 |
| ScFv | DAP10 | NKG2D | CD3ζ |
| ScFv | DAP10 | NKG2D | CD3δ |
| ScFv | DAP10 | NKG2D | CD3γ |
| ScFv | DAP10 | NKG2D | CD3ε |
| ScFv | DAP10 | NKG2D | FcγRI-γ |
| ScFv | DAP10 | NKG2D | FcγRIII-γ |
| ScFv | DAP10 | NKG2D | FcεRIβ |
| ScFv | DAP10 | NKG2D | FcεRIγ |
| ScFv | DAP10 | NKG2D | DAP10 |
| ScFv | DAP10 | NKG2D | DAP12 |
| ScFv | DAP10 | NKG2D | CD32 |
| ScFv | DAP10 | NKG2D | CD79a |
| ScFv | DAP10 | NKG2D | CD79b |
| ScFv | DAP12 | CD28 | CD8 |
| ScFv | DAP12 | CD28 | CD3ζ |
| ScFv | DAP12 | CD28 | CD3δ |
| ScFv | DAP12 | CD28 | CD3γ |
| ScFv | DAP12 | CD28 | CD3ε |
| ScFv | DAP12 | CD28 | FcγRI-γ |
| ScFv | DAP12 | CD28 | FcγRIII-γ |
| ScFv | DAP12 | CD28 | FcεRIβ |
| ScFv | DAP12 | CD28 | FcεRIγ |
| ScFv | DAP12 | CD28 | DAP10 |
| ScFv | DAP12 | CD28 | DAP12 |
| ScFv | DAP12 | CD28 | CD32 |
| ScFv | DAP12 | CD28 | CD79a |
| ScFv | DAP12 | CD28 | CD79b |
| ScFv | DAP12 | CD8 | CD8 |
| ScFv | DAP12 | CD8 | CD3ζ |
| ScFv | DAP12 | CD8 | CD3δ |
| ScFv | DAP12 | CD8 | CD3γ |
| ScFv | DAP12 | CD8 | CD3ε |
| ScFv | DAP12 | CD8 | FcγRI-γ |
| ScFv | DAP12 | CD8 | FcγRIII-γ |
| ScFv | DAP12 | CD8 | FcεRIβ |
| ScFv | DAP12 | CD8 | FcεRIγ |
| ScFv | DAP12 | CD8 | DAP10 |
| ScFv | DAP12 | CD8 | DAP12 |
| ScFv | DAP12 | CD8 | CD32 |
| ScFv | DAP12 | CD8 | CD79a |
| ScFv | DAP12 | CD8 | CD79b |
| ScFv | DAP12 | CD4 | CD8 |
| ScFv | DAP12 | CD4 | CD3ζ |
| ScFv | DAP12 | CD4 | CD3δ |
| ScFv | DAP12 | CD4 | CD3γ |
| ScFv | DAP12 | CD4 | CD3ε |
| ScFv | DAP12 | CD4 | FcγRI-γ |
| ScFv | DAP12 | CD4 | FcγRIII-γ |
| ScFv | DAP12 | CD4 | FcεRIβ |
| ScFv | DAP12 | CD4 | FcεRIγ |
| ScFv | DAP12 | CD4 | DAP10 |
| ScFv | DAP12 | CD4 | DAP12 |
| ScFv | DAP12 | CD4 | CD32 |
| ScFv | DAP12 | CD4 | CD79a |
| ScFv | DAP12 | CD4 | CD79b |
| ScFv | DAP12 | b2c | CD8 |
| ScFv | DAP12 | b2c | CD3ζ |
| ScFv | DAP12 | b2c | CD3δ |
| ScFv | DAP12 | b2c | CD3γ |
| ScFv | DAP12 | b2c | CD3ε |
| ScFv | DAP12 | b2c | FcγRI-γ |
| ScFv | DAP12 | b2c | FcγRIII-γ |
| ScFv | DAP12 | b2c | FcεRIβ |
| ScFv | DAP12 | b2c | FcεRIγ |
| ScFv | DAP12 | b2c | DAP10 |
| ScFv | DAP12 | b2c | DAP12 |
| ScFv | DAP12 | b2c | CD32 |
| ScFv | DAP12 | b2c | CD79a |
| ScFv | DAP12 | b2c | CD79b |
| ScFv | DAP12 | CD137/41BB | CD8 |
| ScFv | DAP12 | CD137/41BB | CD3ζ |
| ScFv | DAP12 | CD137/41BB | CD3δ |
| ScFv | DAP12 | CD137/41BB | CD3γ |
| ScFv | DAP12 | CD137/41BB | CD3ε |
| ScFv | DAP12 | CD137/41BB | FcγRI-γ |
| ScFv | DAP12 | CD137/41BB | FcγRIII-γ |
| ScFv | DAP12 | CD137/41BB | FcεRIβ |
| ScFv | DAP12 | CD137/41BB | FcεRIγ |
| ScFv | DAP12 | CD137/41BB | DAP10 |
| ScFv | DAP12 | CD137/41BB | DAP12 |
| ScFv | DAP12 | CD137/41BB | CD32 |
| ScFv | DAP12 | CD137/41BB | CD79a |
| ScFv | DAP12 | CD137/41BB | CD79b |
| ScFv | DAP12 | ICOS | CD8 |
| ScFv | DAP12 | ICOS | CD3ζ |
| ScFv | DAP12 | ICOS | CD3δ |
| ScFv | DAP12 | ICOS | CD3γ |
| ScFv | DAP12 | ICOS | CD3ε |
| ScFv | DAP12 | ICOS | FcγRI-γ |
| ScFv | DAP12 | ICOS | FcγRIII-γ |
| ScFv | DAP12 | ICOS | FcεRIβ |
| ScFv | DAP12 | ICOS | FcεRIγ |
| ScFv | DAP12 | ICOS | DAP10 |
| ScFv | DAP12 | ICOS | DAP12 |
| ScFv | DAP12 | ICOS | CD32 |
| ScFv | DAP12 | ICOS | CD79a |
| ScFv | DAP12 | ICOS | CD79b |
| ScFv | DAP12 | CD27 | CD8 |
| ScFv | DAP12 | CD27 | CD3ζ |
| ScFv | DAP12 | CD27 | CD3δ |
| ScFv | DAP12 | CD27 | CD3γ |
| ScFv | DAP12 | CD27 | CD3ε |
| ScFv | DAP12 | CD27 | FcγRI-γ |
| ScFv | DAP12 | CD27 | FcγRIII-γ |
| ScFv | DAP12 | CD27 | FcεRIβ |
| ScFv | DAP12 | CD27 | FcεRIγ |
| ScFv | DAP12 | CD27 | DAP10 |
| ScFv | DAP12 | CD27 | DAP12 |
| ScFv | DAP12 | CD27 | CD32 |
| ScFv | DAP12 | CD27 | CD79a |
| ScFv | DAP12 | CD27 | CD79b |
| ScFv | DAP12 | CD28δ | CD8 |
| ScFv | DAP12 | CD28δ | CD3ζ |
| ScFv | DAP12 | CD28δ | CD3δ |
| ScFv | DAP12 | CD28δ | CD3γ |
| ScFv | DAP12 | CD28δ | CD3ε |
| ScFv | DAP12 | CD28δ | FcγRI-γ |
| ScFv | DAP12 | CD28δ | FcγRIII-γ |
| ScFv | DAP12 | CD28δ | FcεRIβ |
| ScFv | DAP12 | CD28δ | FcεRIγ |
| ScFv | DAP12 | CD28δ | DAP10 |
| ScFv | DAP12 | CD28δ | DAP12 |
| ScFv | DAP12 | CD28δ | CD32 |
| ScFv | DAP12 | CD28δ | CD79a |
| ScFv | DAP12 | CD28δ | CD79b |
| ScFv | DAP12 | CD80 | CD8 |
| ScFv | DAP12 | CD80 | CD3ζ |
| ScFv | DAP12 | CD80 | CD3δ |
| ScFv | DAP12 | CD80 | CD3γ |
| ScFv | DAP12 | CD80 | CD3ε |
| ScFv | DAP12 | CD80 | FcγRI-γ |
| ScFv | DAP12 | CD80 | FcγRIII-γ |
| ScFv | DAP12 | CD80 | FcεRIβ |
| ScFv | DAP12 | CD80 | FcεRIγ |
| ScFv | DAP12 | CD80 | DAP10 |
| ScFv | DAP12 | CD80 | DAP12 |
| ScFv | DAP12 | CD80 | CD32 |
| ScFv | DAP12 | CD80 | CD79a |
| ScFv | DAP12 | CD80 | CD79b |
| ScFv | DAP12 | CD86 | CD8 |
| ScFv | DAP12 | CD86 | CD3ζ |
| ScFv | DAP12 | CD86 | CD3δ |
| ScFv | DAP12 | CD86 | CD3γ |
| ScFv | DAP12 | CD86 | CD3ε |
| ScFv | DAP12 | CD86 | FcγRI-γ |
| ScFv | DAP12 | CD86 | FcγRIII-γ |
| ScFv | DAP12 | CD86 | FcεRIβ |
| ScFv | DAP12 | CD86 | FcεRIγ |
| ScFv | DAP12 | CD86 | DAP10 |
| ScFv | DAP12 | CD86 | DAP12 |
| ScFv | DAP12 | CD86 | CD32 |
| ScFv | DAP12 | CD86 | CD79a |
| ScFv | DAP12 | CD86 | CD79b |
| ScFv | DAP12 | OX40 | CD8 |
| ScFv | DAP12 | OX40 | CD3ζ |
| ScFv | DAP12 | OX40 | CD3δ |
| ScFv | DAP12 | OX40 | CD3γ |
| ScFv | DAP12 | OX40 | CD3ε |
| ScFv | DAP12 | OX40 | FcγRI-γ |
| ScFv | DAP12 | OX40 | FcγRIII-γ |
| ScFv | DAP12 | OX40 | FcεRIβ |
| ScFv | DAP12 | OX40 | FcεRIγ |
| ScFv | DAP12 | OX40 | DAP10 |
| ScFv | DAP12 | OX40 | DAP12 |
| ScFv | DAP12 | OX40 | CD32 |
| ScFv | DAP12 | OX40 | CD79a |
| ScFv | DAP12 | OX40 | CD79b |
| ScFv | DAP12 | DAP10 | CD8 |
| ScFv | DAP12 | DAP10 | CD3ζ |
| ScFv | DAP12 | DAP10 | CD3δ |
| ScFv | DAP12 | DAP10 | CD3γ |
| ScFv | DAP12 | DAP10 | CD3ε |
| ScFv | DAP12 | DAP10 | FcγRI-γ |
| ScFv | DAP12 | DAP10 | FcγRIII-γ |
| ScFv | DAP12 | DAP10 | FcεRIβ |
| ScFv | DAP12 | DAP10 | FcεRIγ |
| ScFv | DAP12 | DAP10 | DAP10 |
| ScFv | DAP12 | DAP10 | DAP12 |
| ScFv | DAP12 | DAP10 | CD32 |
| ScFv | DAP12 | DAP10 | CD79a |
| ScFv | DAP12 | DAP10 | CD79b |
| ScFv | DAP12 | DAP12 | CD8 |
| ScFv | DAP12 | DAP12 | CD3ζ |
| ScFv | DAP12 | DAP12 | CD3δ |
| ScFv | DAP12 | DAP12 | CD3γ |
| ScFv | DAP12 | DAP12 | CD3ε |
| ScFv | DAP12 | DAP12 | FcγRI-γ |
| ScFv | DAP12 | DAP12 | FcγRIII-γ |
| ScFv | DAP12 | DAP12 | FcεRIβ |
| ScFv | DAP12 | DAP12 | FcεRIγ |
| ScFv | DAP12 | DAP12 | DAP10 |
| ScFv | DAP12 | DAP12 | DAP12 |
| ScFv | DAP12 | DAP12 | CD32 |
| ScFv | DAP12 | DAP12 | CD79a |
| ScFv | DAP12 | DAP12 | CD79b |
| ScFv | DAP12 | MyD88 | CD8 |
| ScFv | DAP12 | MyD88 | CD3ζ |
| ScFv | DAP12 | MyD88 | CD3δ |
| ScFv | DAP12 | MyD88 | CD3γ |
| ScFv | DAP12 | MyD88 | CD3ε |
| ScFv | DAP12 | MyD88 | FcγRI-γ |
| ScFv | DAP12 | MyD88 | FcγRIII-γ |
| ScFv | DAP12 | MyD88 | FcεRIβ |
| ScFv | DAP12 | MyD88 | FcεRIγ |
| ScFv | DAP12 | MyD88 | DAP10 |
| ScFv | DAP12 | MyD88 | DAP12 |
| ScFv | DAP12 | MyD88 | CD32 |
| ScFv | DAP12 | MyD88 | CD79a |
| ScFv | DAP12 | MyD88 | CD79b |
| ScFv | DAP12 | CD7 | CD8 |
| ScFv | DAP12 | CD7 | CD3ζ |
| ScFv | DAP12 | CD7 | CD3δ |
| ScFv | DAP12 | CD7 | CD3γ |
| ScFv | DAP12 | CD7 | CD3ε |
| ScFv | DAP12 | CD7 | FcγRI-γ |
| ScFv | DAP12 | CD7 | FcγRIII-γ |
| ScFv | DAP12 | CD7 | FcεRIβ |
| ScFv | DAP12 | CD7 | FcεRIγ |
| ScFv | DAP12 | CD7 | DAP10 |
| ScFv | DAP12 | CD7 | DAP12 |
| ScFv | DAP12 | CD7 | CD32 |
| ScFv | DAP12 | CD7 | CD79a |
| ScFv | DAP12 | CD7 | CD79b |
| ScFv | DAP12 | BTNL3 | CD8 |
| ScFv | DAP12 | BTNL3 | CD3ζ |
| ScFv | DAP12 | BTNL3 | CD3δ |
| ScFv | DAP12 | BTNL3 | CD3γ |
| ScFv | DAP12 | BTNL3 | CD3ε |
| ScFv | DAP12 | BTNL3 | FcγRI-γ |
| ScFv | DAP12 | BTNL3 | FcγRIII-γ |
| ScFv | DAP12 | BTNL3 | FcεRIβ |
| ScFv | DAP12 | BTNL3 | FcεRIγ |
| ScFv | DAP12 | BTNL3 | DAP10 |
| ScFv | DAP12 | BTNL3 | DAP12 |
| ScFv | DAP12 | BTNL3 | CD32 |
| ScFv | DAP12 | BTNL3 | CD79a |
| ScFv | DAP12 | BTNL3 | CD79b |
| ScFv | DAP12 | NKG2D | CD8 |
| ScFv | DAP12 | NKG2D | CD3ζ |
| ScFv | DAP12 | NKG2D | CD3δ |
| ScFv | DAP12 | NKG2D | CD3γ |
| ScFv | DAP12 | NKG2D | CD3ε |
| ScFv | DAP12 | NKG2D | FcγRI-γ |
| ScFv | DAP12 | NKG2D | FcγRIII-γ |
| ScFv | DAP12 | NKG2D | FcεRIβ |
| ScFv | DAP12 | NKG2D | FcεRIγ |
| ScFv | DAP12 | NKG2D | DAP10 |
| ScFv | DAP12 | NKG2D | DAP12 |
| ScFv | DAP12 | NKG2D | CD32 |
| ScFv | DAP12 | NKG2D | CD79a |
| ScFv | DAP12 | NKG2D | CD79b |
| ScFv | MyD88 | CD28 | CD8 |
| ScFv | MyD88 | CD28 | CD3ζ |
| ScFv | MyD88 | CD28 | CD3δ |
| ScFv | MyD88 | CD28 | CD3γ |
| ScFv | MyD88 | CD28 | CD3ε |
| ScFv | MyD88 | CD28 | FcγRI-γ |
| ScFv | MyD88 | CD28 | FcγRIII-γ |
| ScFv | MyD88 | CD28 | FcεRIβ |
| ScFv | MyD88 | CD28 | FcεRIγ |
| ScFv | MyD88 | CD28 | DAP10 |
| ScFv | MyD88 | CD28 | DAP12 |
| ScFv | MyD88 | CD28 | CD32 |
| ScFv | MyD88 | CD28 | CD79a |
| ScFv | MyD88 | CD28 | CD79b |
| ScFv | MyD88 | CD8 | CD8 |
| ScFv | MyD88 | CD8 | CD3ζ |
| ScFv | MyD88 | CD8 | CD3δ |
| ScFv | MyD88 | CD8 | CD3γ |
| ScFv | MyD88 | CD8 | CD3ε |
| ScFv | MyD88 | CD8 | FcγRI-γ |
| ScFv | MyD88 | CD8 | FcγRIII-γ |
| ScFv | MyD88 | CD8 | FcεRIβ |
| ScFv | MyD88 | CD8 | FcεRIγ |
| ScFv | MyD88 | CD8 | DAP10 |
| ScFv | MyD88 | CD8 | DAP12 |
| ScFv | MyD88 | CD8 | CD32 |
| ScFv | MyD88 | CD8 | CD79a |
| ScFv | MyD88 | CD8 | CD79b |
| ScFv | MyD88 | CD4 | CD8 |
| ScFv | MyD88 | CD4 | CD3ζ |
| ScFv | MyD88 | CD4 | CD3δ |
| ScFv | MyD88 | CD4 | CD3γ |
| ScFv | MyD88 | CD4 | CD3ε |
| ScFv | MyD88 | CD4 | FcγRI-γ |
| ScFv | MyD88 | CD4 | FcγRIII-γ |
| ScFv | MyD88 | CD4 | FcεRIβ |
| ScFv | MyD88 | CD4 | FcεRIγ |
| ScFv | MyD88 | CD4 | DAP10 |
| ScFv | MyD88 | CD4 | DAP12 |
| ScFv | MyD88 | CD4 | CD32 |
| ScFv | MyD88 | CD4 | CD79a |
| ScFv | MyD88 | CD4 | CD79b |
| ScFv | MyD88 | b2c | CD8 |
| ScFv | MyD88 | b2c | CD3ζ |
| ScFv | MyD88 | b2c | CD3δ |
| ScFv | MyD88 | b2c | CD3γ |
| ScFv | MyD88 | b2c | CD3ε |
| ScFv | MyD88 | b2c | FcγRI-γ |
| ScFv | MyD88 | b2c | FcγRIII-γ |
| ScFv | MyD88 | b2c | FcεRIβ |
| ScFv | MyD88 | b2c | FcεRIγ |
| ScFv | MyD88 | b2c | DAP10 |
| ScFv | MyD88 | b2c | DAP12 |
| ScFv | MyD88 | b2c | CD32 |
| ScFv | MyD88 | b2c | CD79a |
| ScFv | MyD88 | b2c | CD79b |
| ScFv | MyD88 | CD137/41BB | CD8 |
| ScFv | MyD88 | CD137/41BB | CD3ζ |
| ScFv | MyD88 | CD137/41BB | CD3δ |
| ScFv | MyD88 | CD137/41BB | CD3γ |
| ScFv | MyD88 | CD137/41BB | CD3ε |
| ScFv | MyD88 | CD137/41BB | FcγRI-γ |
| ScFv | MyD88 | CD137/41BB | FcγRIII-γ |
| ScFv | MyD88 | CD137/41BB | FcεRIβ |
| ScFv | MyD88 | CD137/41BB | FcεRIγ |
| ScFv | MyD88 | CD137/41BB | DAP10 |
| ScFv | MyD88 | CD137/41BB | DAP12 |
| ScFv | MyD88 | CD137/41BB | CD32 |
| ScFv | MyD88 | CD137/41BB | CD79a |
| ScFv | MyD88 | CD137/41BB | CD79b |
| ScFv | MyD88 | ICOS | CD8 |
| ScFv | MyD88 | ICOS | CD3ζ |
| ScFv | MyD88 | ICOS | CD3δ |
| ScFv | MyD88 | ICOS | CD3γ |
| ScFv | MyD88 | ICOS | CD3ε |
| ScFv | MyD88 | ICOS | FcγRI-γ |
| ScFv | MyD88 | ICOS | FcγRIII-γ |
| ScFv | MyD88 | ICOS | FcεRIβ |
| ScFv | MyD88 | ICOS | FcεRIγ |
| ScFv | MyD88 | ICOS | DAP10 |
| ScFv | MyD88 | ICOS | DAP12 |
| ScFv | MyD88 | ICOS | CD32 |
| ScFv | MyD88 | ICOS | CD79a |
| ScFv | MyD88 | ICOS | CD79b |
| ScFv | MyD88 | CD27 | CD8 |
| ScFv | MyD88 | CD27 | CD3ζ |
| ScFv | MyD88 | CD27 | CD3δ |
| ScFv | MyD88 | CD27 | CD3γ |
| ScFv | MyD88 | CD27 | CD3ε |
| ScFv | MyD88 | CD27 | FcγRI-γ |
| ScFv | MyD88 | CD27 | FcγRIII-γ |
| ScFv | MyD88 | CD27 | FcεRIβ |
| ScFv | MyD88 | CD27 | FcεRIγ |
| ScFv | MyD88 | CD27 | DAP10 |
| ScFv | MyD88 | CD27 | DAP12 |
| ScFv | MyD88 | CD27 | CD32 |
| ScFv | MyD88 | CD27 | CD79a |
| ScFv | MyD88 | CD27 | CD79b |
| ScFv | MyD88 | CD28δ | CD8 |
| ScFv | MyD88 | CD28δ | CD3ζ |
| ScFv | MyD88 | CD28δ | CD3δ |
| ScFv | MyD88 | CD28δ | CD3γ |
| ScFv | MyD88 | CD28δ | CD3ε |
| ScFv | MyD88 | CD28δ | FcγRI-γ |
| ScFv | MyD88 | CD28δ | FcγRIII-γ |
| ScFv | MyD88 | CD28δ | FcεRIβ |
| ScFv | MyD88 | CD28δ | FcεRIγ |
| ScFv | MyD88 | CD28δ | DAP10 |
| ScFv | MyD88 | CD28δ | DAP12 |
| ScFv | MyD88 | CD28δ | CD32 |
| ScFv | MyD88 | CD28δ | CD79a |
| ScFv | MyD88 | CD28δ | CD79b |
| ScFv | MyD88 | CD80 | CD8 |
| ScFv | MyD88 | CD80 | CD3ζ |
| ScFv | MyD88 | CD80 | CD3δ |
| ScFv | MyD88 | CD80 | CD3γ |
| ScFv | MyD88 | CD80 | CD3ε |
| ScFv | MyD88 | CD80 | FcγRI-γ |
| ScFv | MyD88 | CD80 | FcγRIII-γ |
| ScFv | MyD88 | CD80 | FcεRIβ |
| ScFv | MyD88 | CD80 | FcεRIγ |
| ScFv | MyD88 | CD80 | DAP10 |
| ScFv | MyD88 | CD80 | DAP12 |
| ScFv | MyD88 | CD80 | CD32 |
| ScFv | MyD88 | CD80 | CD79a |
| ScFv | MyD88 | CD80 | CD79b |
| ScFv | MyD88 | CD86 | CD8 |
| ScFv | MyD88 | CD86 | CD3ζ |
| ScFv | MyD88 | CD86 | CD3δ |
| ScFv | MyD88 | CD86 | CD3γ |
| ScFv | MyD88 | CD86 | CD3ε |
| ScFv | MyD88 | CD86 | FcγRI-γ |
| ScFv | MyD88 | CD86 | FcγRIII-γ |
| ScFv | MyD88 | CD86 | FcεRIβ |
| ScFv | MyD88 | CD86 | FcεRIγ |
| ScFv | MyD88 | CD86 | DAP10 |
| ScFv | MyD88 | CD86 | DAP12 |
| ScFv | MyD88 | CD86 | CD32 |
| ScFv | MyD88 | CD86 | CD79a |
| ScFv | MyD88 | CD86 | CD79b |
| ScFv | MyD88 | OX40 | CD8 |
| ScFv | MyD88 | OX40 | CD3ζ |
| ScFv | MyD88 | OX40 | CD3δ |
| ScFv | MyD88 | OX40 | CD3γ |
| ScFv | MyD88 | OX40 | CD3ε |
| ScFv | MyD88 | OX40 | FcγRI-γ |
| ScFv | MyD88 | OX40 | FcγRIII-γ |
| ScFv | MyD88 | OX40 | FcεRIβ |
| ScFv | MyD88 | OX40 | FcεRIγ |
| ScFv | MyD88 | OX40 | DAP10 |
| ScFv | MyD88 | OX40 | DAP12 |
| ScFv | MyD88 | OX40 | CD32 |
| ScFv | MyD88 | OX40 | CD79a |
| ScFv | MyD88 | OX40 | CD79b |
| ScFv | MyD88 | DAP10 | CD8 |
| ScFv | MyD88 | DAP10 | CD3ζ |
| ScFv | MyD88 | DAP10 | CD3δ |
| ScFv | MyD88 | DAP10 | CD3γ |
| ScFv | MyD88 | DAP10 | CD3ε |
| ScFv | MyD88 | DAP10 | FcγRI-γ |
| ScFv | MyD88 | DAP10 | FcγRIII-γ |
| ScFv | MyD88 | DAP10 | FcεRIβ |
| ScFv | MyD88 | DAP10 | FcεRIγ |
| ScFv | MyD88 | DAP10 | DAP10 |
| ScFv | MyD88 | DAP10 | DAP12 |
| ScFv | MyD88 | DAP10 | CD32 |
| ScFv | MyD88 | DAP10 | CD79a |
| ScFv | MyD88 | DAP10 | CD79b |
| ScFv | MyD88 | DAP12 | CD8 |
| ScFv | MyD88 | DAP12 | CD3ζ |
| ScFv | MyD88 | DAP12 | CD3δ |
| ScFv | MyD88 | DAP12 | CD3γ |
| ScFv | MyD88 | DAP12 | CD3ε |
| ScFv | MyD88 | DAP12 | FcγRI-γ |
| ScFv | MyD88 | DAP12 | FcγRIII-γ |
| ScFv | MyD88 | DAP12 | FcεRIβ |
| ScFv | MyD88 | DAP12 | FcεRIγ |
| ScFv | MyD88 | DAP12 | DAP10 |
| ScFv | MyD88 | DAP12 | DAP12 |
| ScFv | MyD88 | DAP12 | CD32 |
| ScFv | MyD88 | DAP12 | CD79a |
| ScFv | MyD88 | DAP12 | CD79b |
| ScFv | MyD88 | MyD88 | CD8 |
| ScFv | MyD88 | MyD88 | CD3ζ |
| ScFv | MyD88 | MyD88 | CD3δ |
| ScFv | MyD88 | MyD88 | CD3γ |
| ScFv | MyD88 | MyD88 | CD3ε |
| ScFv | MyD88 | MyD88 | FcγRI-γ |
| ScFv | MyD88 | MyD88 | FcγRIII-γ |
| ScFv | MyD88 | MyD88 | FcεRIβ |
| ScFv | MyD88 | MyD88 | FcεRIγ |
| ScFv | MyD88 | MyD88 | DAP10 |
| ScFv | MyD88 | MyD88 | DAP12 |
| ScFv | MyD88 | MyD88 | CD32 |
| ScFv | MyD88 | MyD88 | CD79a |
| ScFv | MyD88 | MyD88 | CD79b |
| ScFv | MyD88 | CD7 | CD8 |
| ScFv | MyD88 | CD7 | CD3ζ |
| ScFv | MyD88 | CD7 | CD3δ |
| ScFv | MyD88 | CD7 | CD3γ |
| ScFv | MyD88 | CD7 | CD3ε |
| ScFv | MyD88 | CD7 | FcγRI-γ |
| ScFv | MyD88 | CD7 | FcγRIII-γ |
| ScFv | MyD88 | CD7 | FcεRIβ |
| ScFv | MyD88 | CD7 | FcεRIγ |
| ScFv | MyD88 | CD7 | DAP10 |
| ScFv | MyD88 | CD7 | DAP12 |
| ScFv | MyD88 | CD7 | CD32 |
| ScFv | MyD88 | CD7 | CD79a |
| ScFv | MyD88 | CD7 | CD79b |
| ScFv | MyD88 | BTNL3 | CD8 |
| ScFv | MyD88 | BTNL3 | CD3ζ |
| ScFv | MyD88 | BTNL3 | CD3δ |
| ScFv | MyD88 | BTNL3 | CD3γ |
| ScFv | MyD88 | BTNL3 | CD3ε |
| ScFv | MyD88 | BTNL3 | FcγRI-γ |
| ScFv | MyD88 | BTNL3 | FcγRIII-γ |
| ScFv | MyD88 | BTNL3 | FcεRIβ |
| ScFv | MyD88 | BTNL3 | FcεRIγ |
| ScFv | MyD88 | BTNL3 | DAP10 |
| ScFv | MyD88 | BTNL3 | DAP12 |
| ScFv | MyD88 | BTNL3 | CD32 |
| ScFv | MyD88 | BTNL3 | CD79a |
| ScFv | MyD88 | BTNL3 | CD79b |
| ScFv | MyD88 | NKG2D | CD8 |
| ScFv | MyD88 | NKG2D | CD3ζ |
| ScFv | MyD88 | NKG2D | CD3δ |
| ScFv | MyD88 | NKG2D | CD3γ |
| ScFv | MyD88 | NKG2D | CD3ε |
| ScFv | MyD88 | NKG2D | FcγRI-γ |
| ScFv | MyD88 | NKG2D | FcγRIII-γ |
| ScFv | MyD88 | NKG2D | FcεRIβ |
| ScFv | MyD88 | NKG2D | FcεRIγ |
| ScFv | MyD88 | NKG2D | DAP10 |
| ScFv | MyD88 | NKG2D | DAP12 |
| ScFv | MyD88 | NKG2D | CD32 |
| ScFv | MyD88 | NKG2D | CD79a |
| ScFv | MyD88 | NKG2D | CD79b |
| ScFv | CD7 | CD28 | CD8 |
| ScFv | CD7 | CD28 | CD3ζ |
| ScFv | CD7 | CD28 | CD3δ |
| ScFv | CD7 | CD28 | CD3γ |
| ScFv | CD7 | CD28 | CD3ε |
| ScFv | CD7 | CD28 | FcγRI-γ |
| ScFv | CD7 | CD28 | FcγRIII-γ |
| ScFv | CD7 | CD28 | FcεRIβ |
| ScFv | CD7 | CD28 | FcεRIγ |
| ScFv | CD7 | CD28 | DAP10 |
| ScFv | CD7 | CD28 | DAP12 |
| ScFv | CD7 | CD28 | CD32 |
| ScFv | CD7 | CD28 | CD79a |
| ScFv | CD7 | CD28 | CD79b |
| ScFv | CD7 | CD8 | CD8 |
| ScFv | CD7 | CD8 | CD3ζ |
| ScFv | CD7 | CD8 | CD3δ |
| ScFv | CD7 | CD8 | CD3γ |
| ScFv | CD7 | CD8 | CD3ε |
| ScFv | CD7 | CD8 | FcγRI-γ |
| ScFv | CD7 | CD8 | FcγRIII-γ |
| ScFv | CD7 | CD8 | FcεRIβ |
| ScFv | CD7 | CD8 | FcεRIγ |
| ScFv | CD7 | CD8 | DAP10 |
| ScFv | CD7 | CD8 | DAP12 |
| ScFv | CD7 | CD8 | CD32 |
| ScFv | CD7 | CD8 | CD79a |
| ScFv | CD7 | CD8 | CD79b |
| ScFv | CD7 | CD4 | CD8 |
| ScFv | CD7 | CD4 | CD3ζ |
| ScFv | CD7 | CD4 | CD3δ |
| ScFv | CD7 | CD4 | CD3γ |
| ScFv | CD7 | CD4 | CD3ε |
| ScFv | CD7 | CD4 | FcγRI-γ |
| ScFv | CD7 | CD4 | FcγRIII-γ |
| ScFv | CD7 | CD4 | FcεRIβ |
| ScFv | CD7 | CD4 | FcεRIγ |
| ScFv | CD7 | CD4 | DAP10 |
| ScFv | CD7 | CD4 | DAP12 |
| ScFv | CD7 | CD4 | CD32 |
| ScFv | CD7 | CD4 | CD79a |
| ScFv | CD7 | CD4 | CD79b |
| ScFv | CD7 | b2c | CD8 |
| ScFv | CD7 | b2c | CD3ζ |
| ScFv | CD7 | b2c | CD3δ |
| ScFv | CD7 | b2c | CD3γ |
| ScFv | CD7 | b2c | CD3ε |
| ScFv | CD7 | b2c | FcγRI-γ |
| ScFv | CD7 | b2c | FcγRIII-γ |
| ScFv | CD7 | b2c | FcεRIβ |
| ScFv | CD7 | b2c | FcεRIγ |
| ScFv | CD7 | b2c | DAP10 |
| ScFv | CD7 | b2c | DAP12 |
| ScFv | CD7 | b2c | CD32 |
| ScFv | CD7 | b2c | CD79a |
| ScFv | CD7 | b2c | CD79b |
| ScFv | CD7 | CD137/41BB | CD8 |
| ScFv | CD7 | CD137/41BB | CD3ζ |
| ScFv | CD7 | CD137/41BB | CD3δ |
| ScFv | CD7 | CD137/41BB | CD3γ |
| ScFv | CD7 | CD137/41BB | CD3ε |
| ScFv | CD7 | CD137/41BB | FcγRI-γ |
| ScFv | CD7 | CD137/41BB | FcγRIII-γ |
| ScFv | CD7 | CD137/41BB | FcεRIβ |
| ScFv | CD7 | CD137/41BB | FcεRIγ |
| ScFv | CD7 | CD137/41BB | DAP10 |
| ScFv | CD7 | CD137/41BB | DAP12 |
| ScFv | CD7 | CD137/41BB | CD32 |
| ScFv | CD7 | CD137/41BB | CD79a |
| ScFv | CD7 | CD137/41BB | CD79b |
| ScFv | CD7 | ICOS | CD8 |
| ScFv | CD7 | ICOS | CD3ζ |
| ScFv | CD7 | ICOS | CD3δ |
| ScFv | CD7 | ICOS | CD3γ |
| ScFv | CD7 | ICOS | CD3ε |
| ScFv | CD7 | ICOS | FcγRI-γ |
| ScFv | CD7 | ICOS | FcγRIII-γ |
| ScFv | CD7 | ICOS | FcεRIβ |
| ScFv | CD7 | ICOS | FcεRIγ |
| ScFv | CD7 | ICOS | DAP10 |
| ScFv | CD7 | ICOS | DAP12 |
| ScFv | CD7 | ICOS | CD32 |
| ScFv | CD7 | ICOS | CD79a |
| ScFv | CD7 | ICOS | CD79b |
| ScFv | CD7 | CD27 | CD8 |
| ScFv | CD7 | CD27 | CD3ζ |
| ScFv | CD7 | CD27 | CD3δ |
| ScFv | CD7 | CD27 | CD3γ |
| ScFv | CD7 | CD27 | CD3ε |
| ScFv | CD7 | CD27 | FcγRI-γ |
| ScFv | CD7 | CD27 | FcγRIII-γ |
| ScFv | CD7 | CD27 | FcεRIβ |
| ScFv | CD7 | CD27 | FcεRIγ |
| ScFv | CD7 | CD27 | DAP10 |
| ScFv | CD7 | CD27 | DAP12 |
| ScFv | CD7 | CD27 | CD32 |
| ScFv | CD7 | CD27 | CD79a |
| ScFv | CD7 | CD27 | CD79b |
| ScFv | CD7 | CD28δ | CD8 |
| ScFv | CD7 | CD28δ | CD3ζ |
| ScFv | CD7 | CD28δ | CD3δ |
| ScFv | CD7 | CD28δ | CD3γ |
| ScFv | CD7 | CD28δ | CD3ε |
| ScFv | CD7 | CD28δ | FcγRI-γ |
| ScFv | CD7 | CD28δ | FcγRIII-γ |
| ScFv | CD7 | CD28δ | FcεRIβ |
| ScFv | CD7 | CD28δ | FcεRIγ |
| ScFv | CD7 | CD28δ | DAP10 |
| ScFv | CD7 | CD28δ | DAP12 |
| ScFv | CD7 | CD28δ | CD32 |
| ScFv | CD7 | CD28δ | CD79a |
| ScFv | CD7 | CD28δ | CD79b |
| ScFv | CD7 | CD80 | CD8 |
| ScFv | CD7 | CD80 | CD3ζ |
| ScFv | CD7 | CD80 | CD3δ |
| ScFv | CD7 | CD80 | CD3γ |
| ScFv | CD7 | CD80 | CD3ε |
| ScFv | CD7 | CD80 | FcγRI-γ |
| ScFv | CD7 | CD80 | FcγRIII-γ |
| ScFv | CD7 | CD80 | FcεRIβ |
| ScFv | CD7 | CD80 | FcεRIγ |
| ScFv | CD7 | CD80 | DAP10 |
| ScFv | CD7 | CD80 | DAP12 |
| ScFv | CD7 | CD80 | CD32 |
| ScFv | CD7 | CD80 | CD79a |
| ScFv | CD7 | CD80 | CD79b |
| ScFv | CD7 | CD86 | CD8 |
| ScFv | CD7 | CD86 | CD3ζ |
| ScFv | CD7 | CD86 | CD3δ |
| ScFv | CD7 | CD86 | CD3γ |
| ScFv | CD7 | CD86 | CD3ε |
| ScFv | CD7 | CD86 | FcγRI-γ |
| ScFv | CD7 | CD86 | FcγRIII-γ |
| ScFv | CD7 | CD86 | FcεRIβ |
| ScFv | CD7 | CD86 | FcεRIγ |
| ScFv | CD7 | CD86 | DAP10 |
| ScFv | CD7 | CD86 | DAP12 |
| ScFv | CD7 | CD86 | CD32 |
| ScFv | CD7 | CD86 | CD79a |
| ScFv | CD7 | CD86 | CD79b |
| ScFv | CD7 | OX40 | CD8 |
| ScFv | CD7 | OX40 | CD3ζ |
| ScFv | CD7 | OX40 | CD3δ |
| ScFv | CD7 | OX40 | CD3γ |
| ScFv | CD7 | OX40 | CD3ε |
| ScFv | CD7 | OX40 | FcγRI-γ |
| ScFv | CD7 | OX40 | FcγRIII-γ |
| ScFv | CD7 | OX40 | FcεRIβ |
| ScFv | CD7 | OX40 | FcεRIγ |
| ScFv | CD7 | OX40 | DAP10 |
| ScFv | CD7 | OX40 | DAP12 |
| ScFv | CD7 | OX40 | CD32 |
| ScFv | CD7 | OX40 | CD79a |
| ScFv | CD7 | OX40 | CD79b |
| ScFv | CD7 | DAP10 | CD8 |
| ScFv | CD7 | DAP10 | CD3ζ |
| ScFv | CD7 | DAP10 | CD3δ |
| ScFv | CD7 | DAP10 | CD3γ |
| ScFv | CD7 | DAP10 | CD3ε |
| ScFv | CD7 | DAP10 | FcγRI-γ |
| ScFv | CD7 | DAP10 | FcγRIII-γ |
| ScFv | CD7 | DAP10 | FcεRIβ |
| ScFv | CD7 | DAP10 | FcεRIγ |
| ScFv | CD7 | DAP10 | DAP10 |
| ScFv | CD7 | DAP10 | DAP12 |
| ScFv | CD7 | DAP10 | CD32 |
| ScFv | CD7 | DAP10 | CD79a |
| ScFv | CD7 | DAP10 | CD79b |
| ScFv | CD7 | DAP12 | CD8 |
| ScFv | CD7 | DAP12 | CD3ζ |
| ScFv | CD7 | DAP12 | CD3δ |
| ScFv | CD7 | DAP12 | CD3γ |
| ScFv | CD7 | DAP12 | CD3ε |
| ScFv | CD7 | DAP12 | FcγRI-γ |
| ScFv | CD7 | DAP12 | FcγRIII-γ |
| ScFv | CD7 | DAP12 | FcεRIβ |
| ScFv | CD7 | DAP12 | FcεRIγ |
| ScFv | CD7 | DAP12 | DAP10 |
| ScFv | CD7 | DAP12 | DAP12 |
| ScFv | CD7 | DAP12 | CD32 |
| ScFv | CD7 | DAP12 | CD79a |
| ScFv | CD7 | DAP12 | CD79b |
| ScFv | CD7 | MyD88 | CD8 |
| ScFv | CD7 | MyD88 | CD3ζ |
| ScFv | CD7 | MyD88 | CD3δ |
| ScFv | CD7 | MyD88 | CD3γ |
| ScFv | CD7 | MyD88 | CD3ε |
| ScFv | CD7 | MyD88 | FcγRI-γ |
| ScFv | CD7 | MyD88 | FcγRIII-γ |
| ScFv | CD7 | MyD88 | FcεRIβ |
| ScFv | CD7 | MyD88 | FcεRIγ |
| ScFv | CD7 | MyD88 | DAP10 |
| ScFv | CD7 | MyD88 | DAP12 |
| ScFv | CD7 | MyD88 | CD32 |
| ScFv | CD7 | MyD88 | CD79a |
| ScFv | CD7 | MyD88 | CD79b |
| ScFv | CD7 | CD7 | CD8 |
| ScFv | CD7 | CD7 | CD3ζ |
| ScFv | CD7 | CD7 | CD3δ |
| ScFv | CD7 | CD7 | CD3γ |
| ScFv | CD7 | CD7 | CD3ε |
| ScFv | CD7 | CD7 | FcγRI-γ |
| ScFv | CD7 | CD7 | FcγRIII-γ |
| ScFv | CD7 | CD7 | FcεRIβ |
| ScFv | CD7 | CD7 | FcεRIγ |
| ScFv | CD7 | CD7 | DAP10 |
| ScFv | CD7 | CD7 | DAP12 |
| ScFv | CD7 | CD7 | CD32 |
| ScFv | CD7 | CD7 | CD79a |
| ScFv | CD7 | CD7 | CD79b |
| ScFv | CD7 | BTNL3 | CD8 |
| ScFv | CD7 | BTNL3 | CD3ζ |
| ScFv | CD7 | BTNL3 | CD3δ |
| ScFv | CD7 | BTNL3 | CD3γ |
| ScFv | CD7 | BTNL3 | CD3ε |
| ScFv | CD7 | BTNL3 | FcγRI-γ |
| ScFv | CD7 | BTNL3 | FcγRIII-γ |
| ScFv | CD7 | BTNL3 | FcεRIβ |
| ScFv | CD7 | BTNL3 | FcεRIγ |
| ScFv | CD7 | BTNL3 | DAP10 |
| ScFv | CD7 | BTNL3 | DAP12 |
| ScFv | CD7 | BTNL3 | CD32 |
| ScFv | CD7 | BTNL3 | CD79a |
| ScFv | CD7 | BTNL3 | CD79b |
| ScFv | CD7 | NKG2D | CD8 |
| ScFv | CD7 | NKG2D | CD3ζ |
| ScFv | CD7 | NKG2D | CD3δ |
| ScFv | CD7 | NKG2D | CD3γ |
| ScFv | CD7 | NKG2D | CD3ε |
| ScFv | CD7 | NKG2D | FcγRI-γ |
| ScFv | CD7 | NKG2D | FcγRIII-γ |
| ScFv | CD7 | NKG2D | FcεRIβ |
| ScFv | CD7 | NKG2D | FcεRIγ |
| ScFv | CD7 | NKG2D | DAP10 |
| ScFv | CD7 | NKG2D | DAP12 |
| ScFv | CD7 | NKG2D | CD32 |
| ScFv | CD7 | NKG2D | CD79a |
| ScFv | CD7 | NKG2D | CD79b |
| ScFv | BTNL3 | CD28 | CD8 |
| ScFv | BTNL3 | CD28 | CD3ζ |
| ScFv | BTNL3 | CD28 | CD3δ |
| ScFv | BTNL3 | CD28 | CD3γ |
| ScFv | BTNL3 | CD28 | CD3ε |
| ScFv | BTNL3 | CD28 | FcγRI-γ |
| ScFv | BTNL3 | CD28 | FcγRIII-γ |
| ScFv | BTNL3 | CD28 | FcεRIβ |
| ScFv | BTNL3 | CD28 | FcεRIγ |
| ScFv | BTNL3 | CD28 | DAP10 |
| ScFv | BTNL3 | CD28 | DAP12 |
| ScFv | BTNL3 | CD28 | CD32 |
| ScFv | BTNL3 | CD28 | CD79a |
| ScFv | BTNL3 | CD28 | CD79b |
| ScFv | BTNL3 | CD8 | CD8 |
| ScFv | BTNL3 | CD8 | CD3ζ |
| ScFv | BTNL3 | CD8 | CD3δ |
| ScFv | BTNL3 | CD8 | CD3γ |
| ScFv | BTNL3 | CD8 | CD3ε |
| ScFv | BTNL3 | CD8 | FcγRI-γ |
| ScFv | BTNL3 | CD8 | FcγRIII-γ |
| ScFv | BTNL3 | CD8 | FcεRIβ |
| ScFv | BTNL3 | CD8 | FcεRIγ |
| ScFv | BTNL3 | CD8 | DAP10 |
| ScFv | BTNL3 | CD8 | DAP12 |
| ScFv | BTNL3 | CD8 | CD32 |
| ScFv | BTNL3 | CD8 | CD79a |
| ScFv | BTNL3 | CD8 | CD79b |
| ScFv | BTNL3 | CD4 | CD8 |
| ScFv | BTNL3 | CD4 | CD3ζ |
| ScFv | BTNL3 | CD4 | CD3δ |
| ScFv | BTNL3 | CD4 | CD3γ |
| ScFv | BTNL3 | CD4 | CD3ε |
| ScFv | BTNL3 | CD4 | FcγRI-γ |
| ScFv | BTNL3 | CD4 | FcγRIII-γ |
| ScFv | BTNL3 | CD4 | FcεRIβ |
| ScFv | BTNL3 | CD4 | FcεRIγ |
| ScFv | BTNL3 | CD4 | DAP10 |
| ScFv | BTNL3 | CD4 | DAP12 |
| ScFv | BTNL3 | CD4 | CD32 |
| ScFv | BTNL3 | CD4 | CD79a |
| ScFv | BTNL3 | CD4 | CD79b |
| ScFv | BTNL3 | b2c | CD8 |
| ScFv | BTNL3 | b2c | CD3ζ |
| ScFv | BTNL3 | b2c | CD3δ |
| ScFv | BTNL3 | b2c | CD3γ |
| ScFv | BTNL3 | b2c | CD3ε |
| ScFv | BTNL3 | b2c | FcγRI-γ |
| ScFv | BTNL3 | b2c | FcγRIII-γ |
| ScFv | BTNL3 | b2c | FcεRIβ |
| ScFv | BTNL3 | b2c | FcεRIγ |
| ScFv | BTNL3 | b2c | DAP10 |
| ScFv | BTNL3 | b2c | DAP12 |
| ScFv | BTNL3 | b2c | CD32 |
| ScFv | BTNL3 | b2c | CD79a |
| ScFv | BTNL3 | b2c | CD79b |
| ScFv | BTNL3 | CD137/41BB | CD8 |
| ScFv | BTNL3 | CD137/41BB | CD3ζ |
| ScFv | BTNL3 | CD137/41BB | CD3δ |
| ScFv | BTNL3 | CD137/41BB | CD3γ |
| ScFv | BTNL3 | CD137/41BB | CD3ε |
| ScFv | BTNL3 | CD137/41BB | FcγRI-γ |
| ScFv | BTNL3 | CD137/41BB | FcγRIII-γ |
| ScFv | BTNL3 | CD137/41BB | FcεRIβ |
| ScFv | BTNL3 | CD137/41BB | FcεRIγ |
| ScFv | BTNL3 | CD137/41BB | DAP10 |
| ScFv | BTNL3 | CD137/41BB | DAP12 |
| ScFv | BTNL3 | CD137/41BB | CD32 |
| ScFv | BTNL3 | CD137/41BB | CD79a |
| ScFv | BTNL3 | CD137/41BB | CD79b |
| ScFv | BTNL3 | ICOS | CD8 |
| ScFv | BTNL3 | ICOS | CD3ζ |
| ScFv | BTNL3 | ICOS | CD3δ |
| ScFv | BTNL3 | ICOS | CD3γ |
| ScFv | BTNL3 | ICOS | CD3ε |
| ScFv | BTNL3 | ICOS | FcγRI-γ |
| ScFv | BTNL3 | ICOS | FcγRIII-γ |
| ScFv | BTNL3 | ICOS | FcεRIβ |
| ScFv | BTNL3 | ICOS | FcεRIγ |
| ScFv | BTNL3 | ICOS | DAP10 |
| ScFv | BTNL3 | ICOS | DAP12 |
| ScFv | BTNL3 | ICOS | CD32 |
| ScFv | BTNL3 | ICOS | CD79a |
| ScFv | BTNL3 | ICOS | CD79b |
| ScFv | BTNL3 | CD27 | CD8 |
| ScFv | BTNL3 | CD27 | CD3ζ |
| ScFv | BTNL3 | CD27 | CD3δ |
| ScFv | BTNL3 | CD27 | CD3γ |
| ScFv | BTNL3 | CD27 | CD3ε |
| ScFv | BTNL3 | CD27 | FcγRI-γ |
| ScFv | BTNL3 | CD27 | FcγRIII-γ |
| ScFv | BTNL3 | CD27 | FcεRIβ |
| ScFv | BTNL3 | CD27 | FcεRIγ |
| ScFv | BTNL3 | CD27 | DAP10 |
| ScFv | BTNL3 | CD27 | DAP12 |
| ScFv | BTNL3 | CD27 | CD32 |
| ScFv | BTNL3 | CD27 | CD79a |
| ScFv | BTNL3 | CD27 | CD79b |
| ScFv | BTNL3 | CD28δ | CD8 |
| ScFv | BTNL3 | CD28δ | CD3ζ |
| ScFv | BTNL3 | CD28δ | CD3δ |
| ScFv | BTNL3 | CD28δ | CD3γ |
| ScFv | BTNL3 | CD28δ | CD3ε |
| ScFv | BTNL3 | CD28δ | FcγRI-γ |
| ScFv | BTNL3 | CD28δ | FcγRIII-γ |
| ScFv | BTNL3 | CD28δ | FcεRIβ |
| ScFv | BTNL3 | CD28δ | FcεRIγ |
| ScFv | BTNL3 | CD28δ | DAP10 |
| ScFv | BTNL3 | CD28δ | DAP12 |
| ScFv | BTNL3 | CD28δ | CD32 |
| ScFv | BTNL3 | CD28δ | CD79a |
| ScFv | BTNL3 | CD28δ | CD79b |
| ScFv | BTNL3 | CD80 | CD8 |
| ScFv | BTNL3 | CD80 | CD3ζ |
| ScFv | BTNL3 | CD80 | CD3δ |
| ScFv | BTNL3 | CD80 | CD3γ |
| ScFv | BTNL3 | CD80 | CD3ε |
| ScFv | BTNL3 | CD80 | FcγRI-γ |
| ScFv | BTNL3 | CD80 | FcγRIII-γ |
| ScFv | BTNL3 | CD80 | FcεRIβ |
| ScFv | BTNL3 | CD80 | FcεRIγ |
| ScFv | BTNL3 | CD80 | DAP10 |
| ScFv | BTNL3 | CD80 | DAP12 |
| ScFv | BTNL3 | CD80 | CD32 |
| ScFv | BTNL3 | CD80 | CD79a |
| ScFv | BTNL3 | CD80 | CD79b |
| ScFv | BTNL3 | CD86 | CD8 |
| ScFv | BTNL3 | CD86 | CD3ζ |
| ScFv | BTNL3 | CD86 | CD3δ |
| ScFv | BTNL3 | CD86 | CD3γ |
| ScFv | BTNL3 | CD86 | CD3ε |
| ScFv | BTNL3 | CD86 | FcγRI-γ |
| ScFv | BTNL3 | CD86 | FcγRIII-γ |
| ScFv | BTNL3 | CD86 | FcεRIβ |
| ScFv | BTNL3 | CD86 | FcεRIγ |
| ScFv | BTNL3 | CD86 | DAP10 |
| ScFv | BTNL3 | CD86 | DAP12 |
| ScFv | BTNL3 | CD86 | CD32 |
| ScFv | BTNL3 | CD86 | CD79a |
| ScFv | BTNL3 | CD86 | CD79b |
| ScFv | BTNL3 | OX40 | CD8 |
| ScFv | BTNL3 | OX40 | CD3ζ |
| ScFv | BTNL3 | OX40 | CD3δ |
| ScFv | BTNL3 | OX40 | CD3γ |
| ScFv | BTNL3 | OX40 | CD3ε |
| ScFv | BTNL3 | OX40 | FcγRI-γ |
| ScFv | BTNL3 | OX40 | FcγRIII-γ |
| ScFv | BTNL3 | OX40 | FcεRIβ |
| ScFv | BTNL3 | OX40 | FcεRIγ |
| ScFv | BTNL3 | OX40 | DAP10 |
| ScFv | BTNL3 | OX40 | DAP12 |
| ScFv | BTNL3 | OX40 | CD32 |
| ScFv | BTNL3 | OX40 | CD79a |
| ScFv | BTNL3 | OX40 | CD79b |
| ScFv | BTNL3 | DAP10 | CD8 |
| ScFv | BTNL3 | DAP10 | CD3ζ |
| ScFv | BTNL3 | DAP10 | CD3δ |
| ScFv | BTNL3 | DAP10 | CD3γ |
| ScFv | BTNL3 | DAP10 | CD3ε |
| ScFv | BTNL3 | DAP10 | FcγRI-γ |
| ScFv | BTNL3 | DAP10 | FcγRIII-γ |
| ScFv | BTNL3 | DAP10 | FcεRIβ |
| ScFv | BTNL3 | DAP10 | FcεRIγ |
| ScFv | BTNL3 | DAP10 | DAP10 |
| ScFv | BTNL3 | DAP10 | DAP12 |
| ScFv | BTNL3 | DAP10 | CD32 |
| ScFv | BTNL3 | DAP10 | CD79a |
| ScFv | BTNL3 | DAP10 | CD79b |
| ScFv | BTNL3 | DAP12 | CD8 |
| ScFv | BTNL3 | DAP12 | CD3ζ |
| ScFv | BTNL3 | DAP12 | CD3δ |
| ScFv | BTNL3 | DAP12 | CD3γ |
| ScFv | BTNL3 | DAP12 | CD3ε |
| ScFv | BTNL3 | DAP12 | FcγRI-γ |
| ScFv | BTNL3 | DAP12 | FcγRIII-γ |
| ScFv | BTNL3 | DAP12 | FcεRIβ |
| ScFv | BTNL3 | DAP12 | FcεRIγ |
| ScFv | BTNL3 | DAP12 | DAP10 |
| ScFv | BTNL3 | DAP12 | DAP12 |
| ScFv | BTNL3 | DAP12 | CD32 |
| ScFv | BTNL3 | DAP12 | CD79a |
| ScFv | BTNL3 | DAP12 | CD79b |
| ScFv | BTNL3 | MyD88 | CD8 |
| ScFv | BTNL3 | MyD88 | CD3ζ |
| ScFv | BTNL3 | MyD88 | CD3δ |
| ScFv | BTNL3 | MyD88 | CD3γ |
| ScFv | BTNL3 | MyD88 | CD3ε |
| ScFv | BTNL3 | MyD88 | FcγRI-γ |
| ScFv | BTNL3 | MyD88 | FcγRIII-γ |
| ScFv | BTNL3 | MyD88 | FcεRIβ |
| ScFv | BTNL3 | MyD88 | FcεRIγ |
| ScFv | BTNL3 | MyD88 | DAP10 |
| ScFv | BTNL3 | MyD88 | DAP12 |
| ScFv | BTNL3 | MyD88 | CD32 |
| ScFv | BTNL3 | MyD88 | CD79a |
| ScFv | BTNL3 | MyD88 | CD79b |
| ScFv | BTNL3 | CD7 | CD8 |
| ScFv | BTNL3 | CD7 | CD3ζ |
| ScFv | BTNL3 | CD7 | CD3δ |
| ScFv | BTNL3 | CD7 | CD3γ |
| ScFv | BTNL3 | CD7 | CD3ε |
| ScFv | BTNL3 | CD7 | FcγRI-γ |
| ScFv | BTNL3 | CD7 | FcγRIII-γ |
| ScFv | BTNL3 | CD7 | FcεRIβ |
| ScFv | BTNL3 | CD7 | FcεRIγ |
| ScFv | BTNL3 | CD7 | DAP10 |
| ScFv | BTNL3 | CD7 | DAP12 |
| ScFv | BTNL3 | CD7 | CD32 |
| ScFv | BTNL3 | CD7 | CD79a |
| ScFv | BTNL3 | CD7 | CD79b |
| ScFv | BTNL3 | BTNL3 | CD8 |
| ScFv | BTNL3 | BTNL3 | CD3ζ |
| ScFv | BTNL3 | BTNL3 | CD3δ |
| ScFv | BTNL3 | BTNL3 | CD3γ |
| ScFv | BTNL3 | BTNL3 | CD3ε |
| ScFv | BTNL3 | BTNL3 | FcγRI-γ |
| ScFv | BTNL3 | BTNL3 | FcγRIII-γ |
| ScFv | BTNL3 | BTNL3 | FcεRIβ |
| ScFv | BTNL3 | BTNL3 | FcεRIγ |
| ScFv | BTNL3 | BTNL3 | DAP10 |
| ScFv | BTNL3 | BTNL3 | DAP12 |
| ScFv | BTNL3 | BTNL3 | CD32 |
| ScFv | BTNL3 | BTNL3 | CD79a |
| ScFv | BTNL3 | BTNL3 | CD79b |
| ScFv | BTNL3 | NKG2D | CD8 |
| ScFv | BTNL3 | NKG2D | CD3ζ |
| ScFv | BTNL3 | NKG2D | CD3δ |
| ScFv | BTNL3 | NKG2D | CD3γ |
| ScFv | BTNL3 | NKG2D | CD3ε |
| ScFv | BTNL3 | NKG2D | FcγRI-γ |
| ScFv | BTNL3 | NKG2D | FcγRIII-γ |
| ScFv | BTNL3 | NKG2D | FcεRIβ |
| ScFv | BTNL3 | NKG2D | FcεRIγ |
| ScFv | BTNL3 | NKG2D | DAP10 |
| ScFv | BTNL3 | NKG2D | DAP12 |
| ScFv | BTNL3 | NKG2D | CD32 |
| ScFv | BTNL3 | NKG2D | CD79a |
| ScFv | BTNL3 | NKG2D | CD79b |
| ScFv | NKG2D | CD28 | CD8 |
| ScFv | NKG2D | CD28 | CD3ζ |
| ScFv | NKG2D | CD28 | CD3δ |
| ScFv | NKG2D | CD28 | CD3γ |
| ScFv | NKG2D | CD28 | CD3ε |
| ScFv | NKG2D | CD28 | FcγRI-γ |
| ScFv | NKG2D | CD28 | FcγRIII-γ |
| ScFv | NKG2D | CD28 | FcεRIβ |
| ScFv | NKG2D | CD28 | FcεRIγ |
| ScFv | NKG2D | CD28 | DAP10 |
| ScFv | NKG2D | CD28 | DAP12 |
| ScFv | NKG2D | CD28 | CD32 |
| ScFv | NKG2D | CD28 | CD79a |
| ScFv | NKG2D | CD28 | CD79b |
| ScFv | NKG2D | CD8 | CD8 |
| ScFv | NKG2D | CD8 | CD3ζ |
| ScFv | NKG2D | CD8 | CD3δ |
| ScFv | NKG2D | CD8 | CD3γ |
| ScFv | NKG2D | CD8 | CD3ε |
| ScFv | NKG2D | CD8 | FcγRI-γ |
| ScFv | NKG2D | CD8 | FcγRIII-γ |
| ScFv | NKG2D | CD8 | FcεRIβ |
| ScFv | NKG2D | CD8 | FcεRIγ |
| ScFv | NKG2D | CD8 | DAP10 |
| ScFv | NKG2D | CD8 | DAP12 |
| ScFv | NKG2D | CD8 | CD32 |
| ScFv | NKG2D | CD8 | CD79a |
| ScFv | NKG2D | CD8 | CD79b |
| ScFv | NKG2D | CD4 | CD8 |
| ScFv | NKG2D | CD4 | CD3ζ |
| ScFv | NKG2D | CD4 | CD3δ |
| ScFv | NKG2D | CD4 | CD3γ |
| ScFv | NKG2D | CD4 | CD3ε |
| ScFv | NKG2D | CD4 | FcγRI-γ |
| ScFv | NKG2D | CD4 | FcγRIII-γ |
| ScFv | NKG2D | CD4 | FcεRIβ |
| ScFv | NKG2D | CD4 | FcεRIγ |
| ScFv | NKG2D | CD4 | DAP10 |
| ScFv | NKG2D | CD4 | DAP12 |
| ScFv | NKG2D | CD4 | CD32 |
| ScFv | NKG2D | CD4 | CD79a |
| ScFv | NKG2D | CD4 | CD79b |
| ScFv | NKG2D | b2c | CD8 |
| ScFv | NKG2D | b2c | CD3ζ |
| ScFv | NKG2D | b2c | CD3δ |
| ScFv | NKG2D | b2c | CD3γ |
| ScFv | NKG2D | b2c | CD3ε |
| ScFv | NKG2D | b2c | FcγRI-γ |
| ScFv | NKG2D | b2c | FcγRIII-γ |
| ScFv | NKG2D | b2c | FcεRIβ |
| ScFv | NKG2D | b2c | FcεRIγ |
| ScFv | NKG2D | b2c | DAP10 |
| ScFv | NKG2D | b2c | DAP12 |
| ScFv | NKG2D | b2c | CD32 |
| ScFv | NKG2D | b2c | CD79a |
| ScFv | NKG2D | b2c | CD79b |
| ScFv | NKG2D | CD137/41BB | CD8 |
| ScFv | NKG2D | CD137/41BB | CD3ζ |
| ScFv | NKG2D | CD137/41BB | CD3δ |
| ScFv | NKG2D | CD137/41BB | CD3γ |
| ScFv | NKG2D | CD137/41BB | CD3ε |
| ScFv | NKG2D | CD137/41BB | FcγRI-γ |
| ScFv | NKG2D | CD137/41BB | FcγRIII-γ |
| ScFv | NKG2D | CD137/41BB | FcεRIβ |
| ScFv | NKG2D | CD137/41BB | FcεRIγ |
| ScFv | NKG2D | CD137/41BB | DAP10 |
| ScFv | NKG2D | CD137/41BB | DAP12 |
| ScFv | NKG2D | CD137/41BB | CD32 |
| ScFv | NKG2D | CD137/41BB | CD79a |
| ScFv | NKG2D | CD137/41BB | CD79b |
| ScFv | NKG2D | ICOS | CD8 |
| ScFv | NKG2D | ICOS | CD3ζ |
| ScFv | NKG2D | ICOS | CD3δ |
| ScFv | NKG2D | ICOS | CD3γ |
| ScFv | NKG2D | ICOS | CD3ε |
| ScFv | NKG2D | ICOS | FcγRI-γ |
| ScFv | NKG2D | ICOS | FcγRIII-γ |
| ScFv | NKG2D | ICOS | FcεRIβ |
| ScFv | NKG2D | ICOS | FcεRIγ |
| ScFv | NKG2D | ICOS | DAP10 |
| ScFv | NKG2D | ICOS | DAP12 |
| ScFv | NKG2D | ICOS | CD32 |
| ScFv | NKG2D | ICOS | CD79a |
| ScFv | NKG2D | ICOS | CD79b |
| ScFv | NKG2D | CD27 | CD8 |
| ScFv | NKG2D | CD27 | CD3ζ |
| ScFv | NKG2D | CD27 | CD3δ |
| ScFv | NKG2D | CD27 | CD3γ |
| ScFv | NKG2D | CD27 | CD3ε |
| ScFv | NKG2D | CD27 | FcγRI-γ |
| ScFv | NKG2D | CD27 | FcγRIII-γ |
| ScFv | NKG2D | CD27 | FcεRIβ |
| ScFv | NKG2D | CD27 | FcεRIγ |
| ScFv | NKG2D | CD27 | DAP10 |
| ScFv | NKG2D | CD27 | DAP12 |
| ScFv | NKG2D | CD27 | CD32 |
| ScFv | NKG2D | CD27 | CD79a |
| ScFv | NKG2D | CD27 | CD79b |
| ScFv | NKG2D | CD28δ | CD8 |
| ScFv | NKG2D | CD28δ | CD3ζ |
| ScFv | NKG2D | CD28δ | CD3δ |
| ScFv | NKG2D | CD28δ | CD3γ |
| ScFv | NKG2D | CD28δ | CD3ε |
| ScFv | NKG2D | CD28δ | FcγRI-γ |
| ScFv | NKG2D | CD28δ | FcγRIII-γ |
| ScFv | NKG2D | CD28δ | FcεRIβ |
| ScFv | NKG2D | CD28δ | FcεRIγ |
| ScFv | NKG2D | CD28δ | DAP10 |
| ScFv | NKG2D | CD28δ | DAP12 |
| ScFv | NKG2D | CD28δ | CD32 |
| ScFv | NKG2D | CD28δ | CD79a |
| ScFv | NKG2D | CD28δ | CD79b |
| ScFv | NKG2D | CD80 | CD8 |
| ScFv | NKG2D | CD80 | CD3ζ |
| ScFv | NKG2D | CD80 | CD3δ |
| ScFv | NKG2D | CD80 | CD3γ |
| ScFv | NKG2D | CD80 | CD3ε |
| ScFv | NKG2D | CD80 | FcγRI-γ |
| ScFv | NKG2D | CD80 | FcγRIII-γ |
| ScFv | NKG2D | CD80 | FcεRIβ |
| ScFv | NKG2D | CD80 | FcεRIγ |
| ScFv | NKG2D | CD80 | DAP10 |
| ScFv | NKG2D | CD80 | DAP12 |
| ScFv | NKG2D | CD80 | CD32 |
| ScFv | NKG2D | CD80 | CD79a |
| ScFv | NKG2D | CD80 | CD79b |
| ScFv | NKG2D | CD86 | CD8 |
| ScFv | NKG2D | CD86 | CD3ζ |
| ScFv | NKG2D | CD86 | CD3δ |
| ScFv | NKG2D | CD86 | CD3γ |
| ScFv | NKG2D | CD86 | CD3ε |
| ScFv | NKG2D | CD86 | FcγRI-γ |
| ScFv | NKG2D | CD86 | FcγRIII-γ |
| ScFv | NKG2D | CD86 | FcεRIβ |
| ScFv | NKG2D | CD86 | FcεRIγ |
| ScFv | NKG2D | CD86 | DAP10 |
| ScFv | NKG2D | CD86 | DAP12 |
| ScFv | NKG2D | CD86 | CD32 |
| ScFv | NKG2D | CD86 | CD79a |
| ScFv | NKG2D | CD86 | CD79b |
| ScFv | NKG2D | OX40 | CD8 |
| ScFv | NKG2D | OX40 | CD3ζ |
| ScFv | NKG2D | OX40 | CD3δ |
| ScFv | NKG2D | OX40 | CD3γ |
| ScFv | NKG2D | OX40 | CD3ε |
| ScFv | NKG2D | OX40 | FcγRI-γ |
| ScFv | NKG2D | OX40 | FcγRIII-γ |
| ScFv | NKG2D | OX40 | FcεRIβ |
| ScFv | NKG2D | OX40 | FcεRIγ |
| ScFv | NKG2D | OX40 | DAP10 |
| ScFv | NKG2D | OX40 | DAP12 |
| ScFv | NKG2D | OX40 | CD32 |
| ScFv | NKG2D | OX40 | CD79a |
| ScFv | NKG2D | OX40 | CD79b |
| ScFv | NKG2D | DAP10 | CD8 |
| ScFv | NKG2D | DAP10 | CD3ζ |
| ScFv | NKG2D | DAP10 | CD3δ |
| ScFv | NKG2D | DAP10 | CD3γ |
| ScFv | NKG2D | DAP10 | CD3ε |
| ScFv | NKG2D | DAP10 | FcγRI-γ |
| ScFv | NKG2D | DAP10 | FcγRIII-γ |
| ScFv | NKG2D | DAP10 | FcεRIβ |
| ScFv | NKG2D | DAP10 | FcεRIγ |
| ScFv | NKG2D | DAP10 | DAP10 |
| ScFv | NKG2D | DAP10 | DAP12 |
| ScFv | NKG2D | DAP10 | CD32 |
| ScFv | NKG2D | DAP10 | CD79a |
| ScFv | NKG2D | DAP10 | CD79b |
| ScFv | NKG2D | DAP12 | CD8 |
| ScFv | NKG2D | DAP12 | CD3ζ |
| ScFv | NKG2D | DAP12 | CD3δ |
| ScFv | NKG2D | DAP12 | CD3γ |
| ScFv | NKG2D | DAP12 | CD3ε |
| ScFv | NKG2D | DAP12 | FcγRI-γ |
| ScFv | NKG2D | DAP12 | FcγRIII-γ |
| ScFv | NKG2D | DAP12 | FcεRIβ |
| ScFv | NKG2D | DAP12 | FcεRIγ |
| ScFv | NKG2D | DAP12 | DAP10 |
| ScFv | NKG2D | DAP12 | DAP12 |
| ScFv | NKG2D | DAP12 | CD32 |
| ScFv | NKG2D | DAP12 | CD79a |
| ScFv | NKG2D | DAP12 | CD79b |
| ScFv | NKG2D | MyD88 | CD8 |
| ScFv | NKG2D | MyD88 | CD3ζ |
| ScFv | NKG2D | MyD88 | CD3δ |
| ScFv | NKG2D | MyD88 | CD3γ |
| ScFv | NKG2D | MyD88 | CD3ε |
| ScFv | NKG2D | MyD88 | FcγRI-γ |
| ScFv | NKG2D | MyD88 | FcγRIII-γ |
| ScFv | NKG2D | MyD88 | FcεRIβ |
| ScFv | NKG2D | MyD88 | FcεRIγ |
| ScFv | NKG2D | MyD88 | DAP10 |
| ScFv | NKG2D | MyD88 | DAP12 |
| ScFv | NKG2D | MyD88 | CD32 |
| ScFv | NKG2D | MyD88 | CD79a |
| ScFv | NKG2D | MyD88 | CD79b |
| ScFv | NKG2D | CD7 | CD8 |
| ScFv | NKG2D | CD7 | CD3ζ |
| ScFv | NKG2D | CD7 | CD3δ |
| ScFv | NKG2D | CD7 | CD3γ |
| ScFv | NKG2D | CD7 | CD3ε |
| ScFv | NKG2D | CD7 | FcγRI-γ |
| ScFv | NKG2D | CD7 | FcγRIII-γ |
| ScFv | NKG2D | CD7 | FcεRIβ |
| ScFv | NKG2D | CD7 | FcεRIγ |
| ScFv | NKG2D | CD7 | DAP10 |
| ScFv | NKG2D | CD7 | DAP12 |
| ScFv | NKG2D | CD7 | CD32 |
| ScFv | NKG2D | CD7 | CD79a |
| ScFv | NKG2D | CD7 | CD79b |
| ScFv | NKG2D | BTNL3 | CD8 |
| ScFv | NKG2D | BTNL3 | CD3ζ |
| ScFv | NKG2D | BTNL3 | CD3δ |
| ScFv | NKG2D | BTNL3 | CD3γ |
| ScFv | NKG2D | BTNL3 | CD3ε |
| ScFv | NKG2D | BTNL3 | FcγRI-γ |
| ScFv | NKG2D | BTNL3 | FcγRIII-γ |
| ScFv | NKG2D | BTNL3 | FcεRIβ |
| ScFv | NKG2D | BTNL3 | FcεRIγ |
| ScFv | NKG2D | BTNL3 | DAP10 |
| ScFv | NKG2D | BTNL3 | DAP12 |
| ScFv | NKG2D | BTNL3 | CD32 |
| ScFv | NKG2D | BTNL3 | CD79a |
| ScFv | NKG2D | BTNL3 | CD79b |
| ScFv | NKG2D | NKG2D | CD8 |
| ScFv | NKG2D | NKG2D | CD3ζ |
| ScFv | NKG2D | NKG2D | CD3δ |
| ScFv | NKG2D | NKG2D | CD3γ |
| ScFv | NKG2D | NKG2D | CD3ε |
| ScFv | NKG2D | NKG2D | FcγRI-γ |
| ScFv | NKG2D | NKG2D | FcγRIII-γ |
| ScFv | NKG2D | NKG2D | FcεRIβ |
| ScFv | NKG2D | NKG2D | FcεRIγ |
| ScFv | NKG2D | NKG2D | DAP10 |
| ScFv | NKG2D | NKG2D | DAP12 |
| ScFv | NKG2D | NKG2D | CD32 |
| ScFv | NKG2D | NKG2D | CD79a |
| ScFv | NKG2D | NKG2D | CD79b |

TABLE 4
CARs lacking Co-Simulatory Signal (for dual CAR approach)

| | Co-stimulatory | Signal |
|---|---|---|
| ScFv | Signal | Domain |
| ScFv | none | CD8 |
| ScFv | none | CD3ζ |
| ScFv | none | CD3δ |
| ScFv | none | CD3γ |
| ScFv | none | CD3ε |
| ScFv | none | FcγRI-y |
| ScFv | none | FcγRIII-γ |
| ScFv | none | FcεRIβ |
| ScFv | none | FcεRIγ |
| ScFv | none | DAP10 |
| ScFv | none | DAP12 |
| ScFv | none | CD32 |
| ScFv | none | CD79a |
| ScFv | none | CD8 |
| ScFv | none | CD3ζ |
| ScFv | none | CD3δ |
| ScFv | none | CD3γ |
| ScFv | none | CD3ε |
| ScFv | none | FcγRI-γ |

TABLE 5
CARs lacking Signal Domain (for dual CAR approach)

| | Co-stimulatory | Signal |
|---|---|---|
| ScFv | Signal | Domain |
| ScFv | CD28 | none |
| ScFv | CD8 | none |
| ScFv | CD4 | none |
| ScFv | b2c | none |
| ScFv | CD137/41BB | none |
| ScFv | ICOS | none |
| ScFv | CD27 | none |
| ScFv | CD28δ | none |
| ScFv | CD80 | none |
| ScFv | CD86 | none |
| ScFv | OX40 | none |
| ScFv | DAP10 | none |
| ScFv | MyD88 | none |
| ScFv | CD7 | none |
| ScFv | DAP12 | none |
| ScFv | MyD88 | none |
| ScFv | CD7 | none |
| ScFv | BTNL3 | none |
| ScFv | NKG2D | none |

TABLE 6
Third Generation CARs lacking Signal
Domain (for dual CAR approach)

| | Co-stimulatory | Co-stimulatory | Signal |
|---|---|---|---|
| ScFv | Signal | Signal | Domain |
| ScFv | CD28 | CD28 | none |
| ScFv | CD28 | CD8 | none |
| ScFv | CD28 | CD4 | none |
| ScFv | CD28 | b2c | none |
| ScFv | CD28 | CD137/41BB | none |
| ScFv | CD28 | ICOS | none |
| ScFv | CD28 | CD27 | none |
| ScFv | CD28 | CD28δ | none |
| ScFv | CD28 | CD80 | none |
| ScFv | CD28 | CD86 | none |
| ScFv | CD28 | OX40 | none |
| ScFv | CD28 | DAP10 | none |
| ScFv | CD28 | MyD88 | none |
| ScFv | CD28 | CD7 | none |
| ScFv | CD28 | DAP12 | none |
| ScFv | CD28 | MyD88 | none |
| ScFv | CD28 | CD7 | none |
| ScFv | CD8 | CD28 | none |
| ScFv | CD8 | CD8 | none |
| ScFv | CD8 | CD4 | none |
| ScFv | CD8 | b2c | none |
| ScFv | CD8 | CD137/41BB | none |
| ScFv | CD8 | ICOS | none |
| ScFv | CD8 | CD27 | none |
| ScFv | CD8 | CD28δ | none |
| ScFv | CD8 | CD80 | none |
| ScFv | CD8 | CD86 | none |
| ScFv | CD8 | OX40 | none |
| ScFv | CD8 | DAP10 | none |
| ScFv | CD8 | MyD88 | none |
| ScFv | CD8 | CD7 | none |
| ScFv | CD8 | DAP12 | none |
| ScFv | CD8 | MyD88 | none |
| ScFv | CD8 | CD7 | none |
| ScFv | CD4 | CD28 | none |
| ScFv | CD4 | CD8 | none |
| ScFv | CD4 | CD4 | none |
| ScFv | CD4 | b2c | none |
| ScFv | CD4 | CD137/41BB | none |
| ScFv | CD4 | ICOS | none |
| ScFv | CD4 | CD27 | none |
| ScFv | CD4 | CD28δ | none |
| ScFv | CD4 | CD80 | none |
| ScFv | CD4 | CD86 | none |
| ScFv | CD4 | OX40 | none |
| ScFv | CD4 | DAP10 | none |
| ScFv | CD4 | MyD88 | none |
| ScFv | CD4 | CD7 | none |
| ScFv | CD4 | DAP12 | none |
| ScFv | CD4 | MyD88 | none |
| ScFv | CD4 | CD7 | none |
| ScFv | b2c | CD28 | none |
| ScFv | b2c | CD8 | none |
| ScFv | b2c | CD4 | none |
| ScFv | b2c | b2c | none |
| ScFv | b2c | CD137/41BB | none |
| ScFv | b2c | ICOS | none |
| ScFv | b2c | CD27 | none |
| ScFv | b2c | CD28δ | none |
| ScFv | b2c | CD80 | none |
| ScFv | b2c | CD86 | none |
| ScFv | b2c | OX40 | none |
| ScFv | b2c | DAP10 | none |
| ScFv | b2c | MyD88 | none |
| ScFv | b2c | CD7 | none |
| ScFv | b2c | DAP12 | none |
| ScFv | b2c | MyD88 | none |
| ScFv | b2c | CD7 | none |
| ScFv | CD137/41BB | CD28 | none |
| ScFv | CD137/41BB | CD8 | none |
| ScFv | CD137/41BB | CD4 | none |
| ScFv | CD137/41BB | b2c | none |
| ScFv | CD137/41BB | CD137/41BB | none |
| ScFv | CD137/41BB | ICOS | none |
| ScFv | CD137/41BB | CD27 | none |
| ScFv | CD137/41BB | CD28δ | none |
| ScFv | CD137/41BB | CD80 | none |
| ScFv | CD137/41BB | CD86 | none |
| ScFv | CD137/41BB | OX40 | none |
| ScFv | CD137/41BB | DAP10 | none |
| ScFv | CD137/41BB | MyD88 | none |
| ScFv | CD137/41BB | CD7 | none |
| ScFv | CD137/41BB | DAP12 | none |
| ScFv | CD137/41BB | MyD88 | none |
| ScFv | CD137/41BB | CD7 | none |
| ScFv | ICOS | CD28 | none |
| ScFv | ICOS | CD8 | none |
| ScFv | ICOS | CD4 | none |
| ScFv | ICOS | b2c | none |
| ScFv | ICOS | CD137/41BB | none |
| ScFv | ICOS | ICOS | none |
| ScFv | ICOS | CD27 | none |
| ScFv | ICOS | CD28δ | none |
| ScFv | ICOS | CD80 | none |
| ScFv | ICOS | CD86 | none |
| ScFv | ICOS | OX40 | none |
| ScFv | ICOS | DAP10 | none |
| ScFv | ICOS | MyD88 | none |
| ScFv | ICOS | CD7 | none |
| ScFv | ICOS | DAP12 | none |
| ScFv | ICOS | MyD88 | none |
| ScFv | ICOS | CD7 | none |
| ScFv | ICOS | CD28 | none |
| ScFv | ICOS | CD8 | none |
| ScFv | ICOS | CD4 | none |
| ScFv | ICOS | b2c | none |
| ScFv | ICOS | CD137/41BB | none |
| ScFv | ICOS | ICOS | none |
| ScFv | ICOS | CD27 | none |
| ScFv | ICOS | CD28δ | none |
| ScFv | ICOS | CD80 | none |
| ScFv | ICOS | CD86 | none |
| ScFv | ICOS | OX40 | none |
| ScFv | ICOS | DAP10 | none |
| ScFv | ICOS | MyD88 | none |
| ScFv | ICOS | CD7 | none |
| ScFv | ICOS | DAP12 | none |
| ScFv | ICOS | MyD88 | none |
| ScFv | ICOS | CD7 | none |
| ScFv | CD27 | CD28 | none |
| ScFv | CD27 | CD8 | none |
| ScFv | CD27 | CD4 | none |
| ScFv | CD27 | b2c | none |
| ScFv | CD27 | CD137/41BB | none |
| ScFv | CD27 | ICOS | none |
| ScFv | CD27 | CD27 | none |
| ScFv | CD27 | CD28δ | none |
| ScFv | CD27 | CD80 | none |
| ScFv | CD27 | CD86 | none |
| ScFv | CD27 | OX40 | none |
| ScFv | CD27 | DAP10 | none |
| ScFv | CD27 | MyD88 | none |
| ScFv | CD27 | CD7 | none |
| ScFv | CD27 | DAP12 | none |
| ScFv | CD27 | MyD88 | none |
| ScFv | CD27 | CD7 | none |
| ScFv | CD28δ | CD28 | none |
| ScFv | CD28δ | CD8 | none |
| ScFv | CD28δ | CD4 | none |
| ScFv | CD28δ | b2c | none |
| ScFv | CD28δ | CD137/41BB | none |
| ScFv | CD28δ | ICOS | none |
| ScFv | CD28δ | CD27 | none |
| ScFv | CD28δ | CD28δ | none |
| ScFv | CD28δ | CD80 | none |
| ScFv | CD28δ | CD86 | none |
| ScFv | CD28δ | OX40 | none |
| ScFv | CD28δ | DAP10 | none |
| ScFv | CD28δ | MyD88 | none |
| ScFv | CD28δ | CD7 | none |
| ScFv | CD28δ | DAP12 | none |
| ScFv | CD28δ | MyD88 | none |
| ScFv | CD28δ | CD7 | none |
| ScFv | CD80 | CD28 | none |
| ScFv | CD80 | CD8 | none |
| ScFv | CD80 | CD4 | none |
| ScFv | CD80 | b2c | none |
| ScFv | CD80 | CD137/41BB | none |
| ScFv | CD80 | ICOS | none |
| ScFv | CD80 | CD27 | none |
| ScFv | CD80 | CD28δ | none |
| ScFv | CD80 | CD80 | none |
| ScFv | CD80 | CD86 | none |
| ScFv | CD80 | OX40 | none |
| ScFv | CD80 | DAP10 | none |
| ScFv | CD80 | MyD88 | none |
| ScFv | CD80 | CD7 | none |
| ScFv | CD80 | DAP12 | none |
| ScFv | CD80 | MyD88 | none |
| ScFv | CD80 | CD7 | none |
| ScFv | CD86 | CD28 | none |
| ScFv | CD86 | CD8 | none |
| ScFv | CD86 | CD4 | none |
| ScFv | CD86 | b2c | none |
| ScFv | CD86 | CD137/41BB | none |
| ScFv | CD86 | ICOS | none |
| ScFv | CD86 | CD27 | none |
| ScFv | CD86 | CD28δ | none |
| ScFv | CD86 | CD80 | none |
| ScFv | CD86 | CD86 | none |
| ScFv | CD86 | OX40 | none |
| ScFv | CD86 | DAP10 | none |
| ScFv | CD86 | MyD88 | none |
| ScFv | CD86 | CD7 | none |
| ScFv | CD86 | DAP12 | none |
| ScFv | CD86 | MyD88 | none |
| ScFv | CD86 | CD7 | none |
| ScFv | OX40 | CD28 | none |
| ScFv | OX40 | CD8 | none |
| ScFv | OX40 | CD4 | none |
| ScFv | OX40 | b2c | none |
| ScFv | OX40 | CD137/41BB | none |
| ScFv | OX40 | ICOS | none |
| ScFv | OX40 | CD27 | none |
| ScFv | OX40 | CD28δ | none |
| ScFv | OX40 | CD80 | none |
| ScFv | OX40 | CD86 | none |
| ScFv | OX40 | OX40 | none |
| ScFv | OX40 | DAP10 | none |
| ScFv | OX40 | MyD88 | none |
| ScFv | OX40 | CD7 | none |
| ScFv | OX40 | DAP12 | none |
| ScFv | OX40 | MyD88 | none |
| ScFv | OX40 | CD7 | none |
| ScFv | DAP10 | CD28 | none |
| ScFv | DAP10 | CD8 | none |
| ScFv | DAP10 | CD4 | none |
| ScFv | DAP10 | b2c | none |
| ScFv | DAP10 | CD137/41BB | none |
| ScFv | DAP10 | ICOS | none |
| ScFv | DAP10 | CD27 | none |
| ScFv | DAP10 | CD28δ | none |
| ScFv | DAP10 | CD80 | none |
| ScFv | DAP10 | CD86 | none |
| ScFv | DAP10 | OX40 | none |
| ScFv | DAP10 | DAP10 | none |
| ScFv | DAP10 | MyD88 | none |
| ScFv | DAP10 | CD7 | none |
| ScFv | DAP10 | DAP12 | none |
| ScFv | DAP10 | MyD88 | none |
| ScFv | DAP10 | CD7 | none |
| ScFv | DAP12 | CD28 | none |
| ScFv | DAP12 | CD8 | none |
| ScFv | DAP12 | CD4 | none |
| ScFv | DAP12 | b2c | none |
| ScFv | DAP12 | CD137/41BB | none |
| ScFv | DAP12 | ICOS | none |
| ScFv | DAP12 | CD27 | none |
| ScFv | DAP12 | CD28δ | none |
| ScFv | DAP12 | CD80 | none |
| ScFv | DAP12 | CD86 | none |
| ScFv | DAP12 | OX40 | none |
| ScFv | DAP12 | DAP10 | none |
| ScFv | DAP12 | MyD88 | none |
| ScFv | DAP12 | CD7 | none |
| ScFv | DAP12 | DAP12 | none |
| ScFv | DAP12 | MyD88 | none |
| ScFv | DAP12 | CD7 | none |
| ScFv | MyD88 | CD28 | none |
| ScFv | MyD88 | CD8 | none |
| ScFv | MyD88 | CD4 | none |
| ScFv | MyD88 | b2c | none |
| ScFv | MyD88 | CD137/41BB | none |
| ScFv | MyD88 | ICOS | none |
| ScFv | MyD88 | CD27 | none |
| ScFv | MyD88 | CD28δ | none |
| ScFv | MyD88 | CD80 | none |
| ScFv | MyD88 | CD86 | none |
| ScFv | MyD88 | OX40 | none |
| ScFv | MyD88 | DAP10 | none |
| ScFv | MyD88 | MyD88 | none |
| ScFv | MyD88 | CD7 | none |
| ScFv | MyD88 | DAP12 | none |
| ScFv | MyD88 | MyD88 | none |
| ScFv | MyD88 | CD7 | none |
| ScFv | CD7 | CD28 | none |
| ScFv | CD7 | CD8 | none |
| ScFv | CD7 | CD4 | none |
| ScFv | CD7 | b2c | none |
| ScFv | CD7 | CD137/41BB | none |
| ScFv | CD7 | ICOS | none |
| ScFv | CD7 | CD27 | none |
| ScFv | CD7 | CD28δ | none |
| ScFv | CD7 | CD80 | none |
| ScFv | CD7 | CD86 | none |
| ScFv | CD7 | OX40 | none |
| ScFv | CD7 | DAP10 | none |
| ScFv | CD7 | MyD88 | none |
| ScFv | CD7 | CD7 | none |
| ScFv | CD7 | DAP12 | none |
| ScFv | CD7 | MyD88 | none |
| ScFv | CD7 | CD7 | none |
| ScFv | BTNL3 | CD28 | none |
| ScFv | BTNL3 | CD8 | none |
| ScFv | BTNL3 | CD4 | none |
| ScFv | BTNL3 | b2c | none |
| ScFv | BTNL3 | CD137/41BB | none |
| ScFv | BTNL3 | ICOS | none |
| ScFv | BTNL3 | CD27 | none |
| ScFv | BTNL3 | CD28δ | none |
| ScFv | BTNL3 | CD80 | none |
| ScFv | BTNL3 | CD86 | none |
| ScFv | BTNL3 | OX40 | none |
| ScFv | BTNL3 | DAP10 | none |
| ScFv | BTNL3 | MyD88 | none |
| ScFv | BTNL3 | CD7 | none |
| ScFv | BTNL3 | DAP12 | none |
| ScFv | BTNL3 | MyD88 | none |
| ScFv | BTNL3 | CD7 | none |
| ScFv | NKG2D | CD28 | none |
| ScFv | NKG2D | CD8 | none |
| ScFv | NKG2D | CD4 | none |
| ScFv | NKG2D | b2c | none |
| ScFv | NKG2D | CD137/41BB | none |
| ScFv | NKG2D | ICOS | none |
| ScFv | NKG2D | CD27 | none |
| ScFv | NKG2D | CD28δ | none |
| ScFv | NKG2D | CD80 | none |
| ScFv | NKG2D | CD86 | none |
| ScFv | NKG2D | OX40 | none |
| ScFv | NKG2D | DAP10 | none |
| ScFv | NKG2D | MyD88 | none |
| ScFv | NKG2D | CD7 | none |
| ScFv | NKG2D | DAP12 | none |
| ScFv | NKG2D | MyD88 | none |
| ScFv | NKG2D | CD7 | none |

In some embodiments, the binding agent is single chain variable fragment (scFv) antibody. The affinity/specificity of an scFv is driven in large part by specific sequences within complementarity determining regions (CDRs) in the heavy (V_{H}) and light (V_{L}) chain. Each V_{H} and V_{L} sequence will have three CDRs (CDR1, CDR2, CDR3).

In some embodiments, the binding agent is derived from natural antibodies, such as monoclonal antibodies. In some cases, the antibody is human. In some cases, the antibody has undergone an alteration to render it less immunogenic when administered to humans. For example, the alteration comprises one or more techniques selected from the group consisting of chimerization, humanization, CDR-grafting, deimmunization, and mutation of framework amino acids to correspond to the closest human germline sequence.

Also disclosed are bi-specific CARs that at least one additional tumor antigen. Tumor antigens are proteins that are produced by tumor cells that elicit an immune response, particularly T-cell mediated immune responses. The additional antigen binding domain can be an antibody or a natural ligand of the tumor antigen. The selection of the additional antigen binding domain will depend on the particular type of cancer to be treated. Tumor antigens are well known in the art and include, for example, a glioma-associated antigen, carcinoembryonic antigen (CEA), EGFRvIII, IL-IIRa, IL-13Ra, EGFR, FAP, B7H3, Kit, CA LX, CS-1, MUC1, BCMA, bcr-abl, HER2, β-human chorionic gonadotropin, alphafetoprotein (AFP), ALK, CD19, CD123, cyclin BI, lectin-reactive AFP, Fos-related antigen 1, ADRB3, thyroglobulin, EphA2, RAGE-1, RUI, RU2, SSX2, AKAP-4, LCK, OY-TESI, PAX5, SART3, CLL-1, fucosyl GM1, GloboH, MN-CA IX, EPCAM, EVT6-AML, TGS5, human telomerase reverse transcriptase, plysialic acid, PLAC1, RUI, RU2 (AS), intestinal carboxyl esterase, lewisY, sLe, LY6K, mut hsp70-2, M-CSF, MYCN, RhoC, TRP-2, CYPIBI, BORIS, prostase, prostate-specific antigen (PSA), PAX3, PAP, NY-ESO-1, LAGE-la, LMP2, NCAM, p53, p53 mutant, Ras mutant, gplOO, prostein, OR51E2, PANX3, PSMA, PSCA, Her2/neu, hTERT, HMWMAA, HAVCR1, VEGFR2, PDGFR-beta, survivin and telomerase, legumain, HPV E6,E7, sperm protein 17, SSEA-4, tyrosinase, TARP, WT1, prostate-carcinoma tumor antigen-1 (PCTA-1), ML-IAP, MAGE, MAGE-A1,MAD-CT-1, MAD-CT-2, MelanA/MART 1, XAGE1, ELF2M, ERG (TMPRSS2 ETS fusion gene), NA17, neutrophil elastase, sarcoma translocation breakpoints, NY-BR-1, ephnnB2, CD20, CD22, CD24, CD30, TIM3, CD38, CD44v6, CD97, CD171, CD179a, androgen receptor, FAP, insulin growth factor (IGF)-I, IGFII, IGF-I receptor, GD2, o-acetyl-GD2, GD3, GM3, GPRCSD, GPR20, CXORF61, folate receptor (FRa), folate receptor beta, ROR1, Flt3, TAG72, TN Ag, Tie 2, TEM1, TEM7R, CLDN6, TSHR, UPK2, and mesothelin. In a preferred embodiment, the tumor antigen is selected from the group consisting of folate receptor (FRa), mesothelin, EGFRvIII, IL-13Ra, CD123, CD19, TIM3, BCMA, GD2, CLL-1, CA-IX, MUCI, HER2, and any combination thereof.

Non-limiting examples of tumor antigens include the following: Differentiation antigens such as tyrosinase, TRP-1, TRP-2 and tumor-specific multilineage antigens such as MAGE-1, MAGE-3, BAGE, GAGE-1, GAGE-2, pi 5; overexpressed embryonic antigens such as CEA; overexpressed oncogenes and mutated tumor-suppressor genes such as p53, Ras, HER-2/neu; unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, pl85erbB2, pl80erbB-3, c-met, nm-23H1, PSA, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3†CA 27.29†BCAA, CA 195, CA 242, CA-50, CAM43, CD68†P1, CO-029, FGF-5, G250, Ga733†EpCAM, HTgp-175, M344, MA-50, MG7-Ag, MOV18, NB/70K, NY-CO-1, RCASI, SDCCAG1 6, TA-90†Mac-2 binding protein†cyclophilm C-associated protein, TAAL6, TAG72, TLP, TPS, GPC3, MUC16, LMP1, EBMA-1, BARF-1, CS1, CD319, HER1, B7H6, L1CAM, IL6, and MET.

Also disclosed are polynucleotides and polynucleotide vectors encoding the disclosed CARs that allow expression of the CARs in the disclosed immune effector cells.

Nucleic acid sequences encoding the disclosed CARs, and regions thereof, can be obtained using recombinant methods known in the art, such as, for example by screening libraries from cells expressing the gene, by deriving the gene from a vector known to include the same, or by isolating directly from cells and tissues containing the same, using standard techniques. Alternatively, the gene of interest can be produced synthetically, rather than cloned.

Expression of nucleic acids encoding CARs is typically achieved by operably linking a nucleic acid encoding the CAR polypeptide to a promoter and incorporating the construct into an expression vector. Typical cloning vectors contain transcription and translation terminators, initiation sequences, and promoters useful for regulation of the expression of the desired nucleic acid sequence.

The disclosed nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al. (2001, Molecular Cloning: A Laboratory Manual, Cold Spring Harbor Laboratory, New York), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. In some embodiments, the polynucleotide vectors are lentiviral or retroviral vectors.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, MND (myeloproliferative sarcoma virus) promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. The promoter can alternatively be an inducible promoter. Examples of inducible promoters include, but are not limited to a metallothionine promoter, a glucocorticoid promoter, a progesterone promoter, and a tetracycline promoter.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another.

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene. Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

Methods of introducing and expressing genes into a cell are known in the art. In the context of an expression vector, the vector can be readily introduced into a host cell, e.g., mammalian, bacterial, yeast, or insect cell by any method in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2001, Molecular Cloning: A Laboratory Manual, Cold Spring Harbor Laboratory, New York).

Biological methods for introducing a polynucleotide of interest into a host cell include the use of DNA and RNA vectors. Viral vectors, and especially retroviral vectors, have become the most widely used method for inserting genes into mammalian, e.g., human cells.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle).

In the case where a non-viral delivery system is utilized, an exemplary delivery vehicle is a liposome. In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes. Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc, (Birmingham, Ala.).

Also disclosed are immune effector cells that are engineered to express the disclosed CARs (also referred to herein as “CAR-T cells.” These cells are preferably obtained from the subject to be treated (i.e. are autologous). However, in some embodiments, immune effector cell lines or donor effector cells (allogeneic) are used. Immune effector cells can be obtained from a number of sources, including peripheral blood mononuclear cells, bone marrow, lymph node tissue, cord blood, thymus tissue, tissue from a site of infection, ascites, pleural effusion, spleen tissue, and tumors. Immune effector cells can be obtained from blood collected from a subject using any number of techniques known to the skilled artisan, such as Ficoll™ separation. For example, cells from the circulating blood of an individual may be obtained by apheresis. In some embodiments, immune effector cells are isolated from peripheral blood lymphocytes by lysing the red blood cells and depleting the monocytes, for example, by centrifugation through a PERCOLL™ gradient or by counterflow centrifugal elutriation. A specific subpopulation of immune effector cells can be further isolated by positive or negative selection techniques. For example, immune effector cells can be isolated using a combination of antibodies directed to surface markers unique to the positively selected cells, e.g., by incubation with antibody-conjugated beads for a time period sufficient for positive selection of the desired immune effector cells. Alternatively, enrichment of immune effector cells population can be accomplished by negative selection using a combination of antibodies directed to surface markers unique to the negatively selected cells.

In some embodiments, the immune effector cells comprise any leukocyte involved in defending the body against infectious disease and foreign materials. For example, the immune effector cells can comprise lymphocytes, monocytes, macrophages, dentritic cells, mast cells, neutrophils, basophils, eosinophils, or any combinations thereof. For example, the immune effector cells can comprise T lymphocytes.

T cells or T lymphocytes can be distinguished from other lymphocytes, such as B cells and natural killer cells (NK cells), by the presence of a T-cell receptor (TCR) on the cell surface. They are called T cells because they mature in the thymus (although some also mature in the tonsils). There are several subsets of T cells, each with a distinct function.

T helper cells (T_{H} cells) assist other white blood cells in immunologic processes, including maturation of B cells into plasma cells and memory B cells, and activation of cytotoxic T cells and macrophages. These cells are also known as CD4+ T cells because they express the CD4 glycoprotein on their surface. Helper T cells become activated when they are presented with peptide antigens by MHC class II molecules, which are expressed on the surface of antigen-presenting cells (APCs). Once activated, they divide rapidly and secrete small proteins called cytokines that regulate or assist in the active immune response. These cells can differentiate into one of several subtypes, including T_{H}1, T_{H}2, T_{H}3, T_{H}17, T_{H}9, or T_{FH}, which secrete different cytokines to facilitate a different type of immune response.

Cytotoxic T cells (Tc cells, or CTLs) destroy virally infected cells and tumor cells, and are also implicated in transplant rejection. These cells are also known as CD8+ T cells since they express the CD8 glycoprotein at their surface. These cells recognize their targets by binding to antigen associated with MHC class I molecules, which are present on the surface of all nucleated cells. Through IL-10, adenosine and other molecules secreted by regulatory T cells, the CD8+ cells can be inactivated to an anergic state, which prevents autoimmune diseases.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory cells may be either CD4⁺ or CD8⁺. Memory T cells typically express the cell surface protein CD45RO.

Regulatory T cells (T_{reg} cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus. Two major classes of CD4⁺ T_{reg} cells have been described—naturally occurring T_{reg} cells and adaptive T_{reg} cells.

Natural killer T (NKT) cells (not to be confused with natural killer (NK) cells) bridge the adaptive immune system with the innate immune system. Unlike conventional T cells that recognize peptide antigens presented by major histocompatibility complex (MHC) molecules, NKT cells recognize glycolipid antigen presented by a molecule called CD1d.

In some embodiments, the T cells comprise a mixture of CD4+ cells. In other embodiments, the T cells are enriched for one or more subsets based on cell surface expression. For example, in some cases, the T comprise are cytotoxic CD8⁺ T lymphocytes. In some embodiments, the T cells comprise γδ T cells, which possess a distinct T-cell receptor (TCR) having one γ chain and one δ chain instead of α and β chains.

Natural-killer (NK) cells are CD56+CD3⁻ large granular lymphocytes that can kill virally infected and transformed cells, and constitute a critical cellular subset of the innate immune system (Godfrey J, et al. Leuk Lymphoma 2012 53:1666-1676). Unlike cytotoxic CD8+T lymphocytes, NK cells launch cytotoxicity against tumor cells without the requirement for prior sensitization, and can also eradicate MHC-1-negative cells (Narni-Mancinelli E, et al. Int Immunol 2011 23:427-431). NK cells are safer effector cells, as they may avoid the potentially lethal complications of cytokine storms (Morgan R A, et al. Mol Ther 2010 18:843-851), tumor lysis syndrome (Porter D L, et al. N Engl J Med 2011 365:725-733), and on-target, off-tumor effects. Although NK cells have a well-known role as killers of cancer cells, and NK cell impairment has been extensively documented as crucial for progression of MM (Godfrey J, et al. Leuk Lymphoma 2012 53:1666-1676; Fauriat C, et al. Leukemia 2006 20:732-733), the means by which one might enhance NK cell-mediated anti-MM activity has been largely unexplored prior to the disclosed CARs.

Epstein-Barr virus (EBV)-induced lymphoproliferative diseases (EBV-LPDs) are a significant cause of morbidity and mortality for recipients of allogeneic hematopoietic cell transplantation (HCT), particularly in those who have received certain T-cell reactive Abs to prevent or treat GVHD. Prophylaxis and treatment by the adoptive transfer of EBV-specific T cells and the subsequent long-term restoration of immunity against EBV-associated lymphoproliferation have provided positive outcomes in the management of this uniformly fatal complication of bone marrow transfer. Therefore, in some embodiments, the disclosed immune effector cells expressing the CARs of the present invention are allogeneic or autologous EBV-specific cytotoxic T lymphocytes (CTLs). For example, this can involve isolating PBMCs from of an autologous or allogenic donor and enriching them for T cells by depletion of monocytes and NK cells. For example, the donor can be an EBV-seropositive donor. These T cells can then be stimulated with autologous EBV-seropostive or transformed lymphocytes. EBV antigens include latent membrane protein (LMP) and EBV nuclear antigen (EBNA) proteins, such as LMP-1, LMP-2A, and LMP-2B and EBNA-1, EBNA-2, EBNA-3A, EBNA-3B, EBNA-3C and EBNA-LP. These methods are described, for example, in Wilkie et al., J. Immunother. 27(4):309-316 (2004), Barker et al., Blood 2010 116(23):5045-49; Doubrovina, et al., Blood 2012 119(11):2644-56; Koehne, et al. Blood 2002 99(5):1730-40; and Smith et al. Cancer Res 2012 72(5):1116-25, which are incorporated by reference for these teachings.

Immune effector cells expressing the disclosed CARs can elicit an anti-tumor immune response against CD99- or CLEC12A-expressing cancer cells. The anti-tumor immune response elicited by the disclosed CAR-modified immune effector cells may be an active or a passive immune response. In addition, the CAR-mediated immune response may be part of an adoptive immunotherapy approach in which CAR-modified immune effector cells induce an immune response specific to CD99.

Adoptive transfer of immune effector cells expressing chimeric antigen receptors is a promising anti-cancer therapeutic. Following the collection of a patient's immune effector cells, the cells may be genetically engineered to express the disclosed CD99-specific CARs, then infused back into the patient.

The disclosed CAR-modified immune effector cells may be administered either alone, or as a pharmaceutical composition in combination with diluents and/or with other components such as IL-2, IL-15, or other cytokines or cell populations. Briefly, pharmaceutical compositions may comprise a target cell population as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions for use in the disclosed methods are in some embodiments formulated for intravenous administration. Pharmaceutical compositions may be administered in any manner appropriate treat MM. The quantity and frequency of administration will be determined by such factors as the condition of the patient, and the severity of the patient's disease, although appropriate dosages may be determined by clinical trials.

When “an immunologically effective amount”, “an anti-tumor effective amount”, “an tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, such as 105 to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

In certain embodiments, it may be desired to administer activated T cells to a subject and then subsequently re-draw blood (or have an apheresis performed), activate T cells therefrom according to the disclosed methods, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Using this multiple blood draw/multiple reinfusion protocol may serve to select out certain populations of T cells. The administration of the disclosed compositions may be carried out in any convenient manner, including by injection, transfusion, or implantation. The compositions described herein may be administered to a patient subcutaneously, intradermally, intratumorally, intranodally, intramedullary, intramuscularly, by intravenous (i.v.) injection, or intraperitoneally. In some embodiments, the disclosed compositions are administered to a patient by intradermal or subcutaneous injection. In some embodiments, the disclosed compositions are administered by i.v. injection. The compositions may also be injected directly into a tumor, lymph node, or site of infection.

In certain embodiments, the disclosed CAR-modified immune effector cells are administered to a patient in conjunction with (e.g., before, simultaneously or following) any number of relevant treatment modalities, including but not limited to thalidomide, dexamethasone, bortezomib, and lenalidomide. In further embodiments, the CAR-modified immune effector cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. In some embodiments, the CAR-modified immune effector cells are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions of the present invention are administered following B-cell ablative therapy such as agents that react with CD20, e.g., Rituxan. For example, in some embodiments, subjects may undergo standard treatment with high dose chemotherapy followed by peripheral blood stem cell transplantation. In certain embodiments, following the transplant, subjects receive an infusion of the expanded immune cells of the present invention. In an additional embodiment, expanded cells are administered before or following surgery.

The cancer of the disclosed methods can be any CD99- and/or CLEC12A-expressing cell in a subject undergoing unregulated growth, invasion, or metastasis. Cancers that express CD99 and/or CLEC12A include prostate cancer, ovarian cancer, adenocarcinoma of the lung, breast cancer, endometrial cancer, gastric cancer, colon cancer, and pancreatic cancer. In some aspects, the cancer is a gallbladder cancer, exocrine adenocarcinoma, or apocrine adenocarcinomas. In some cases, the cancer comprises myelodysplastic syndrome, acute myeloid leukemia, or bi-phenotypic leukemia.

In some aspects, the cancer can be any neoplasm or tumor for which radiotherapy is currently used. Alternatively, the cancer can be a neoplasm or tumor that is not sufficiently sensitive to radiotherapy using standard methods. Thus, the cancer can be a sarcoma, lymphoma, leukemia, carcinoma, blastoma, or germ cell tumor. A representative but non-limiting list of cancers that the disclosed compositions can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, endometrial cancer, cervical cancer, cervical carcinoma, breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer, and pancreatic cancer.

The disclosed CARs can be used in combination with any compound, moiety or group which has a cytotoxic or cytostatic effect. Drug moieties include chemotherapeutic agents, which may function as microtubulin inhibitors, mitosis inhibitors, topoisomerase inhibitors, or DNA intercalators, and particularly those which are used for cancer therapy.

The disclosed CARs can be used in combination with a checkpoint inhibitor. The two known inhibitory checkpoint pathways involve signaling through the cytotoxic T-lymphocyte antigen-4 (CTLA-4) and programmed-death 1 (PD-1) receptors. These proteins are members of the CD28-B7 family of cosignaling molecules that play important roles throughout all stages of T cell function. The PD-1 receptor (also known as CD279) is expressed on the surface of activated T cells. Its ligands, PD-L1 (B7-H1; CD274) and PD-L2 (B7-DC; CD273), are expressed on the surface of APCs such as dendritic cells or macrophages. PD-L1 is the predominant ligand, while PD-L2 has a much more restricted expression pattern. When the ligands bind to PD-1, an inhibitory signal is transmitted into the T cell, which reduces cytokine production and suppresses T-cell proliferation. Checkpoint inhibitors include, but are not limited to antibodies that block PD-1 (Nivolumab (BMS-936558 or MDX1106), CT-011, MK-3475), PD-L1 (MDX-1105 (BMS-936559), MPDL3280A, MSB0010718C), PD-L2 (rHIgM12B7), CTLA-4 (Ipilimumab (MDX-010), Tremelimumab (CP-675,206)), IDO, B7-H3 (MGA271), B7-H4, TIM3, LAG-3 (BMS-986016).

Human monoclonal antibodies to programmed death 1 (PD-1) and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

In some embodiments, the PDL1 inhibitor comprises an antibody that specifically binds PDL1, such as BMS-936559 (Bristol-Myers Squibb) or MPDL3280A (Roche). In some embodiments, the PD1 inhibitor comprises an antibody that specifically binds PD1, such as lambrolizumab (Merck), nivolumab (Bristol-Myers Squibb), or MED14736 (AstraZeneca). Human monoclonal antibodies to PD-1 and methods for treating cancer using anti-PD-1 antibodies alone or in combination with other immunotherapeutics are described in U.S. Pat. No. 8,008,449, which is incorporated by reference for these antibodies. Anti-PD-L1 antibodies and uses therefor are described in U.S. Pat. No. 8,552,154, which is incorporated by reference for these antibodies. Anticancer agent comprising anti-PD-1 antibody or anti-PD-L1 antibody are described in U.S. Pat. No. 8,617,546, which is incorporated by reference for these antibodies.

The disclosed CARs can be used in combination with other cancer immunotherapies. There are two distinct types of immunotherapy: passive immunotherapy uses components of the immune system to direct targeted cytotoxic activity against cancer cells, without necessarily initiating an immune response in the patient, while active immunotherapy actively triggers an endogenous immune response. Passive strategies include the use of the monoclonal antibodies (mAbs) produced by B cells in response to a specific antigen. The development of hybridoma technology in the 1970s and the identification of tumor-specific antigens permitted the pharmaceutical development of mAbs that could specifically target tumor cells for destruction by the immune system. Thus far, mAbs have been the biggest success story for immunotherapy; the top three best-selling anticancer drugs in 2012 were mAbs. Among them is rituximab (Rituxan, Genentech), which binds to the CD20 protein that is highly expressed on the surface of B cell malignancies such as non-Hodgkin's lymphoma (NHL). Rituximab is approved by the FDA for the treatment of NHL and chronic lymphocytic leukemia (CLL) in combination with chemotherapy. Another important mAb is trastuzumab (Herceptin; Genentech), which revolutionized the treatment of HER2 (human epidermal growth factor receptor 2)-positive breast cancer by targeting the expression of HER2.

Generating optimal “killer” CD8 T cell responses also requires T cell receptor activation plus co-stimulation, which can be provided through ligation of tumor necrosis factor receptor family members, including OX40 (CD134) and 4-1BB (CD137). OX40 is of particular interest as treatment with an activating (agonist) anti-OX40 mAb augments T cell differentiation and cytolytic function leading to enhanced anti-tumor immunity against a variety of tumors.

In some embodiments, such an additional therapeutic agent may be selected from an antimetabolite, such as methotrexate, 6-mercaptopurine, 6-thioguanine, cytarabine, fludarabine, 5-fluorouracil, decarbazine, hydroxyurea, asparaginase, gemcitabine or cladribine.

In some embodiments, such an additional therapeutic agent may be selected from an alkylating agent, such as mechlorethamine, thioepa, chlorambucil, melphalan, carmustine (BSNU), lomustine (CCNU), cyclophosphamide, busulfan, dibromomannitol, streptozotocin, dacarbazine (DTIC), procarbazine, mitomycin C, cisplatin and other platinum derivatives, such as carboplatin.

In some embodiments, such an additional therapeutic agent may be selected from an anti-mitotic agent, such as taxanes, for instance docetaxel, and paclitaxel, and vinca alkaloids, for instance vindesine, vincristine, vinblastine, and vinorelbine.

In some embodiments, such an additional therapeutic agent may be selected from a topoisomerase inhibitor, such as topotecan or irinotecan, or a cytostatic drug, such as etoposide and teniposide.

In some embodiments, such an additional therapeutic agent may be selected from a growth factor inhibitor, such as an inhibitor of ErbBI (EGFR) (such as an EGFR antibody, e.g. zalutumumab, cetuximab, panitumumab or nimotuzumab or other EGFR inhibitors, such as gefitinib or erlotinib), another inhibitor of ErbB2 (HER2/neu) (such as a HER2 antibody, e.g. trastuzumab, trastuzumab-DM I or pertuzumab) or an inhibitor of both EGFR and HER2, such as lapatinib).

In some embodiments, such an additional therapeutic agent may be selected from a tyrosine kinase inhibitor, such as imatinib (Glivec, Gleevec ST1571) or lapatinib.

Therefore, in some embodiments, a disclosed antibody is used in combination with ofatumumab, zanolimumab, daratumumab, ranibizumab, nimotuzumab, panitumumab, hu806, daclizumab (Zenapax), basiliximab (Simulect), infliximab (Remicade), adalimumab (Humira), natalizumab (Tysabri), omalizumab (Xolair), efalizumab (Raptiva), and/or rituximab.

In some embodiments, a therapeutic agent for use in combination with a CARs for treating the disorders as described above may be an anti-cancer cytokine, chemokine, or combination thereof. Examples of suitable cytokines and growth factors include IFNγ, IL-2, IL-4, IL-6, IL-7, IL-10, IL-12, IL-13, IL-15, IL-18, IL-23, IL-24, IL-27, IL-28a, IL-28b, IL-29, KGF, IFNa (e.g., INFa2b), IFN, GM-CSF, CD40L, Flt3 ligand, stem cell factor, ancestim, and TNFa. Suitable chemokines may include Glu-Leu-Arg (ELR)-negative chemokines such as IP-10, MCP-3, MIG, and SDF-la from the human CXC and C—C chemokine families. Suitable cytokines include cytokine derivatives, cytokine variants, cytokine fragments, and cytokine fusion proteins.

In some embodiments, a therapeutic agent for use in combination with a CARs for treating the disorders as described above may be a cell cycle control/apoptosis regulator (or “regulating agent”). A cell cycle control/apoptosis regulator may include molecules that target and modulate cell cycle control/apoptosis regulators such as (i) cdc-25 (such as NSC 663284), (ii) cyclin-dependent kinases that overstimulate the cell cycle (such as flavopiridol (L868275, HMR1275), 7-hydroxystaurosporine (UCN-01, KW-2401), and roscovitine (R-roscovitine, CYC202)), and (iii) telomerase modulators (such as BIBR1532, SOT-095, GRN163 and compositions described in for instance U.S. Pat. Nos. 6,440,735 and 6,713,055). Non-limiting examples of molecules that interfere with apoptotic pathways include TNF-related apoptosis-inducing ligand (TRAIL)/apoptosis-2 ligand (Apo-2L), antibodies that activate TRAIL receptors, IFNs, and anti-sense Bcl-2.

In some embodiments, a therapeutic agent for use in combination with a CARs for treating the disorders as described above may be a hormonal regulating agent, such as agents useful for anti-androgen and anti-estrogen therapy. Examples of such hormonal regulating agents are tamoxifen, idoxifene, fulvestrant, droloxifene, toremifene, raloxifene, diethylstilbestrol, ethinyl estradiol/estinyl, an antiandrogene (such as flutaminde/eulexin), a progestin (such as such as hydroxyprogesterone caproate, medroxy-progesterone/provera, megestrol acepate/megace), an adrenocorticosteroid (such as hydrocortisone, prednisone), luteinizing hormone-releasing hormone (and analogs thereof and other LHRH agonists such as buserelin and goserelin), an aromatase inhibitor (such as anastrazole/arimidex, aminoglutethimide/cytraden, exemestane) or a hormone inhibitor (such as octreotide/sandostatin).

In some embodiments, a therapeutic agent for use in combination with an CARs for treating the disorders as described above may be an anti-cancer nucleic acid or an anti-cancer inhibitory RNA molecule.

Combined administration, as described above, may be simultaneous, separate, or sequential. For simultaneous administration the agents may be administered as one composition or as separate compositions, as appropriate.

In some embodiments, the disclosed CARs are administered in combination with radiotherapy. Radiotherapy may comprise radiation or associated administration of radiopharmaceuticals to a patient is provided. The source of radiation may be either external or internal to the patient being treated (radiation treatment may, for example, be in the form of external beam radiation therapy (EBRT) or brachytherapy (BT)). Radioactive elements that may be used in practicing such methods include, e.g., radium, cesium-137, iridium-192, americium-241, gold-198, cobalt-57, copper-67, technetium-99, iodide-123, iodide-131, and indium-111.

In some embodiments, the disclosed CARs are administered in combination with surgery.

CAR-T cells may be designed in several ways that enhance tumor cytotoxicity and specificity, evade tumor immunosuppression, avoid host rejection, and prolong their therapeutic half-life. TRUCK (T-cells Redirected for Universal Cytokine Killing) T cells for example, possess a CAR but are also engineered to release cytokines such as IL-12 that promote tumor killing. Because these cells are designed to release a molecular payload upon activation of the CAR once localized to the tumor environment, these CAR-T cells are sometimes also referred to as ‘armored CARs’. Several cytokines as cancer therapies are being investigated both pre-clinically and clinically, and may also prove useful when similarly incorporated into a TRUCK form of CAR-T therapy. Among these include IL-2, IL-3. IL-4, IL-5, IL-6, IL-7, IL-10, IL-12, IL-13, IL-15, IL-18, M-CSF, GM-CSF, IFN-α, IFN-γ, TNF-α, TRAIL, FLT3 ligand, Lymphotactin, and TGF-β (Dranoff 2004). “Self-driving” or “homing” CAR-T cells are engineered to express a chemokine receptor in addition to their CAR. As certain chemokines can be upregulated in tumors, incorporation of a chemokine receptor aids in tumor trafficking to and infiltration by the adoptive T-cell, thereby enhancing both specificity and functionality of the CAR-T (Moon 2011). Universal CAR-T cells also possess a CAR, but are engineered such that they do not express endogenous TCR (T-cell receptor) or MHC (major histocompatibility complex) proteins. Removal of these two proteins from the signaling repertoire of the adoptive T-cell therapy prevents graft-versus-host-disease and rejection, respectively. Armored CAR-T cells are additionally so named for their ability to evade tumor immunosuppression and tumor-induced CAR-T hypofunction. These particular CAR-Ts possess a CAR, and may be engineered to not express checkpoint inhibitors. Alternatively, these CAR-Ts can be co-administered with a monoclonal antibody (mAb) that blocks checkpoint signaling. Administration of an anti-PDL1 antibody significantly restored the killing ability of CAR TILs (tumor infiltrating lymphocytes). While PD1-PDL1 and CTLA-4-CD80/CD86 signaling pathways have been investigated, it is possible to target other immune checkpoint signaling molecules in the design of an armored CAR-T including LAG-3, Tim-3, IDO-1, 2B4, and KIR. Other intracellular inhibitors of TILs include phosphatases (SHP1), ubiquitin-ligases (i.e., cbl-b), and kinases (i.e., diacylglycerol kinase). Armored CAR-Ts may also be engineered to express proteins or receptors that protect them against or make them resistant to the effects of tumor-secreted cytokines. For example, CTLs (cytotoxic T lymphocytes) transduced with the double negative form of the TGF-β receptor are resistant to the immunosuppression by lymphoma secreted TGF-β. These transduced cells showed notably increased antitumor activity in vivo when compared to their control counterparts.

Tandem and dual CAR-T cells are unique in that they possess two distinct antigen binding domains. A tandem CAR contains two sequential antigen binding domains facing the extracellular environment connected to the intracellular costimulatory and stimulatory domains. A dual CAR is engineered such that one extracellular antigen binding domain is connected to the intracellular costimulatory domain and a second, distinct extracellular antigen binding domain is connected to the intracellular stimulatory domain. Because the stimulatory and costimulatory domains are split between two separate antigen binding domains, dual CARs are also referred to as “split CARs”. In both tandem and dual CAR designs, binding of both antigen binding domains is necessary to allow signaling of the CAR circuit in the T-cell. Because these two CAR designs have binding affinities for different, distinct antigens, they are also referred to as “bi-specific” CARs.

One primary concern with CAR-T cells as a form of “living therapeutic” is their manipulability in vivo and their potential immune-stimulating side effects. To better control CAR-T therapy and prevent against unwanted side effects, a variety of features have been engineered including off-switches, safety mechanisms, and conditional control mechanisms. Both self-destruct and marked/tagged CAR-T cells for example, are engineered to have an “off-switch” that promotes clearance of the CAR-expressing T-cell. A self-destruct CAR-T contains a CAR, but is also engineered to express a pro-apoptotic suicide gene or “elimination gene” inducible upon administration of an exogenous molecule. A variety of suicide genes may be employed for this purpose, including HSV-TK (herpes simplex virus thymidine kinase), Fas, iCasp9 (inducible caspase 9), CD20, MYC TAG, and truncated EGFR (endothelial growth factor receptor). HSK for example, will convert the prodrug ganciclovir (GCV) into GCV-triphosphate that incorporates itself into replicating DNA, ultimately leading to cell death. iCasp9 is a chimeric protein containing components of FK506-binding protein that binds the small molecule AP1903, leading to caspase 9 dimerization and apoptosis. A marked/tagged CAR-T cell however, is one that possesses a CAR but also is engineered to express a selection marker. Administration of a mAb against this selection marker will promote clearance of the CAR-T cell. Truncated EGFR is one such targetable antigen by the anti-EGFR mAb, and administration of cetuximab works to promotes elimination of the CAR-T cell. CARs created to have these features are also referred to as sCARs for ‘switchable CARs’, and RCARs for ‘regulatable CARs’. A “safety CAR”, also known as an “inhibitory CAR” (iCAR), is engineered to express two antigen binding domains. One of these extracellular domains is directed against a tumor related antigen and bound to an intracellular costimulatory and stimulatory domain. The second extracellular antigen binding domain however is specific for normal tissue and bound to an intracellular checkpoint domain such as CTLA4, PD1, or CD45. Incorporation of multiple intracellular inhibitory domains to the iCAR is also possible. Some inhibitory molecules that may provide these inhibitory domains include B7-H1, B7-1, CD160, PIH, 2B4, CEACAM (CEACAM-1. CEACAM-3, and/or CEACAM-5), LAG-3, TIGIT, BTLA, LAIR1, and TGFβ-R. In the presence of normal tissue, stimulation of this second antigen binding domain will work to inhibit the CAR. It should be noted that due to this dual antigen specificity, iCARs are also a form of bi-specific CAR-T cells. The safety CAR-T engineering enhances specificity of the CAR-T cell for tumor tissue, and is advantageous in situations where certain normal tissues may express very low levels of a tumor associated antigen that would lead to off target effects with a standard CAR (Morgan 2010). A conditional CAR-T cell expresses an extracellular antigen binding domain connected to an intracellular costimulatory domain and a separate, intracellular costimulator. The costimulatory and stimulatory domain sequences are engineered in such a way that upon administration of an exogenous molecule the resultant proteins will come together intracellularly to complete the CAR circuit. In this way, CAR-T activation can be modulated, and possibly even ‘fine-tuned’ or personalized to a specific patient. Similar to a dual CAR design, the stimulatory and costimulatory domains are physically separated when inactive in the conditional CAR; for this reason these too are also referred to as a “split CAR”.

In some embodiments, two or more of these engineered features may be combined to create an enhanced, multifunctional CAR-T. For example, it is possible to create a CAR-T cell with either dual- or conditional-CAR design that also releases cytokines like a TRUCK. In some embodiments, a dual-conditional CAR-T cell could be made such that it expresses two CARs with two separate antigen binding domains against two distinct cancer antigens, each bound to their respective costimulatory domains. The costimulatory domain would only become functional with the stimulatory domain after the activating molecule is administered. For this CAR-T cell to be effective the cancer must express both cancer antigens and the activating molecule must be administered to the patient; this design thereby incorporating features of both dual and conditional CAR-T cells.

Typically, CAR-T cells are created using α-β T cells, however γ-δ T cells may also be used. In some embodiments, the described CAR constructs, domains, and engineered features used to generate CAR-T cells could similarly be employed in the generation of other types of CAR-expressing immune cells including NK (natural killer) cells, B cells, mast cells, myeloid-derived phagocytes, and NKT cells. Alternatively, a CAR-expressing cell may be created to have properties of both T-cell and NK cells. In an additional embodiment, the transduced with CARs may be autologous or allogeneic to the patient to whom they are administered.

Several different methods for CAR expression may be used including retroviral transduction (including γ-retroviral), lentiviral transduction, transposon/transposases (Sleeping Beauty and PiggyBac systems), and messenger RNA transfer-mediated gene expression. Gene editing (gene insertion or gene deletion/disruption) has become of increasing importance with respect to the possibility for engineering CAR-T cells as well. CRISPR-Cas9, ZFN (zinc finger nuclease), and TALEN (transcription activator like effector nuclease) systems are three potential methods through which CAR-T cells may be generated.

A number of embodiments of the invention have been described. Nevertheless, it will be understood that various modifications may be made without departing from the spirit and scope of the invention. Accordingly, other embodiments are within the scope of the following claims.

### EXAMPLES

### Example 1: CD99 Hybridoma Screening

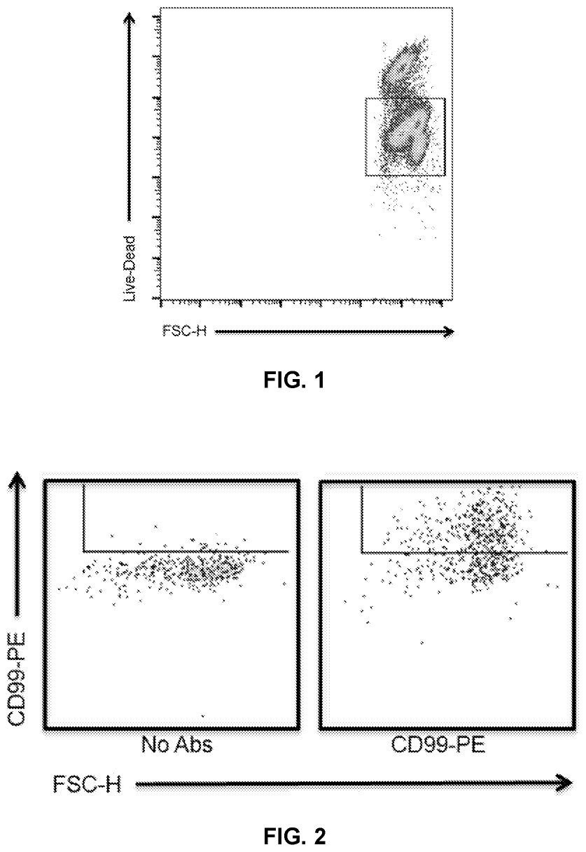

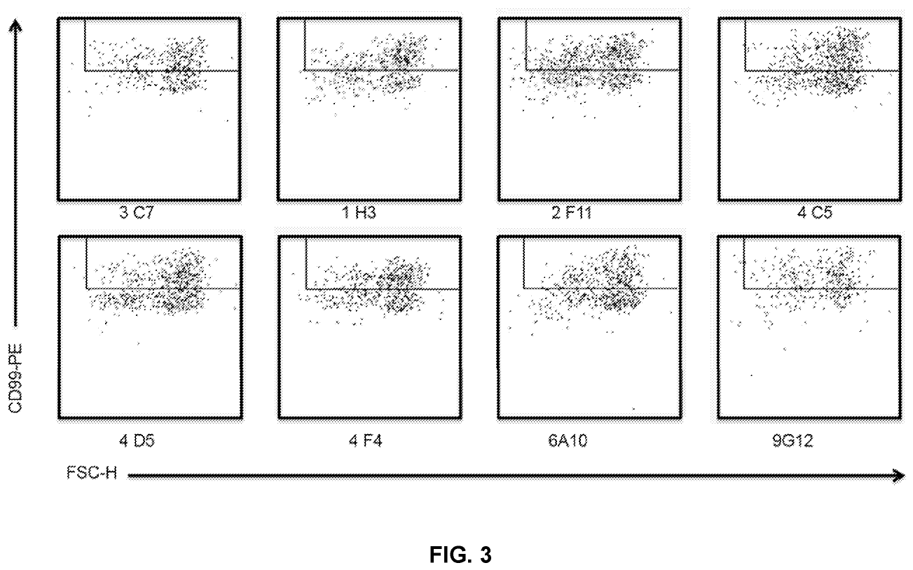

EL4-hCD99 Cells were incubated with antibodies/supernatant from hybridomas. Cells were then stained with Live dead Zombie Green dye and stained with F(ab2) anti-mlgG-PE. INTELLICYT high throughput screener (iQue) was used to assess the cells positive for CD99 PE. FIG. 1 contains a flow cytometry plot showing gate used for live cells in CD99-PE analysis. FIG. 2 contains flow cytometry plots showing positive (right) and negative (left) controls used for CD99-PE analysis. The left histogram is of a control sample in which no supernatant, i.e antibodies(abs) was used. The right histogram is of a positive control in which PE labeled CD99 antibody was used. The gate represents CD99-PE positive population. FIG. 3 contains flow cytometry plots showing hybridomas positive for CD99. Numbers on the bottom of the histogram represent wells/hybridomas.

Hybridomas selected from primary screening were sub cloned. ELISA Plates were coated with CD99 antigen (Origene, Sku #TP304058, lot ##105470), 0.5 ug/ml in DPBS (Lonza cat #17-512F, lot #0000615334), 50 ul/well, at room temperature for 1 hour, and then blocked with 1% BSA/DPBS 100 ul/well, room temperature for 1 hour. Supernatant from monoclonal hybridomas were then added to the coated plates (50 ul/well). Antibody was detected using goat anti Mouse Ig-HRP (1010-05), 1:4000 in TBST, 50 ul/well, room temperature for 40 mins, followed by ABTs/H₂O₂ for 10 mins. Tables 7 to 12 show the results of this screen.

TABLE 7
1H3 - A subclone screening

| | 1 | 2 | 3 | 4 | 5 | 6 | 7 | 8 | 9 | 10 | 11 | 12 |
|---|---|---|---|---|---|---|---|---|---|---|---|---|
| A | 0.231 | 0.883 | 0.853 | 0.835 | 0.779 | 0.709 | 0.125 | 0.751 | 0.493 | 0.324 | 0.143 | 0.088 |
| B | 0.152 | 0.877 | 0.827 | 0.891 | 0.077 | 0.717 | 0.693 | 0.713 | 0.363 | 0.597 | 0.481 | 0.085 |
| C | 0.17 | 0.091 | 0.894 | 0.776 | 0.096 | 0.141 | 0.727 | 0.715 | 0.703 | 0.632 | 0.51 | 0.498 |
| D | 0.096 | 0.83 | 0.715 | 0.832 | 0.84 | 0.837 | 0.419 | 0.804 | 0.266 | 0.689 | 0.569 | 0.071 |
| E | 0.914 | 0.842 | 0.816 | 0.14 | 0.809 | 0.782 | 0.233 | 0.122 | 0.832 | 0.084 | 0.554 | 0.546 |
| F | 0.777 | 0.149 | 0.112 | 0.828 | 0.797 | 0.898 | 0.8 | 0.736 | 0.343 | 0.434 | 0.09 | 0.573 |
| G | 0.342 | 0.134 | 0.744 | 0.781 | 0.074 | 0.529 | 0.206 | 0.748 | 0.162 | 0.046 | 0.045 | 0.041 |
| H | 0.404 | 0.816 | 0.083 | 0.258 | 0.742 | 0.811 | 0.72 | 0.088 | 0.658 | 0.652 | 0.711 | 0 |

TABLE 8
4D5 - A subclone screening

| | 1 | 2 | 3 | 4 | 5 | 6 | 7 | 8 | 9 | 10 | 11 | 12 |
|---|---|---|---|---|---|---|---|---|---|---|---|---|
| A | 0.006 | 0.016 | 0.008 | 0.016 | 0.02 | 0.009 | 0.002 | −0.005 | −0.007 | −0.008 | −0.006 | −0.001 |
| B | 0.006 | 0.008 | 0.01 | 0.009 | 0.019 | 0.004 | −0.001 | 0.003 | −0.004 | 0.001 | −0.012 | −0.009 |
| C | 0.025 | 0.01 | 0.002 | 0.01 | −0.006 | 0.004 | 0.004 | −0.005 | 0.005 | −0.012 | −0.006 | 0.006 |
| D | 0.018 | 0.005 | 0.006 | 0.025 | 0.004 | 0.003 | 0 | −0.005 | 0.002 | −0.016 | −0.015 | −0.022 |
| E | 0.009 | 0.002 | 0 | 0.002 | 0.001 | 0.005 | −0.003 | −0.003 | −0.01 | −0.016 | −0.014 | −0.017 |
| F | 0.008 | −0.003 | 0.005 | −0.012 | 0.007 | 0 | −0.003 | −0.008 | −0.007 | −0.011 | −0.01 | −0.016 |
| G | 0.009 | 0 | 0.005 | 0.003 | 0.015 | −0.001 | −0.004 | −0.004 | −0.009 | 0.009 | −0.018 | −0.018 |
| H | 0 | 0.005 | 0.003 | 0.013 | 0.008 | −0.004 | −0.004 | −0.012 | −0.005 | −0.011 | −0.021 | −0.02 |

TABLE 9
4C5 - A subclone screening

| | 1 | 2 | 3 | 4 | 5 | 6 | 7 | 8 | 9 | 10 | 11 | 12 |
|---|---|---|---|---|---|---|---|---|---|---|---|---|
| A | 0.802 | 0.85 | 0.655 | 0.758 | 0.699 | 0.638 | 0.707 | 0.741 | 0.578 | 0.535 | 0.584 | 0.521 |
| B | 0.61 | 0.647 | 0.662 | 0.572 | 0.719 | 0.639 | 0.583 | 0.585 | 0.587 | 0.599 | 0.571 | 0.476 |
| C | 0.806 | 0.79 | 0.776 | 0.742 | 0.648 | 0.671 | 0.659 | 0.618 | 0.597 | 0.44 | 0.525 | 0.483 |
| D | 0.727 | 0.754 | 0.761 | 0.739 | 0.669 | 0.635 | 0.745 | 0.583 | 0.487 | 0.378 | 0.541 | 0.459 |
| E | 0.806 | 0.735 | 0.622 | 0.646 | 0.682 | 0.773 | 0.626 | 0.627 | 0.399 | 0.514 | 0.412 | 0.468 |
| F | 0.788 | 0.678 | 0.694 | 0.752 | 0.638 | 0.625 | 0.641 | 0.661 | 0.394 | 0.443 | 0.424 | 0.471 |
| G | 0.573 | 0.712 | 0.736 | 0.72 | 0.742 | 0.683 | 0.658 | 0.626 | 0.488 | 0.551 | 0.495 | 0.462 |
| H | 0.561 | 0.738 | 0.769 | 0.758 | 0.808 | 0.842 | 0.748 | 0.741 | 0.619 | 0.533 | 0.502 | 0 |

TABLE 10
4C5 - B subclone screening

| | 1 | 2 | 3 | 4 | 5 | 6 | 7 | 8 | 9 | 10 | 11 | 12 |
|---|---|---|---|---|---|---|---|---|---|---|---|---|
| A | 0.087 | 0.855 | 0.662 | 0.891 | 0.077 | 0.058 | 0.034 | 0.141 | 0.026 | 0.065 | 0.041 | 0.215 |
| B | 0.897 | 0.129 | 0.056 | 0.911 | 0.876 | 0.08 | 0.269 | 0.688 | 0.024 | 0.067 | 0.63 | 0.043 |
| C | 0.263 | 0.914 | 0.055 | 0.06 | 0.082 | 0.044 | 0.046 | 0.744 | 0.437 | 0.027 | 0.672 | 0.388 |
| D | 0.123 | 0.803 | 0.302 | 0.599 | 0.105 | 0.047 | 0.069 | 0.219 | 0.115 | 0.03 | 0.089 | 0.683 |
| E | 0.907 | 0.892 | 0.276 | 0.892 | 0.043 | 0.937 | 0.552 | 0.367 | 0.032 | 0.189 | 0.028 | 0.512 |
| F | 0.662 | 0.757 | 0.161 | 0.043 | 0.868 | 0.054 | 0.038 | 0.787 | 0.052 | 0.078 | 0.107 | 0.026 |
| G | 0.891 | 0.048 | 0.15 | 0.964 | 0.88 | 0.048 | 0.378 | 0.048 | 0.062 | 0.089 | 0.08 | 0.035 |
| H | 0.065 | 0.155 | 0.974 | 0.095 | 0.232 | 0.788 | 0.197 | 0.112 | 0.164 | 0.756 | 0.146 | 0 |

TABLE 11
9G12 - A subclone screening

| | 1 | 2 | 3 | 4 | 5 | 6 | 7 | 8 | 9 | 10 | 11 | 12 |
|---|---|---|---|---|---|---|---|---|---|---|---|---|
| A | 0.663 | 0.722 | 0.904 | 0.847 | 1.016 | 0.999 | 0.924 | 0.788 | 0.829 | 0.94 | 0.851 | 0.771 |
| B | 0.479 | 0.67 | 0.705 | 0.806 | 0.969 | 0.746 | 0.839 | 0.933 | 0.858 | 0.885 | 0.754 | 0.92 |
| C | 0.553 | 0.567 | 0.726 | 0.683 | 0.71 | 0.739 | 0.872 | 0.95 | 0.926 | 0.915 | 0.801 | 0.758 |
| D | 0.609 | 0.722 | 0.649 | 0.671 | 0.78 | 0.685 | 0.83 | 0.822 | 1.003 | 0.79 | 0.93 | 0.856 |
| E | 0.78 | 0.545 | 0.663 | 0.787 | 0.618 | 0.767 | 0.813 | 0.794 | 0.922 | 0.853 | 0.814 | 0.825 |
| F | 0.708 | 0.553 | 0.695 | 0.702 | 0.598 | 0.707 | 0.734 | 1.041 | 0.944 | 0.785 | 0.724 | 0.893 |
| G | 0.586 | 0.586 | 0.555 | 0.755 | 0.764 | 0.739 | 0.802 | 0.949 | 0.827 | 0.718 | 0.86 | 0.796 |
| H | 0.728 | 0.709 | 0.742 | 0.597 | 0.765 | 0.723 | 0.702 | 0.909 | 0.834 | 0.903 | 0.995 | 0 |

TABLE 12
9G12 - B subclone screening

| | 1 | 2 | 3 | 4 | 5 | 6 | 7 | 8 | 9 | 10 | 11 | 12 |
|---|---|---|---|---|---|---|---|---|---|---|---|---|
| A | 1.229 | 1.273 | 1.262 | 0.084 | 1.236 | 0.092 | 0.26 | 1.108 | 0.102 | 0.845 | 0.268 | 0.245 |
| B | 1.03 | 0.993 | 0.58 | 0.088 | 1.239 | 1.193 | 1.17 | 0.184 | 0.746 | 0.108 | 0.149 | 0.989 |
| C | 1.284 | 1.091 | 1.164 | 0.552 | 1.196 | 1.237 | 0.889 | 0.08 | 1.124 | 0.337 | 0.057 | 0.958 |
| D | 1.289 | 1.039 | 0.097 | 0.095 | 0.157 | 0.09 | 1.14 | 1.053 | 0.639 | 0.081 | 0.304 | 0.933 |
| E | 1.179 | 0.721 | 0.14 | 0.122 | 0.136 | 1.211 | 0.152 | 0.129 | 0.09 | 0.647 | 0.962 | 0.737 |
| F | 0.119 | 0.116 | 1.189 | 0.239 | 1.206 | 1.199 | 1.22 | 1.092 | 1.055 | 0.309 | 0.983 | 0.09 |
| G | 1.175 | 0.104 | 1.129 | 0.093 | 1.14 | 1.126 | 1.184 | 0.087 | 0.282 | 1.016 | 0.047 | 0.272 |
| H | 1.193 | 1.261 | 0.159 | 0.188 | 0.244 | 0.608 | 1.109 | 1.169 | 0.677 | 0.174 | 1.004 | 0 |

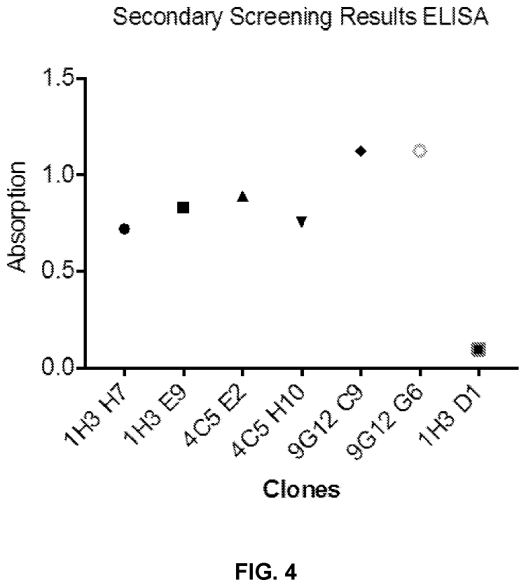

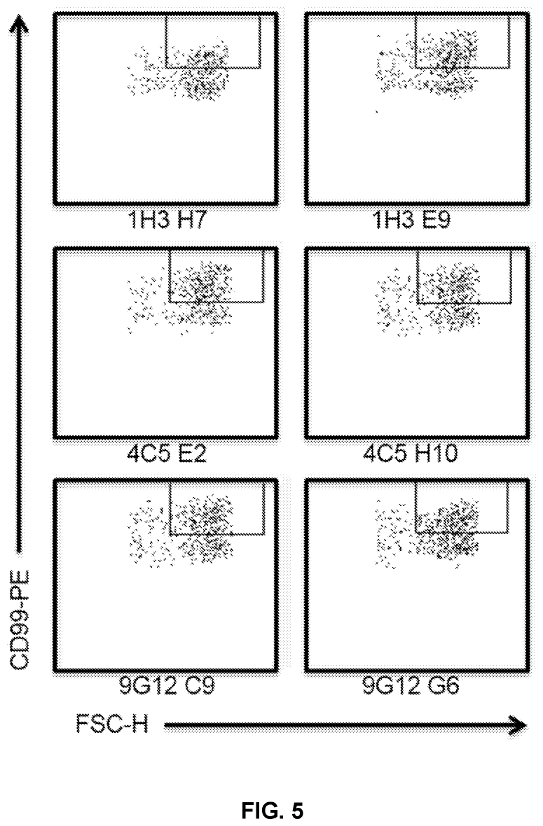

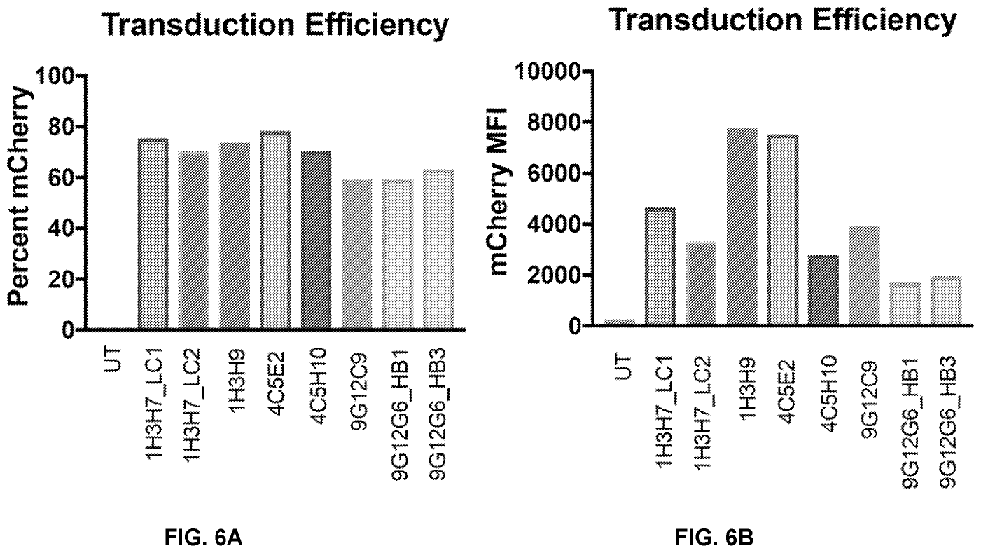

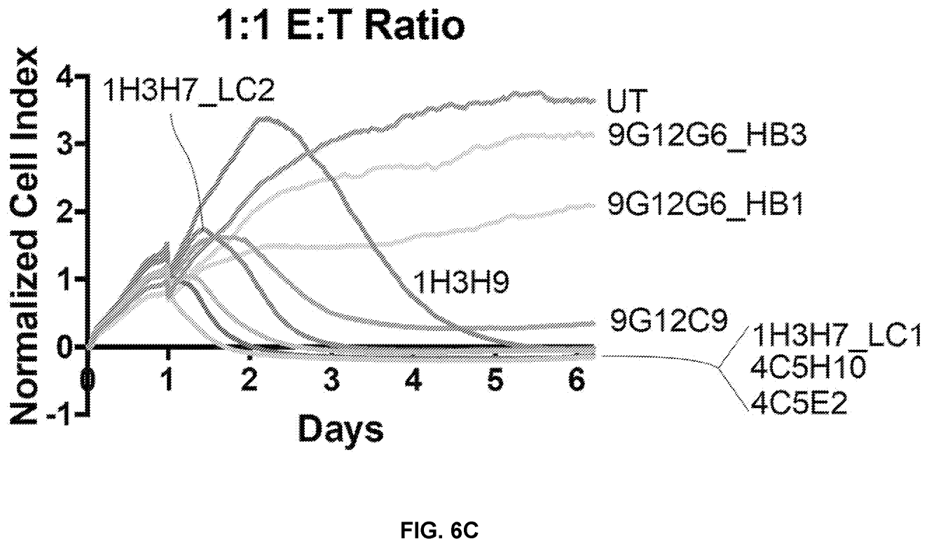

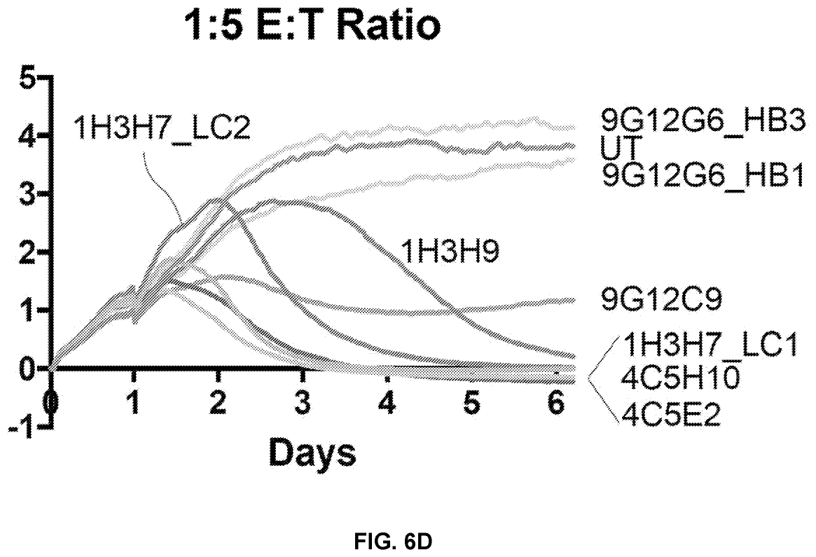

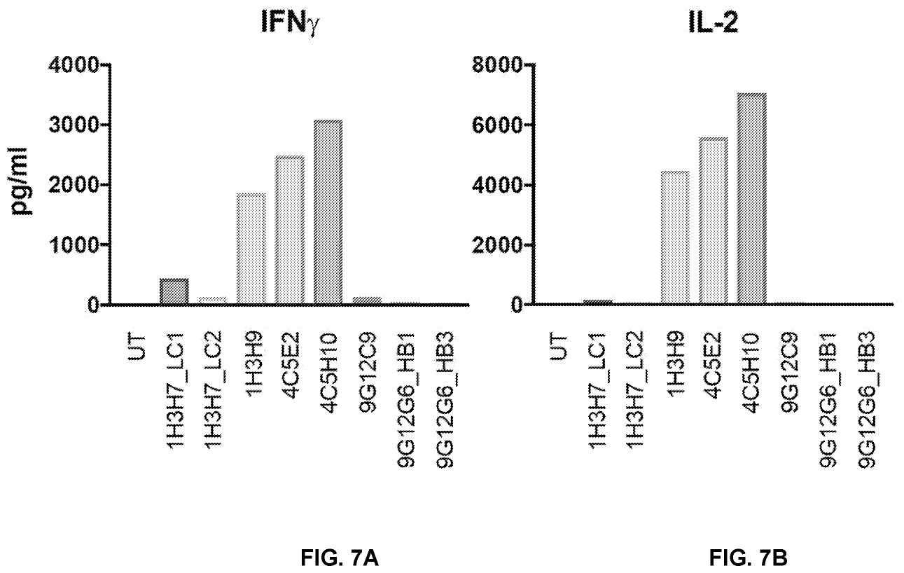

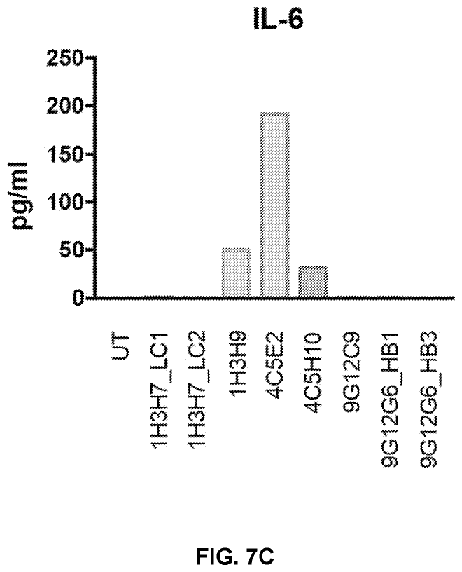

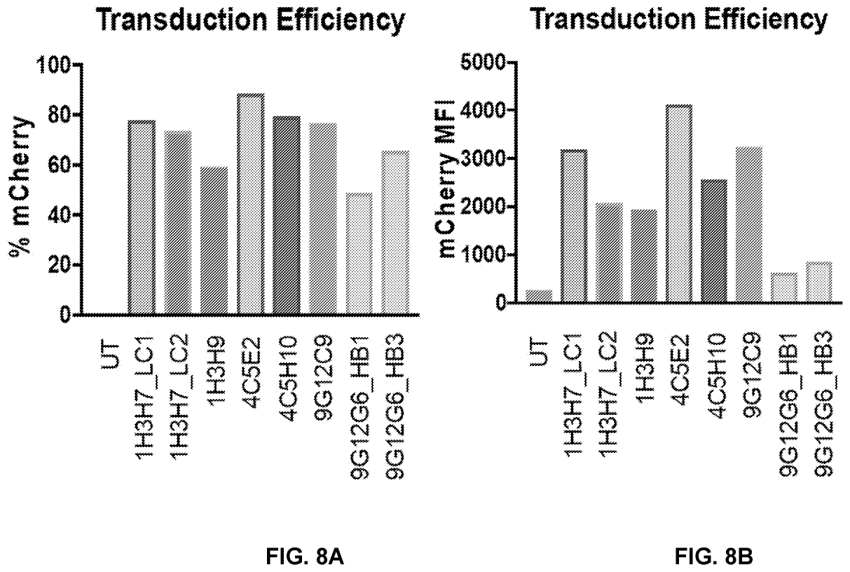

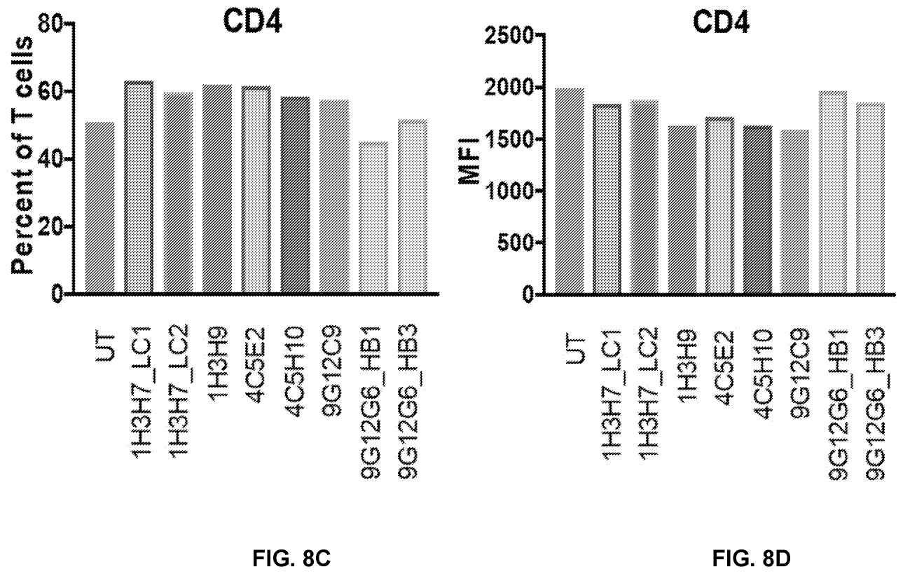

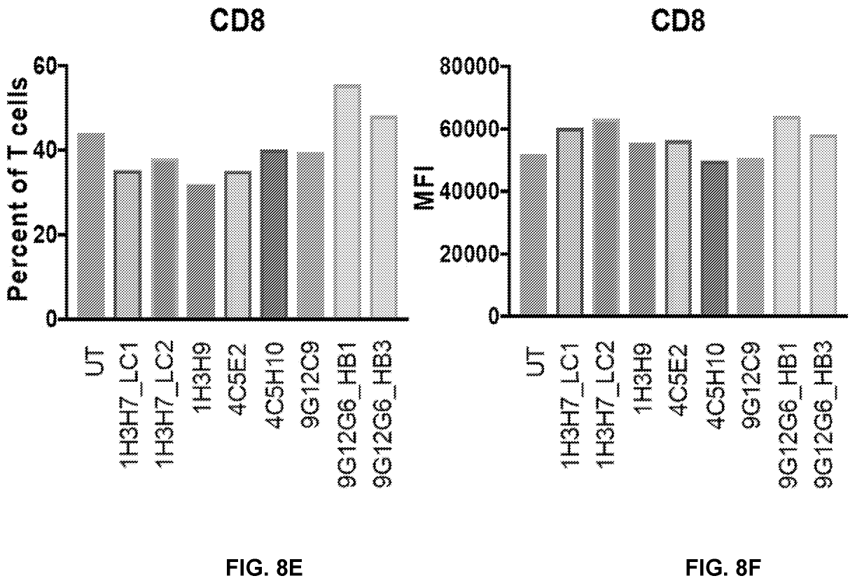

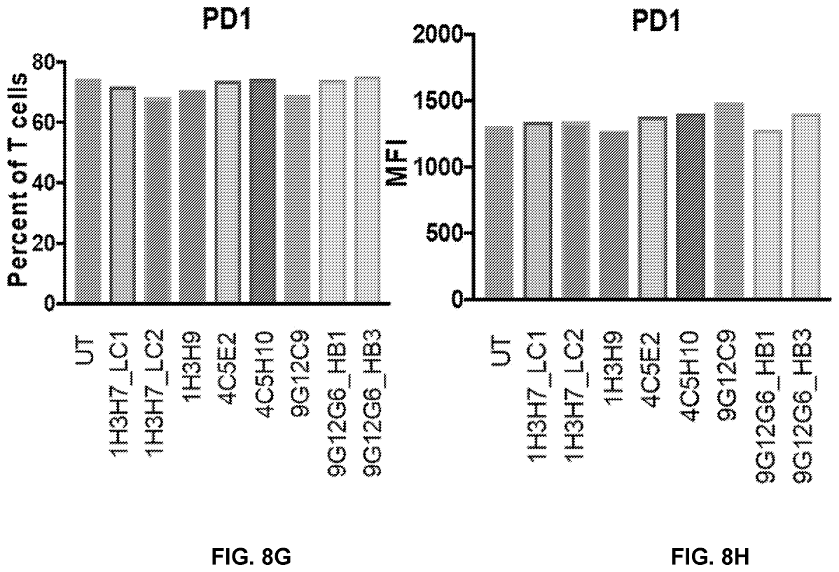

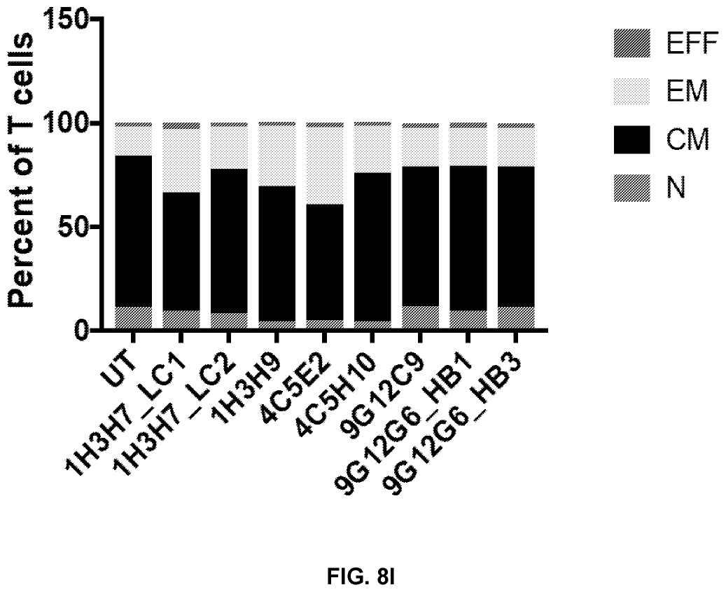

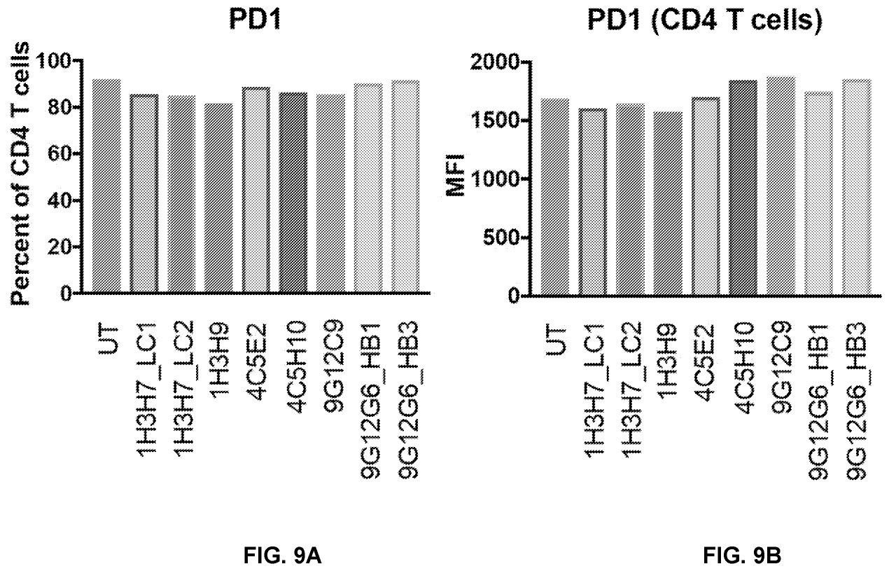

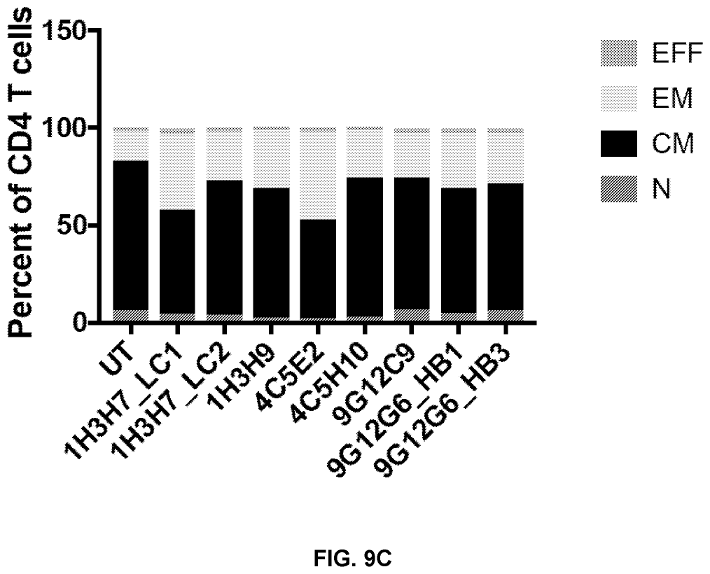

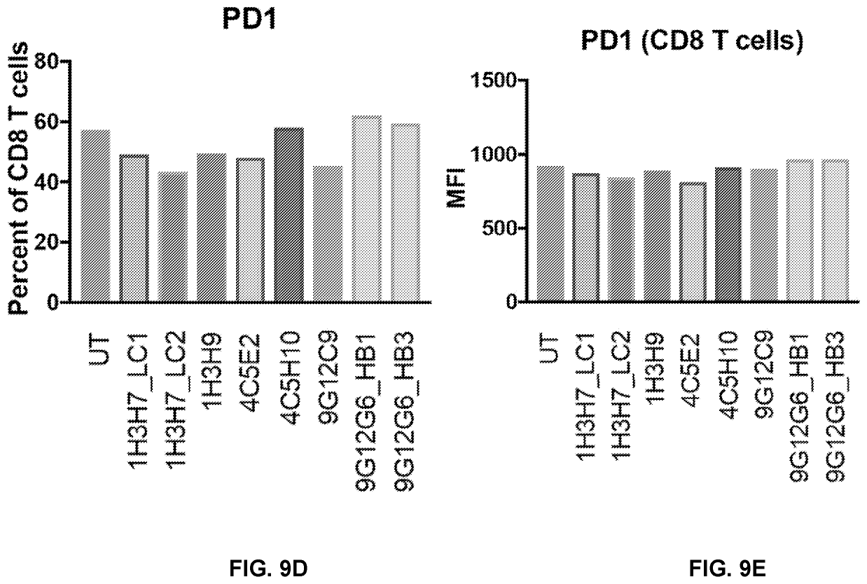

FIG. 4 contains a plot depicting clones that were positive with ELISA and selected for IgH/lgL cloning. Clone 1H3 D1 is negative/low for CD99. FIG. 5 contains flow cytometry plots showing secondary screening of 1H3H7, IH3E9, 4C5E2, 4C5H10, 9G12C9, and 9G12G6.

Using Primary screening with iQUe and Secondary screening with iQue and ELISA, hybridomas and clones were selected that produced monoclonal CD99 antibodies. Monoclonal hybridomas from secondary screening are further subcloned and gene rearrangements of Heavy chain and Light chain of the antibody are determined for designing CD99 Chimeric antigen receptor (CAR).

### Example 2: CLEC12A Hybridoma Screening

Hybridomas selected from primary screening were sub cloned. ELISA Plates were coated with CLEC12A antigen (Thermo Fisher, cat #11896H07H50, lot #LCL07JL0401) diluted with DPBS (LONZA, cat #17-512F, lot #0000615334) to 1 ug/ml at RT for 1 hour, and then blocked with 1% BSA/DPBS 100 μl/well, room temperature for 1 hour. Supernatant from monoclonal hybridomas were then added to the coated plates (50 ul/well). Antibody was detected using goat anti Mouse Ig-HRP (1010-05), 1:4000 in TBST, 50 ul/well, room temperature for 45 mins, followed by ABTs/H2O2 for 10 mins. Clones 1F3, 1F8, 1G3, 2A10, 3F12, 4E3, 4E10, 5B2, 5F10, 6C7, 9A2, 11C7, 11H1, and 12D6 showed positive binding to CLEC12A.

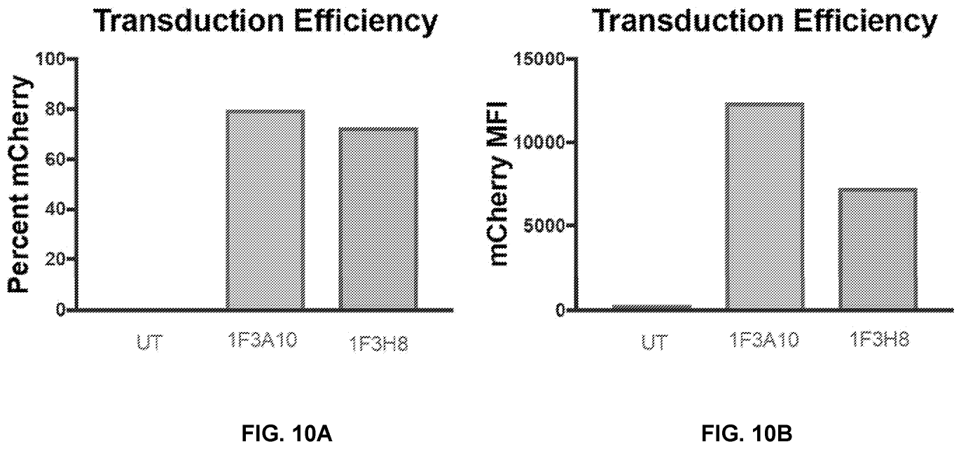

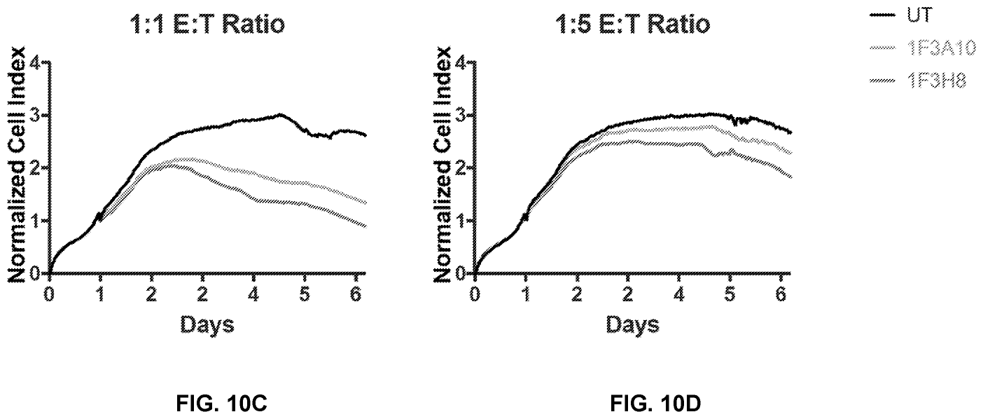

FIGS. 10A to 10D shows CHO cells overexpressing CLEC12A (CHO-CLEC12A) were used as target cells. Gammaretroviruses expressing anti-CLEC12A CARs were transduced into primary T cells isolated from healthy PBMCs. Transduction efficiency of each CAR was determined by flow cytometric analysis of mCherry expression (FIGS. 10A and 10B). CAR positive cells were added to target cells at effector to target ratios of either 1:1 (FIG. 10C) or 1:5 (FIG. 1D). UT=Untransduced, MFI=median fluorescent intensity.

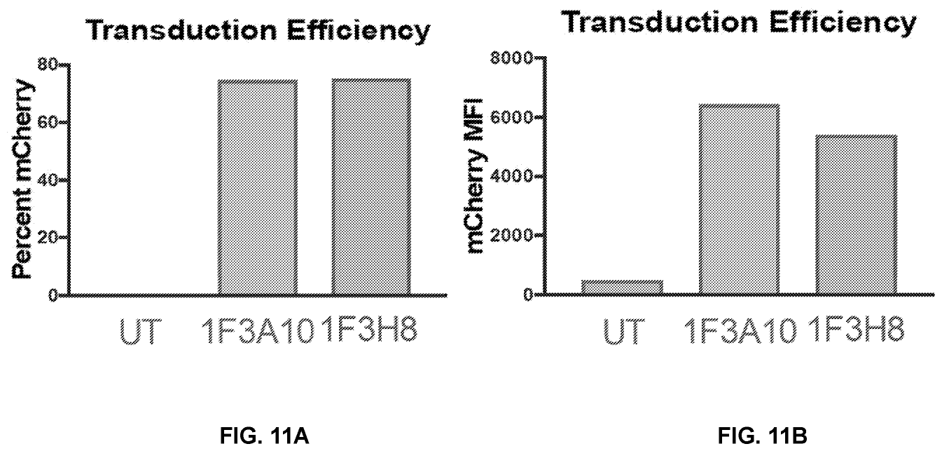

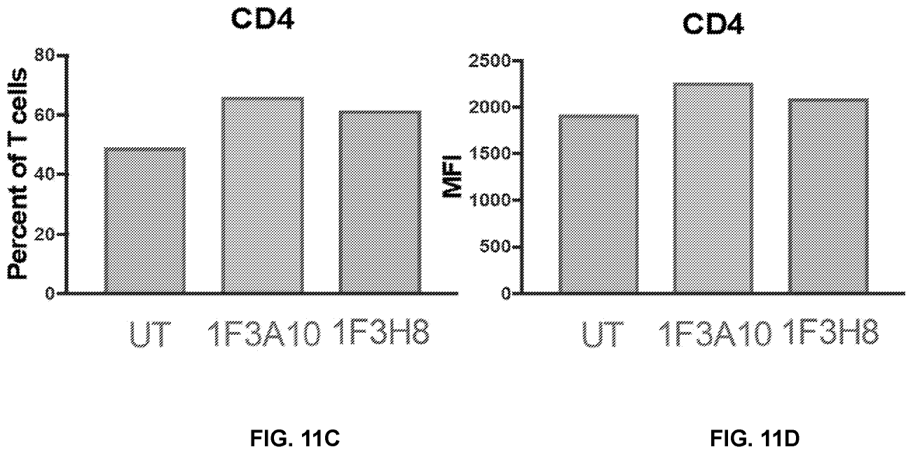

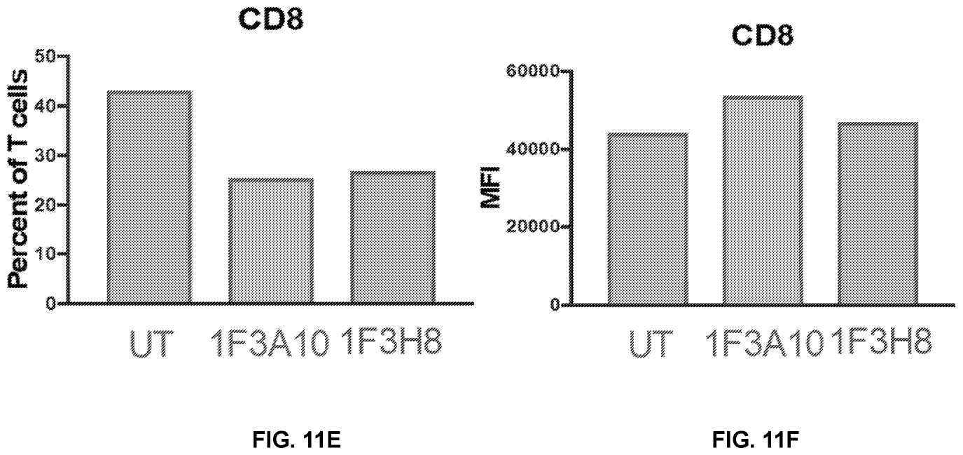

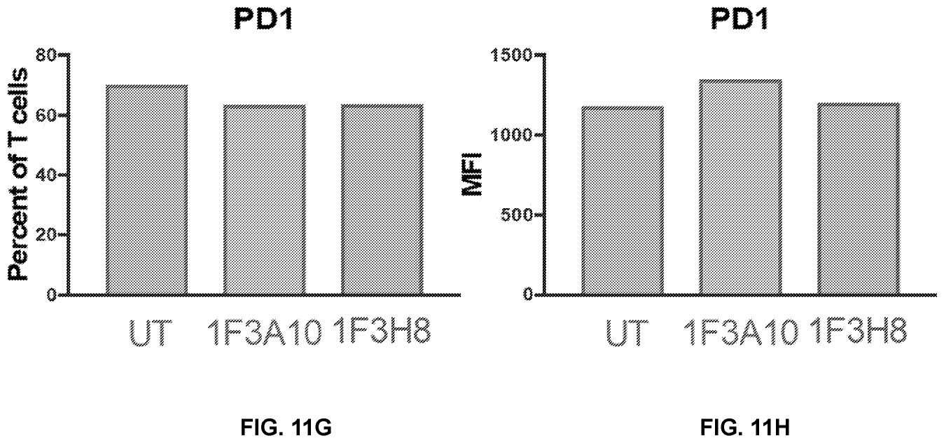

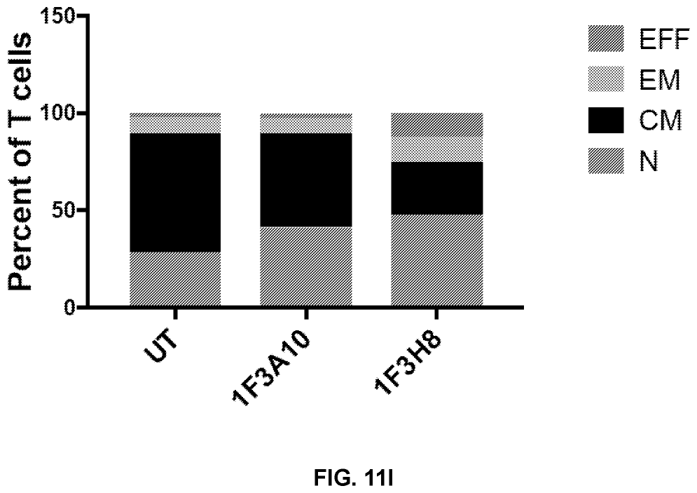

FIGS. 11A to 11I show immunephenotype of anti-CLEC12A CARs. Healthy T cells isolated from PBMCs were transduced with anti-CLEC12A CARs. Following 1-week of culturing without antigen stimulation, cells were stained for CD3, CD4, CD8, PD1, CCR7, and CD45RA, and data were collected on a flow cytometer. Transduction efficiency was determined based on mCherry expression (FIGS. 11A and 11B). Live, CAR positive T cells were analyzed for CD4, CD8, and PD1 expression (FIGS. 11C to 11H). T cells subsets were also analyzed based on CCR7 and CD45RA expression (FIG. 111). EFF=effector, EM=effector memory, CM=central memory, N=Naïve.

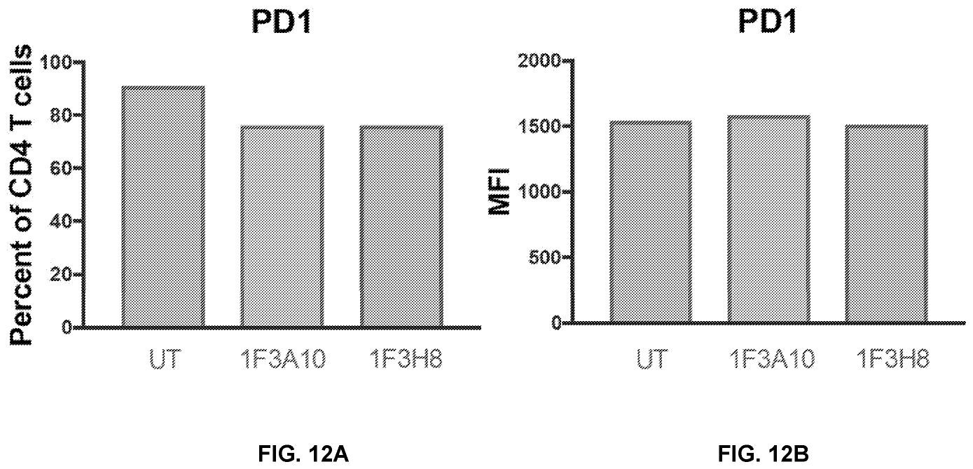

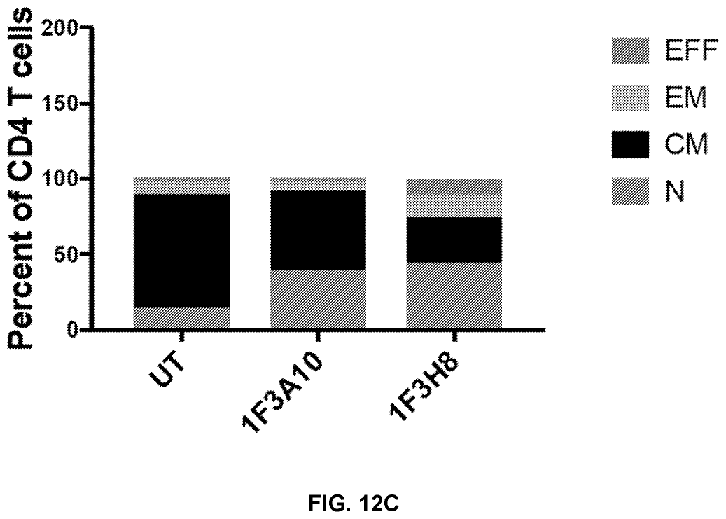

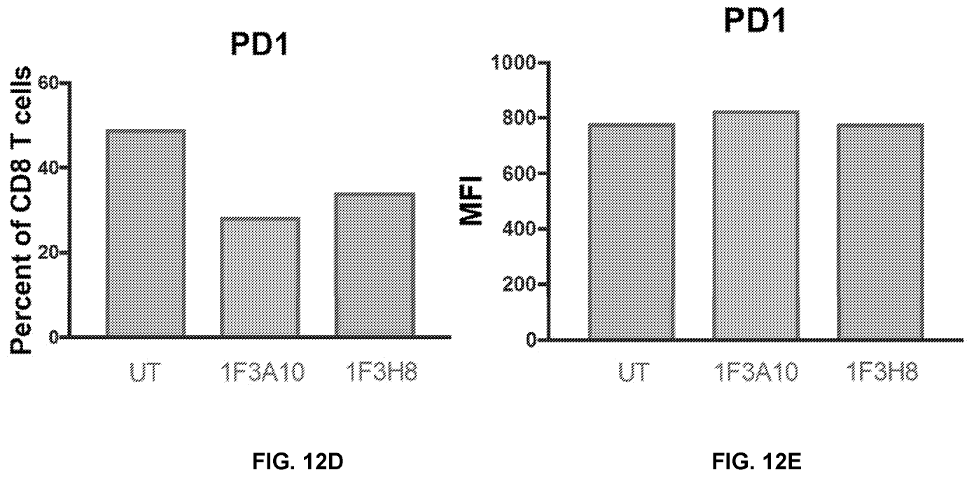

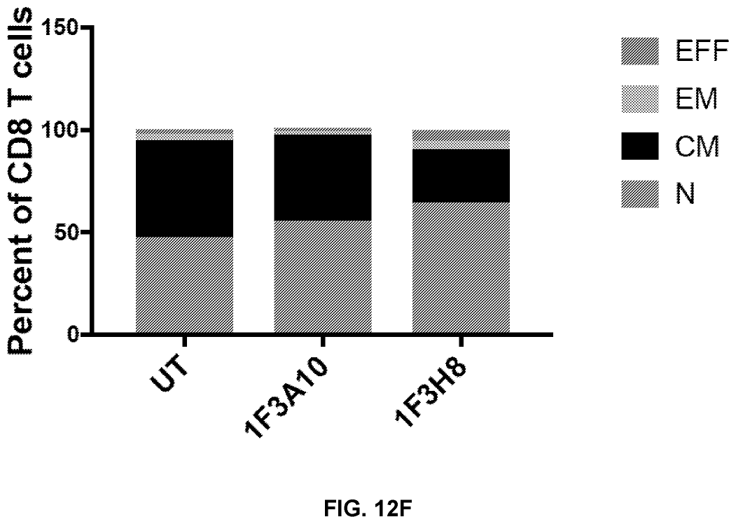

FIGS. 12A to 12F show CD4 and CD8 immunephenotype of anti-CLEC12A CARs. CD4 and CD8 T cells were analyzed for expression of PD1 (FIGS. 12A & 12B, 12D & 12E) and for T cells subsets (FIGS. 12C & 12F). EFF=effector, EM=effector memory, CM=central memory, N=Naïve.

### Example 3: Dual-Targeted CLEC12a and CD99 CAR-T Cells to Avoid On-Target, Off-Tumor Toxicity

Dual-targeted (CLEC12A and CD99) and gated CAR-T cells were produced to avoid on-target, off-tumor toxicity. A proprietary mut06 costimulatory domain (see WO 2019/010383) was used with potential to include 41BB/mut06 co-stimulation.

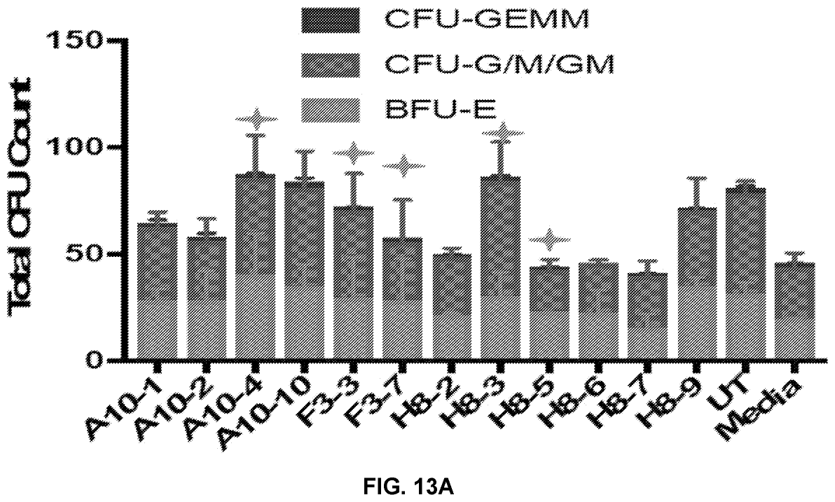

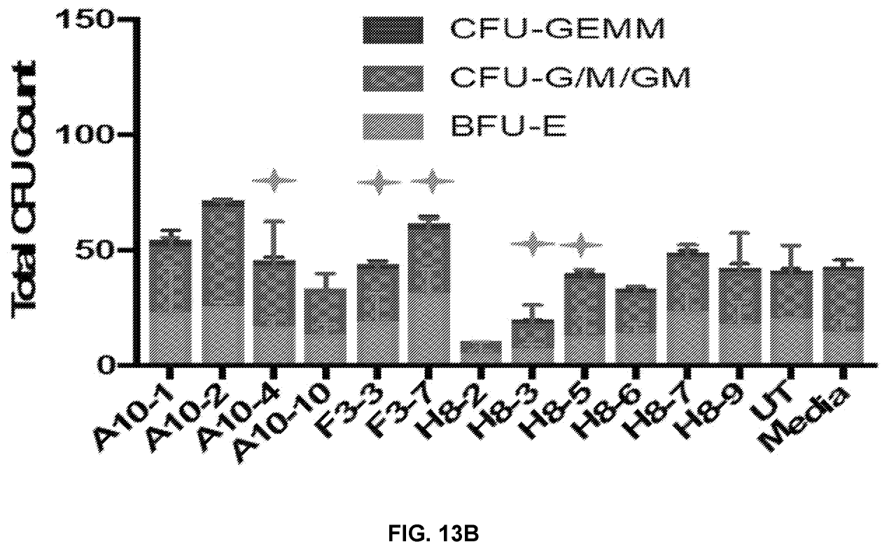

FIGS. 13A and 13B show hematopoietic stem cell compartment safety assay results for the various AML CAR-T candidates tested. Experimental Design: CD34+ cells were co-cultured with CAR T cells (normalized to percentage of cells positive for CAR) for 4 hours at 37° C., 5% CO₂ at an E:T ratio of 10:1. Cells were plated in MethoCult medium and incubated in 6-well plates for 14 days at 37° C., 5% CO₂. BFU=erythroid progenitor cells, CFU-GM/G/M=granulocyte-macrophage progenitor cells, CFU-GEMM=multipotential granulocyte, erythroid, macrophage and megakaryocyte progenitor cells

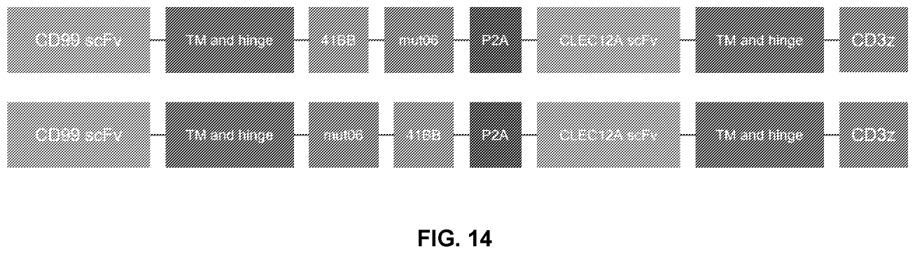

Table 13 and FIG. 14 illustrate dual-targeted AML CAR-T constructs using proprietary scFvs and multiple costimulatory domains nominated for in vivo experiments. The complete amino acid sequence of the H8-5 fusion protein is shown herein as SEQ ID NO:138 and the complete amino acid sequence of the H8-7 fusion protein is shown herein as SEQ ID NO:140.

TABLE 13

| | | Proximal | |
|---|---|---|---|
| | | Costimulatory | |
| Construct | CD99 scFv | Domain | CLEC12A scFv |
| A10-4 | 1H3H9 | mut06 | 1F3A10 |
| A10-10 | 9G12C9 | mut06 | 1F3A10 |
| F3-7 | 4C5H10 | 41BB | 1F3F3 |
| H8-5 | 4C5E2v2 | 41BB | 1F3H8v1 |
| | (SEQ ID NO: 92) | | (SEQ ID NO: 132) |
| H8-7 | 4C5H10v2 | 41BB | 1F3H8v1 |
| | (SEQ ID NO: 93) | | (SEQ ID NO: 132) |

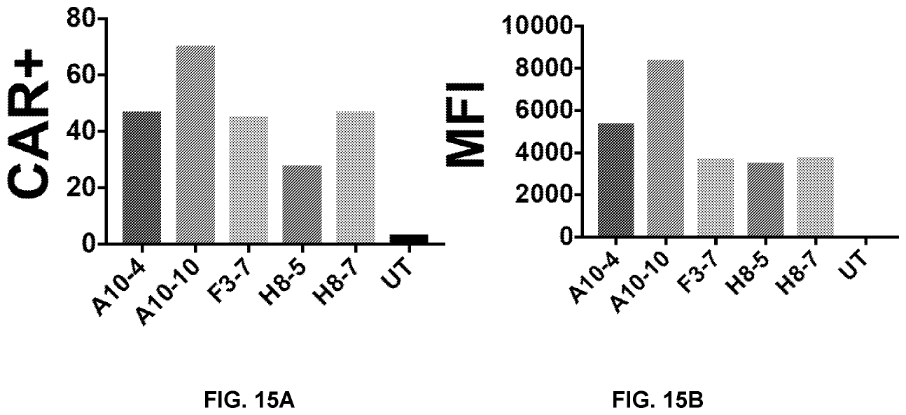

FIGS. 15A and 15B show dual-targeted AML CAR-T IND candidates show good transduction efficiency and surface expression.

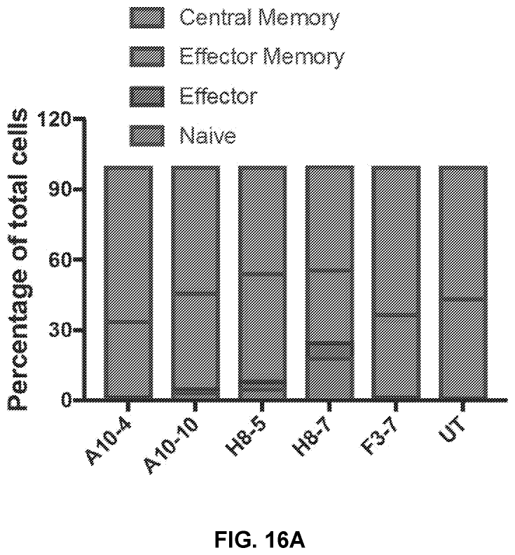

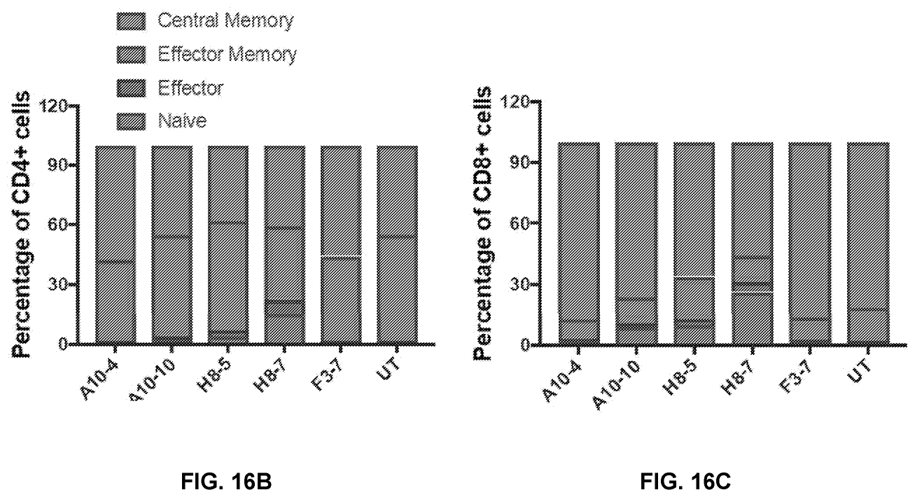

FIGS. 16A to 16C show dual-targeted AML CAR-T IND candidates demonstrate differential retention of central memory phenotype.

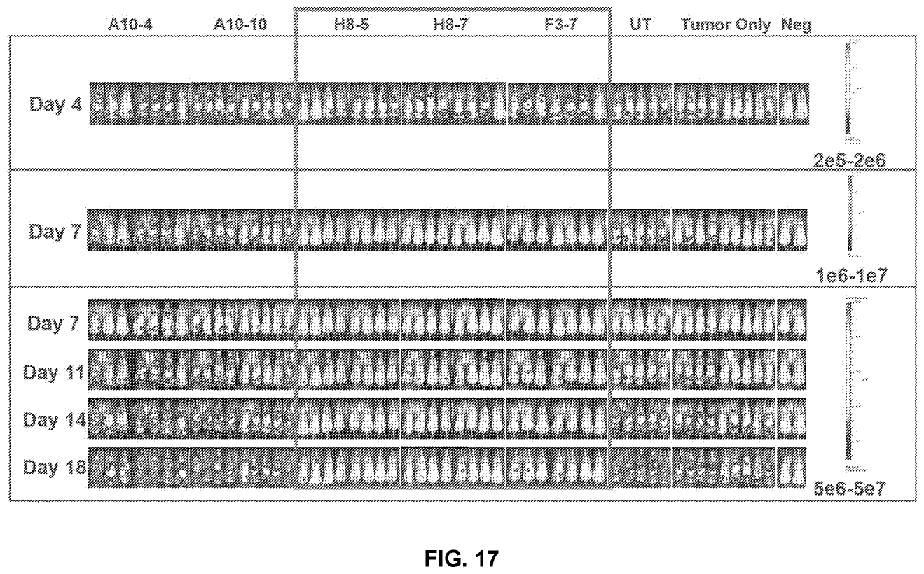

FIG. 17 shows three IND candidates for dual-targeted AML CAR demonstrate tumor stasis in AML model.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An immune effector cell genetically modified to express a first chimeric antigen receptor (CAR) polypeptide and a second CAR, wherein the first CAR comprises a CD99 binding domain, a transmembrane domain, an intracellular signaling domain, and a co-stimulatory signaling region, and wherein the second CAR comprises a CLEC12A binding domain, a transmembrane domain, an intracellular signaling domain, and a co-stimulatory signaling region.

2. The immune effector cell of claim 1, wherein the CD99 binding domain is a single-chain variable fragment (scFv) of an antibody that specifically binds CD99.

3. The immune effector cell of claim 2, wherein the scFv that binds to CD99 comprises a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid sequence SEQ ID NO:1, SEQ ID NO:2, SEQ ID NO:3, or SEQ ID NO:4; the CDR2 sequence of the V_{H} domain comprises the amino acid sequence SEQ ID NO:5, SEQ ID NO:6, or SEQ ID NO:7; the CDR3 sequence of the V_{H} domain comprises the amino acid sequence SEQ ID NO:8, SEQ ID NO:9, SEQ ID NO:10, or SEQ ID NO:11; the CDR1 sequence of the V_{L} comprises the amino acid sequence SEQ ID NO:12, SEQ ID NO:13, SEQ ID NO:14, or SEQ ID NO:15; the CDR2 sequence of the V_{L} domain comprises the amino acid sequence SEQ ID NO:16, SEQ ID NO:17, or SEQ ID NO:18; and the CDR3 sequence of the V_{L} domain comprises the amino acid sequence SEQ ID NO:19, SEQ ID NO:20, or SEQ ID NO:21.

4. The immune effector cell of claim 1, wherein the CD99 binding domain comprises an scFv comprising an amino acid sequence selected from the group consisting of SEQ ID NO:47, SEQ ID NO:56, SEQ ID NO:62, SEQ ID NO:69, SEQ ID NO:85, SEQ ID NO:92, SEQ ID NO:93, and SEQ ID NO:100.

5. The immune effector cell of claim 1, wherein the CLEC12A binding domain is a single-chain variable fragment (scFv) of an antibody that specifically binds CLEC12A.

6. The immune effector cell of claim 5, wherein the scFv that binds to CLEC12A comprises a variable heavy (V_{H}) domain having CDR1, CDR2 and CDR3 sequences and a variable light (V_{L}) domain having CDR1, CDR2 and CDR3 sequences, wherein the CDR1 sequence of the V_{H} domain comprises the amino acid SEQ ID NO:109, SEQ ID NO:110, or SEQ ID NO:111; the CDR2 sequence of the V_{H} domain comprises the amino acid sequence SEQ ID NO:112, SEQ ID NO:113, or SEQ ID NO:114); the CDR3 sequence of the V_{H} domain comprises the amino acid sequence SEQ ID NO:115, SEQ ID NO:116, or SEQ ID NO: 117; the CDR1 sequence of the V_{L} comprises the amino acid sequence SEQ ID NO:118, SEQ ID NO: 119, or SEQ ID NO:120; the CDR2 sequence of the V_{L} domain comprises the amino acid sequence SEQ ID NO:121 or SEQ ID NO:122; and the CDR3 sequence of the V_{L} domain comprises the amino acid sequence SEQ ID NO:123.

7. The immune effector cell of claim 1, wherein the CLEC12A binding domain comprises an scFv comprising an amino acid sequence selected from the group consisting of SEQ ID NO:132, SEQ ID NO:133, SEQ ID NO:134, SEQ ID NO:135, SEQ ID NO:136, and SEQ ID NO:137.

8. The immune effector cell of claim 1, wherein (i) the CD99 binding domain comprises an scFv comprising an amino acid sequence selected from the group consisting of SEQ ID NO:47, SEQ ID NO:56, SEQ ID NO:62, SEQ ID NO:69, SEQ ID NO:85, SEQ ID NO:92, SEQ ID NO:93, and SEQ ID NO:100, and (ii) the CLEC12A binding domain comprises an scFv comprising an amino acid sequence selected from the group consisting of SEQ ID NO:132, SEQ ID NO:133, SEQ ID NO:134, SEQ ID NO:135, SEQ ID NO:136, and SEQ ID NO:137.

9. The immune effector cell of claim 1, wherein the costimulatory signaling region comprises the cytoplasmic domain of a costimulatory molecule selected from the group consisting of CD27, CD28, mut06, 4-1BB, OX40, CD30, CD40, PD-1, ICOS, lymphocyte function-associated antigen-1 (LFA-1), CD2, CD7, LIGHT, NKG2C, B7-H3, and any combination thereof.

10. The immune effector cell of claim 1, wherein the intracellular signaling domain comprises a native CD3 zeta (CD3ζ) signaling domain or a 1XX signaling domain.

11. The immune effector cell of claim 1, wherein the first CAR and second CAR are co-expressed as a single fusion protein defined by the formula:
SP-CD99V_{L}-CLV_{H}-HG-TM-CSD-scp-SP-CD99V_{H}-CLV_{L}-HG-TM-CD3ζ-SD;
SP-CD99V_{L}-CLV_{H}-HG-TM-CD3ζ-scp-SP-CD99V_{H}-CLV_{L}-HG-TM-CSD-SD;
SP-CD99V_{H}-CLV_{L}-HG-TM-CSD-scp-SP-CD99V_{L}-CLV_{H}-HG-TM-CD3ζ-SD;
SP-CD99V_{H}-CLV_{L}-HG-TM-CD3ζ-scp-SP-CD99V_{L}-CLV_{H}-HG-TM-CSD-SD;
SP-CD99V_{H}-CLV_{H}-HG-TM-CSD-scp-SP-CD99V_{L}-CLV_{L}-HG-TM-CD3ζ-SD;
SP-CD99V_{L}-CLV_{L}-HG-TM-CD3ζ-scp-SP-CD99V_{H}-CLV_{H}-HG-TM-CSD-SD;
SP-CD99V_{L}-CLV_{L}-HG-TM-CSD-scp-SP-CD99V_{H}-CLV_{H}-HG-TM-CD3ζ-SD;
SP-CD99V_{H}-CLV_{H}-HG-TM-CD3ζ-scp-SP-CD99V_{L}-CLV_{L}-HG-TM-CSD-SD;
SP-CLV_{H}-CD99V_{L}-HG-TM-CSD-scp-SP-CLV_{L}-CD99V_{H}-HG-TM-CD3ζ-SD;
SP-CLV_{H}-CD99V_{L}-HG-TM-CD3ζ-scp-SP-CLV_{L}-CD99V_{H}-HG-TM-CSD-SD;
SP-CLV_{L}-CD99V_{H}-HG-TM-CSD-scp-SP-CLV_{H}-CD99V_{L}-HG-TM-CD3ζ-SD;
SP-CLV_{L}-CD99V_{H}-HG-TM-CD3ζ-scp-SP-CLV_{H}-CD99V_{L}-HG-TM-CSD-SD;
SP-CLV_{H}-CD99V_{H}-HG-TM-CSD-scp-SP-CLV_{L}-CD99V_{L}-HG-TM-CD3ζ-SD;
SP-CLV_{L}-CD99V_{L}-HG-TM-CD3ζ-scp-SP-CLV_{H}-CD99V_{H}-HG-TM-CSD-SD;
SP-CLV_{L}-CD99V_{L}-HG-TM-CSD-scp-SP-CLV_{H}-CD99V_{H}-HG-TM-CD3ζ-SD; or
SP-CLV_{H}-CD99V_{H}-HG-TM-CD3ζ-scp-SP-CLV_{L}-CD99V_{L}-HG-TM-CSD-SD;
wherein “SP” represents an optional signal peptide,
wherein “CD99V_{H}” represents a CD99 variable heavy domain,
wherein “CD99V_{L}” represents a CD99 variable light domain,
wherein “CLV_{H}” represents a CLEC12A variable heavy domain,
wherein “CLV_{L}” represents a CLEC12A variable light domain,
wherein “HG” represents an optional hinge domain,
wherein “TM” represents a transmembrane domain,
wherein “scp” represents a self-cleaving peptide domain,
wherein “CD3ζ” represents a CD3ζ domain,
wherein “CSD” represents a costimulatory-domain, and
wherein “-” represents a peptide bond or linker.

12. The immune effector cell of claim 11, wherein the fusion protein comprises the amino acid sequence SEQ ID NO:138 or SEQ ID NO:140.

13. The immune effector cell of claim 1, wherein the cell is selected from the group consisting of an αβT cell, γδT cell, a Natural Killer (NK) cell, a Natural Killer T (NKT) cell, a B cell, an innate lymphoid cell (ILC), a cytokine induced killer (CIK) cell, a cytotoxic T lymphocyte (CTL), a lymphokine activated killer (LAK) cell, a regulatory T cell, a pluripotent stem cell capable of differentiating into a cytotoxic T cell, or any combination hereof.

14. A method of providing an anti-cancer immunity in a subject with a CD99- and/or CLEC12A-expressing cancer, the method comprising administering to the subject an effective amount of the immune effector cell of claim 1, thereby providing an anti-tumor immunity in the mammal.

15. The method of claim 14, further comprising administering to the subject a checkpoint inhibitor.

16. The method of claim 15, wherein the checkpoint inhibitor comprises an anti-PD-1 antibody, anti-PD-L1 antibody, anti-CTLA-4 antibody, or a combination thereof.

17. The method of claim 14, wherein the cancer comprises myelodysplastic syndromes, acute myeloid leukemia, or bi-phenotypic leukemia.


SEQUENCE LISTING (141 sequences)

SEQ ID NO: 1 - protein, 8 aa, Artificial Sequence
  Synthetic Construct
GFDIKDTY

SEQ ID NO: 2 - protein, 5 aa, Artificial Sequence
  Synthetic Construct
TYAMY

SEQ ID NO: 3 - protein, 4 aa, Artificial Sequence
  Synthetic Construct
TFWM

SEQ ID NO: 4 - protein, 5 aa, Artificial Sequence
  Synthetic Construct
TFWMQ

SEQ ID NO: 5 - protein, 8 aa, Artificial Sequence
  Synthetic Construct
IDPANGDT

SEQ ID NO: 6 - protein, 22 aa, Artificial Sequence
  Synthetic Construct
RIRSKVNNYATYYADSVKDRFT

SEQ ID NO: 7 - protein, 20 aa, Artificial Sequence
  Synthetic Construct
TIYPGDDDTRYTQKFKGRAT

SEQ ID NO: 8 - protein, 7 aa, Artificial Sequence
  Synthetic Construct
ARRGGLS

SEQ ID NO: 9 - protein, 5 aa, Artificial Sequence
  Synthetic Construct
DPMDY

SEQ ID NO: 10 - protein, 12 aa, Artificial Sequence
  Synthetic Construct
SGYERGPYYFDS

SEQ ID NO: 11 - protein, 10 aa, Artificial Sequence
  Synthetic Construct
SGYERGPYYF

SEQ ID NO: 12 - protein, 6 aa, Artificial Sequence
  Synthetic Construct
GNIHNY

SEQ ID NO: 13 - protein, 16 aa, Artificial Sequence
  Synthetic Construct
GSSKSLLHSNGNTYLY

SEQ ID NO: 14 - protein, 17 aa, Artificial Sequence
  Synthetic Construct
KSSQSLLCRSNQKNYLA

SEQ ID NO: 15 - protein, 17 aa, Artificial Sequence
  Synthetic Construct
KSSQSLLYRSNQKNYLA

SEQ ID NO: 16 - protein, 4 aa, Artificial Sequence
  Synthetic Construct
  MISC_FEATURE (4)..(4): Xaa any amino acid
NAKX

SEQ ID NO: 17 - protein, 4 aa, Artificial Sequence
  Synthetic Construct
  misc_feature (4)..(4): Xaa can be any naturally occurring amino acid
NAKX

SEQ ID NO: 18 - protein, 7 aa, Artificial Sequence
  Synthetic Construct
WASTRES

SEQ ID NO: 19 - protein, 9 aa, Artificial Sequence
  Synthetic Construct
QHFWSTPWT

SEQ ID NO: 20 - protein, 9 aa, Artificial Sequence
  Synthetic Construct
MQHLEYPYT

SEQ ID NO: 21 - protein, 9 aa, Artificial Sequence
  Synthetic Construct
QQYYSYPLT

SEQ ID NO: 22 - protein, 114 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 23 - DNA, 342 bp, Artificial Sequence
  Synthetic Construct
gaggttcaactgcaacagtctggggcagagcttgtgaagccaggggcctcagtcaagttgtcctgcacagcttctggcttcgacattaaagacacctatatccactgggtgaaacagaggcctgaacagggcctggagtggattggaaggattgatcctgcgaatggtgatactagatatgacccggaattccagggcaaggcctctctaacagctgacacatcctccaatacagcctacctacaattcagcaacctgacatctgaagacactgccgtctattactgtgctagaagaggcggcctctcctggggccaaggcaccactctcacagtctcctca

SEQ ID NO: 24 - protein, 116 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 25 - DNA, 348 bp, Artificial Sequence
  Synthetic Construct
gaggtgcagctggaggagtctggtggaggattggtgcagcctaaaggatcattgaaactctcatgtgccgcctccggtttcaccttcaatacctatgccatgtactgggtctgccaggctccaggaaagggtttgaaatgggttgctcgcataagaagtaaagttaataattatgcaacatattatgccgattcagtgaaagacagattcaccatctccagagatgattcacaaaacatgctctttctgcacatgaacaacctgaaaactgaggacactgccatatatttctgtgtgagagatcctatggactactggggtcaaggaatctcagtcaccgtctcctca

SEQ ID NO: 26 - protein, 116 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 27 - DNA, 348 bp, Artificial Sequence
  Synthetic Construct
gaggtgcagcttgttgagtctggtggaggattggtgcagcctaaaggatcattgaaactctcatgtgccgcctccggtttcaccttcaatacctatgccatgtactgggtctgccaggctccaggaaagggtttgaaatgggttgctcgcataagaagtaaagttaataattatgcaacatattatgccgattcagtgaaagacagattcaccatctccagagatgattcacaaaacatgctctttctgcacatgaacaacctgaaaactgaggacactgccatatatttctgtgtgagagatcctatggactactggggtcaaggaatctcagtcaccgtctcctca

SEQ ID NO: 28 - protein, 121 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 29 - DNA, 363 bp, Artificial Sequence
  Synthetic Construct
caggttcagctccagcagtctggggctgagctggcaagacctggggcttcagtgaagttgtcctgcaaggcttctggctacacgtttactactttctggatgcagtgggtaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 30 - protein, 121 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 31 - DNA, 363 bp, Artificial Sequence
  Synthetic Construct
gatgtgaagcttcaggagtctggggctgagctggcaagacctggggcttcagtgaagttgtcctgcaaggcttctggctacacgtttactactttctggatgcagcgggtaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 32 - protein, 121 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 33 - DNA, 363 bp, Artificial Sequence
  Synthetic Construct
caggtgcagctgaaggagtctggggctgagctggcaagacctggggcttcagtgaagttgtcctgcaaggcttctggctacacgtttactactttctggatgcagtgggcaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctca

SEQ ID NO: 34 - protein, 107 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 35 - DNA, 321 bp, Artificial Sequence
  Synthetic Construct
gacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaa

SEQ ID NO: 36 - protein, 112 aa, Artificial Sequence
  Synthetic Construct
GNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 37 - protein, 112 aa, Artificial Sequence
  Synthetic Construct
GNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 38 - protein, 112 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 39 - DNA, 336 bp, Artificial Sequence
  Synthetic Construct
gatattgtgatgactcaggctgcaccctctgtacctgtcactcctggggagtcagtatccatctcctgcgggtctagtaagagtctcctgcatagtaatggcaacacttacttgtattggttcctgcagaggccaggccagtctcctcagctcctgatatatcgggtgtccaaccttgcctcaggagtcccagacaggttcagtggcagtgggtcaggaactgctttcacactgagaatcagtagagtggaggctgaggatgtgggtgtttattactgtatgcaacatctagaatatccgtacacgttcggaggggggaccaggctggaaataaaa

SEQ ID NO: 40 - protein, 112 aa, Artificial Sequence; identical to SEQ ID NO: 38
  Synthetic Construct

SEQ ID NO: 41 - DNA, 336 bp, Artificial Sequence; identical to SEQ ID NO: 39
  Synthetic Construct

SEQ ID NO: 42 - protein, 113 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 43 - DNA, 339 bp, Artificial Sequence
  Synthetic Construct
gacactgtgatgtcacagtccccatcctccctagctgtttcagttggagagaagataactatgagctgcaagtccagtcagagtcttttatgtcgtagcaatcaaaagaactacttggcctggtaccagcagaaaccagggcagtctcctaaacagctgatttactgggcatctactagggaatctggggtccctgatcgcttcacaggcagtggatctgggacagatttcactctcaccatcagcagtgtgaaggctgaagacctggcagtttattactgtcagcaatattatagttatccgctcacgttcggtgctggcaccaagctggagctgaaa

SEQ ID NO: 44 - protein, 113 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 45 - DNA, 339 bp, Artificial Sequence
  Synthetic Construct
gacactgtgatgtcacagtccccatcctccctagctgtttcagttggagagaagataactatgagctgcaagtccagtcagagtcttttatatcgtagcaatcaaaagaactacttggcctggtaccagcagaaaccagggcagtctcctaaacagctgatttactgggcatctactagggaatctggggtccctgatcgcttcacaggcagtggatctgggacagatttcactctcaccatcagcagtgtgaaggctgaagacctggcagtttattactgtcagcaatattatagttatccgctcacgttcggtgctggcaccaagctggagctgaaa

SEQ ID NO: 46 - protein, 15 aa, Artificial Sequence
  Synthetic Construct
GGGGSGGGGSGGGGS

SEQ ID NO: 47 - protein, 236 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 48 - protein, 241 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 49 - protein, 241 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 50 - protein, 241 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 51 - protein, 242 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 52 - protein, 242 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 53 - protein, 238 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 54 - protein, 243 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 55 - protein, 243 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 56 - protein, 243 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 57 - protein, 244 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 58 - protein, 244 aa, Artificial Sequence
  Synthetic Construct
EVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 59 - protein, 238 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 60 - protein, 243 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 61 - protein, 243 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 62 - protein, 243 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 63 - protein, 244 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 64 - protein, 244 aa, Artificial Sequence
  Synthetic Construct
EVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 65 - protein, 243 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 66 - protein, 248 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 67 - protein, 248 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 68 - protein, 248 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 69 - protein, 249 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 70 - protein, 249 aa, Artificial Sequence
  Synthetic Construct
QVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 71 - protein, 243 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 72 - protein, 248 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 73 - protein, 248 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 74 - protein, 248 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 75 - protein, 248 aa, Artificial Sequence; identical to SEQ ID NO: 74
  Synthetic Construct

SEQ ID NO: 76 - protein, 249 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 77 - protein, 249 aa, Artificial Sequence
  Synthetic Construct
DVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 78 - protein, 243 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 79 - protein, 248 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWSHSLRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 80 - protein, 248 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSGNSWRHSPRSLSVTIGQPASISCKSSQSLLDGNGKTYLNWLLQRPGQSPKRLLYLVSKLDSGVPDRFTGSGSGTDFTLKISRVEAEDLGIYYCWQGTHFPRTFGGGTKLEIK

SEQ ID NO: 81 - protein, 248 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIK

SEQ ID NO: 82 - protein, 248 aa, Artificial Sequence; identical to SEQ ID NO: 81
  Synthetic Construct

SEQ ID NO: 83 - protein, 249 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 84 - protein, 249 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELK

SEQ ID NO: 85 - protein, 236 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 86 - protein, 238 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 87 - protein, 238 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 88 - protein, 243 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 89 - protein, 243 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 90 - protein, 243 aa, Artificial Sequence
  Synthetic Construct
DIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 91 - protein, 241 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 92 - protein, 243 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 93 - protein, 243 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 94 - protein, 248 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 95 - protein, 248 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 96 - protein, 248 aa, Artificial Sequence
  Synthetic Construct
DIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 97 - protein, 242 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 98 - protein, 244 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 99 - protein, 244 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 100 - protein, 249 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 101 - protein, 249 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 102 - protein, 249 aa, Artificial Sequence
  Synthetic Construct
DTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 103 - protein, 242 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSS

SEQ ID NO: 104 - protein, 244 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 105 - protein, 244 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSS

SEQ ID NO: 106 - protein, 249 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLQQSGAELARPGASVKLSCKASGYTFTTFWMQWVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 107 - protein, 249 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSDVKLQESGAELARPGASVKLSCKASGYTFTTFWMQRVKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 108 - protein, 249 aa, Artificial Sequence
  Synthetic Construct
DTVMSQSPSSLAVSVGEKITMSCKSSQSLLYRSNQKNYLAWYQQKPGQSPKQLIYWASTRESGVPDRFTGSGSGTDFTLTISSVKAEDLAVYYCQQYYSYPLTFGAGTKLELKGGGGSGGGGSGGGGSQVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSS

SEQ ID NO: 109 - protein, 8 aa, Artificial Sequence
  Synthetic Construct
GFTFSSFA

SEQ ID NO: 110 - protein, 5 aa, Artificial Sequence
  Synthetic Construct
SFAVS

SEQ ID NO: 111 - protein, 5 aa, Artificial Sequence
  Synthetic Construct
SHDMS

SEQ ID NO: 112 - protein, 8 aa, Artificial Sequence
  Synthetic Construct
ISSGGAYT

SEQ ID NO: 113 - protein, 20 aa, Artificial Sequence
  Synthetic Construct
TISSGGAYTFYKDSVKGRFT

SEQ ID NO: 114 - protein, 20 aa, Artificial Sequence
  Synthetic Construct
YISGGGTNIYYSDTVKGRFT

SEQ ID NO: 115 - protein, 16 aa, Artificial Sequence
  Synthetic Construct
ARHSGYDGYYLYAMDY

SEQ ID NO: 116 - protein, 14 aa, Artificial Sequence
  Synthetic Construct
HSGYDGYYLYAMDY

SEQ ID NO: 117 - protein, 12 aa, Artificial Sequence
  Synthetic Construct
PNYNYGGSWFAY

SEQ ID NO: 118 - protein, 5 aa, Artificial Sequence
  Synthetic Construct
SSVHY

SEQ ID NO: 119 - protein, 9 aa, Artificial Sequence
  Synthetic Construct
ASSSVHYMH

SEQ ID NO: 120 - protein, 10 aa, Artificial Sequence
  Synthetic Construct
SASSSVHYMH

SEQ ID NO: 121 - protein, 4 aa, Artificial Sequence
  Synthetic Construct
  MISC_FEATURE (4)..(4): Xaa is any amino acid
DTSX

SEQ ID NO: 122 - protein, 7 aa, Artificial Sequence
  Synthetic Construct
DTSKLAS

SEQ ID NO: 123 - protein, 9 aa, Artificial Sequence
  Synthetic Construct
QQWTSNPPT

SEQ ID NO: 124 - protein, 123 aa, Artificial Sequence
  Synthetic Construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 125 - DNA, 369 bp, Artificial Sequence
  Synthetic Construct
gaactaatactggtggagtctgggggaggcttagtgaagcctggagggtccctgaaactctcctgtgcagtctctggattcactttcagttcctttgccatgtcttgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 126 - protein, 127 aa, Artificial Sequence
  Synthetic Construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 127 - DNA, 381 bp, Artificial Sequence
  Synthetic Construct
ggtgtccagtgtgaactaatactggtggagtctgggggaggcttagtgaagcctggagggtccctgaaactctcctgtgcagtctctggattcactttcagttcctttgccgtgtcctgggttcgccagactccggagaagaggctggagtgggtcgcaaccattagtagtggtggagcttacaccttctataaagacagtgtgaaggggcgattcaccatctccagagacaatgccaagaataccctgtacctgcaaatgagcagtctgaggtctgaggactcggccatgtattactgtgcaagacatagcggctatgatggttactacctctatgctatggactactggggtcaaggaacctcagtcaccgtctcctca

SEQ ID NO: 128 - protein, 121 aa, Artificial Sequence
  Synthetic Construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 129 - DNA, 363 bp, Artificial Sequence
  Synthetic Construct
gaggtgcagctggaggagtctgggggaggcttagtgcagccgggagggtccctgaaagtctcctgtgcagtttccggactcgctttcagcagccatgacatgtcttgggttcgccagactccggagaagcggctggagtgggtcgcatacattagtggaggtggtactaatatctattattcagacactgtgaagggccgattcaccatctccagagacaatgccaagaacaccctgtacctgcaaatgagcagtctgaagtctgaagacacagccatttattactgtgcaagacccaattataattacggcggttcctggtttgcttactggggccaagggactctggtcactgtctctgca

SEQ ID NO: 130 - protein, 106 aa, Artificial Sequence
  Synthetic Construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 131 - DNA, 320 bp, Artificial Sequence
  Synthetic Construct
caaattgttctcacccagtctccagaaatcatgtctgcatctccaggggagaaggtcaccatgacctgcagtgccagctcaagtgtacattacatgcactggtaccagcagaagtcaggcacctcccccaaaagatggatttatgacacatccaaactggcttctggagtccctggtcgcttcagtggcagtgggtctgggacctcttactctctcacaatcagcagcatggagtctgaagatgctgccacttattactgccagcagtggactagtaacccacccacgttcggaggggggaccaagctggaaattaaacg

SEQ ID NO: 132 - protein, 244 aa, Artificial Sequence
  Synthetic Construct
ELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 133 - protein, 248 aa, Artificial Sequence
  Synthetic Construct
GVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 134 - protein, 242 aa, Artificial Sequence
  Synthetic Construct
EVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSAGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIK

SEQ ID NO: 135 - protein, 244 aa, Artificial Sequence
  Synthetic Construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 136 - protein, 248 aa, Artificial Sequence
  Synthetic Construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSGVQCELILVESGGGLVKPGGSLKLSCAVSGFTFSSFAVSWVRQTPEKRLEWVATISSGGAYTFYKDSVKGRFTISRDNAKNTLYLQMSSLRSEDSAMYYCARHSGYDGYYLYAMDYWGQGTSVTVSS

SEQ ID NO: 137 - protein, 242 aa, Artificial Sequence
  Synthetic Construct
QIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKGGGGSGGGGSGGGGSEVQLEESGGGLVQPGGSLKVSCAVSGLAFSSHDMSWVRQTPEKRLEWVAYISGGGTNIYYSDTVKGRFTISRDNAKNTLYLQMSSLKSEDTAIYYCARPNYNYGGSWFAYWGQGTLVTVSA

SEQ ID NO: 138 - protein, 890 aa, Artificial Sequence
  Synthetic Construct
MALPVTALLLPLALLLHAARPDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLEESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDFMNMTARRAGPTRKHYQPYAPPRDFAAYRSGSGATNFSLLKQAGDVEENPGPMALPVTALLLPLALLLHAARPELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKAAATTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - DNA, 8977 bp, Artificial Sequence
  Synthetic Construct
ccatggctttacccgttaccgctctgctgctgcctctggctctgctgctccacgctgctaggcccgacatcgtgatgacacaagctgccccctccgtgcccgttacacccggtgagagcgtcagcatctcttgtggctccagcaagtctttactgcacagcaacggcaacacctatttatactggtttctgcagaggcccggccagagccctcagctgctgatttacagagtcagcaatttagccagcggcgtgcccgatagattctccggcagcggcagcggcacagccttcactttaaggatctctcgtgtcgaggccgaagacgtgggagtgtactattgcatgcagcatttagagtacccttacaccttcggcggaggcaccagactggagatcaagggcggcggcggcagcggcggtggcggcagcggaggaggcggtagcgaggtgcagctggaagagtccggcggaggtttagtgcagcccaaaggatctttaaagctgagctgcgccgccagcggcttcaccttcaacacctacgccatgtactgggtgtgccaagctcccggtaagggtttaaagtgggtggctcgtattcgttccaaggtgaacaactacgccacctactacgccgattccgtcaaggatcgtttcaccatctctcgtgacgacagccagaacatgctgtttttacacatgaacaatttaaagaccgaggacaccgccatctatttctgcgtgagggaccctatggattactggggacaaggtatcagcgtgacagtgtcctccgcggccgccacaacaacccccgctcctagacctcctacccccgcccccaccatcgccagccaacctctgtctctgagacccgaagcttgtagacccgccgctggcggagccgtgcatacaagaggactggactttgcttgcgacatctacatttgggctcccctcgccggaacatgtggcgtgctgctgctgtctctggtgatcacactctattgcaagaggggaagaaaaaagctgctgtacatctttaagcagcccttcatgaggcccgtccagaccacacaagaagaggatggctgcagctgcagattccccgaggaagaagagggcggatgtgagctgaggtccaaaagatctagactgctgcacagcgacttcatgaacatgaccgctagaagggccggacctacaaggaaacattaccagccttacgccccccctagagactttgccgcctacagatccggatccggagccaccaacttttctctgctgaagcaagccggagatgtggaagagaatcccggccctatggccctccccgtgacagctctgctgctgcctctggctctgctgctccatgccgctaggcccgagctgattctggtcgaaagcggcggaggactggtcaagcccggaggatctctgaaactgagctgcgccgtgagcggcttcaccttctccagctttgctatgagctgggtgagacagacacccgaaaagaggctggaatgggtggccacaatttccagcggcggagcctacaccttttacaaggattccgtgaagggaagattcaccatctctagagacaatgctaaaaacacactgtatctgcagatgtcctctctgaggtccgaagactccgccatgtattactgcgccagacacagcggctacgacggctactatctgtacgctatggactactggggccaaggcacaagcgtcacagtcagctccggaggcggaggcagcggaggaggaggaagcggaggcggaggcagccagattgtgctgacccagagccccgaaatcatgtccgcttcccccggcgagaaagtcacaatgacatgttccgcctccagcagcgtccactacatgcactggtaccaacagaagagcggcaccagccccaagaggtggatttacgacacctccaaactcgcctccggcgtgcccggaagattcagcggaagcggctccggcaccagctactccctcacaatctccagcatggagagcgaagatgccgctacctactactgccagcaatggacctccaacccccctaccttcggaggcggcaccaaactggagattaaagccgccgctaccaccacccccgcccctaggccccctacacccgcccccacaatcgcttcccaacctctgtctctgagacccgaggcttgtagacccgccgctggaggagccgtccatacaagaggcctcgactttgcttgcgacatttacatctgggcccctctggctggcacatgcggagtgctgctgctgtctctggtcatcacactgtactgcagagtgaagttcagcagatccgccgatgcccccgcttataagcaaggccagaaccagctgtacaacgagctgaatctgggaaggagggaggagtatgacgtgctggataaaaggagaggaagagatcccgagatgggcggcaagcctagaagaaaaaacccccaagagggcctctacaacgaactgcagaaggacaagatggccgaggcctactccgagatcggcatgaagggcgaaaggagaagaggcaagggacacgatggcctctaccaaggcctctccaccgccacaaaggacacatacgacgccctccatatgcaagctctgccccccagataactcgagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgccca

SEQ ID NO: 140 - protein, 890 aa, Artificial Sequence
  Synthetic Construct
MALPVTALLLPLALLLHAARPDIVMTQAAPSVPVTPGESVSISCGSSKSLLHSNGNTYLYWFLQRPGQSPQLLIYRVSNLASGVPDRFSGSGSGTAFTLRISRVEAEDVGVYYCMQHLEYPYTFGGGTRLEIKGGGGSGGGGSGGGGSEVQLVESGGGLVQPKGSLKLSCAASGFTFNTYAMYWVCQAPGKGLKWVARIRSKVNNYATYYADSVKDRFTISRDDSQNMLFLHMNNLKTEDTAIYFCVRDPMDYWGQGISVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRSKRSRLLHSDFMNMTARRAGPTRKHYQPYAPPRDFAAYRSGSGATNFSLLKQAGDVEENPGPMALPVTALLLPLALLLHAARPELILVESGGGLVKPGGSLKLSCAVSGFTFSSFAMSWVRQTPEKRLEWVATISSGGAYTFYKDSVKGRFTISRDNAKNTLYLQMSSLRSEDSAMYYCARHSGYDGYYLYAMDYWGQGTSVTVSSGGGGSGGGGSGGGGSQIVLTQSPEIMSASPGEKVTMTCSASSSVHYMHWYQQKSGTSPKRWIYDTSKLASGVPGRFSGSGSGTSYSLTISSMESEDAATYYCQQWTSNPPTFGGGTKLEIKAAATTTPAPRPPTPAPTIASQPLSLRPEACRPAAGGAVHTRGLDFACDIYIWAPLAGTCGVLLLSLVITLYCRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - DNA, 8977 bp, Artificial Sequence
  Synthetic Construct
ccatggctctgcccgttaccgctttactgctgcctctggctctgctgctgcatgccgctaggcccgacatcgtgatgacccaagctgctccttccgtgcccgtgacacccggtgagagcgtctccatctcttgtggcagcagcaaatctttactgcactccaacggcaacacctatctgtattggtttttacagaggcccggtcagtccccccagctgctgatctatcgtgtgagcaatctcgctagcggcgtgcccgacagattttccggcagcggcagcggaacagccttcactttaaggatctccagagtggaggccgaggacgtgggcgtgtactactgcatgcagcatttagagtacccctacacctttggaggcggcactcgtctggaaattaagggcggcggcggctccggcggaggcggctccggaggcggaggaagcgaagtgcagctggtggaaagcggaggcggtttagtgcagcccaaaggatctttaaaactgtcttgtgccgccagcggcttcaccttcaacacctacgctatgtactgggtgtgccaagctcccggtaagggtttaaaatgggtggctcgtattcgtagcaaagtgaacaactacgccacctactacgccgacagcgtgaaagataggttcaccatctctcgtgacgactcccagaacatgctgtttttacacatgaacaatctcaaaacagaggacaccgccatctacttctgcgtgagggaccctatggattactggggccaaggtattagcgtgaccgtgtccagcgcggccgccacaacaacccccgctcctagacctcctacccccgcccccaccatcgccagccaacctctgtctctgagacccgaagcttgtagacccgccgctggcggagccgtgcatacaagaggactggactttgcttgcgacatctacatttgggctcccctcgccggaacatgtggcgtgctgctgctgtctctggtgatcacactctattgcaagaggggaagaaaaaagctgctgtacatctttaagcagcccttcatgaggcccgtccagaccacacaagaagaggatggctgcagctgcagattccccgaggaagaagagggcggatgtgagctgaggtccaaaagatctagactgctgcacagcgacttcatgaacatgaccgctagaagggccggacctacaaggaaacattaccagccttacgccccccctagagactttgccgcctacagatccggatccggagccaccaacttttctctgctgaagcaagccggagatgtggaagagaatcccggccctatggccctccccgtgacagctctgctgctgcctctggctctgctgctccatgccgctaggcccgagctgattctggtcgaaagcggcggaggactggtcaagcccggaggatctctgaaactgagctgcgccgtgagcggcttcaccttctccagctttgctatgagctgggtgagacagacacccgaaaagaggctggaatgggtggccacaatttccagcggcggagcctacaccttttacaaggattccgtgaagggaagattcaccatctctagagacaatgctaaaaacacactgtatctgcagatgtcctctctgaggtccgaagactccgccatgtattactgcgccagacacagcggctacgacggctactatctgtacgctatggactactggggccaaggcacaagcgtcacagtcagctccggaggcggaggcagcggaggaggaggaagcggaggcggaggcagccagattgtgctgacccagagccccgaaatcatgtccgcttcccccggcgagaaagtcacaatgacatgttccgcctccagcagcgtccactacatgcactggtaccaacagaagagcggcaccagccccaagaggtggatttacgacacctccaaactcgcctccggcgtgcccggaagattcagcggaagcggctccggcaccagctactccctcacaatctccagcatggagagcgaagatgccgctacctactactgccagcaatggacctccaacccccctaccttcggaggcggcaccaaactggagattaaagccgccgctaccaccacccccgcccctaggccccctacacccgcccccacaatcgcttcccaacctctgtctctgagacccgaggcttgtagacccgccgctggaggagccgtccatacaagaggcctcgactttgcttgcgacatttacatctgggcccctctggctggcacatgcggagtgctgctgctgtctctggtcatcacactgtactgcagagtgaagttcagcagatccgccgatgcccccgcttataagcaaggccagaaccagctgtacaacgagctgaatctgggaaggagggaggagtatgacgtgctggataaaaggagaggaagagatcccgagatgggcggcaagcctagaagaaaaaacccccaagagggcctctacaacgaactgcagaaggacaagatggccgaggcctactccgagatcggcatgaagggcgaaaggagaagaggcaagggacacgatggcctctaccaaggcctctccaccgccacaaaggacacatacgacgccctccatatgcaagctctgccccccagataactcgagatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgatgacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtgaatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgccca